(19) United States
(12) Patent Application Publication
(10) Pub. No.: US 2025/0085788 A1
(43) Pub. Date: Mar. 13, 2025

(54) Multi-Task Hand Support Device for Operating Multi-Task Interactive Electronic Devices and System for Displaying Sideline Operation

(71) Applicant: Jesus Perea-OcHoa, Montebello, CA (US)

(72) Inventor: Jesus Perea-OcHoa, Montebello, CA (US)

(21) Appl. No.: 18/775,377

(22) Filed: Jul. 17, 2024

Related U.S. Application Data
(63) Continuation-in-part of application No. 16/514,856, filed on Jul. 17, 2019, now Pat. No. 12,089,733.
(63) Continuation-in-part of application No. 17/847,151, filed on Jun. 22, 2022.
(63) Continuation-in-part of application No. 18/670,675, filed on May 21, 2024.

(51) Int. Cl. G06F 3/02

(52) U.S. Cl. CPC G06F 3/0202

(57) ABSTRACT

A hand support device, for an input apparatus such as typing apparatus like keyboard, notebook, laptop, or the like, includes an elongated hand supporting bridge unit, a support base assembly and a pillar and bar joint assembly. The support base assembly supports the hand supporting bridge unit directly over the keyboard along the keyboard from the right side to the left side of the keyboard so that the user can rest on and work, wherein the support base assembly comprises a first base unit and a second base unit adapted for positioning at two sides of the keyboard. The pillar and bar joint assembly is arranged to movably support by the first base unit and the second base unit in order for the hand supporting bridge unit to provide a transversal support for the user's upper extremities over the keyboard.

### CROSS REFERENCE OF RELATED APPLICATION

This is a Continuation-In-Part application that claims the benefit of priority under 35 U.S.C. § 120 to a non-provisional application, application Ser. No. 16/514,856, filed Jul. 17, 2019, a non-provisional application, application Ser. No. 17/857,151, filed Jun. 22, 2022, and a non-provisional application, application Ser. No. 18/670,675, filed May 21, 2024, which is a Continuation application that claims the benefit of priority under 35 U.S.C. § 120 to a non-provisional application, application Ser. No. 17/539,177, filed Nov. 30, 2021. The afore-mentioned non-provisional applications are hereby incorporated by references in their entireties.

### BACKGROUND OF THE PRESENT INVENTION

### Field of Invention

The present invention relates to hand support device for an input apparatus, such as typing apparatus like keyboard and touch screen panel, laptop accessory and/or built-in system thereof, and more particularly to a hand support device, adapted for operating in a multi-task interactive system and system for displaying sideline operation, providing assistance, support and therapeutic treatments to the typist while working on an input apparatus like keyboard, laptop or other communication input apparatus, or gaming device, including augmented reality devices (AR) and/or virtual reality devices (VR) for the purpose of the user supporting his or her uppers extremities, in particular hands and wrist during repetitive task and or for prolong cervical spine muscular posture tension, in a wrist support typing posture.

### Description of Related Arts

With the continuous development of new computer applications and functions, people are increasingly unable to give up the computer. Almost all people work on the computer to type, like editing codes, writing reports, sending emails or accessing to information, etc. In other words, the office worker may always sit in the office and spend all working time to type with a keyboard or laptop. The reports say that people are sitting in an average of 9 hours a day, 60% of which are at work facing the computer, which is harmful to the health of people, especially when people sit in a wrong posture. All computing devices like laptops, keyboard computer gaming devices, (AV/AR) devices, communication devices, require a particular level of human's physical effort and stamina to produce a task and for enjoyment.

In addition, most electronic devices are powerful enough to run two applications at the same time for multi-tasking. For example, the player is able to play via a game application and chat with friends via another chat application. However, these applications are controlled by one single input. In other words, when the player wants to control the game, he or she must switch the input corresponding to the game application. When the player wants to chat with friends, he or she must switch the input back to the chat application from the game application. This switching manner will interfere the flow of the game and the chatting as well

When people sit and type, they need to withstand a muscle fatigue due to gravitational forces and an opposed support of the chair and the desk, and an alignment force which forces people to face the computer in a fix posture. Gravity and lack of physical endurance preset most people to sit in the wrong posture while typing, their cervical and thoracic vertebras are in the wrong posture of forward flexion and their muscles of the posterior neck are also strained due to long-term inflexibility, causing cervical spondylosis. At the same time, prolong poor ergonomic sitting and typing posture can cause gravity and weight to be pressed against a thoracic lumbar region, wherein a pressure bearing surface is unevenly distributed, which causes a kyphosis and the waist, abdomen and back muscles to sag and produce pain. Teenagers whose spines and neck are in a growth should pay more attention to above problems. Due to a variety of homeworks, computer games and projects the teenagers spend lots of time in a compromise positional posture, which is not less than the office worker.

It is important and necessary to maintain a correct sitting and typing position while using the computer. When we sit and type, our hands will rise high in the air over the keyboard which even time fatigue causes the wrong siting and typing posture, especially our cervical and thoracic vertebras in a wrong state of forward flexion. Even if our elbows and forearms are supported, these problems will not be solved.

It is important to mention prolong (AR/VR) utilization will lead to numerous orthopedic stress injuries and additionally the physical kinetic energy requirement will be too demanding for the general public. The computer keyboard platform will not get replace by the (AR/VR) technology but the (AR/VR) technology will enhance the keyboard computer platform. In other words, our invention will physical support the upper extremities and technologically interacted with typing computing apparatus that will interact with multiple systems of electronic devices and computer programs including (AR/VR) technology.

In addition to the hunchback and the cervical spondylosis, the poor sitting and typing posture also causes thoracic enthesopathy, upper crossed syndrome which manifests as inhibited neck flexors, inhibited rhomboids and serratus anterior, tight deltoid and tight upper trapezius and levator scapulae muscles, and tension headache can all manifest with or without (AR/VR) device utilization as a Durable Medical Equipment (DME).

Continues over utilization of the computer has been linked to increase insomnia. Insomnia can increase depression and disrupt the normal circadian rhythm. Our goal is to provide a hand wrist support that works in conjunction with other FDA approved modalities to deliver a system of wellness during work hours, or recreational time. The user will be able to use computers, laptops, keyboards, augmented reality and virtual reality devices all while the hand and wrist are supported and the support device delivers additional medical therapeutic care.

During physical demand on a specific body part over utilization of the muscles will cause soreness and strain, while maintaining the correct sitting and typing posture without any auxiliary equipment for a long time. And some people may not know the correct sitting and typing posture, or the wrong sitting and typing posture has been their habit which is incorrigible behavior. In other words, it is toilsome for people to maintain the correct sitting and typing posture without any auxiliary equipment.

Furthermore, even with the proper auxiliary equipment millions of people around the world live and work with chronic pain and mental health needs. At the same time around the word the dependence on the over utilization of computers or similar devices has increases exponentially. No other hand wrist supporting system provides Transcutaneous electrical nerve stimulation (Tens) unit, Iontophoresis, Neuromuscular Electric Stimulation (NMES), Interferential therapy (IF), H wave therapy, Microcurrent Electrical Therapy (MET), and etc. Where the user and/or the medical provider can designate the proper required treatment modalities on the internet/intranet. Where the medical provider and the patient may monitor the treatment and control the duration, intensity and strength of the applicable medical care. In other words, the medical provider will have direct contact with the patient through the internet or intranet.

Furthermore, no conventional hand wrist support encompasses a system where the structural support houses an array of multiple various medical treatments. In other words, the user buys a specific treatment cartridge with specific connecters designated for a specific treatment to be operated by the hand therapeutic support device for the specific illness that is being treated. Additionally, the treatment controls can be on the hand therapeutic support device, cell phone, physical computer, laptop, AR/VR, or on the keyboard. Were the hand wrist support system can be also be control through the internet, intranet and to include a computer application (AP) and not exclusive to any combination.

Multiple illnesses like Raynaud's disease, neuromuscular disease, chronic and acute neuropathic pain, nociception pain, carpal tunnel syndrome, sprain, strain, tendinitis, arthritis, gout, pseudogout or other injuries of the hand can affect mental health like tress, depression, anxiety and insomnia. Additionally, with illness and fatigue proprioception is diminish reducing the user's ability to complete typing task.

In addition, most electronic devices are powerful enough to run two applications at the same time for multi-tasking. For example, the player is able to play via a game application and chat with friends via another chat application. However, these applications are controlled by one single input. In other words, when the player wants to control the game, he or she must switch the input corresponding to the game application. When the player wants to chat with friends, he or she must switch the input back to the chat application from the game application. This switching manner will interfere the flow of the game and the chatting as well.

Such switching burden also exists in processing software applications and/or computer languages, such as processing a word or graphic document, by opening one window and chatting with friends with a communication software or APP by opening another window. The user is required to switch between the windows from time to time too. Specially, in users with physical medical tremors, it can be severely limiting to operate programs like these that require continues dexterity without any hand therapeutic support device.

Operating multi-task interactive electronic devices may also be executed through gesture operation on input apparatus. The most commonly used gesture in operating a program on a display screen is, for example, the zoom-in gesture and zoom-out gesture. The zoom-in and zoom-out gestures on the display screen can respectively help to zoom in and zoom out an object, such as a picture, a document or a figure, displayed on the screen which can be the same display screen or another displaying screen. The zoom-in gesture or the zoom-out gesture is simply executed through two fingers of a user touching on the display screen of an electronic device.

Currently in the market, there is no organization for program operating gesture that can control the operating program while inputting at the same time. However, it is a great demand to the user for operating one or more programs and/or on one or more display screens of an electronic device such as a notebook computer, a tablet, a smart phone, or a slot machine. However, multiple programs or applications are developed and installed in an electronic device and two or more programs or applications (APPs) are able to be opened at the same time to function in two or more different windows but, generally, only one program is operational for physical control while the other programs or applications may merely display without the capacity to be operated at the same time. These programs or applications share a single input device (one to one input capacity), that is only one inputting can control one of the display screen programs. The user is forced to shift the physical activities of inputting between two programs or APPs, that substantially increases the workload by having to alternate from one program to the other program, decreases productivity with numerous (back and forth) physical control movements for each separated program, and increases time consumption when multitasking under current operation system. In other words, the mere input device only controls one primary display program or only one primary display application at one time.

With the development of metaverse (meta universe/simulate environment), virtual reality (VR), mixed reality (MR), extended reality (XR), and augmented reality (AR) technologies are able to be interacted with the reality world through the VR, MR, XR or AR apparatus that the virtual reality and the reality, the reality and the augmented reality, or multiple virtual realities are being displayed at the same time via a displaying apparatus (such as VR/AR/XR/MR glasses or goggles) of the user. The input apparatus, such as touch screen panel, keyboard, mouse pad, and etc., are capable of configured to allow the user to operate the multi-task interactive electronic devices with the input apparatus with gestures.

However, when a user has to continue to work on the input apparatus, even simply using gestures to operate the multi-task interactive electronic devices, the user still needs to withstand muscle, wrists and finger joints fatigue due to gravitational force. It is a urge demand for an effective hand support device for operating multi-task interactive electronic devices and system for displaying sideline operation to all users, especially to the children, senior people and hand injured patients.

### SUMMARY OF THE PRESENT INVENTION

The invention is advantageous in that it provides a hand support device for input apparatus, such as a keyboard, mouse, mouse pad, touch panel, laptop, notebook, or the like, for operating electronic devices, multi-task interactive electronic devices and/or system for displaying sideline operation, which provides assistance and support for a user to rest his hands and wrists, so that the user needs less efforts while input working like typing and repetitive fingers or wrist movements while additional therapeutic modalities can be provided.

It is an advantage of the invention in that it provides a hand support device which not only support and treat existing injuries but also provide treatment prophylactically for a better quality of life.

One advantage of the invention is to provide a hand support therapeutic device for operating multi-task electronic device that the users have the option to purchase interchangeable therapeutic cartridges for equipping with the hand support therapeutic multi-task device. The user will be able to buy any electrotherapeutic modality and administer more than one therapeutic modality on multiple body parts at the same time through the use of the hand support therapeutic multi-task device.

Another advantage of the invention is that the parallel adjustable bar or bars of the hand support device adapted for running over the input apparatus such as typing apparatus like keyboard, on the side or underneath the input apparatus such as input apparatus such as typing apparatus like keyboard like keyboard, computer, laptop or gaming device. The hand support device of the invention is able to give the user meaningful structural stability hovering over the input apparatus with minimal effort. In other words, existing hand support structures are elementary and lack adjustments specifications that are different to every individual needs. Traditionally current hand supports are made of nylon, cloth rubber soft material and all existing hand supports are located distal to the keyboard or laptop demanding the user to hyperextend all digits to overcome the separation between the input apparatus such as input apparatus such as typing apparatus like keyboard and the hand wrist support, wherein the hand of the user has to bent upwardly from his or her wrist to a up bending typing posture that substantially hurting the wrist for long time typing with such posture. In addition, the lack of available adjustments on current existing hand support compounds the problem. This problem is exceptionally evident with the existing traditional hand/wrist matt support and current laptop combination where the user continues to experience discomfort.

Another advantage of the invention is that while the bar or bars are supporting the hand and wrist at the same time the typist is able to be given multiple types of Food and Drug administration (FDA) approved treatments like electrotherapy treatment, iontophoresis treatment, topical treatments, compound medications and occlusion therapy, these are well known treatment modalities, where currently patients must stop activities on the computer or keyboard activities to enable acquisition of such medical care. According to the present invention, the user will be able to get all treatment modalities without stopping their computer activities.

Another advantage of the invention is to provide a hand support device for input apparatus such as typing apparatus like keyboard or laptop, providing assistance and support for a user to rest his hands and wrists and type with a wrist support typing posture that the hands of the user are naturally hanging down from his or her wrists to reach the typing apparatus to type, while the wrists are well supported by the hand support device, so that the user needs less efforts while working like typing and repetitive fingers or wrist movements while provided additional therapeutic modalities.

Another advantage of the invention is to provide a hand support device for input apparatus such as typing apparatus like keyboard, notebook or laptop, which supports the hands of the user to promote the user to sit and type in a correct posture and avoid poor cervical/thoracic vertebra posture alinement and reduce straining the upper extremities due to a wrong posture of forward flexion. In other words, the user needs less efforts in maintain the correct sitting and typing posture with the hand support device.

Another advantage of the invention is to provide a hand support device for input apparatus such as typing apparatus like keyboard, notebook, laptop, or the like, which helps the user with upper extremities injuries to work or play on the keyboard, laptop or computer. In other words, the hand support device is arranged to better support the injured hands or wrists of the user and enable the hands of the user naturally hanging down from his or her wrists which are well supported by the present invention to helps reduce pain exacerbation from over compensating due to the pre-existing injury while enabling natural, easy and smooth movement of the hands and fingers during typing.

Another advantage of the invention is to have interchangeable therapeutic cartridges kits adaptable to a hand support device for specific medical and prophylactic care. Where the controls for the therapy can also be interchangeable on the hand support device, computer monitor, laptop monitor, keyboard, AR/VR and on the users smart phone internet and or intranet computer application.

Another advantage of the invention is to provide a hand support device for input apparatus such as typing apparatus like keyboard, notebook, laptop, or the like, which radiates heat or cold, and/or frequency vibration to the fingers, hands and wrists of the user.

Another advantage of the invention is to provide a hand support device for input apparatus such as typing apparatus like keyboard, notebook, laptop, or the like with radiates a magnetic field for therapy and diagnostics.

Another advantage of the invention is to provide medicine and electrotherapy to the user of a hand support device while the user is engaging in work, and personal activities on input apparatus such as typing apparatus like keyboard or laptop. In other words, the bars, sleeves or braces on the bars can provide the source of contact for topical medicine, and electrotherapy, and furthermore the hand support therapeutic device can provide additional (wireless and wire) body leads, pads, patches, and braces etc. for a more complete body care.

Another advantage of the invention is to provide various types of gloves, straps and bracing for comfort and to deliver various treatments modalities of a hand support device for input apparatus such as typing apparatus like keyboard, notebook, laptop, or the like.

Another advantage of the invention is to provide a hand support device for input apparatus such as typing apparatus like keyboard, notebook, laptop, or the like which comprises an additional various bar sleeves and wrists brace on to the bar or bars for additional added support, treatment and comfort.

Another advantage of the invention is to provide a portable adaptable or built in input apparatus such as typing apparatus like keyboard hand support device to any electronic computing device, which the user can move to a desire location for additional comfort.

Another advantage of the invention is to provide a hand support device for input apparatus such as typing apparatus like keyboard, notebook, laptop, or the like, which enables the user having micro movements in all directions while hovering over the input apparatus such as typing apparatus like keyboard at a desire location.

Another advantage of the invention is to provide a hand support device for input apparatus such as typing apparatus like keyboard, notebook, laptop, or the like, which provides an up and down motion support, a left to right distance support, and oblique angles left to right horizontal plane reaching support and a bilateral vertical angular finger distant position supporting to facilitate a support control.

Another advantage of the invention is to provide a hand support device for input apparatus such as typing apparatus like keyboard, notebook, laptop, or the like, comprising a bar or set of bars under, over or on the side for the support of the users hands and to mount an additional hand wrist brace or sleeves for additional support and treatment options.

Another advantage of the invention is to provide a hand support device for input apparatus such as typing apparatus like keyboard, notebook, laptop, or the like, adapted for the bar, bars, and or the additional hand wrist support to work in conjunction with hand gloves, elbow brace and other support wear for the purpose of treating the user illness.

Another advantage of the invention is to provide a hand support device to provide additional multiple therapeutic leads for additional body parts and where the treatment can be the same or different from the upper extremity on going treatment modality, at the same time.

Another advantage of the invention is to provide a hand support device for input apparatus such as typing apparatus like keyboard, notebook, laptop, or the like, which provides the desire adjustable positions for comfort in relation to the upper extremities, such as the plantar surface, volar medial, lateral carpal and metacarpal region of both wrists.

Another advantage of the invention is to provide a hand support device for input apparatus such as typing apparatus like keyboard, notebook, laptop, or the like, which will minimize upper extremities energy consumption and maximizes productivity, additionally one can overcome complex time consuming work or games, through the use of the hand support device on the upper extremities.

Another advantage of the invention is to provide a hand support device for input apparatus such as typing apparatus like keyboard, notebook, laptop, or the like, wherein a plurality of sleeves can be manufactured and provided for additional sliding motion, pressure support, topical medications and various additional medical treatments. Like hypoallergenic sleeves to decreasing contact dermatitis etc.

Another advantage of the invention is to provide a hand support device, which is adapted for a pre-existing keyboard, notebook or laptop.

Another advantage is to provide the existing keyboard a build in premanufactured portable collapsible hand support device accessory.

Another advantage is to provide the existing laptop a build in premanufactured portable collapsible hand support device accessory.

Another advantage is to provide a center midline alphabetic system and numeric system input apparatus such as typing apparatus like keyboard that will have a symbiotic utilization for the hand support device as an accessory or as a single unit in other words the key lettering and numbers on the input apparatus such as typing apparatus like keyboard will be center in relation to the hand support device.

Another advantage is to provide future keyboard, laptops and computers a build-in premanufactured diagnostics and recordings of temperature, heart rate, blood pressure, therapy duration, intensity and level of function. Where the user data can be store, transfer, and or evaluated.

Another advantage of the invention is to provide a hand support device for input apparatus such as typing apparatus like keyboard, notebook, laptop, or the like, which can be used for military applications for the soldier to have vital muscular skeletal support system to enable immediate and prolong communication and for the overall clerical cyber keyboard demands of military duties.

Another advantage of the invention is to provide a hand support device for input apparatus such as typing apparatus like keyboard, notebook, laptop, or the like, which can be operational in the battle fields as well in the support units for the purpose of providing our soldiers the very best equipment with the latest technology break throughs to complete the mission. In the support units, soldiers are expose to long hours, tedious repetition, and are required to have sharp focus reaction to both incoming threats and during ongoing offensive attacks. The hand support device provides the soldier a support system for the wrist, hands, shoulder and throughout their body without restricting the natural movement of the soldier upper and lower extremities, while maximizing the user productivity time and response time.

Another advantage of the invention is to provide a hand support device for input apparatus such as typing apparatus like keyboard, notebook, laptop, or the like, which reduces the soldier muscular fatigue on bilateral upper extremities by providing the soldier direct support over the keyboard all while the user is in a natural neutral position. Through the hand support device, the user can take a break, like a posture stretch, all while maintaining tactile contact with the keyboard to reacted and act on any threat in less than a second notice. It is importance to emphasize the importance of hovering over the keyboard during critical tactical moment in war for immediate response time. In the past, soldiers fighting fatigue had to completely remove their upper extremities away from the keyboard in order to ensure not to produce erroneous information, or even worse accidently destroy the mission.

Another advantage of the invention is to provide a hand support device for input apparatus such as typing apparatus like keyboard, notebook, laptop, or the like, wherein while the user is at rest no other hand support device provides the soldiers an unsurpassed ability to immediately scramble during long cycles of down time giving the user the ability to produce immediate commands, responses or codes. Presently a soldier would have to completely remove their hands/wrist off the keyboard in order to alleviate posture tiredness. The time it takes to reposition from a brake like this, of removing the hands and arms away from the keyboard, induce an upper extremity posture stretch, and back on to the same keyboard, are precious minutes lost in the battlefield that can mean life or death scenarios.

Another advantage of the invention is to provide a hand support device for input apparatus such as typing apparatus like keyboard, notebook, laptop, or the like, wherein military support units benefit from the hand support device in relation to the physical time demands requirement on the soldiers. In other words, one can go minutes to hours without any action and soldiers can easily get distracted during long time-outs. Where soldiers are forced to endure chronic fatigue and at the same time are require to spontaneously generated typing transcriptions on command, where speed and reaction are essential to the mission.

Another advantage of the invention is to provide a hand support device for input apparatus such as typing apparatus like keyboard, notebook, laptop, or the like, wherein other scenario is during short critical times that are adrenalin driven the soldiers can become exponentially exhausted without realizing. This will leaded to increase risk of human errors where time is not the directed contributor to the soldier fatigue and where psychological immediate situational stressors are the directed source of fatigue on to the soldier. The more mental stressors like multi-tasking on the individual the greater the physical fatigue the soldier will experience. Anxiety, insomnia, PTSD, MDD, and stress along with physical fatigue are cumulative malignant factors in relation to combat deployment and can manifest into tremors and erode the linguistic utterance demands required to communicated in order to win a war. In other words, the stressors of war can produce upper extremity tremors reduce dexterity and thereby reduces any soldier efficiency, and that is the biggest sympathetic response problem all humans must overcome during immediate emergency scenarios. The hand support device of the present invention provides the support to improve upper extremity dexterity and reduce upper extremity mental transient physical movements thereby improving typing on the keyboard during time of crisis.

Another advantage of the invention is to provide a hand support device which is the interactions in relation to the VR/AR technology for example while in a moving vehicle a soldier will be able to type and read their messages on the AR/VR system all while maintaining visual surveillance for enemies from the inside of the vehicle through the use of an (AR)/night vision device.

Another advantage of the invention is to provide a hand support device for input apparatus such as typing apparatus like keyboard, notebook, laptop, or the like, which enables soldiers being more productive just in general whether they had a sleepless night before, or where out drinking late last night, or suffered from headaches, or have a common cold, the user will have the ergonomic option to utilize a support device on their hand and wrist while providing additional therapy throughout their body at work for longer and faster work production.

Another advantage is to provide a method and system of operating multi-task interactive electronic devices with an attached or a detached hand support deice as an accessory built in or adaptable. Where the bars, sleeves, and braces can be locked in position to provide stability to the user in moving vehicle, tanks, boats, environment, etc.

Another advantage is to provide sports betting as a function of the electronic device with a hand therapeutic support device of the present invention.

Another advantage is to provide multiple gaming devices with a hand therapeutic support device, where the user can select from various therapeutic options, while gaming or competing in Esports.

Another advantage of the present invention is to provide a method and arrangement of operating multi-task interactive electronic device with a hand support device for playing games or processing software's or applications such as processing word or graphic documents, in which the user is able to control at least two applications simultaneously by at least two input modules in a real time manner without interfering with the applications on an electric device.

Another advantage of the present invention is to provide a method and arrangement of operating multi-task interactive electronic devices with a hand support device, including but not limited to augmented reality and virtual reality (AR/VR) devices, working independently or in conjunction with at least an electronic device for playing games or processing softwares or applications such as processing word or graphic documents, in which the user is able to control at least two applications simultaneously by at least two input modules in a real time manner without interfering with the applications.

Another advantage of the present invention is to provide a method and arrangement of operating multi-task interactive electronic device(s) with a hand support device for playing games, wherein the players of the game are able to remotely play the selected game while being able to play against one or more of other real players playing through the communication network in a real time manner, like Esports, Casino tournaments.

Another advantage of the present invention is to provide a method and arrangement of playing games or processing softwares or applications such as processing word or graphic documents in an electronic device with a hand support device, wherein the players or users are able to play the games or process the softwares or applications anywhere anytime via remotely linking the individual electronic devices to the communication network.

Another advantage of the present invention is to provide a method and arrangement of playing games or processing softwares or applications, wherein the players or users are able to remotely play the game or process the software or application while communicatively chatting with other real players or users through the communication network of an electronic device with a hand therapeutic support device.

Another advantage of the present invention is to provide an arrangement of operating multi-task interactive electronic devices with one or more hand support devices for playing games, wherein the communication network of the casino is able to link to a plurality of individual electronic devices with hand support devices, such as slot machines, electronic roulettes, and electronic poker games, so that the players are able to remotely bet or play the gambling game against another real player and/or the dealer, which may be the machine, via the individual electronic devices with hand support devices through the communication network. Therefore, the players have no need to physically go to the crowded gambling table of the table type games or sit in front of the machines while being able to play against the real players.

Another advantage of the present invention is to provide a method and arrangement of operating multi-task interactive electronic devices with a hand support device for playing games, wherein the player is able to active a sideline program for operating multi-task while interacting with one or more electronic devices by inputting membership information such as by inserting a membership card which may digitally store the player information and the prepaid money therein or by means of face or fingerprint recognition to verify the player's information and open the player's prepaid money account stored in the central control of the host of the game, such as the casino, for actuating the games. Thereby, the arrangement may also eliminate the precisely and accurately counting process of physical cash or betting chips after each round of the betting or wagering of the game, so as to enhance the security of the casino and players. Accordingly, the arrangement will be designed primarily for the use of the guest of an establishment such as a hotel. Gambling atmosphere will be fully provided for the establishment guest to play the game anywhere.

Another advantage of the present invention is to provide a method and arrangement of operating multi-task interactive electronic device(s) with a hand support device for playing game, wherein the information of the player, including credit card information and personal information, will be encrypted and stored in blockchain, such as cryptographic, tokenization, for enhancing the data security. Where the cyber security will focus on preventing, detecting, and responding to ever changing cyber-attacks.

Additional advantages and features of the invention will become apparent from the description which follows, and may be realized by the following description of the instrumentalities and combinations particular pointing out in the appended claims.

According to the present invention, the foregoing and other objects and advantages are attained by providing a hand support device for an input apparatus such as typing apparatus like keyboard, notebook, laptop, or the like, which comprises:
an elongated hand supporting bridge unit;
a support base assembly, wherein the support base assembly supports the hand supporting bridge unit directly over the keyboard along the keyboard from the right side to the left side of the keyboard so that the user can rest on and work, wherein the support base assembly comprises a first base unit and a second base unit adapted for positioning at two sides of the keyboard; and
a pillar and bar joint assembly, which is arranged to movably support by the first base unit and the second base unit in order for the hand supporting bridge unit to provide a transversal support for the user's upper extremities over the keyboard.

The pillar and bar joint assembly comprises a first pillar and bar joint and a second pillar and bar joint respectively arranged at the first base unit and the second base unit.

The hand supporting bride unit comprises one or more elongated bars, preferably a pair of parallel bars, wherein the bars is supported between the first pillar and bar joint and the second pillar and bar joint which are positioned respectably on opposite side of the keyboard.

At the same time the bars sitting over the keyboard, the user can control the desire direction for comfort thereof. In other words, the hand supporting bridge unit provide an upward and downward motion support, a left to right distance support, and oblique angles left to right horizontal plane reaching support and a bilateral vertical angular finger distant positioning support. Preferably, the hand supporting bridge unit sits over the keyboard where the height is adjustable for the different users to work and rest. Additionally, the hand supporting bridge unit can be under or on the side of the input apparatus such as typing apparatus like keyboard to support a brace visa versa as another example.

In one embodiment, the hand supporting bridge unit comprises one or more elongated bars. It is important to mention the bar or bars can be of any shape like hollow bars, I beam, L beam, C channel, U channel, formatted truss etc.

In one embodiment, the present invention provides an arrangement of playing games, which comprises:
a plurality of individual electronic devices with hand support devices respectively for being activated by at least one player of the game to controllably and selectively play games therethrough; and
a networking system electrically linking to each of the individual electronic devices with hand support devices and being accessible by at least one casino for managing the arrangement of playing games, wherein the communication network system comprises:
a communication network, which comprises an information storage for storing at least one of the player's information therein, and a plurality of game programs stored in the communication network; and
a remote connecting module for electrically linking the individual electronic devices with a hand support devices with the communication network to form the networking system, so that when the player controllably activates the individual electronic device with hand therapeutic support devices in an authorized manner to electrically link the communication network through the remote connecting module, the player is able to play the casino games in a remotely gambling manner.

In one embodiment, the game players may activate the individual electronic devices with hand support devices thereof by inputting a membership information, for example, via a membership card provided by the casino, so that when the membership card is electrically connected to the individual electronic device with a hand support devices to authorize the player accessing the computer program for the communication network of the casino, the player is able to selectively play the games for betting or wagering.

In one embodiment, the communication network further comprises an online chatting program electrically linking with the remote connecting module, so that the players are able to remotely play the games against or with other real players while being able to chat with other players in a real-time manner. Therefore, the arrangement of playing game does not require the players physically go to the gambling table, and meanwhile, provides the players remotely play against or with other real players in a real-time manner.

In one embodiment, the communication network may be wirely or wirelessly linked to the individual electronic device with a hand support device in the remote control manner. In other words, the individual electronic devices with a hand support device are able to access communication network remotely, preferably, within a predetermined distance.

In accordance with another aspect of the invention, the present invention provides a method of operating a multi-task interactive electronic devices with hand support devices respectively for playing games or processing softwares or applications, which comprises the steps of:
(a) providing a plurality of individual electronic devices with hand support devices, such as notebook, tablet, smart device, cellular phone, portable computer, image capturing device, voice capturing device, motion sensing device, augmented reality (AR) device, and/or virtual reality (VR) device, installed with a sideline program for operating multi-task interaction for a plurality of players respectively for playing games via each of the individual electronic devices; and
(b) remotely linking the individual electronic devices to a networking system, wherein the networking system comprises a communication network having a remote connecting module to electrically connect the individual electronic devices with a hand support devices and with the networking system in a remotely connection manner, and a central control device for managing the networking system.

In accordance with another aspect of the invention, the present invention provides an electronic device equipped with a hand support device for playing games or processing softwares or applications, comprising:
a processor that executes at least a first task application and a second related task application with different interactive programs at the same time;
a screen module operatively linked to the processor, wherein the screen module comprises a display screen having two or more display areas for displaying the first application and the second related application thereon respectively; and
one or more input modules operatively linked to the processor, wherein the first application and the second related application are simultaneously controlled by the input modules respectively, such that the first application and the second related application are executed to be displayed on the display areas respectively and are independently controlled by the input modules at the same time for enabling a user to play a game of the first application via one of the input modules and operate the second related application at the same time via the another input module without interfering the first application.

In one embodiment, the electronic device equipped with the hand support device further comprises a control module operating a sideline program to generate one or more sidelines displayed on the display screen to split the display screen into the two or more display areas.

In one embodiment, a sidelines program is generated as one of a horizontal sideline and a vertical sideline being movable on the display screen to split the display screen into the one or more display areas and to adjust a size of each of the display areas.

In one embodiment, the electronic device of the present invention further provides a control module which comprises a color code generator that generates different color codes for the one or more sidelines to indicate the different interactive programs of the game related application. It is also important to note that each of the sidelines is able to be controlled by voice command and audible programs. It is important to mention the electrical device with the hand support device is not dependent on the sideline program.

In one embodiment, a control panel of the electronic device equipped with the hand support device of the present invention is coupled at the display screen, wherein the input modules are provided at the control panel to independently control the first application and the second related application displayed on the display areas of the display screen.

In one embodiment, at least a visual sideline is slidably formed and displaced at the display screen of the electronic device equipped with the hand support device of the present invention to separate the first application and the second related application displayed on the display areas respectively and at least another control sideline is slidably formed and displayed at the control panel to separate the input modules thereon.

In one embodiment, at least one of the input modules is automatically selected to match with the first application and at least one of the input modules is manually selected by the user to match with the second related application.

In one embodiment, the electronic device equipped with the hand support device of the present invention further includes a phone connection station for connecting with a user smart phone of the user, wherein the verification module comprises a verification program application to generate a verification code in order to activate the electronic device with a hand support devices and also to executed the user smart phone settings and preferences down to the electronic device automatically.

In one or more embodiments, the electronic device equipped with the hand support devices of the present invention supported by the sideline program to provide the sideline(s) at the display screen and/or the control panel is embodied as an innovative portable electronic device with a hand support device, a traditional notebook, tablet or smart device, an AR device, a VR device, or AR/VR device.

In one embodiment, the communication network used by the electronic device equipped with the hand support device of the present invention is a closed communication network module being executed by the processor for wirelessly linking to a private networking system, wherein the first application is restricted for being accessed only through the private networking system, preferably when the electronic device with or without the hand support device is the property of the establishment.

In one embodiment, the electronic device equipped with the hand support device of the present invention further includes a positioning unit for detecting a location of the electronic device, such as notebook, smart device, tablet, portable computer, AR device, VR device, or AR/VR device, wherein the electronic device may or may not be disable for being outside a facility perimeter area in a real time manner via the positioning unit.

In one embodiment, the electronic device equipped with the hand support device of the present invention further includes a chatting module, wherein the second related application comprises a chatting application being executed for enabling the user to chat with another user of another portable electronic device with a hand support devices via the chatting module when the users play or process the same first application.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of according to a first preferred embodiment of the present invention.

FIG. 2 is a partial perspective view of the above first preferred embodiment of the present invention.

FIG. 3 is a schematic view of the above first preferred embodiment of the present invention.

FIG. 4 is an application view of the above first preferred embodiment of the present invention.

FIG. 5 is a side view of the above first preferred embodiment of the present invention.

FIG. 6 is a side view of the above first preferred embodiment of the present invention.

FIG. 7 is a side view of the above first preferred embodiment of the present invention.

FIG. 8 is a perspective view of according to a second embodiment of the present invention.

FIG. 9 is a partial perspective view of according to the above second embodiment of the present invention.

FIG. 10 is a section schematic view of according to the above second embodiment of the present invention.

FIG. 11 is a section schematic view of according to the above second embodiment of the present invention.

FIG. 12 is a perspective view of according to a third embodiment of the present invention.

FIG. 13 is a partial perspective view of according to a fourth embodiment of the present invention.

FIG. 14A is a section schematic view of according to the above fourth embodiment of the present invention.

FIG. 14B is a section schematic view of according to the above fourth embodiment of the present invention.

FIG. 15 is a section schematic view of according to the above fourth embodiment of the present invention.

FIG. 16 is a section schematic view of according to the above fourth embodiment of the present invention.

FIG. 17 is a section schematic view of according to the above fourth embodiment of the present invention.

FIG. 18 is a section schematic view of according to another embodiment of the present invention.

FIG. 19 is a section schematic view of according to another embodiment of the present invention.

FIG. 20 is a partial view of according to a pillar and bar joint assembly of the present invention.

FIG. 21 is a section schematic view of according to another embodiment of the present invention.

FIG. 22 is a section schematic view of according to another embodiment of the present invention.

FIG. 23 is a section schematic view of according to another embodiment of the present invention.

FIG. 24 is a section schematic view of according to another embodiment of the present invention.

FIG. 24A is a perspective view of a hand support device according to another embodiment of the present invention.

FIG. 25 is a partial view of according to another embodiment of the present invention.

FIG. 26 is a schematic view of according to another embodiment of the present invention.

FIG. 27 is a schematic side view of according to another embodiment of the present invention.

FIG. 28 is a schematic view of according to another embodiment of the present invention.

FIG. 29 is a schematic view of according to another embodiment of the present invention.

FIGS. 30A to 30C illustrate the hovering position of the hand to the input apparatus such as typing apparatus like keyboard in relation to the bar or bars and the bars to the sleeve and brace with and without additional therapeutic gloves.

FIG. 30D illustrates the brace movement of the hand support device according to according to the preferred embodiments of the present invention.

FIG. 30E illustrates a CES (Cranial Electrotherapy Stimulation) lead earpiece adapted to equip with the hand support device according to the above preferred embodiments of the present invention.

FIG. 31A is a flow chart of a method of playing games according to a preferred embodiment of the present invention.

FIG. 31B is a block diagram of an operating system of two or more electronic devices according to the preferred embodiment of the present invention.

FIG. 32 is a perspective view of the electronic device equipped with the hand support device according to the preferred embodiment of the present invention

FIG. 33 is a partial exploded perspective view illustrating the electronic device and the hand support device according to the preferred embodiment of the present invention.

FIG. 33A is a perspective view of the hand support device equipped with built-in electronic device according to the preferred embodiment of the present invention.

FIG. 33B illustrates an augmented reality and virtual reality (AR/VR) device according to the preferred embodiment of the present invention.

FIG. 33C illustrates the image of an AR/VR operator to be seen in the AR/VR device of the FIG. 33B in relative to the operation and functions according to the preferred embodiment of the present invention.

FIG. 33D is a block diagram illustrating the interacting of the electronic device, the AR/VR device, the smart device, and the networking system according to the preferred embodiment of the present invention.

FIG. 34A is a block diagram illustrating a modification of the electronic device according to the preferred embodiment of the present invention.

FIG. 34B is a block diagram illustrating another modification of the electronic device according to the preferred embodiment of the present invention.

FIG. 35 illustrates the electronic device as a tablet computerized device according to the preferred embodiment of the present invention.

FIGS. 36A to 36C illustrate various settings of the electronic device as a laptop-like computerized device according to the preferred embodiment of the present invention.

FIG. 37 illustrates the size adjustment of each of the display areas via the movement of the sideline according to the preferred embodiment of the present invention.

FIGS. 38A to 38C illustrate different sideline configurations of the electronic device and how to reset or reposition the sidelines of the electronic device according to the preferred embodiment of the present invention.

FIGS. 39A to 39C illustrate the electronic device interacting with the AR/VR device according to the above preferred embodiment of the present invention.

FIGS. 40A and 40B illustrate the usage of two electronic devices together according to the preferred embodiment of the present invention.

FIG. 41 is a block diagram illustrating the interacting of the electronic device and the AR/VR device according to the preferred embodiment of the present invention.

FIG. 42 is a schematic diagram illustrating the arrangement of the multi-task interactive electronic device and AR/VR device according to the preferred embodiment of the present invention.

FIG. 43 is a flow chart illustrating the interacting of the smart cellular phone, the gaming electronic device and the AR/VR device according to the preferred embodiment of the present invention.

FIG. 44 illustrates elevation view, plan view, end view, and perspective view of the hand support device as shown in FIG. 33 and FIG. 33A according to the preferred embodiment of the present invention.

FIG. 45 is a schematic view of the computer usable medium illustrating portions of the cloud server for operating one or more gestures on one or more display screens according to the preferred embodiment of the present invention.

FIG. 46 is a schematic view illustrating the system of the computer usable medium for operating one or more gesture on one or more display screens and the electronic device according to the preferred embodiment of the present invention.

FIG. 47 is a schematic view illustrating the portions of the cloud server for operating one or more gestures on one or more display screens according to the preferred embodiment of the present invention.

FIGS. 48A-48B are schematic views illustrating an adjuster guiding an operation of gesture for moving on the electronic device according to the preferred embodiment of the present invention.

FIGS. 48C-48D are schematic views illustrating the adjuster for guiding an operation of gesture for zooming on the electronic device according to the preferred embodiment of the present invention.

FIGS. 49A-49C are schematic views illustrating an alternative mode of the adjuster for guiding gesture operation on the electronic device according to the preferred embodiment of the present invention.

FIG. 50 is a schematic view illustrating another alternative mode of the adjuster interface and the interface for guiding gesture operations on the electronic device according to the preferred embodiment of the present invention.

FIG. 51 is a schematic view illustrating portion of a gesture dictionary of various gestures for operating the programs of the electronic device according to the preferred embodiment of the present invention.

FIGS. 52A-52B are schematic views illustrating the adjuster for guiding an operation of gesture for power off of the electronic device according to the preferred embodiment of the present invention.

FIGS. 53A-53C are schematic views illustrating an operation of a sideline element on the display screen of the electronic device according to the preferred embodiment of the present invention.

FIG. 54A is a schematic view illustrating the display screen viewed from a goggle embodied as the electronic device according to the preferred embodiment of the present invention.

FIGS. 54B-54D are schematic views illustrating the display screen of a VR/MR/XR/VR apparatus according to the preferred embodiment of the present invention.

FIG. 55 is a schematic view illustrating the projecting display screen of the VR/MR/XR/VR apparatus according to the preferred embodiment of the present invention.

FIGS. 56A to 56C are schematic views illustrating the display screen of the VR/MR/XR/VR apparatus according to the preferred embodiment of the present invention.

FIGS. 57A to 57B are perspective views of the electronic device with two display screens according to the preferred embodiment of the present invention.

FIG. 58 is a perspective view of the electronic device having a display screen and a control display screen according to the preferred embodiment of the present invention.

FIGS. 59A to 59B are perspective views illustrating the electronic with three display screens according to the preferred embodiment of the present invention.

FIG. 60 is a block diagram illustrating a method of generating adjuster according to the preferred embodiment of the present invention.

FIG. 61 is a schematic view of the adjuster interface with the adjuster according to the preferred embodiment of the present invention.

FIG. 62 is a schematic view illustrating the incorporation of the EMG and EEG/ECOG with BCI/BMI of the electronic device according to the above preferred embodiment of the present invention.

FIG. 63 is a schematic view illustrating the interoperability of performed and imagined signal acquisition on various electronic devices for operating through the internet and/or cloud servers, with or without a processor, according to the preferred embodiment of the present invention.

FIG. 64 is a diagram illustrating various neurological impulses in a signal acquisition created by human body, being divided by the sideline element for multitasking operations on the same device at the same time in a timely manner, according to the preferred embodiment of the present invention.

FIG. 65A is a schematic view illustrating two displaying contents displaying in two displaying portions in responsive to sideline gesture for operating one or more VR/MR/XR/VR apparatuses according to the preferred embodiment of the present invention.

FIG. 65B is a schematic view illustrating multiple sideline arrangement operating the VR/MR/XR/VR apparatus through a physical display screen and/or through a non-physical display medium according to the preferred embodiment of the present invention.

FIG. 66 is a schematic view illustrating multiple inputting devices equipped with camera operating software and infrared and finger detection programs in the internet and/or cloud operating VR/MR/XR/VR devices with or without the brain computer interface according to the preferred embodiment of the present invention.

FIG. 67 is a schematic view illustrating hands and fingers operating through the hand and finger software detection program and/or through the infrared camera sign language program without a glove or sleeve or with a transparent mesh glove and/or sleeve with the fiber optic enhancement, the motion sensor and/or the (EMG) sensors according to the preferred embodiment of the present invention.

FIG. 67A is a schematic view illustrating the transparent soft gel lens for tactile support, with or without fiber optics and micro light source for operating the display screen, infrared camera, camera finger software detection in sign language, or tactile operations according to the preferred embodiment of the present invention.

FIG. 68 is a schematic view illustrating the transparent mesh material for the glove or the sleeve where the mesh supports the various inputting systems according to the preferred embodiment of the present invention.

FIG. 69 is a schematic view illustrating the positioning of the light source and the fiber optic in relation to the finger and the mesh used to produce the glove or sleeve for various inputting systems according to the preferred embodiment of the present invention.

FIG. 69A is a schematic view illustrating the light source and fiber optic over the mesh material or under the mesh material on the proximal tip of the finger, with or without the soft gel transparent lens on the distal tip of the finger, according to the preferred embodiment of the present invention.

FIG. 70 is a chart diagram illustrating the method and various options the user has for creating gestures in the gesture dictionary, with one or more touch points (fingers), according to the preferred embodiment of the present invention.

FIG. 71 is a chart diagram illustrating an animation posture list program according to the preferred embodiment of the present invention.

FIG. 72 is a chart diagram illustrating a voice recording list program according to the preferred embodiment of the present invention.

FIG. 73 is a chart diagram illustrating a paired program list according to the preferred embodiment of the present invention.

FIG. 74 is a chart diagram illustrating inputting options for gesture pairing with their corresponding sign acquisition when performed or imagined to generate a selected response according to the preferred embodiment of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

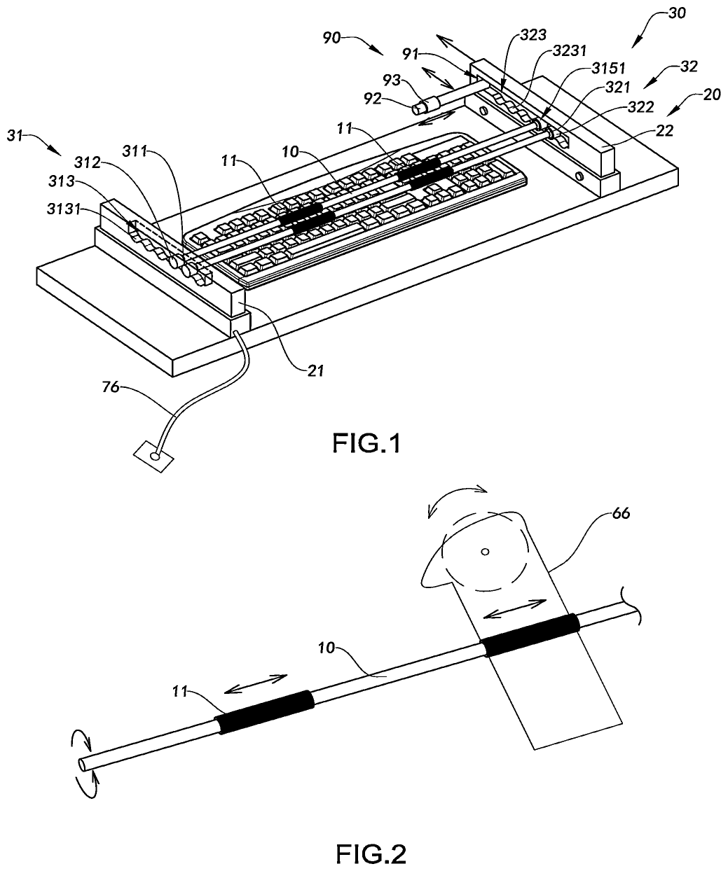

Referring to FIG. 1 to FIG. 7 of the drawings, a hand support device for input apparatus such as typing apparatus like keyboard, notebook, laptop, or the like according to a preferred embodiment of the present invention is illustrated, wherein the hand support device can be mounted with a keyboard to provide assistance with work like typing and repetitive fingers or wrist movements, and other activities of hands or wrists such as playing games and recreational repetitive activities, while providing various therapeutic and medicinal treatments.

The hand support device according to the preferred embodiment of the present invention comprises an elongated hand supporting bridge unit 1 and a support base assembly 20. The hand supporting bridge unit 1 comprises one or more elongated bars 10 and the support base assembly 20 supports the bars 10 of the hand supporting unit 1 directly over the keyboard along the keyboard from the right side to the left side of the keyboard so that the user can rest on and work.

At the same time the bars 10 sit over the keyboard, the user can control the desire direction for comfort thereof. In other words, the bars 10 provide an upward and downward motion support, a left to right distance support, and oblique angles left to right horizontal plane reaching support and a bilateral vertical angular finger distant positioning support. Preferably, the bars 10 sits over the keyboard where the height is adjustable for the different users to work and rest. Additionally, the bar or bars 10 can be under or on the side of the input apparatus such as typing apparatus like keyboard to support a brace 66 visa versa as another example. It is important to mention the bar or bars 10 can be of any shape like hollow bars, I beam, L beam, C channel, U channel, formatted truss etc.

In other words, the user will type with the controlled support of the bars 10, which will enable the user to sit in a correct posture and avoid his cervical vertebra are in a wrong state of forward flexion. In other words, the user needs less efforts in maintain the correct sitting and typing posture for longer periods of time with the keyboard hand therapeutic support device.

Preferably, the bars 10 have different diameter and length with various functions and can be sold separately to meet the user, necessities and to meets most size of the keyboard in the market. The bars 10 can be made of fiber organic composites, glass, wood, plastic, ceramic composites, rubber, carbon, metallic alloys, or any of these combinations together as a hold or in sections throughout the bars 10. The bars 10 can have various designated shapes with various designated contours, such as being circular, triangular, rectangular or star configuration. The number of the bars 10 shown in the figures is an example not limited, where the hand support device is preferred to comprise a pair of bars 10 adapted for the wrists of the user's hands resting between the pair of bars 10 while typing or using the keyboard or the like.

The bars 10 of the hand supporting bridge unit 1 can be positioned together working as one unit or separately apart to provide the desire position for comfort in relation to the upper extremities. And the bars 10 can be elevated or declined in relation each of the other adjacent bars for additional comfort. Preferably, the separation between the bars 10 can be up to 8 inches. The bars 10 can also radiate heat or cold to the fingers, hands and wrists of the user. In addition, vibrating frequency, Transcutaneous Electrical Nerve Stimulation (TENS), Percutaneous Electrical Nerve Stimulator (PENS), Microcurrent Electrical Therapy (MET), and Cranial Electrotherapy Stimulation (CES, topical medication, transdermal/transcutaneous therapy can be provided while working or playing. The treatment and efficacy can also be monitor and recorded through the hand support device via intranet or internet. The bars 10 can be solid, hollow, fabricated with single or multiple materials for multiple conductions, and as a structural facilitator, for the present invention for and future treatments options, recreational and or medicinal.

The support base assembly 20 comprises a first base unit 21 and a second base unit 22 which arranged respectively on the left and the right sides of the keyboard to support the motion of the hand supporting bridge unit 1. A first end and a second end of each of the bars 10 of the hand supporting bridge unit 1 are arranged at the first base unit 21 and the second base unit 22 respectively so as to extend along the length of the keyboard and perpendicular to the left and right margins of the keyboard. According to the preferred embodiment of the present invention, the hand supporting bridge unit 1 comprises a pair of elongated bars 10 arranged in parallel and to be moving up and down across the keyboard between the first base unit 21 and the second base unit 22 of the support base assembly 20.

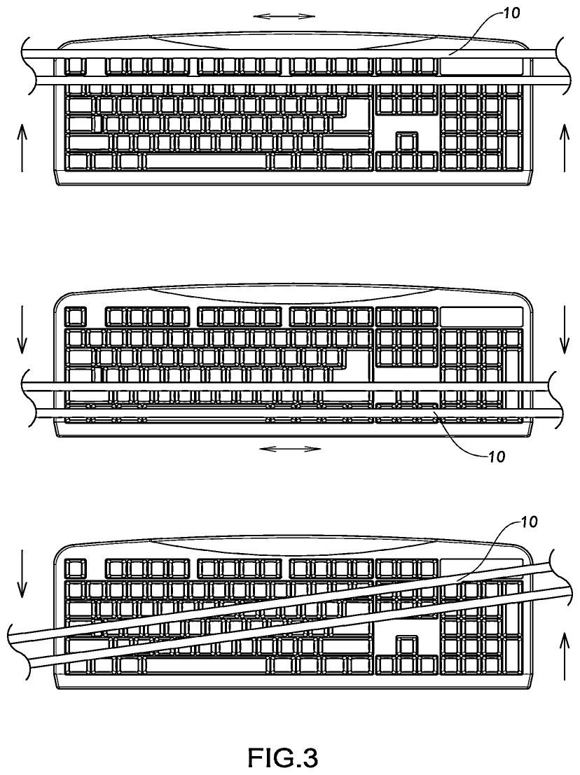

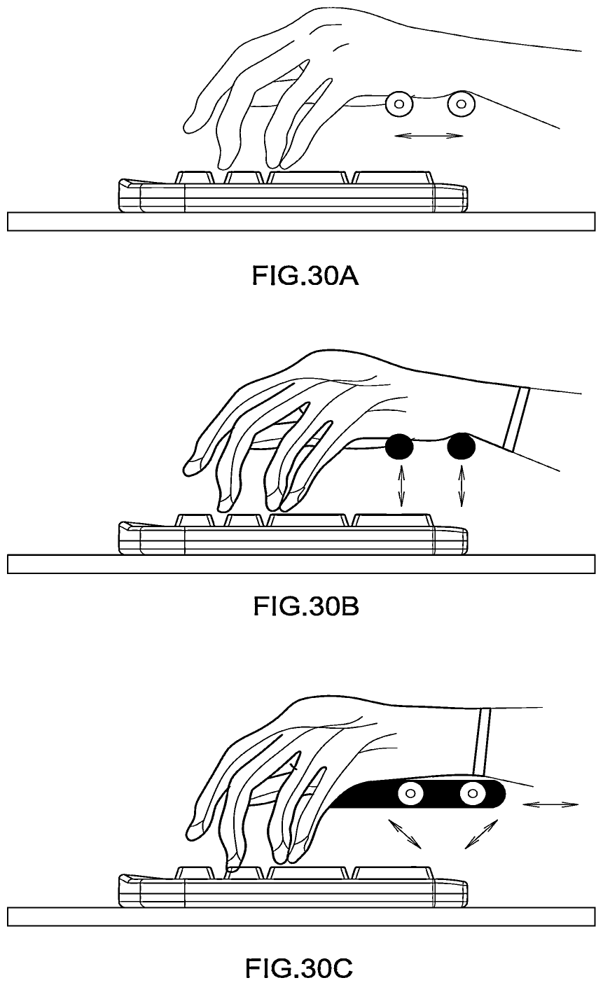

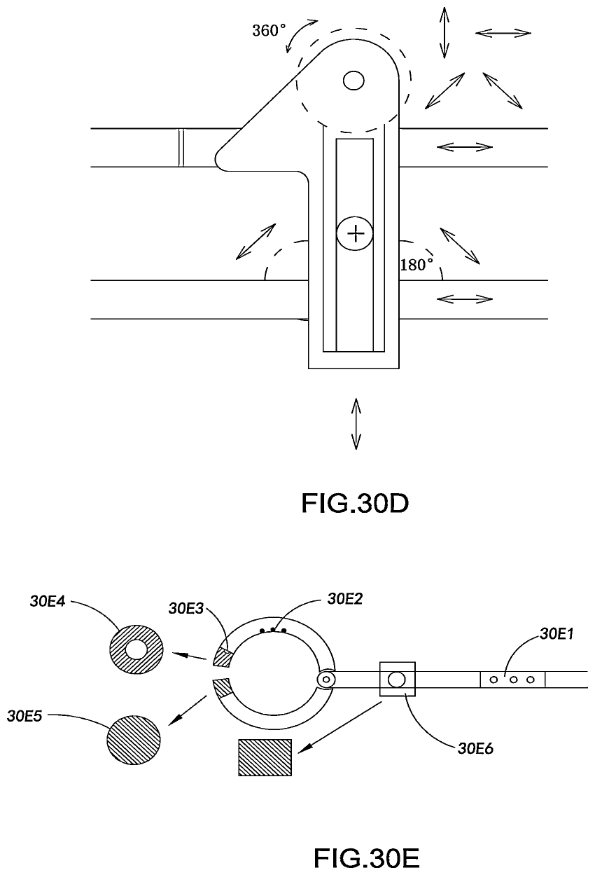

The first base unit 21 and the second base unit 22 provide the vertical and structural support the bars 10 adapted for the function of effortless reaching to minimize energy consumption and maximize productivity through the support on the upper extremities, such as plantar surface, volar medial, lateral ulnar and metacarpal region of both wrist, flexor retinaculum or distal ulnar or radius anterior or lateral region of the hands. All of these are accomplished while the hands and the wrists are at the most neutral relaxing position for the user to hover over the input apparatus such as typing apparatus like keyboard. Accordingly, the user may use the input apparatus, such as typing, with a wrist support typing posture, as shown in FIGS. 3 and 30, that the wrists of the user are well supported by the bars 10 of the hand supporting bridge unit 1 enabling his or her hands naturally hanging down from the wrists to reach the typing apparatus, so that the user may move his or her fingers freely and naturally without stresses applied on his or her wrists.

The first base unit 21 and the second base unit 22 can be made of fiber organic composites, glass, wood, plastic, ceramic composites, rubber, carbon, metallic ally, or any of these combinations together as a hold or in sections throughout the first base unit 21 and the second base unit 22. Preferably the first or second base unit 21 or 22 is equal or higher than an input apparatus such as typing apparatus like keyboard.

Further, the hand support device comprises a pillar and bar joint assembly 30, which is arranged to movably insert the bars 10 into the first base unit 21 and a second base unit 22 in order for the bars 10 to provide a transversal support for the user's upper extremities over the input apparatus such as keyboard. The pillar and bar joint assembly 30 comprises a first pillar and bar joint 31 and a second pillar and bar joint 32 respectively arranged at the first base unit 21 and the second base unit 22, wherein the bars 10 is supported between the first pillar and bar joint 31 and the second pillar and bar joint 32 which are positioned respectably on opposite side of the keyboard.

Referring to FIGS. 1 to 7, a ball-and-socket joint is implemented according to the preferred embodiment, which comprises a pair of first bearing members 311, a pair of second bearing members 321, a pair of first bearing sockets 312, and a pair of second bearing sockets 322. The first bearing members 311 are respectively arranged at first ends of the bars 10 and the second bearing members 321 are respectively arranged at second ends of the bars 10, wherein the bars 10 of the hand supporting bridge unit 1 are extended between the first bearing members 311 and the second bearing members 321. A first bearing socket 312 corresponding to the first bearing members 311 is mounted at a first guiding slot 313 defined in the first base unit 21 in a movable manner, wherein the first bearing members 311 are mounted actively in the first bearing socket 312. One skilled in the art will understand the operating principle and basic structure of the ball-and-socket joint which is not repeated here.

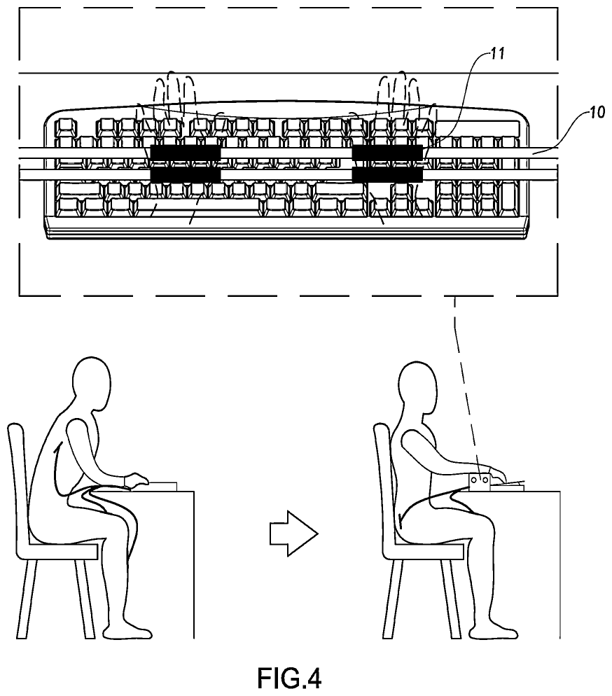

By means of the ball-and-socket joint, each first end of the bars 10 can be rotated to move forward, backward, upward and downward freely and independently. Likely, the second bearing socket 322 corresponding to the second bearing members 321 is mounted at a second guiding slot 323 defined in the second base unit 22 in a movable manner, wherein the second bearing member 321 is mounted actively in the second bearing socket 322, wherein each second end of the bars 10 can be rotated to move forward, backward, upward and downward freely and independently. Accordingly, the wrists of the user resting and being supported on the bars 10 of the hand support bridging unit 1 in the wrist support typing posture are able to move up-and-down and left-and-right above the typing apparatus, as shown in FIGS. 4 and 30, so that the user has no need to bend his or her wrist left and right and to stretch or bend his or her fingers to reach different keys on the keyboard that is the main cause of the joint injury to the wrists and fingers of those users who need to type for relative long time continuously. Instead, the user may naturally hang down his hands and fingers from his or her wrists which are well supported by the hand support bridging unit 1 during typing naturally and neutrally while moving the whole hands vertically and horizontally above the typing apparatus by means of the hand support device of the present invention.

The first guiding slot 313 is formed in an inner side of the first base unit 21, wherein the first bearing socket 312 is mounted in the first guiding slot 313 in a movable manner. The first guiding slot 313 has a first gear rail 3131 arranged at a bottom of the first guiding slot 311, wherein the first bearing socket 312 has a first periphery matched to the first gear rail 3131. In other words, the size of the first bearing socket 312 matches each slot of the first rail 3141 to allow the first bearing socket 312 with the first end of bars rotating e forward and backward and locking at the desire position.

Likely, the second guiding slot 323 is formed in an inner side of the second base unit 22, wherein the second bearing socket 322 is mounted in the second guiding slot 323 in a movable manner. The second guiding slot 323 has a second gear rail 3231 arranged at a bottom of the second guiding slot 312, wherein the second bearing socket 322 has a second periphery 3151 coupled to each slot of the second gear rail 3231. The second bearing socket 322 and the second gear rail 3231 couple to allow the second bearing socket 322 with the second end of bars rotating forward and backward and locking at the desire position, so the desire bar separation between two neighboring bars 10 can be fixed. Or, the first periphery and the second periphery 3151 are arranged with gears around meshed with the first gear rail 3131 to achieve the objects above.

Due to the first bearing socket 312 and the second bearing socket 322 freely moving and not limiting the ball bearing moving, any end of the bars 10 can move in desire direction. Especially, the first end of the bars can move distally away from the user while the second end of the same bars can be moving proximally closer to the user and vice versa, so the user can move his hand for typing freely.

The support base assembly 20 and the pillar and bar joint assembly 30 provide the rolling motion of the bars 10 in a clockwise motion and counterclockwise motion while providing the surface area for the hands and/or wrists to rest thereon. The pillar and bar joint assembly 30 also supports and provides the housing length to the bars 10 for the horizontal angular plane displacement of the bars 10.

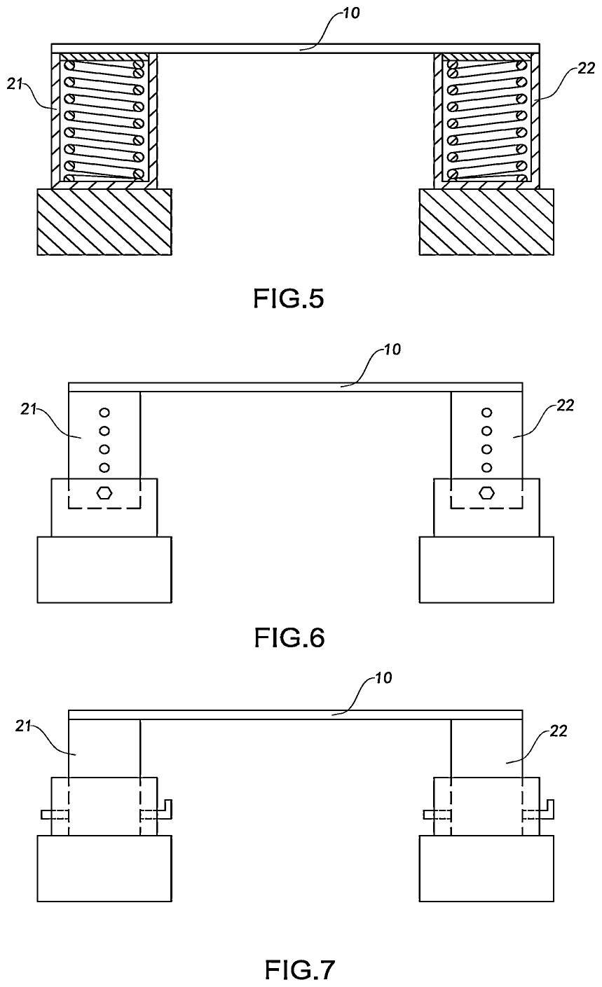

In addition, the first base unit 21 and the second base unit 22 are arranged to be adjustable in height, referring to FIG. 5 to FIG. 7, so as to provide an adjustable vertical support structure while maintaining a vertical parallel height between the bars 10 of the hand supporting bridge unit 1 and the keyboard to the user's desire fix height position and at the same time to maintain the desire fix separation between the bars 10 in relation to the upper extremities. The first base unit 21 and the second base unit 22 are adjustable to height size by clamp, push pin, spring torque, screw positional adjustment, track trial, hydraulic pressure, spring tension or air compression in combination or independent. Or the first base unit 21 and a second base unit 22 can be collapsible, when not in used and the entire device is portable.

In the preferred embodiment, the first base unit 21 and the second base unit 22 are implemented as a telescopic structure as shown in FIGS. 5 to 7. An inner pillar is mounted to slide in an outer pillar, wherein a screw is mounted near the top edge of the outer pillar to control and lock the slide of the inner pillar. When the screw is tightened, the inner pillar is locked at the desire position and when the screw is loosed, the user can slide the inner pillar to the desire position. One skilled in the art will understand that the structure in the preferred embodiment is an example, they can use other structure to control and lock the height of the first base unit 21 and the second base unit 22 mentioned above.

Moreover, as shown on FIG. 4, the hand supporting bridge unit 1 further comprises a plurality of sleeves 11 provided around the bars 10 for additional motion in relation to the existing motion of the bars 10. For example, if the bars 10 are in a rolling motion clockwise or counterclockwise, the sleeves 11 increase additional rolling motion clockwise and counterclockwise in relation to the existing clockwise motion or counterclockwise motion of the bars 10. The motion of the sleeves 11 is like the motion of the bars 10 of the hand supporting bridge unit 1 which can be locked in either motion or completely fix to no motion either way, such as forward and backward rotation.

In addition, the bars 10, sleeves 11, and the braces 66 are the end points where therapeutic build-in leads carry the specific therapeutic currents from the hand support device to the user's skin to provide therapy. In other words, the hand support device also has multiple non built-in leads that attach to an electro path independently from the built-in leads of the bars 10, sleeves 11 and braces 66, and therefore deliver additional desire therapy on any other body part where the user requires treatment. For example, while the user is treating his or her upper extremity through the bar, sleeve, or brace, the user can connect the non built-in leads on the hand support device and also place corresponding electros pad onto the corresponding leads to use in additional body parts. Ultimately, the user may position the electro pads on the skin surface of the back and legs and therefore get a more complete body care, for example an external bone stimulator.

Further, the sleeves 11, bars 10, and braces 66 can be spring loaded to achieve desire placement at all times of the sleeve, brace and bars in combination or separated in relation to making contact with the user's skin. This will also give the user proprioception in relation to the home run keys on the input apparatus such as typing apparatus like keyboard and on the overall hand support device location.

The additional function of the sleeves 11 and brace 66 are to decrease contact dermatitis and provide shock support. The sleeves 11 and braces 66 can have various shapes and sizes. The sleeves 11 and braces 66 can be made of fiber organic composites, glass, wood, plastic, ceramic composites, rubber, carbon, metallic alloy, or any of these combinations together as a hold or in sections throughout the sleeves 11 or brace 66. The sleeves 11 is mounted on the parallel bars 10 in a sliding manner, wherein the sleeves 11 provide the user additional left to right sliding motion on the bars in relation to the keyboard and vice versa. The brace 66 can be mounted on the sleeve or without the sleeve on to the bars providing the same sliding function as the sleeve 11, as mention above, with additional treatments options and comfort. For example, the brace 66 can transfer current or heat on to a glove, body sleeve, split or a garment as other means of delivering therapy and where the bars and sleeves can have the same function of the brace, as mention above.

The support base assembly 20 further comprises a mouse device 90. The mouse device 90 is configured in the inner side of the second base unit 22. The mouse devices 90 is installed in front of the bars 10, as shown on figured 1. Preferably, the mouse device 90 perpendicular in front of hands in relation to the bars for convenience and effortless repetitive motions. The mouse device 90 further comprises a detecting bar 92 and a detecting sleeve 93. The detecting sleeve 93 is provided around the detecting bar 92. The second base unit 22 further has a slot 91. One end of the detecting bar 92 is installed in the slot 93 so as to the detecting bar 92 is able to move along the slot 91. When the detecting bar 92 moving along the slot 91, the detecting bar 92 detects the displacement between the original place of the slot 91 to the current place of slot 92, and sends a moving data regarding to the displacement to a computer. The computer processes the moving data and control the mouse to move. When the detecting sleeve 93 moving along the detecting bar 92, the detecting sleeve 93 detects the displacement between the original place of the detecting bar 92 and the current place of the detecting bar 92, and then sends the moving data regarding the displacement of the detecting sleeve 93 to the computer. The computer processes the moving data and control the mouse to move. It is important to mention the mouse devices moves with the pillar assembly 20, so that the user is capable of having minimal finger reach to active the mouse devices 90 at all time with minimal reaching effort. The mouse devices 90 is capable of being installed in the first base unit 21 and/or the second base unit 22. Preferably, the mouse device 90 is attached and installed on the second base unit 22 if user is right hand dominant per drawing schematic. In another embodiment, the mouse device 90 is a trackball. The track ball has at least one LED (light emitting diode). The mouse device 90 can be a touch flat pad or touch screen. The touch flat pad and the touch screen have various designated shapes, such as rectangle, triangle, square, circle, cylinder, tube, or sphere. The user is able to control to mouse through the touch flat pad or touch screen, such as, single tap, double tap and other operation. The mouse device 90 can be a nipple mouse. The nipple mouse has the same functions as the computer mouse. It is also important to mention that motion hologram technology can also be applied to the keyboard and mouse controls.

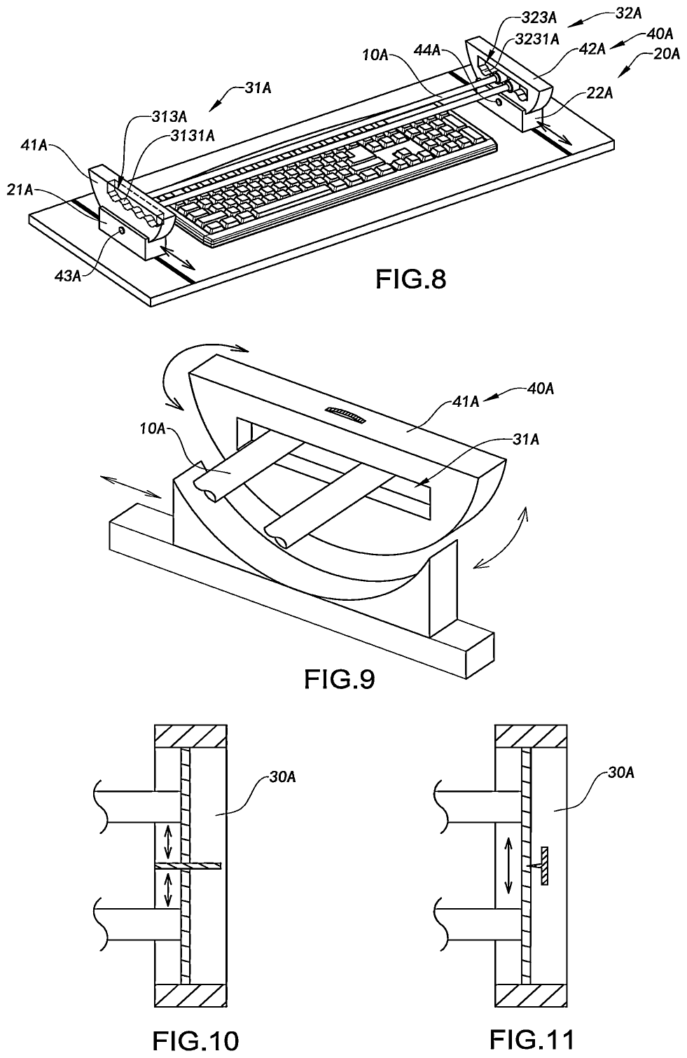

Referring to FIGS. 8 to 11, the hand support device according to a second preferred embodiment of the present invention is illustrated. The hand support device further comprises a pillar joint assembly 40A which is rotatably mounted at the support base assembly 20A for the angular motion of the bars 10.

The pillar joint assembly 40A comprises a first swing pillar 41A and a second swing pillar 42A, which are respectively mounted rotatably at the first base unit 21A and the second base unit 22A on both sides of the keyboard respectively. The pillar and bar joint assembly 30A is formed at the pillar joint assembly 40A, wherein the first pillar and bar joint 31A is arranged at the first swing pillar 41A and the second pillar and bar joint 32A is arranged at the second swing pillar 42A.

The first guiding slot 313A with the first gear rail 3131A is defined at the first swing pillar 41A and the second guiding slot 323A with the second gear rail 3231A is defined at the second swing pillar 42A, wherein the first periphery of the first bearing socket 312A couples with the first gear rail 3131A and the second periphery of the second bearing socket couples with the second gear rail 3231A, so the ends of the bars 10A of the hand supporting bridge unit 1A mounted with balls can move relative to the first swing pillar 41A and the second swing pillar 42A.

The pillar joint assembly 40A further comprises a first rotation and fix component 43A and a second rotation and fix component 44A, wherein the first swing pillar 41A is mounted at the first base unit 21A in a swing manner by the first rotation and fix component 43A and the second swing pillar 42A is mounted at the second base unit 22A in a swing manner by the second rotation and fix component 44A. When the first swing pillar 41A and the second swing pillar 42A rotate at the desire position, a surface of the bars 10A defined has a desire angle with the keyboard which increases additional comfort for the hand and wrist. When the first swing pillar 41A and the second swing pillar 42A rotate at the desire position, the first rotation and fix component 43A and the second rotation and fix component 44A lock the rotation.

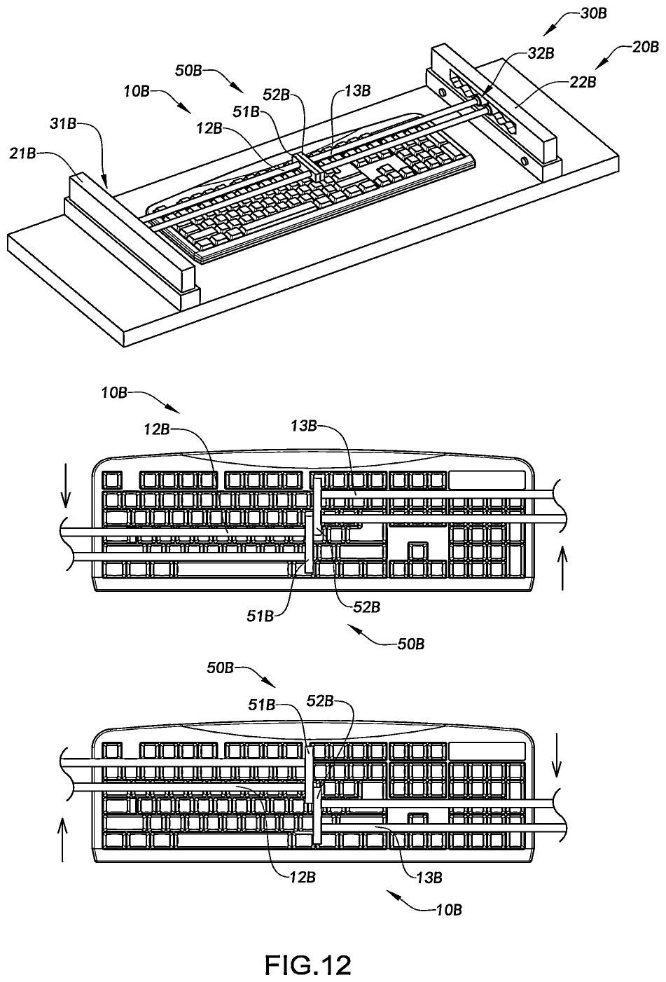

Referring to FIG. 12, the hand support device according to a third preferred embodiment of the present invention is illustrated. Each of the bars 10B of the hand supporting bridge unit 1B is divided into two half bars, including a first half bar 12B and a second half bar 13B, wherein a mid-bar divider 50B is arranged in the middle of the first half bar 12B and the second half bar 13B arranged in a same extension line.

The mid-bar divider 50B comprises a first mid-bar divider 51B and a second mid-bar divider 52B, wherein the first mid-bar divider 51B and the second mid-bar divider 52B are arranged to slide on each other to permit the first mid-bar divider 51B and the second mid-bar divider 52B to move forward and backward independently. According to this embodiment, the first mid-bar divider 51B has a sliding slot which couples with a raised sliding joint of the second mid-bar divider 52B, wherein the raised sliding joint is inserted in the sliding slot so that the first mid-bar divider 51B and the second mid-bar divider 52B can slide independently.

One end of the first half bar 12B is arranged at the first base unit 21B and the other end of the first half bar 12B is inserted to one side of the first mid-bar divider 51B opposite to the sliding slot, while one end of the second half bar 13B is arranged at the second base unit 22B and the other end of the second half bar 13B is inserted to the other side of the second mid-bar divider 52B opposite to the raised sliding joint. In order to adjust and fix the separation of the half bars, a guiding slot with a gear rail can be arranged in the side of the first mid-bar divider 51B or the second mid-bar divider 52B the half bar inserted.

The mid-bar divider 50B can also permit the half bars 12B, 13B of the bars 10B to move upward and downward by a frictionless surface that will be telescope perpendicular or a sliding connective symbiotic pressure cancelation, which will continue to provide support on the bars 10B. And be able to resist gravity and support the apply forces by both user hands on opposite sides of the mid-bar divider 50B. In other words, the movement of moving the hand up and down independent from each other can be also accomplished by the mid-bar divider 50B between the first half bar 12B and the second half bar 13B.

It is worth to mention that the mid-bar divider 50B is situated directly in the middle between the first half bar 12B and the second half bar 13B, wherein each of the first mid-bar divider 51B and the second mid-bar divider 52B have an opposite frictionless surface parallel to each other. Both the frictionless surfaces of the first mid-bar divider 51B and the second mid-bar divider 52B are parallel and have proportionally length to maintain contact throughout the frictionless surface telescoping connector at all times throughout the require up and down motions. Furthermore, both the frictionless surfaces stay in contact directly or indirectly to opposed and cancel the downward pressure apply by the hands on the bars 10B and to provide the independent up and down frictionless motion on the hands on the bars. Because of mid-bar divider 50B is arranged in the middle between the bars 10, so that it does not get in the way from the keyboard operation being conducted by the user.

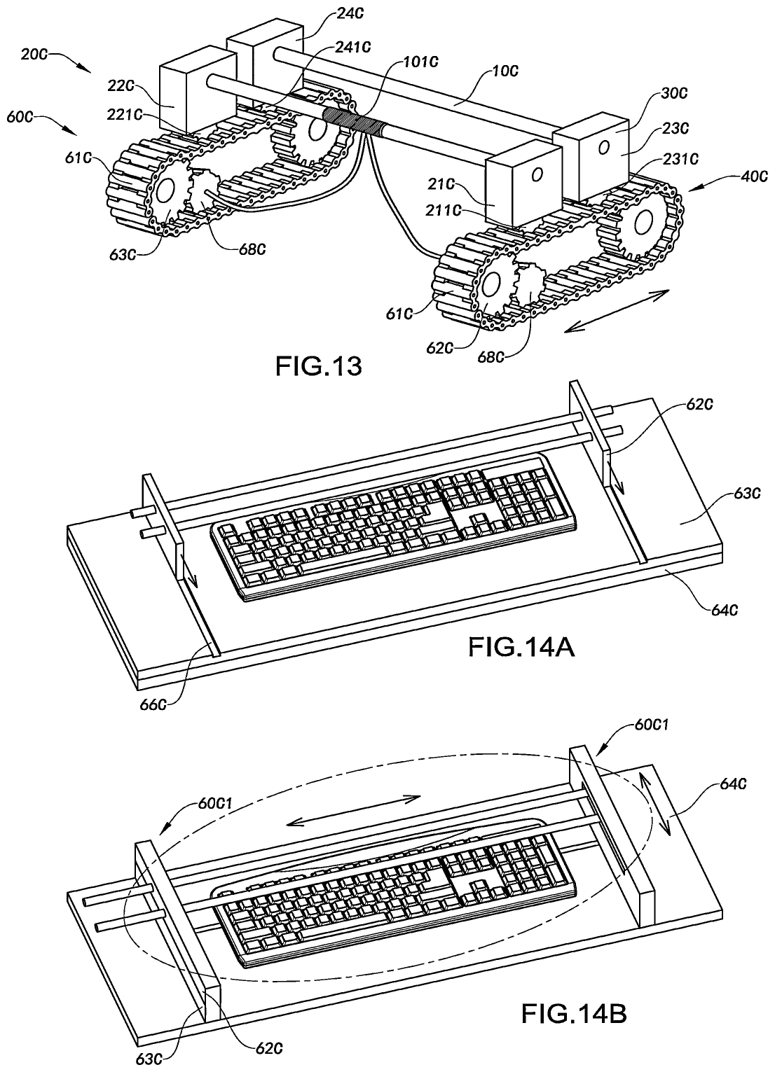

Each of the bars may be supported by a single pillar or a pillar may be arranged with multiple bars. Referring to FIG. 13, the hand support device according to a fourth preferred embodiment of the present invention is illustrated, wherein one pillar is only related to one bar. In other words, the support base assembly 20C further comprises a third pillar 23C arranged at the same side as the first base unit 21C to the keyboard, and a fourth pillar 24C arranged at the same side as the second base unit 22C to the keyboard, wherein one of the bars 10C is inserted and fixed into the first base unit 21C and the second base unit 22C, and another one of the of the bars 10C is inserted and fixed into the third pillar 23C and the fourth pillar 24C over the keyboard. One skilled in the art will understand that the number of the pillars and the bars is just an example not limited.

The hand support device further comprises a base assembly 60C which supports the pillar and bar joint assembly 30C to have the motion by being mounted on the base assembly 60C. The base assembly 60C provides a tandem motion between the support base assembly 20C on to the bars 10C over the keyboard, with or without the horizontal angular motion. Preferably, the base assembly 60C is a liner frictionless track base related to both sides of the keyboard.

According to the fourth preferred embodiment of the present invention, the base assembly 60C can be implemented as a band pulley system, a belt pulley system, a tooth pulley system, a Boston pulley system, or a timing belt system, wherein an upper track 61C is adjustable for each other separation of the bars 10C which locks the bars 10C of the hand supporting bridge unit IC in the desire bar separation between the bars 10, and locks the location of the bars 10C in relation to the location of the first base unit 21C and the second base unit 22C to the keyboard.

The base assembly 60C comprises a first base 62C and a second base 63C, wherein the first base unit 21C and the third pillar 23C sit movably on the first base 62C, wherein the second base unit 22C and the fourth pillar 24C sit movably on the second base 63C. According to the fourth preferred embodiment, the first base 62C and the second base 63C are implemented as the band pulley system for example, wherein a first wheel 211C and a third wheel 231C mounted at the bottom of the first base unit 21C and the third pillar 23C mesh with the first base 62C for moving and freely and independently, wherein a second wheel 221C and a fourth wheel 241C mounted at the bottom of the second base unit 22C and the four pillar 24C mesh with the second base 62C for moving freely and independently, so the separation between bars 10C will be adjustable and locked according to the separation and the location of the formers. Along the track 61C, moving direction of the pillars is guided for the base assembly 60C to work as a unit.

In addition, the first base 62C and the second base 63C also support the upward and downward independent motion of the support base assembly 20C simultaneously, independent of the pillar and bar joint assembly 30C and the pillar joint assembly 40C. For example, a second lower chain pulley band is controlled by the user for an up and down gliding motion. It is in the mechanical layout that different gears types can be assembled for motorize electrical automatic sensory uses like spur gear, helical gear, herring gear, herring bone gear, rack and pinon gear, revel gear, spiral bevel gear, screw gear, worm and worm wheel gear, miter gear, and internal gear. Preferably, the band tension of the first base 62C and the second base 63C is adjustable to provide the user additional comfort.

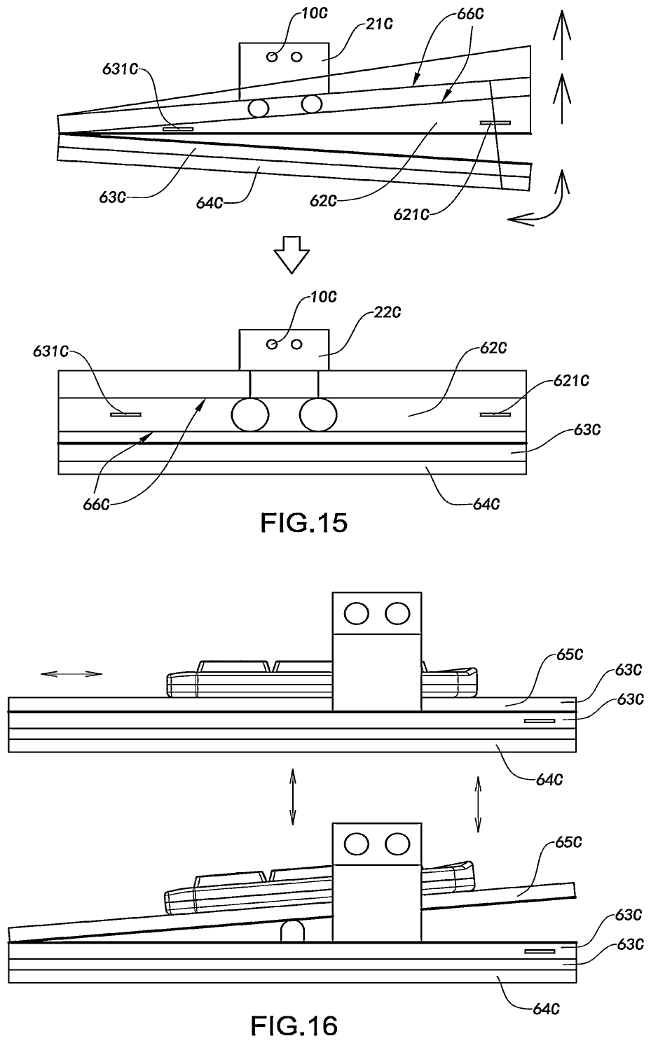

Or, in another embodiment according to FIG. 14 and FIG. 15, the first base 62C comprises a first lift control 621C, while the second base 63C comprises a second lift control 631C to respectively support the first base 62C and the second base 63C to incline. By the first lift control 621C and the second lift control 631C, the distal ends of the first base 62C and the second base 63C related to the user may be higher than the proximal end to make the first base 62C and the second base 63C inclining and vice versa, wherein when the user can incline the first base 62C and the second base 63C in the opposite direction which forces the support base assembly 20C inclines away from the user in an automatic effortless manner.

Preferably, the first lift 621C and the second lift 631C provide at less a 0 to 60 degrees downgrading slope and upgradient slope. When the bars 10C of the hand supporting bridge unit IC are over the keyboard and the users releases the bars 10C on an upper region of the keyboard, it will automatically have a gravitational down grade, wherein the support base assembly 20C with the bars 10C have a decline gradient at all times which leads the bars 10C closer to the user at a faster time automatically and effortlessly. In other words, the first lift control 621C and the second lift control 631C provide a faster and automatic gravitational direction of the support base assembly 20C and bars 10C related to the users and the operator upper extremities.

In other embodiments, an electronic device or a control device is arranged with the first base 62C and the second base 63C by any types of mechanical set ups, as shown in FIG. 25. As shown on FIG. 13, the base assembly 60C further comprises at least one pressure sensor 101C. The pressure sensor is provided on the bar 10. The base assembly 60C further comprises an electric motor 68C. The electric motor 68C is installed between the first base 62C and the second base 63C. The pressure sensor 101C is electric connected the electric motor 68C. When the pressure sensor 101C received pressure, the pressure sensor 101C sends a signal to the electric motor 68C for trigging the electric motor 68C to work. The electric motor 68C drivers the first base 62C and the second base 63C to move. According to the received pressure position of the pressure sensor 101C. The electric motor 68C is electrically connected a power source. By providing a pressure sensor, the hand support device will read and activated the control sensors to perform the desire actions electronically.

The base assembly 60C, as shown in FIG. 14, bears the weight of the support base assembly 20C, that provides a bilateral vertical support, a horizontal support cross sectional support and a lateral support. The base assembly 60C may or may not connect to the keyboard at the right and left opposite side of the keyboard. When the base assembly 60C connects the keyboard, it provides the keyboard stability in response to the forces being apply during normal usage. In another embodiment, as shown in FIG. 13, the electrical component and the motorize engine are inside the base assembly of 60C.

Preferably, the first base 62C and the second base 63C are made of fiber organic composites, glass, wood, plastic, ceramic composites, rubber, carbon, metallic ally, or any of these combinations together as a hold or in sections throughout the first base 62C and the second base 63C.

Further, a first incline slope control is arranged at the first base 62C and a second incline slope control is arranged at the second base 63C for additional direction guide at the first base 62C and the second base 63C. The first incline slope control and the second incline slope control can be a mechanical slide, a linear guide rail or track, a vertical wheel on a rail mount, dualvee motion technology guide wheel linear rack, wheel bearing and/or liquid nitrogen super conduction frictionless surface referring to FIG. 23.

In another embodiment, as shown on FIG. 15, one or more first guide rails 66C are supported by the first base 62C to predetermine the motions of the pillars sitting thereon that preset the motion rang of the bars 10C for the user, wherein the first guide rails 66C provide the pillars the movement and provide synchronization between the user, the keyboard and the hand support device. The guide rails can be made of fiber organic composites, glass, wood, plastic, ceramic composites, rubber, carbon, metallic alloys, or any of these combinations together as a hold or in sections throughout the guide rails. Likely, the first base 62C is supported by the second base 63C.

It is important to mention that the base units are arranged to house the mechanical movement mainly to prevent the user's clothing from getting stuck by or the user getting injured from all the moving parts. It is appreciated that the base units may also house other additional pressures components, electrical components and mechanical components for the delivery of the invention movements and functions. In addition, the each of the first and second base units can be mounted on the corresponding back brace for the movement where the base units are the barrier to prevent the movement of the hand support device from interfering with external objects. Furthermore, the base units can be mounted with additional bracing to provided additional support to taller individuals forearms and elbows from the sides of the base or from on top of the base unit on any and all embodiments of the present invention, including other embodiment that also includes a fix mount on a swivel.

As shown FIG. 14, the base assembly 60C further comprises a back brace 64C supporting the first base 62C and the second base 63C, wherein the back brace 64C connects the first base 62C and the second base 63C. The back brace 64C can be made of fiber organic composites, glass, wood, plastic, ceramic composites, rubber, carbon, metallic alloys, or any of these combinations together as a hold or in sections throughout the back brace 64C. The back brace 64 can be or have various shapes and layouts to include different sizes. It is important to mention inside the back brace is the physical location for housing the electrical, cyber software programs, interfaces connectors, interactive software, artificial intelligence, security programs, smart phone cavity, internal and external therapeutic leads, biometric and biomarkers software, financial programs, smart phone computer program (APP), sideline program, system communication programs, the on/off function wire and wireless connectors etc.

The back brace 64C is an underneath plane surface area where all elements of the device and the working parts in the device are housed and supported thereon. In other words, the back brace 64C is a surface tension area that makes direct contacts with a flush flat opposing a counter surface area where the hand support device sits on, such as a table. The back brace 64C is 1/16 to 3 inches in thickness, 1 to 46 inches in length and 1 to 46 inches in width.

A mounted plate 65C attaches directly over the back brace 64C, wherein the mounted plated 65C is used to house the keyboard or laptop for stabilization, to the first base 62C and the second base 63C. The mounted plate 65C sits adjacent, side to side or slightly elevated in relation to the first incline slope control and the second incline slope control. The mounted plate 65C is arranged to rise and fall, wherein two elevators 651C are respectively arranged at the middle of the left edge and the right edge thereof next to the first base 62C and the second base 63C, which lock the mounted plate 65C at a neutral elevation. By elevating the two elevators 651C, the center of the left and right sides of the mount plate 65C, it provides an inclination and a declination independent from the first base 62C and the second base 63C which provide the inclination or declination for the pillars up or down grade movement independent from the position of the keyboard or laptop. the higher the keyboard or laptop is elevated with the mount plate 65C, the deeper the angle of inclination and vice versa the higher the angle for decline in relation to the bars 10C and pillars 21 and 22.

The mount plate 65C further comprises a block 652C to fix the keyboard, wherein the block 652C can be universal clips, screw, or straps in combination or through on process. It is important to mention that the elevators 651C work independent of the support base assembly 20C, the first base 62C and the second base 63C, wherein the mount plate 65C allows the keyboard to have a distal decline, distal elevation, or a neutral adjustment independently, when the keyboard or laptop are securely mounted on the mount plate 65C.

Preferably, the mounted plate 65C has the same length and width as the keyboard, wherein the mounted plate 65C has equal distance from the left side to the right side and from the top to the bottom of the base brace 64C. The base brace 64C connects the first base 62C and the second base 63C on the right and left side of the keyboard or laptop, to include the top and bottom sides of the keyboard or laptop in relation to the user. In another embodiment, the first base unit and the first base are integrally arranged and the second base unit and the second base are arranged integrally arranged as a base and pillar fix structure. The base and pillar fix structure may or may not connect to the back brace, or even be arranged without the back brace. The base and pillar fix structure can be made of fiber organic composites, glass, wood, plastic, ceramic composites, rubber, carbon, metallic alloys, or any of these combinations together as a hold or in sections throughout the base and pillar fix structure. The back brace may or may not connect the base and pillar fix structure on all sides the hand support device. In other words, the top is the horizontal most distal keyboard margin in relation to the user and the bottom of the keyboard is the proximal most closes to horizontal margin of the keyboard in relation to the user.

In another embodiment according to the FIG. 18 and FIG. 19, the first pillar and bar joint and the second pillar and bar joint are implemented as spring close casings that provides additional freedom for horizontal angular motion support on the bars, while maintaining the desire bar separation or bar union between the bars throughout the time of usage. The first pillar and bar joint and the second pillar and bar joint in a different open casing configuration are also the location where the bars can slide form the opposites side to provide an escape sliding bar release system for the purpose of moving the bar and the user's upper extremities together from side to side, from right to left and vice versa.

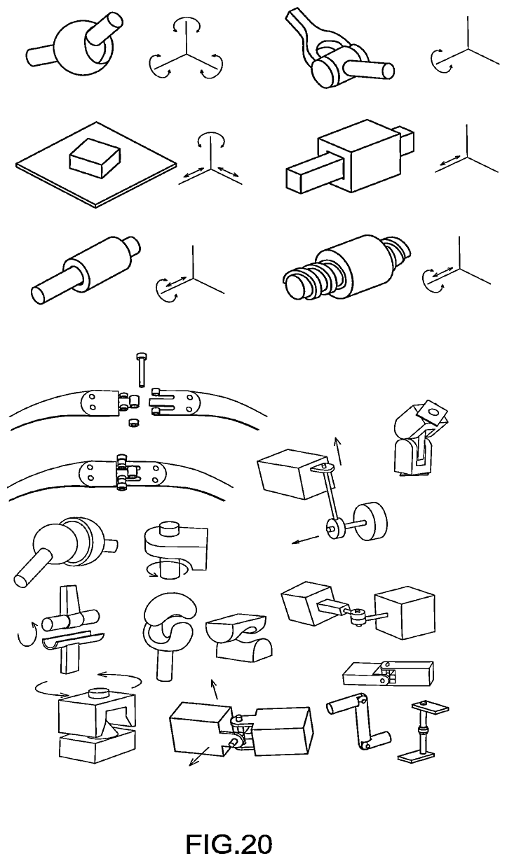

In addition to the embodiments above, the first pillar and bar joint and the second pillar and bar joint can be implemented as any mechanical structure likely shown in the FIG. 20. In other words, the embodiments above are example not limited. In other words, in some embodiments, the bars can freely spin clockwise and counter clockwise so that the bars can spin forward or away from the user applied any mechanical structure likely shown in the FIG. 20. In addition, the bars can be locked to stop the free spin motion in either direction or completely which is convenient for the direction and posture adjustment.

In another embodiment, the pillar joint assembly is implemented as a partial or complete concave or convex ball and socket joint movement system position on the pillar. Furthermore, the pillar joint assembly can be in any axis up or down side to side in relation to the middle central equator axis between the concave and the convex union. In other words, the pillar joint assembly provide the bars the ability to have angular horizontal motion in relation to the first base unit and the second base unit on each side of the opposite side on the keyboard or laptop. The system permits the user to move any hand up and down independent of the other hand and fingers for positioning because the ball and socket is free to rotated side to side in respect to the perpendicular horizontal plane on each side.

According to the hand support device in the present invention, the pre-existing keyboard or laptop as a premanufacture device attaches to the hand support device, wherein the present invention of the hand support device does not need to produce the keyboard, input apparatus such as typing apparatus like keyboard or laptop. The premanufacture keyboard or laptop comes in different sizes, wherein the hand support devices in the present invention are arranged in various specific ranges of measurements that provides just the right levels of additional adjustments to provide optimal utilization, and more convenience to the user. The hand support device of the present invention takes into account numerous additional adjustments, each user will need. It is important to mention the invention, in no way is made to be limited from future potentials of being produce as a built in single device unit where the hand support device are build-in, onto the keyboards, laptops, game devices and input apparatus such as typing apparatus like keyboard or any other computing devices where typing or repetitive finger, hand movements are required.

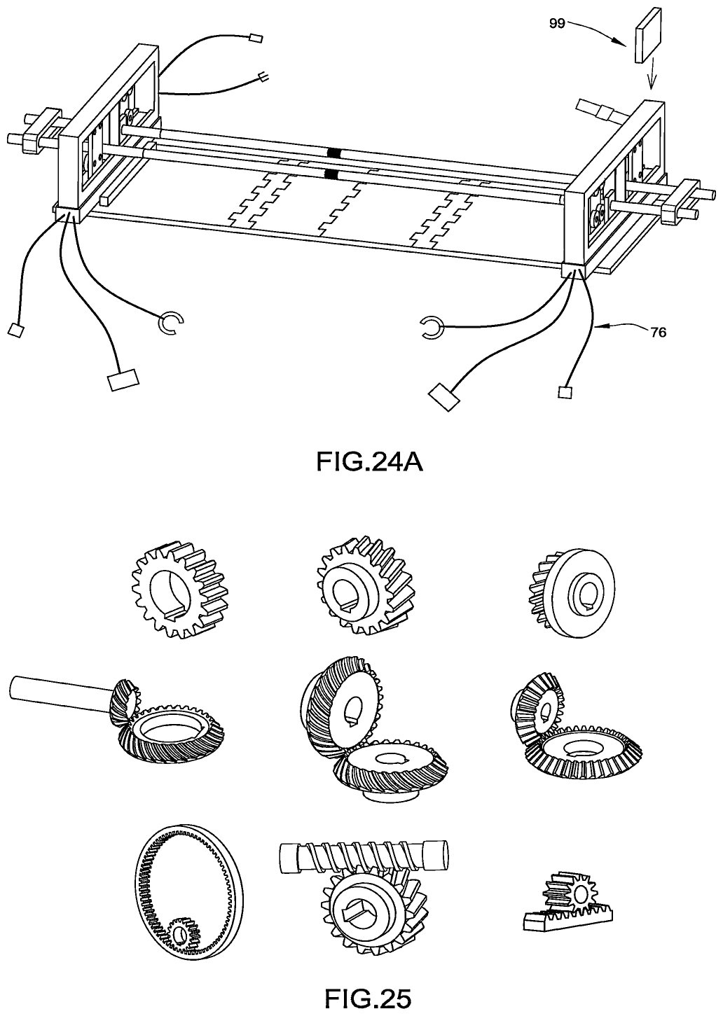

According to the base assembly 60, it can elevate on either end and is connected to the support base assembly 20 that can move up and down in relation to the input apparatus such as typing apparatus like keyboard, it is important to mention the keyboard or laptop position and location may or may not be fixed without any additional movements, The present invention mainly illustrates an attachable hand support device for the purpose of describing, and explaining details of the present invention. As shown in FIG. 23, the hand support device is built in the keyboard, as shown in FIG. 23. FIG. 24 illustrates that the hand support device is built in onto the laptop with electrical wiring connecting to power source or other electrical appliances such as therapeutic devices.

FIG. 24A illustrates another embodiment of the hand support device of the present invention, wherein electrical wires or leads are extended therefrom to electrically connect with external devices, such as USB, mouse, therapeutic cartridge, therapeutic electro pads or deahs, earphones, camera, internal therapeutic device, and the like.

Referring to FIG. 25 of the drawings, in regards to the numerous gears for movement and adjustments in relation to comfort and performance of the hand support device.

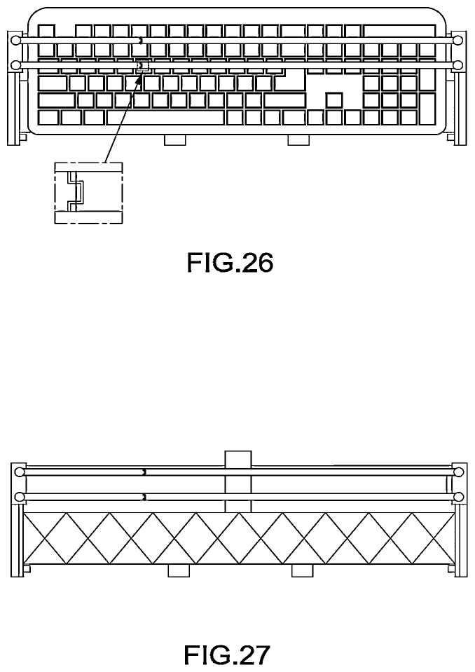

Referring to FIG. 26 of the drawings, the bar/pillar/base system in the hand support device in relation to the keyboard, top view.

Referring to FIG. 27 of the drawings, the bar/pillar/base system in the hand support device without the keyboard, top view.

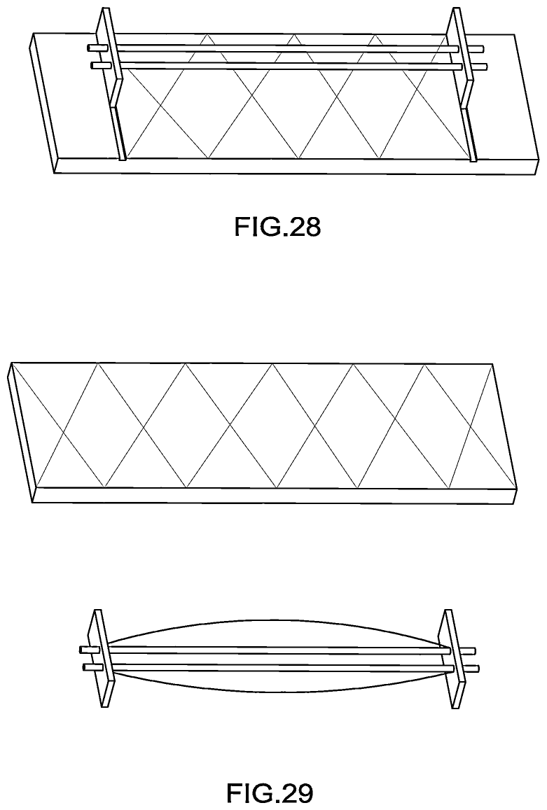

Referring to FIG. 28 of the drawings, the bar/pillar/base system top view and bottom view of the same hand support device.

Referring to FIG. 29 of the drawings, a detach bar/base unit is illustrated, where the bar or bars can be cover by a pad like material that is soft, elastic, rubbery, durable, and sturdy as another example of the present invention.

Referring to FIGS. 30A to 30E of the drawings, FIG. 30A illustrates a cross view of the hand support device without the pillars, illustrating the position of the hand without gloves hovering neutrally over the bars in regards to the location of the input apparatus such as typing apparatus like keyboard, where the bar and or sleeve is making contact with the users skin, for the purpose of support and providing optional treatment care. FIG. 30. illustrates a cross view of the hand support device without the pillars, illustrating the position of the hand with gloves hovering neutrally over the bars and or sleeves in regards to the location of the input apparatus such as typing apparatus like keyboard, where the bar and or sleeves are making contact with the users gloves for the purpose of delivering additional therapy throughout the glove and providing additional shock support. FIG. 30C illustrates a cross view of the hand support device without the pillars, illustrating the position of the hand with gloves hovering neutrally over the bars and or sleeves in regards to the location of the input apparatus such as typing apparatus like keyboard, where the brace is mounted on the bar or sleeve for additional therapy and where the glove outside volar surface are in contact with the brace to transfer current onto the inside of the glove that is in contact with the users skin for the purpose of providing additional optional treatment care and support. It is important to mention the brace can be used without the glove and the user will also be able to receive the optional therapeutic modalities.

FIG. 30D illustrates the various shapes, sizes, and composition of the brace and more important the synergy function the bar or bars and the braces have in common for the purpose of supporting the hand while therapy is in process. The shape can be rectangular, circular, triangular, and or square, in any combination. The sizes will range from small, medium and large. The brace will give the user a 180 degrees rotation reach where the brace can be moved and locked anywhere throughout the horizontal length of the bar or bars while supporting the user's hands. The brace also functions with a 360 degrees rotational circular support under the user wrist for additional wrist reaching movements. It is important to mention the brace can have the electrodes on any location on the brace, the bar or bars are the means by which the current can get to the brace and make contact with the user skin to deliver different therapeutic modalities. Any and all rotations on the brace in relation to the mechanical support on the upper extremities can be locked independent of the location of the brace on the bar or bars. The braces can also have and up and down vertical motion and can also be locked at a proximal or distal position in relation to the vertical distance between the user and the keyboard. The brace can be made of fiber organic composites, glass, wood, plastic, ceramic composites, rubber, carbon, metallic alloys, or any of these combinations together as a hold or in sections throughout the brace.

It is important to mention the circular micromotion of the braces and bars have due to the vertical up and down sliding pillars motion with the horizontal right and left over extension motion of the bar or bars on the pillar joints together, the vertical and horizontal motions, including up-and-down motions and left-and-right motions, enable the bars/brace system to have an elliptical and/or circular motion in relation to the keyboard or input apparatus such as typing apparatus like keyboard.

The keyboard or input apparatus such as typing apparatus like keyboard can have a neutral parallel elevated plane or a flat decline in relation to the counter surface. The keyboard or input apparatus such as typing apparatus like keyboard can also be elevated on the proximal bottom edge of the keyboard to create a forward decline grade or be elevated on the distal top edge of the keyboard to create a backwards decline grade at 70 degrees. The user can also give the right and left side of the keyboard or input apparatus such as typing apparatus like keyboard an elevation to create a tilt on either side of the keyboard or input apparatus such as typing apparatus like keyboard. The keyboard or input apparatus such as typing apparatus like keyboard can be provided all of these movements through the use of the mount plate, as mention before or through the use of structural wedge support under the keyboard or the input apparatus such as typing apparatus like keyboard. Accordingly, the wrists of the user resting and being supported on the bars of the hand support bridging unit in the wrist support typing posture are able to move up-and-down and left-and-right above the typing apparatus, as shown in FIGS. 30A to 30C, so that the user has no need to bend his or her wrist left and right and to stretch or bend his or her fingers to reach different keys on the keyboard that is the main cause of the joint injury to the wrists and fingers of those users who need to type for relative long time continuously. Instead, the user may naturally hang down his hands and fingers from his or her wrists which are well supported by the hand support bridging unit 1 during typing naturally and neutrally while moving the whole hands vertically and horizontally above the typing apparatus by means of the hand support device of the present invention.

Referring to FIG. 30E, the Cranial Electrotherapy Stimulation (CES) lead will provide an earpiece with acupressure capabilities to be place on specific trigger points on the outer ear throughout the helix and the antihelix for acupressure areas and to provide treatment of anxiety, insomnia, and depression at the same time. Additionally, the Cranial Electrotherapy Stimulation (CES) leads can be place on the mastoid process with an electro patch with or without the earpiece for acupressure. Specifically, the Cranial Electrotherapy Stimulation (CES) leads can have a build-in speaker and the earpiece can have a audio hearing capacity to enable the user to multi-task as previously mention in this invention. It is important to mention the metallic tip provides the current treatment on to the conductor piece, and on to the pressure conductor, and all three pieces are interchangeable parts for the comfort and delivery on to different users. The conductor piece surrounds the metallic tip that provides current and counter pressure to the pressure conductor, where both the conductor piece and the pressure conductor can be saturated in a liquid solution to enhance electrical conduction.

In general, the present invention dose various efforts to provide adjustment for positioning as the means to providing a quality tool to provide support assistances and wellness to the consumer for repetitive hand, wrist and fingers movements. According to the present invention, the keyboard hand therapeutic support device is an attachment unit for an existing keyboard or laptop and\or to have itself on a fix keyboard or laptop premanufactured. A set of ranges in all the measurements, lengths, diameters, width, sizes and horizontal/vertical angular degrees have been proven to provide the maximum result to the majority of the human population dimensions.

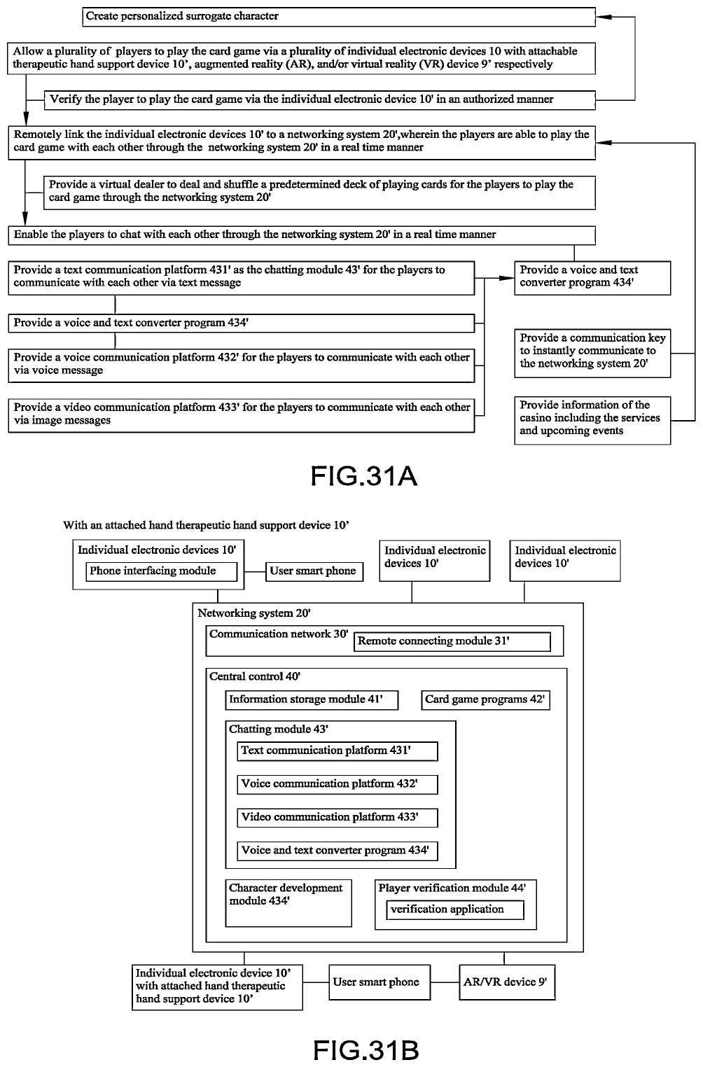

Referring to FIG. 31 of the drawings, a method of operating an electronic device with a detachable or attachable hand therapeutic support device for playing games, especially for card games, according to a preferred embodiment of the present invention is provided, wherein the method comprises the following steps.
(a) Allow a plurality of players at least to play a game or process of at least a software or application via a plurality of individual electronic devices with a detachable or attachable therapeutic hand support device 10′ as described preferred embodiments respectively.
(b) Remotely link one or more of the individual electronic devices with the detachable or attachable therapeutic hand support device 10′ as described in the above preferred embodiments to a networking system 20′, wherein the players or users are able to play the same game or process the same software or application with each other through the networking system 20′ in a real time manner.
(c) Enable the players to chat with each other through the networking system 20 in a real time manner, wherein the players or users are able to remotely and simultaneously play the game or process the software or application and chat with each other via the individual electronic devices with the detachable or attachable therapeutic hand support device 10′ as described in the above preferred embodiments through the networking system 20′, such that the networking system 20′ forms not only a playing or processing channel for the player or user to play the game or to process the software or application but also a communication channel for the players or users to chat so as to enhance the entertainment of the game or the software or application.

Accordingly, the networking system 20′ may further comprise at least a central control 40′ for centrally managing the networking system 20′. The central control 40′ preferably comprises an information storage module 41′ for storing a plurality of information of players or users, and a plurality of game, software and/or application programs 42′ electrically and digitally stored in the central control 40′ for providing variety of games, softwares or applications being remotely and virtually played or processed by the players or users.

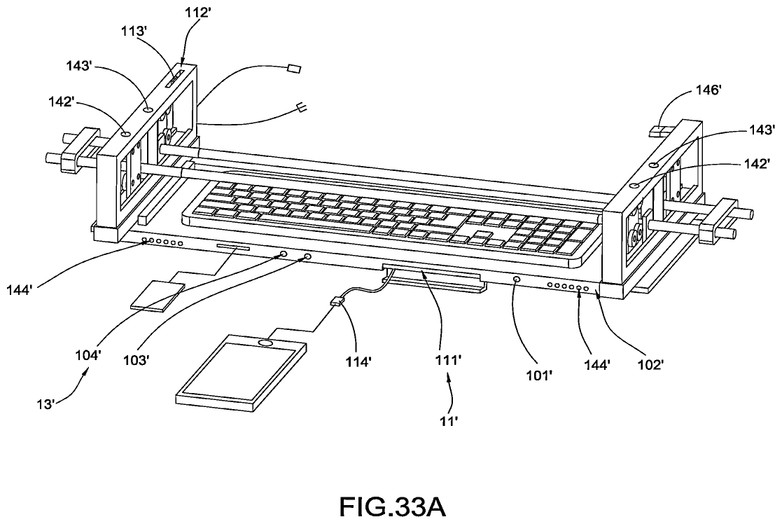

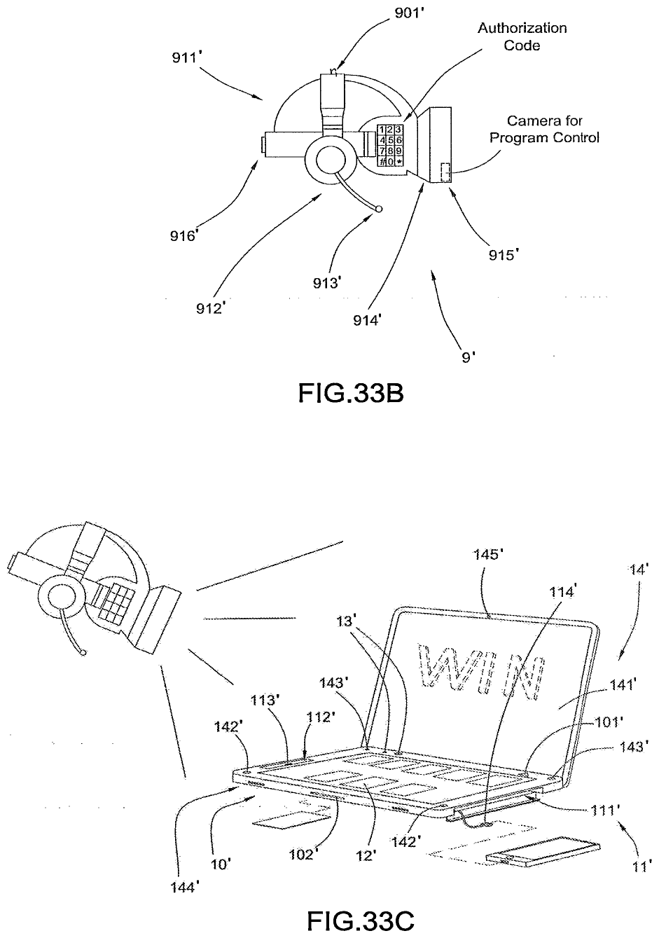

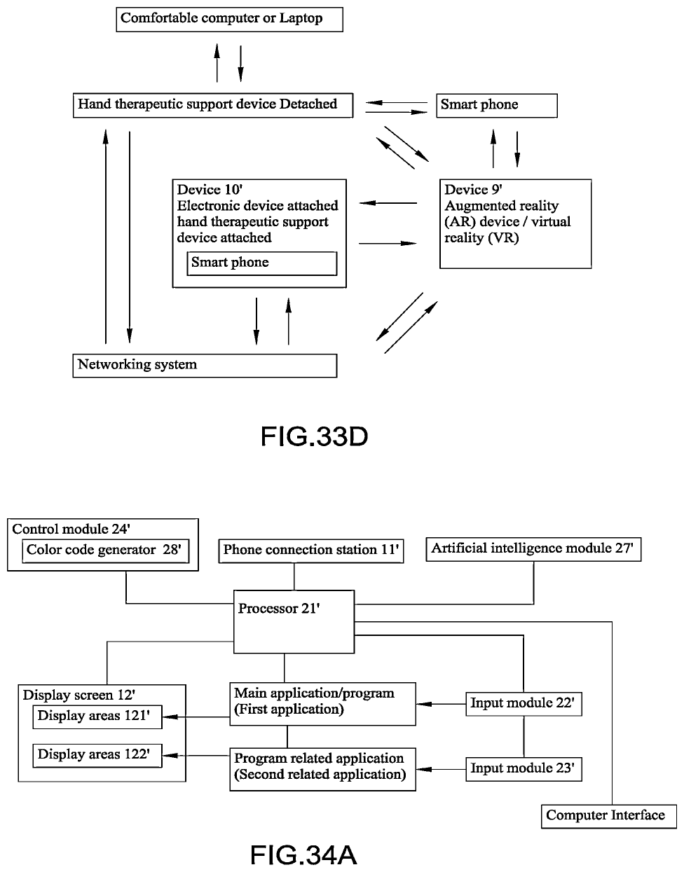

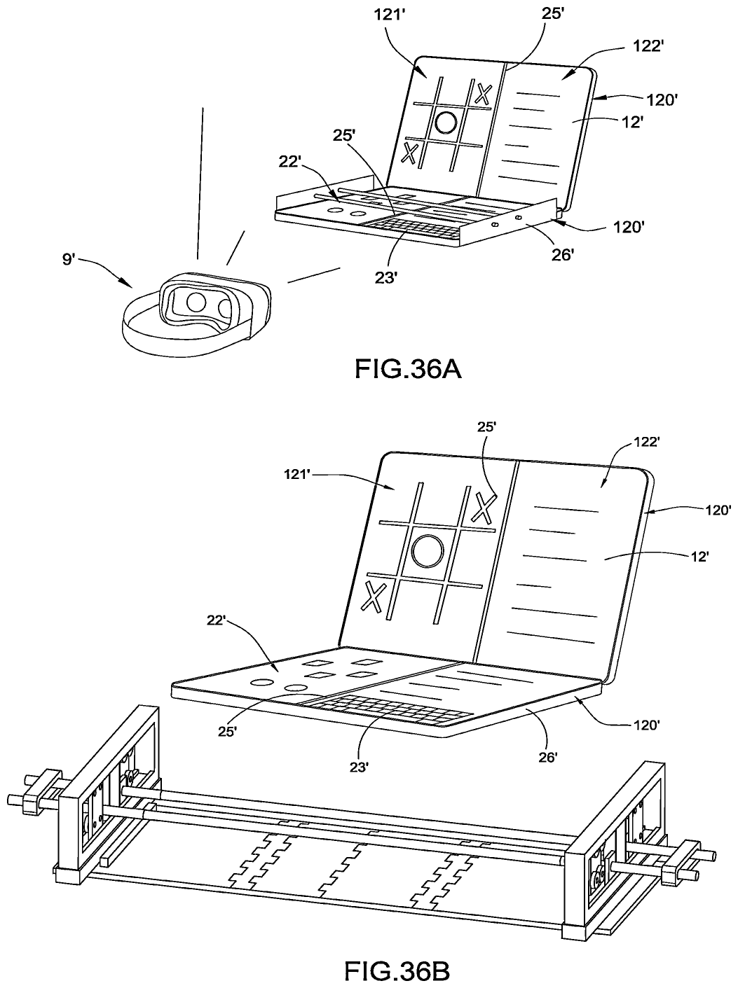

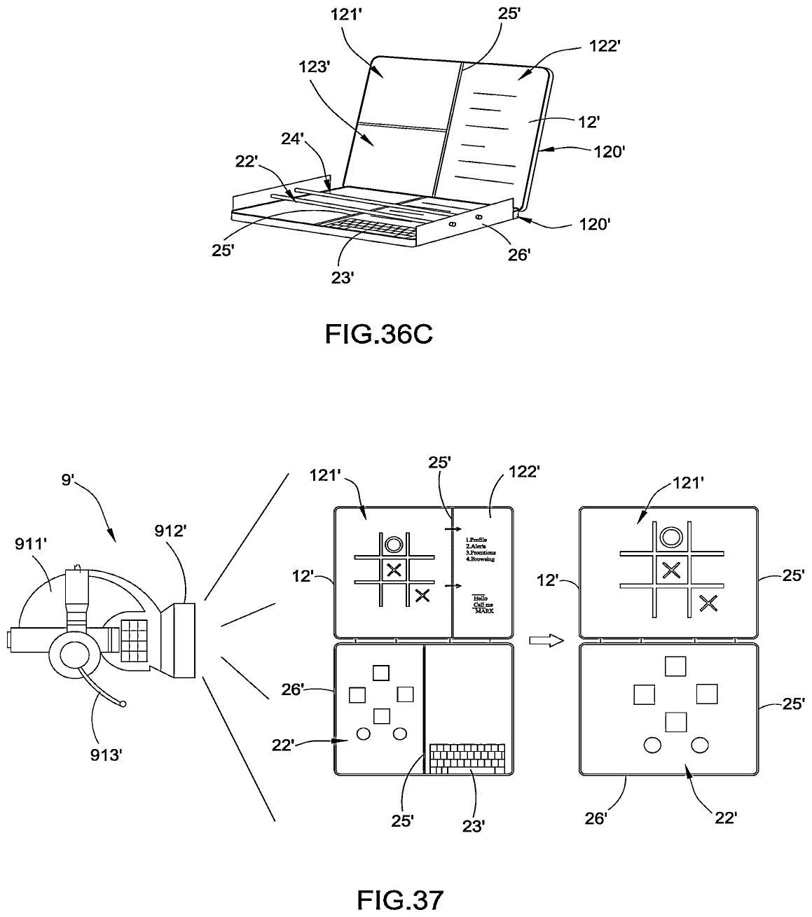

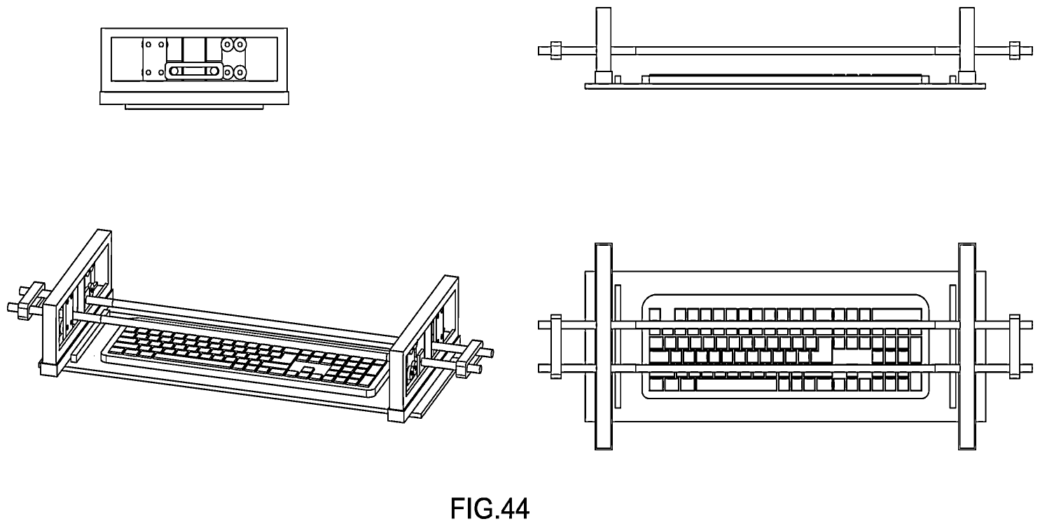

According to the preferred embodiment of the present invention, the electronic device with the detachable or attachable therapeutic hand support device 10′ as described in the above preferred embodiments can be a notebook type electronic device with a detachable or attachable hand therapeutic support device as illustrated in FIG. 33, FIG. 33A and FIG. 44, an augmented reality and virtual reality (AR/VR) device 9′ as illustrated in FIGS. 33 and 33C, a tablet type electronic device as illustrated in FIG. 35, an innovative electronic device with a detachable or attachable hand therapeutic support device as illustrated in FIGS. 36A to 36C, and/or any other like electronic device that can be operated with a sideline program to form and display a slidable sideline on the display screen and/or the control panel of the electronic device.

It is important to mention that FIG. 33A and FIG. 44 illustrate a detachable therapeutic hand support device 10′, with all the same embodiments of the electronic device with the attachable hand therapeutic device 10′, as shown in FIG. 33. Where all the functions and futures are physically located on the detachable hand therapeutic support devices to interface (wireless or wire) with compatible atypical electronic computing devices. As a means, to provide the present invention to and existing computerize market, and not just an all in one new attachable electronic device. In other words, part of the invention is to incorporated most of the current existing electronic devices, like lap tops, (AR/VR) devices, computer monitors, slot machines, table electronic machines, kiosk machines, and etc., where the hand therapeutic support device can interface and link some or all of the functions of the present invention to the existing computer industry.

According to the preferred embodiment of the present invention, when the game to play is a gambling game such as card game, the central control 40′ remotely linked with the individual electronic devices with the detachable or attachable therapeutic hand support device 10′, as described in the above preferred embodiments, to form the virtual dealer to deal and shuffle a predetermined deck of playing cards for the players to play the game through the networking system 20′. Accordingly, the players will play the game through the networking system 20′ as if they are playing the real table game.

Therefore, the players or users are able to remotely connect the notebook type or tablet type individual electronic device with the detachable or attachable therapeutic hand support device 10′ and/or the AR/VR device 9′ to the remote connecting module 31′ for communicatively accessing the communication network 30′ and for playing the games or processing the softwares or applications that the communication network 30′ provided in an authorized manner. The communication network 30 can be any networks, such as a computer network, International Mobile Telecommunication network, other private internal network, and etc., so that through connecting to the designated communication network 30′, the players or users are able to play the games or process the softwares or applications in a remote manner.

The arrangement preferably further comprises a chatting module 43′ linked to the networking system 20′ for enabling the players or users to chat with each other through the networking system 20′ in a real time manner through video, image, audio, and/or text communication via the electronic devices with the detachable or attachable therapeutic hand support device 10′ as described in the above preferred embodiments through the networking system 20′. The networking system 20′ forms not only a playing or processing channel for the players or users to play the game or process the software or application but also a communication channel for the players or users to chat so as to enhance the entertainment of the game or the processing of the software or application, such that the players or users are able to remotely and simultaneously play the same game or process the same software or application and chat with each other via the notebook type or tablet type individual electronic devices with the detachable or attachable therapeutic hand support device 10′ and/or in the AR/VR devices 9′ through the networking system 20′.

It is worth mentioning that the player or user is able to search other players through the chatting module 43′. For example, the player or user is able to input the player or username in the electronic device with the detachable or attachable therapeutic hand support device 10 (including for example the AR/VR device 9′) for searching a particular player or user. Likewise, the player or user is able to search different players or users within a predetermined radial range of the electronic device with the detachable or attachable therapeutic hand support device 10′. The player or user is able to set a private group for one or more designated players or users to join or a public group for any player or user to join.

According to the preferred embodiment of the present invention, the step (b) preferably further comprises a step of providing a virtual dealer to deal and shuffle a predetermined deck of playing cards for the players to play the game through the networking system 20′ when the game is a card game.

The method of the present invention may further comprise a step of activating the notebook type or tablet type individual electronic devices with the detachable or attachable therapeutic hand support device 10′ as described in the above preferred embodiments and/or the AR/VR devices 9′ by the players or users to communicate between the players or users through the communication network 30, so that after the individual electronic device with the detachable or attachable therapeutic hand support device 10′ as described in the above preferred embodiments is being activated by the player or user in the authorized manner, the player or user is able to monitor the selected game or the selected software or application on a display screen of the individual electronic device 10′ and to controllably interact with the game, such as entering a common or betting for gambling type games, or the softwares or applications.

According to the preferred embodiment of the present invention, the central control 40′ further comprises a verification module 44′ for verifying the player or user to play the game or to process the software or application via the individual electronic device with the detachable or attachable therapeutic hand support device 10′ as described in the above preferred embodiments in the authorized manner, wherein each of the players or users is able to sign in the networking system 20′ to participate the games or the softwares or applications and to sign out the networking system 20′ to quit the games or the softwares or applications at any time.

According to the preferred embodiment of the present invention, the verification module 44′ can use the biometric technology, such as fingerprint, face recognition, hand geometry, iris recognition, or the like, to verify the person whether he or she is the authorized player or user. Preferably, a photo ID is used at the time when the player or user signs in and signs out the game or the software or application while the date and playing or processing time will be recorded.

Therefore, the step (a) may further comprise a step of verifying the player or user to play the game or to process the software or application via the individual electronic device with the detachable or attachable therapeutic hand support device 10′ as described in the above preferred embodiments in an authorized manner. Each of the players or users may be able to sign in the networking system 20′ to participate the game or the software or application and to sign out the networking system 20′ to quit the game or the software or application at any time, so as to activate the individual electronic devices with the detachable or attachable therapeutic hand support device 10′ in the authorized manner.

Therefore, the networking system 20′ is able to remotely connect two or more real players at the same time for the player competing or playing against other real players or users simultaneously over the communication network 30′. It should be noted that, through the networking system 20′, the players or users have no need to physically go to a predetermined gathering location, such as in a gaming room, or a gaming table, while still being able to enjoy the fun and realistic of playing games or processing softwares or applications with other real players or users.

Accordingly, the surrogate character can be created by the player or user that the surrogate character will be displayed during the playing or processing time. Therefore, other players or users will see the surrogate character instead of the actual image of the player or user. In addition, the username can also be created by the player or user such that the username will be displayed instead of the actual name of the player or user. Therefore, the player or user will be given a player ID and profiled photo ID during the game or the application. Of course, the player or user must use his or her truth person information to register and sign in the central control 40′ in order to play the games or to process the softwares or applications.

A character development module 434′ will be provided for the players or users to create their personalized surrogate character, wherein a plurality of fantastic computerized characters will be provided for the players or users to select in order to enhance the entertainment, processing and/or gambling experience. In addition, the character development module 434′ further includes digital programs to convert drawings and voice speech into text messages or into digital tool characters.

The central control 40′ may further comprises a chatting program of the chatting module 43′ stored thereat, so that when two or more players or users are linked to the communication network 30′ of the networking system 20′ via each of the notebook type or tablet type individual electronic devices with the detachable or attachable therapeutic hand support device 10′ as described in the above preferred embodiments as described in the above preferred embodiments and/or the AR/VR devices 9′, the players or users are further being able to chat and talk to each other while playing the games or processing the softwares or applications over the communication network 30. Therefore, the reality of the virtual games is further enhanced.

According to the preferred embodiment of the present invention, the step (c) of the method further comprises a step of providing a text communication platform 431′ as the chatting module 43′ for the players or users to communicate with each other via text message through the individual electronic devices with the detachable or attachable therapeutic hand support device 10′ as described in the above preferred embodiments, such as notebook, tablet, smart device, and/or AR/VR device, so as to allow the players or users to exchange written messages with each other during playing the games or processing the softwares or applications.

In addition, in order to enhance the reality of chatting with other players or users, the step (c) is preferred to comprise a step of providing a voice communication platform 432′ for the players or users to communicate with each other via voice message through the individual electronic devices with the detachable or attachable therapeutic hand support device 10′ as described in the above preferred embodiments, such as notebook, tablet, smart device, and/or AR/VR device, so as to allow the players or users to talk to each other during playing the games or processing the softwares or applications.

Also, the step (c) is preferred to comprise a step of providing a video communication platform 433′ for the players or users to communicate with each other via image messages through the individual electronic devices with a detachable or attachable therapeutic hand support device 10′ as described in the above preferred embodiments, such as notebook, tablet, smart device and/or AR/VR device, so as to allow the player or users to view each other during playing the games or processing the softwares or applications and to further enhance the reality as of playing the games or processing the softwares or applications face-to-face with other players, users and/or dealers in the real time manner.

Therefore, in order words, the chatting module 43′ preferably comprises the text communication platform 431′, the voice communication platform 432′, and the video communication platform 433′ that optimizes the reality of playing games or processing softwares or applications without physically showing up at the predetermined location, so that the player or user has relatively more freedom to play the games or to process the softwares or applications anywhere anytime.

A step of posting a record of competence in the game or the software or application for each of the players or users at the respective individual electronic device with a detachable or attachable therapeutic hand support device 10′ as described in the above preferred embodiments is preferably provided. The record contains winning/losing records or working records and tax information of each of the players or users in response to the winning/losing records or working records. For example, the remains or totally amount of the cash or gambling chips of each of the players at the same game may be showed on the display screen of each of the individual electronic devices with the detachable or attachable therapeutic hand support device 10′ as described in the above preferred embodiments.

Accordingly, for example, in the entertainment industrial, there are plenty of casinos or gambling rooms have the gambling type games provided for the players playing the game for betting against each other or wagering against the dealer. However, during the peak of gambling, all the table type games in the casino may be full of the players or tourists, so that the casino may face the shortage of game tables or the shortage of dealers. Therefore, the players in the casino may leave with no choose but instead going to play the machine type gambling games. However, the machine type gambling games in the casino, such as slot machines or roulette gambling machines, does not have the function to allow the players to gamble against the other real players.

Therefore, for example, the communication network 30′ is further embodied as a local or private casino communication network 30′ for electrically connecting two or more individual electronic devices with a detachable or attachable therapeutic hand support device 10′ as described in the above preferred embodiments of one or more casino's customers, so that the players of the casino customer are able to remotely link to the communication network 30′ for playing the games remotely through the individual electronic devices with a detachable or attachable therapeutic hand support device 10′ as described in the above preferred embodiments. Thus, the players are able to gamble anywhere and anytime that he or she is able to connect to the casino communication network 30. In other words, the players not only have their own privacy to play the game at their own desired locations but also have communication and interaction, sociability among other players. Preferably, the electronic device with a detachable or attachable therapeutic hand support device 10′ as described in the above preferred embodiments comprises a GPS unit for identifying the location of the electronic device with a detachable or attachable therapeutic hand support device 10′ as described in the above preferred embodiments. Once the electronic device with a detachable or attachable therapeutic hand support device 10′ as described in the above preferred embodiments is out of the casino area or outside of the range of the private casino communication network 30′, the electronic device with the detachable or attachable therapeutic hand support device 10′ will be disabled.

It is important to understand that the disablement of the electronic devices with the detachable or attachable therapeutic hand support device 10′ is a completely disablement of all the functions of all the devices. That means no gaming, no processing, no texting, and no internet is allowed. It is not a partial disablement of a program or set of programs and, furthermore, the disablement will activate a security dormant system that is activated when the electronic device with a detachable or attachable therapeutic hand support device 10′ as described in the above preferred embodiments is removed from working perimeter boundaries and or tamper within its working perimeter. It will also interact with the surrounding environment.

In a case of removal of the electronic device with the detachable or attachable therapeutic hand support device 10′, the electronic device with the detachable or attachable therapeutic hand support device 10′ will stop working the panic system, turn on the GPS coordinates, and be given to law enforcement. In addition, the smart phone on file will be called and also the GPS will be activated to locate the player or user that the cell towers can triangulate the smart phone position and the credit card will be charged additional penalty fee.

If the smart phone is in the phone cavity 111′, the smart phone will not be released by the portable electronic device with the detachable or attachable therapeutic hand support device 10′ even when the corrected password is input. Furthermore, the Internet-Of-Things (IoT) can triangulate the device as to the direction, speed and the location of the electronic device with the detachable or attachable therapeutic hand support device 10′. Lastly, the electronic device with IoT technology equipped will interact with other interactive devices, such as switching on light bulbs, turning on radios or like, announcing the location of the damaged or out-of-place electronic device with the detachable or attachable therapeutic hand support device 10′ by a dedicated (IoT) speaker near an exit or around the establishment. The same will apply to any tampering of the electronic devices within the facility.

Preferably, no electronic device nor the detachable or attachable therapeutic hand support device 10′ can be turn off/on. All interactive electronic devices with the detachable or attachable therapeutic hand support device 10′ will be on stand-by mode at all times, charging or in used. When any of the electronic devices with a detachable or attachable therapeutic hand support device 10′ is outside its charging dock and needs to be charged, the detachable or attachable therapeutic hand support device 10′ will send a high pitch sound and the security features discussed above will take place except for charging the credit card. In other words, all electronic devices including the detachable or attachable therapeutic hand support device 10′ that are completely off are during repairing or being locked away.

It is worth mentioning that the preferred embodiment of the present invention is not to be confused for a temporary partial authorization of a particular function in an electronic device and or in the functions of the AR/VR devices or program applications for these devices. In other words, the portable electronic devices with the detachable or attachable therapeutic hand support device 10′ will be disable completely. The user will not be able to operate any function and, in addition, the dormant security embodiments mentioned before will be activated as part of the total security system. It is important to mention that the disablement of these electronic devices with a detachable or attachable therapeutic hand support device 10′ are not for the management and functions of these electronic devices with a detachable or attachable therapeutic hand support device 10′, but for the purpose of stopping and preventing illegal operation and crime activity in relation to when the electronic device with the detachable or attachable therapeutic hand support device 10′ is provided by the establishment.

In another example, the establishment can also be embodied as an education establishment, such as a tutorial school or college, and the communication network 30′ is further embodied as an educational communication network 30′ for electrically connecting two or more individual electronic devices with the detachable or attachable therapeutic hand support device 10′ of one or more student users thereof, so that the student users are able to remotely link to the communication network 30′ for processing education softwares or applications, such as doing a joint project or homework, remotely through the individual electronic devices with the detachable or attachable therapeutic hand support device 10′. Thus, the student users are able to discuss, communicate, and doing work anywhere and anytime that he or she is able to connect to the educational communication network 30′. In other words, the student users not only have their own privacy to do the work at their own desired locations but also have communication and interaction, sociability among other group students, all while the user is receiving various therapeutic modalities medically or leisurely.

In another example, the establishment can also be embodied as a working establishment, such as a working facility, the communication network 30′ is further embodied as a working communication network 30′ for electrically connecting two or more individual electronic devices with the detachable or attachable therapeutic hand support device 10′ of one or more employed workers thereof, so that the worker users are able to remotely link to the communication network 30′ for processing working softwares or applications, such as doing a joint project or program, remotely through the individual electronic devices with the detachable or attachable therapeutic hand support device 10′. Thus, the worker users are able to discuss, communicate, and doing work anywhere and anytime even they are not at the seats as long as he or she is able to connect to the working communication network 30′. In other words, the worker users not only have their own privacy to do the work at their own desired locations but also have communication and interaction, sociability among other workers or their supervisors. Preferably, each of the electronic devices with the detachable or attachable therapeutic hand support device 10′ comprises a GPS unit for identifying the location of the electronic device 10′. For example, once the electronic device with the detachable or attachable therapeutic hand support device 10′ is located outside the working area or facility or is out of the range of the working communication network 30′, the particular electronic device along with the detachable or attachable therapeutic hand support device will be disabled for security reasons, like military installations etc.

For instance, the players or users may be able to connect the individual electronic devices with the detachable or attachable therapeutic hand support device 10′ to the communication network 30′ of the establishment while staying in the establishment, such as hotel of the casino, school or working facility, while obtaining optional therapeutic modalities through the therapeutic hand support device 10′.

Accordingly, for example, the game players are able to play the casino games to wager against the virtual dealer of game program 42′ installed in the central control 40′ via plugging into a communication plug provided in a hotel room to wirely connect to the communication network 30′ of the casino or wirelessly link to the communication network 30′ of the casino with a provided password. Therefore, the players are able to remotely gamble via playing the virtual games over the communication network 30′ against other real players in a real time manner.

Also, the student users are able to do their homeworks or projects through the designated softwares or applications installed in the central control 40′ of the school via plugging into a communication plug provided in their dormitory or library to wirely connect to the communication network 30′ of the educational facility or wirelessly link to the communication network 30′ of the educational facility with a provided password. Therefore, the student users are able to remotely and jointly work via the softwares or applications over the communication network 30′ with other student users in a real time manner.

Similarly, the worker users are able to do their works or projects through the designated software or application installed in the central control 40′ of the working facility via plugging into a communication plug provided in the work station, meeting room, cafeteria, or etc. to wirely connect to the communication network 30′ of the working facility or wirelessly link to the communication network 30′ of the working facility with a provided password. Therefore, the worker users are able to remotely and jointly work via the softwares or applications over the communication network 30′ with other users in a real time manner.

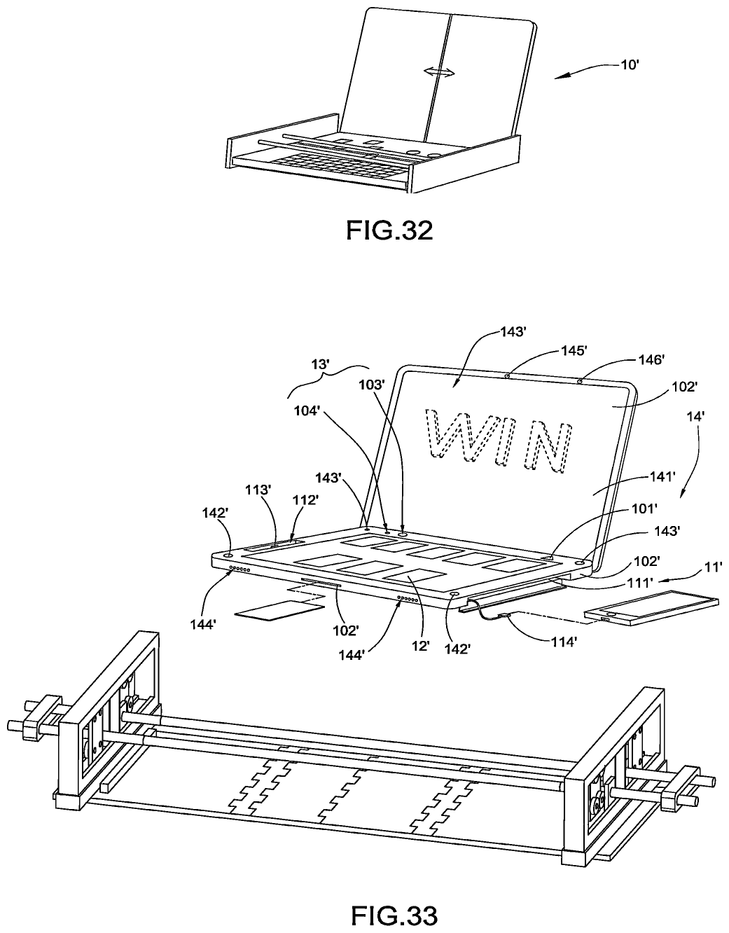

Referring to FIG. 32 of the drawings, the present invention further provides an arrangement of operating multi-task interactive electronic devices for playing games or processing softwares or applications through a networking system 20′ thereof. The arrangement comprises the networking system 20′ which preferably comprises the communication network 30 and the central control 40′ as mentioned above, and the plurality of individual electronic devices with the detachable or attachable therapeutic hand support device 10′, such as a notebook type electronic device as illustrated in FIG. 33, an augmented reality and virtual reality (AR/VR) device 9′ as illustrated in FIGS. 33A and 33B, a tablet type electronic device with the detachable or attachable therapeutic hand support device as illustrated in FIG. 35, an innovative electronic device as illustrated in FIGS. 36A to 36B, and/or any other like electronic device adapted to be operated with a sideline program to form and display a slidable sideline on the display screen and/or the control panel of the electronic device with the detachable or attachable therapeutic hand support device 10′.

As an example, when the present invention is embodied as a method of playing games in a casino or the like as the establishment, the individual electronic devices with the detachable or attachable therapeutic hand support device 10′, such as a personal computer, a notebook, a tablet, an iPad, a smart phone, a portable personal electronic device, an AR/VR device, or any other electronic devices which is able to link to the networking system 20′ wired or wirelessly in the remotely connection manner, wherein the casino players are able to use the individual electronic devices with the detachable or attachable therapeutic hand support device 10′ as the portable game consoles to remotely joint the networking system 20′ for selectively monitoring and interacting with the games displayed on the game consoles with other real gambling players in the real time manner. In other words, each of the individual electronic devices (game consoles) with the detachable or attachable therapeutic hand support device 10′ are programmed to be able to interact with the other paired game consoles with the detachable or attachable therapeutic hand support device 10′ for playing the same game in real time manner.

It is worth mentioning that the casino players are able to select variety games provided in an electronic computing device with the detachable or attachable therapeutic hand support device 10′, so that there is no need for players physically going to each of the gambling table for different types of games. Also, the players do not have to join the crowded gaming tables of the games while being able to enjoy playing with other players, to socialize with other players, and enjoy the entertaining environment in the casino.

The game console (electronic device) and the detachable or attachable therapeutic hand support device 10′ as described in the above preferred embodiments in the casino remotely linked to the networking system 20′ to communicate with the central control 40′ is also programmed that the game console with the detachable or attachable therapeutic hand support device 10′ is able to automatically count the winning or losing of each round of the game, so that the players may be able to collect the credit of winning or losing and cash the credit after the finishing the games. Therefore, the game consoles and the detachable or attachable therapeutic hand support device 10′ are able to enhance the safety of the players by eliminating the need of carrying large amount of chips or cash and also solve the problem of the dealers shortage in casino.

In the preferred embodiment of the present invention, the method of operating multi-task interactive electronic devices for playing games may further comprise a step of providing a plurality of the game consoles as the individual electronic devices with the detachable or attachable therapeutic hand support device 10′ as described in the above preferred embodiments which can be interacting with each other for playing the same game and/or chatting with each other, so that the players of the casino customers are able to operate multiple tasks with their game consoles with the detachable or attachable therapeutic hand support device 10′. For example, the players can enjoy the games simply via the display of their game consoles with the detachable or attachable therapeutic hand support device 10′ to bet, monitor the games, and to remotely play with other real players who's being automatically arranged to the same virtual game room of the game via the central control 40′. Therefore, the players are able to stay in the same seat for selectively playing the games without moving around to different gaming tables of the games. Accordingly, casino will increase value of clients without having to expand casino facility size or increase extra wages for additional employees. By saving construction cost and waging cost, casino will be able to develop better services and to maintain high level entertainment quality.

As mentioned above, the arrangement may further comprise the chatting module 43′ electrically stored in the central control 40′, so that the players are able to play against the other players remotely connected to the networking system 20′ while talking and chatting with each other, so as to enhance the reality as of playing in a real gaming table of game. In other words, the player, who is the casino resort quest, can play game any time anywhere even he or she is not presently stay in the casino resort.

The information storage module 41′ of the central control 40′ may store a plurality of personal information of the players, such as the ID numbers, pictures, phone numbers, names, mail and email addresses, passwords, and/or any activity records regarding the playing of the games, such as gaming dates, winning or losing thereof, and any other activities in casino. The information storage module 41′ may further digitally store the cash flow information of the players, such that the players are able to directly bet against the virtual dealer of the networking system 20′ and to deduct or add the amount of the cash stored in the information storage module 41′ in responsive to the winning or losing of the games.

Accordingly, the information storage module 41′ will store the surrogate character created by each of the players. In addition, the information storage module 41′ will also store the tax information of each of the players. When the player plays each game, the gambling tax will be determined when the player wins the game. In other words, when the player logs out the game, the gambling tax will be determined and recorded in responsive to the gambling winning or losing. The gambling tax will be accumulated for a time period, such as yearly, wherein a gambling tax statement will be printed and sent to each player for tax purpose.

The communication network 30′ of the arrangement is preferably embodied as a private casino communication network 30′ provided for the players of the casino customers accessibly link with the communication network 30′ in the authorized manner for entertaining the customers thereof. The game consoles are electrically and remotely linking to the casino communication network 30′ to form the networking system 20′ for remotely playing games through the game consoles with the detachable or attachable therapeutic hand support device 10′ or any other individual electronic devices with the detachable or attachable therapeutic hand support device 10′, such as AR/VR devices 9′, in the real time manner.

The individual electronic devices with the detachable or attachable therapeutic hand support device 10′ as described in the above preferred embodiments in the preferred embodiment of the arrangement, such as personal computer, notebook, tablet, or AR/VR device loaded with the sideline program as described below, may be able to activate in the hotel room of the casino wirely or wirelessly as mentioned above, so that the players of the casino customers are able to simply relax in their own rooms without physically coming out to the main gambling area of the casino, so as to further provide another way to enjoy gambling and playing games in the casino.

According to the preferred embodiment of the present invention, the players are able to remotely link to the individual electronic devices with the detachable or attachable therapeutic hand support device 10′ as described in the above preferred embodiments via the casino communication network 30′ through the remote connecting module 31′ thereof, wherein the casino may provide a membership card or the like to each of individual players of casino customers, wherein the membership card is preferred to include a magnetic strip or smart chip that stores the corresponding personal information of the customer, so that when the membership card is being inserted into the individual electronic devices with the detachable or attachable therapeutic hand support device 10′, the preloaded sideline program in the electronic device with a detachable or attachable hand therapeutic support device is activated and the communication network 30′ is linking the individual electronic device with the detachable or attachable therapeutic hand support device 10′ to the information storage module 41′ of the central control 40′ automatically, so that after confirming the players information by matching with the stored information in the information storage module 41′ to authorize the activation request from the player, the central control 40′ will automatically and remotely activate the computer program and the sideline program in the electronic device with the detachable or attachable therapeutic hand support device 10′ to remotely link to the networking system 20′. Preferably, the photo ID is required to sign off to the individual electronic device with the detachable or attachable therapeutic hand support device 10′ with date and time recorded. Therefore, the individual storage module 41′ is able to link to the individual electronic devices with the detachable or attachable therapeutic hand support device 10′ for posting the record of the competence in the game for each of the players at the respective individual electronic device with the detachable or attachable therapeutic hand support device 10′.

Alternatively, the individual electronic device (game consoles) with the detachable or attachable therapeutic hand support device 10′ as described in the above preferred embodiments may be activated by providing the predetermined account, the user name and the corresponding passwords thereof, so that the players are able to enter the user names and passwords to submit to the central control 40′ for being authorized to activate the individual electronic devices through the utilization of the detachable or attachable therapeutic hand support device 10′.

Alternatively, the electronic device (game console) with the detachable or attachable therapeutic hand support device 10′ as described in the above preferred embodiments can also be activated by inserting a financial card, such as credit card, debit card, or the likes, so that the private casino communication network 30′ may direct the players to the central control 40′ for accessing the game programs 41′ to remotely play the games through the individual electronic devices with the detachable or attachable therapeutic hand support device 10′ supported by the sideline program and to automatically deduct all charges from playing the games directly from player's financial card.

Alternatively, the individual electronic device (game console) with the detachable or attachable therapeutic hand support device 10′ as described in the above preferred embodiments is provided with input devices, including but not limited to a front/back camera 145′ for image capturing and face recognition or, alternatively, a motion sensor 146 for motion detection, an image (holograph/light) projector 143′ for non-touch interactive display motion and voice command, a microphone 142′ for voice capturing, voice recognition, voice command, and/or a fingerprint scanner for fingerprint recognition, to function as activation tool of the electronic devices as well as the sideline program loaded in the electronic device with the detachable or attachable therapeutic hand support device 10′, so that the individual electronic devices with the detachable or attachable therapeutic hand support device 10′ and the sideline program can be activated by recognizing the player's face, voice and/or fingerprint which is submitted to the central control 40′ for being authorized to activate the individual electronic devices with the detachable or attachable therapeutic hand support device 10′ for accessing the game programs 41′ to remotely play the games through the individual electronic devices, to operate the individual electronic devices with the detachable or attachable therapeutic hand support device 10′ with the supporting sideline program, and to automatically deduct all charges from playing the games directly from the player's final card.

It should be noted that the membership card can also integrate with the room card key of the casino hotel, so that the casino can easily charge the fee of the entertainment of playing game through the room payment account of the players. The membership card can have variety of shapes, such as traditional flat rectangular shaped magnetic card or IC card, or chips shaped having magnetic sensor therein for electrically communicating with the individual electronic devices with the detachable or attachable therapeutic hand support device 10′, in such a manner that the players are able to conveniently activate the individual electronic devices with the detachable or attachable therapeutic hand support device 10′ and saving or transferring a predetermined amount of cash or credit therein without carrying around actual casino gambling chips or cash. It is worth mentioning that the players can also pay or paid through their smart phone application such as “Wallet”, “ApplePay”, “Alipay”, “Bitcoin”, or the like. The casino is able to securely manage the authorization of the players to enhance the security of casino. The information of the player, including credit card information and personal information, will be encrypted and stored, such tokenization, for enhancing the data security.

Therefore, the method of operating multi-task interactive electronic devices for playing games may further comprise a step of authorizing the remotely submitted activation requests by the players via each of the individual electronic devices with the detachable or attachable therapeutic hand support device 10′ as described in the above preferred embodiments. Accordingly, the players are able to securely activate the individual electronic devices with the detachable or attachable therapeutic hand support device 10′ for linking with the communication network 30′, such as the private casino communication network 30′ via the remote connecting module 31′, so as to enhance the safety of the players and the casino. The authorizing of the activation requests of the players may be performed via providing the membership cards, or any of the above mentioned methods to accomplish the authority of the players via the individual electronic devices with the detachable or attachable therapeutic hand support device 10′.

It is worth mentioning that the electronic device with the detachable or attachable therapeutic hand support device 10′, i.e. the portable game console, may be provided in every guest room as standard casino equipment ready to be utilized by the hotel guests of that particular room or requested on demand. For example, the same credit card of the user for the hotel will link to electronic device with a detachable or attachable therapeutic hand support device 10′ as described in the above preferred embodiments for security purpose so as to provide the casino guests the correct financial statement. Criminals will be less likely to stay overnight. It is also important to mention GPS is in every electronic device with the detachable or attachable therapeutic hand support device 10′. This is why GP is so important to the collective operation of the present invention. Another safety feature that is link with the GPS is that the electronic device with the detachable or attachable therapeutic hand support device 10′ will stop all operations when the electronic device is located outside its predetermine intranet working area. It is also worth mentioning when the electronic device with the detachable or attachable therapeutic hand support device 10′ is back in its working territory, the electronic device will not return to operational capacity. The electronic device with the detachable or attachable therapeutic hand support device 10′ must be return to the casino for evaluation and maintenance for a complete total restart-up of all devices. When and where all electrical devices are property of the casino or agents of the casino.

As will be readily appreciated by one skilled in the art, the arrangement of playing game preferably further comprises a private casino communication networking platform for remotely linking with the individual electronic devices with the detachable or attachable therapeutic hand support device 10′ in the authorized manner and for providing an interface for the player managing their private account information and selectively controlling and monitoring the games through the individual electronic devices with the detachable or attachable therapeutic hand support device 10′.

Accordingly, the step (b) of the method of playing games may further comprises a step of providing the private casino communication networking platform for remotely linking the communication network 30′ with the individual electronic devices with the detachable or attachable therapeutic hand support device 10′ as described in the above preferred embodiments and providing a game interface for the players to interact with games and other registered players of the networking system 20′. It is worth to mention that the central control 40′ may further electrically link to any other facilities or service system attached to the casino, so that the players of the casino customers are able to link to the interface of the casino communication networking platform for selectively and remotely placing an order, such as ordering room services, or reserve the restaurant reservation.

Accordingly, each of the individual electronic devices with the detachable or attachable therapeutic hand support device 10′ as described in the above preferred embodiments further comprises a communication key to instantly communicate to the networking system 20′. For example, the communication key can be a service request button arranged in such a manner that when the service request button is pressed, the operator of the central control 40′ will be notified to provide high quality service to the player. Therefore, even the player stays at the hotel room or sits in front of the individual electronic device with the detachable or attachable therapeutic hand support device 10′ in the casino, the player will get the service immediately. The communication key can also be a panic button arranged in such a manner that the operator of the central control 40 will be notified in case of emergency. For example, when other players are cheating during the game, the player is able to notify the operator immediately. Or when the players are in dangerous, such as someone breaking in his hotel room, the operator of the central control 40′ will be notified immediately.

Accordingly, the step (a) of the method may further comprise a step of providing the plurality of game console in the casino as electronic devices with the detachable or attachable therapeutic hand support device 10′, so that the casino is able to provide the total gaming experience to their customers through another way of enjoying therapy and playing games or gambling as mentioned above.

According to the preferred embodiment, the method of the present invention further comprises a step of providing information of the casino including the services and upcoming events such as game competition, room and restaurant reservation, casino schedule, promotion, and etc.

The present invention enables multiple ways of communication in the arrangement, which includes texting, chatting, audio, video, photos, and the like in combination or selective modes without interfering in the flow of the game, among all guests and service personnel of the facility, such as the casino and the hotel or those who mutual accepted to form a communication group. There is an external component for surrounding people not playing with the users and there is an internal component for players playing with the users in the device.

In the external component, there are (1) general and surrounding personal surrogate greeting, (2) personal profile, (3) filter profile system, (4) save match, (5) line of sight, (6) save profile, (7) on and off system of communication of any or all external components. It is important to note that, in the external component, you can only save match and save profile information of other surrounding players, and only in the internal component, the user can communicate live. In save match mode, the computer will tell you when that particular person is able to play with you and in save match mode the user can put a numerical priority pending the other player availability. It is important to note the general personal profile can be of the same nature with the personal profile or they can be totally different. In the save profile, the line of sight and general profiles will be save. To be review now or later with the save profile, one must ask permission to the general profile or the line of sight profile to play together (which is different from save match). And the pictures/photos can be real or of a fantasy in nature.

In the internal components, user will select from novice mode, knowledgeable mode, skillful mode, and expert mode. These modes select the time function for all texting, chatting, pictures, videos, and audio during game time. It is important to note the user can have his or her picture displayed or the surrogate picture in the game to represent his or he physical table location in the game. It is important to note during the game only the players will see each other's selection of communication and all the pictures of the other players in the table. Some may want only using audio while other may only want pictures and text to communicate. And, it is in this window where each other past statistical analysis because this game will be recorded and updated no matter what mode the user in, for future reference.

It is important to note when playing in the skillful mode or expert mode, players can designate the table limits over, for example, 100 dollars and when two or more players want to share each other's general profile or personal profile and still play, they must request each other's approval to change game to novice mode or knowledgeable mode this will aloud for better flow of the game.

The electronic device with the detachable or attachable therapeutic hand support device 10′ as described in the above preferred embodiments of the present invention will only save players that have both accepted to be saved and, in the future, if any of those players is in the same casino, it will automatically inform the user and the save match will let the user prioritize any preference of payers.

According to the preferred embodiment, the arrangement of the present invention can be set or modified to be used and customized for a facility or establishment, such as casino, resort, hotel, business entity, amusement park, university, resort, convention center, cruise, and the like. To a casino, the game will be the gambling game, such as card game. To a business entity, the method and arrangement of the present invention can be used for playing training games or processing softwares or applications for works or projects. To an education establishment such as university, the method and arrangement of the present invention can be used for playing educational games or processing softwares or applications for homeworks, teaching projects, league projects, and etc. To an amusement park, the method and arrangement of the present invention can be used for playing introduction games or processing softwares or applications for tourist guide or conducting sightseeing tour. Person skilled in the art will find easily applying the method and arrangement with the electronic devices disclosed in the present invention to play games or process softwares and applications within a facility or establishment for specific purpose.

The electronic device with the detachable or attachable therapeutic hand support device 10′ as described in the above preferred embodiments is preferred to be a portable electronic device, such as notebook, tablet, console, smart phone, PDA, AR/VR device, and the like, provided by the facility or establishment for the users to individually access respectively, wherein each of the electronic devices with the detachable or attachable therapeutic hand support device 10′ comprises a plurality of different applications preloaded therein, a positioning unit such as a GPS unit and a phone connection station 11 for connecting with an identification means. According to the preferred embodiment of the present invention, the identification means is embodied as a user smart phone of the user. It is appreciated that the identification means can be any other means as described above, including but not limited to ID card, smart chip, memory card, flash drive memory, face recognition means, voice recognition means, fingerprint recognition means, and etc.

The present invention further provides a method of communicatively linking the electronic devices and the detachable or attachable therapeutic hand support device 10′ as described in the above preferred embodiments, they are executed by a computing system, such as electronic computer, quantum computer, liquid computer, server, IoT (internet of things), blockchains, and etc.

It is worth mentioning that blockchains are secure by design and are an example of a distributed computing system with high Byzantine fault tolerance, wherein decentralized consensus has therefore been achieved with a blockchain. This makes blockchains potentially suitable for the recording of events, medical records, video and/or casino games, and other records management activities, such as identity management, documenting provenance, food traceability or voting. In other words, a blockchain is a continuously growing list of records, called blocks, which are linked and secured using cryptography, wherein each block typically contains a cryptographic hash of the previous block, a timestamp and transaction data.

The method of communicatively linking the electronic devices with the detachable or attachable therapeutic hand support device 10′ as described in the above preferred embodiments comprises the following steps:
(1) Remotely link the electronic devices with the detachable or attachable therapeutic hand support device 10′ in a closed communication network 30′, wherein the electronic devices with the detachable or attachable therapeutic hand support device 10′ and the closed communication network 30′ are provided by the facility.
(2) Verify each user for the respective electronic device with the detachable or attachable therapeutic hand support device 10′ in an authorized manner in order to activate the electronic device with the detachable or attachable therapeutic hand support device 10′ by connecting a user smart phone of the user to the electronic device with the detachable or attachable therapeutic hand support device 10′.
(3) Restrict each user to remotely access the electronic device with the detachable or attachable therapeutic hand support device 10′ in limited locations within an area of the facility via the GPS unit, the cell tower and/or the internet of things (IoT) technology built-in with the electronic device with the detachable or attachable therapeutic hand support device 10′ and within a range of the closed communication network 30′.
(4) Connect the electronic devices with the detachable or attachable therapeutic hand support devices 10′ with each other through the closed networking system 20′ that allows the user to selectively communicate with each other and to selectively execute different applications preloaded in the electronic device with the detachable or attachable therapeutic hand support device 10′ through the closed networking system 20′ in a real time manner.

According to the preferred embodiment, the electronic device with the detachable or attachable therapeutic hand support device 10′ as described in the above preferred embodiments is arranged for connecting to the user smart phone. The electronic device with the detachable or attachable therapeutic hand support device 10′ can be connected to the user smart phone wirelessly or by wire. Accordingly, through the connection between the electronic device with the detachable or attachable therapeutic hand support device 10′ and the user smart phone, the record of the competence is sent to the user smart phone when it is authorized by the user. According to the preferred embodiment of the present invention, the electronic device with the detachable or attachable therapeutic hand support device 10′ is wirelessly connected to the user smart phone by means of “Bluetooth”, “NFC”, “WiFi”, LTE, 4G, 5G, ECT, or the closed communication network. It is worth mentioning that when the electronic device with the detachable or attachable therapeutic hand support device 10′ is connected to the user smart phone by wire, the user smart phone is also charged by the electronic device, so as to prevent the user smart phone out of battery. Alternatively, the electronic device with the detachable or attachable therapeutic hand support device 10′ may also provide with a charging pad for electrical charging the user smart phone. For wireless connection, the phone connection station 11′ can be a wireless connection module having a predetermined connection range, such as 10 feet, of the electronic device with the detachable or attachable therapeutic hand support device 10′, wherein the user smart phone should be located within the connection range of the electronic device. When the user smart phone is out of the connection range, the electronic device will disconnect with the user smart phone.

As shown in FIG. 33, the phone connection station 11′ is arranged for holding the user smart phone in position. The phone connection station 11′ can be a storage cavity 111′ that the user smart phone can be received therein. Once the user smart phone is stored in the storage cavity 111′, the electronic device and the detachable or attachable therapeutic hand support device 10′ as described in the above preferred embodiments can be connected to the user smart phone wirelessly or by wire. In one embodiment, the user smart phone is received in the storage cavity 111′ of the phone connection station 11′ and is connected to the electronic device via a connection cable 114′ extended from the storage cavity 111′. Therefore, the user smart phone is charged by the electronic device. In other words, the connection cable 114′ is connected directly to the user smart phone or a charging pad for electrical charging the user smart phone and for activating the electronic device as well as its supporting sideline program. The electronic device with the detachable or attachable therapeutic hand support device 10′ may or may not disconnected and deactivated when the connection cable 114′ is disconnected to the user smart phone. In the secure holding, the electronic device with the detachable or attachable therapeutic hand support device 10′ will activate an alarm system that will not release the storage cavity lock to the smart phone without a correct password. In other words, the user must enter a correct password, such as preset by the user, in order to release the storage cavity smart phone from the electronic device with the detachable or attachable therapeutic hand support device 10′ once the user smart phone is inserted at the storage cavity 111′. It is also important as part of the security system if any of the electric devices is moved out of the facility, the electronic device will also not release the smart phone in the storage cavity 111′ even though the correct password is enter, and furthermore the dormant security system will be activated.

Accordingly, each of the electronic devices with the detachable or attachable therapeutic hand support device 10′ as described in the above preferred embodiments further comprises a phone interfacing module for screen-imaging the user smart phone on the electronic device. When the electronic device with the detachable or attachable therapeutic hand support device 10′ can be connected to the user smart phone, the display screen of the electronic device will interface with the user smart phone. For example, the display screen 12′ of the electronic device will be synchronized to image the screen of the user smart phone, such that the information displayed on the screen of the user smart phone will be displayed on the display screen 12′ of the electronic device. Even though the user smart phone is received in the storage cavity 111′ of the phone connection station 11′, the user is able to view the information to be displayed on the user smart phone through the display screen 12′ of the electronic device or by opening a new window on the display screen 12′ of the electronic device to display the synchronized image of the screen of the user smart phone without removing the user smart phone from the electronic device. It is worth mentioning that the electronic device with the detachable or attachable therapeutic hand support device 10′ cannot obtain or share any personal data from the user smart phone, such that no phone data will be transferred or stored in the electronic device 10′ when it is connected to the electronic device.

It is worth mentioning that the phone connection station 113′ is for the user smart phone and is held at the docking station 112′, wherein a terminal plug 113′ is provided at the docking station 112′, such that when the user smart phone is docked at the docking station 112′, the terminal plug 113′ is inserted into a terminal slot of the user smart phone to connect to the user smart phone to connect with the electronic device with the detachable or attachable therapeutic hand support device 10′ for data transmission. Therefore, the user smart phone can be charged by the electronic device.

In other words, the terminal plug 113′ is connected to the user smart phone for charging the user smart phone and for activation of the sideline program. The electronic device with the detachable or attachable therapeutic hand support device 10′ is disconnected and deactivated when the terminal 113′ is disconnected to the user smart phone. According to the preferred embodiment of the present invention, the electronic device is embodied to be activated and to activate the sideline program by an identification card, such as a membership card, issued by the facility by inserting the identification card into a card reader slot 102′ of the electronic device with the detachable or attachable therapeutic hand support device 10′.

In one embodiment, the closed communication network 30′ is a private casino communication network provided by the casino, such that the electronic devices with the detachable or attachable therapeutic hand support device 10′ as described in the above preferred embodiments are connected with each other through the private casino communication network 30′. The identification card can also be the casino room card, such that the electronic device with the detachable or attachable therapeutic hand support device 10′ and the sideline program in the electronic device are activated by the casino room card. In addition, the electronic device with the detachable or attachable therapeutic hand support device 10′ and its sideline program can also be activated by the user smart phone. The verification module 44′ further comprises a verification application arranged for being downloaded to the user smart phone, such that when the verification application is executed by the user smart phone, the electronic device with the detachable or attachable therapeutic hand support device 10′ and its sideline program are automatically activated. The user is able to input the user' name and hotel room number, as an example, after the verification application is executed by the user smart phone. Then, the verification module 44′ will verify the user when the user smart phone is linked to the networking system 20′ through the mobile communication network or private casino network. For example, the verification application will generate a verification code at the user smart phone, such that when the verification code is sent to the verification module 44′ by the electronic device with a detachable or attachable therapeutic hand support device 10′ through the private casino communication network for verification, the electronic device with a detachable or attachable therapeutic hand support device 10′ will be automatically activated. Likewise, the user is able to input the verification code in the electronic device for activation. It is worth mentioning that since the electronic device with the detachable or attachable therapeutic hand support device 10′ is connected to the user smart phone, the verification code can be directly sent by the electronic device to the verification module 44′ through the private casino communication network for activation. Once the user smart phone is disconnected from the electronic device, the electronic device will be automatically deactivated and locked. Therefore, the player requires re-activation of the electronic device with the detachable or attachable therapeutic hand support device 10′ by the room card, player card and/or the user smart phone, wherein it is worth mentioning that the smart phone application can have all the biometric security features like iris, facial geometry, hand-voice recognition, facial pictures, finger print, and etc.

The electronic device with the detachable or attachable therapeutic hand support device 10′ as described in the above preferred embodiments further comprises a connection indicator 13′ generating a connection signal for indicating a connection between the electronic device with the detachable or attachable therapeutic hand support device 10′ and the user smart phone. In one embodiment, the connection indicator 13′ comprises a light indicator 103′, which comprises at least a LED light, and/or an audio indicator 104′, which comprises at least a speaker 144′, provided on the electronic device with the detachable or attachable therapeutic hand support device 10′, wherein the light indicator 103′ is on for generating a light signal, such as a green light, and/or the audio indicator generates an “On” sound signal, such as a beep sound through the audio indicator 104′, when there is the connection between the electronic device with the detachable or attachable therapeutic hand support device 10′ and the user smart phone. Therefore, the player is notified that the user smart phone is received in the storage cavity 111′ of the phone connection station 11′. In other words, the connection indicator 13′ will generate the notifying signal that also indicates the presence of the user smart phone in the phone connection station 11′.

It is worth mentioning that the audio indicator 104′ of the electronic device with the detachable or attachable therapeutic hand support device 10′ can be activated for additional notification and voice command from the facility, playing music, voice communication with the employees of the facility, other users, or sound information during the playing of the games or processing of the softwares or applications.

When the electronic device with the detachable or attachable therapeutic hand support device 10′ is disconnected from the user smart phone, the light indicator will be off and the audio indicator will generate an “Off” sound signal, such as two beep sounds. In other words, once the user smart phone is removed in the storage cavity 111′ of the phone connection station 11′ by unplugging the connection cable, the light indicator will be off and the audio indicator will generate the “Off” sound signal.

Softwares and/or applications of games, functions and different amenities are preloaded in the electronic device with the detachable or attachable therapeutic hand support device 10′ as described in the above preferred embodiments, wherein the users are able to acquire different games, functions and services provided by the facility via the applications of amenities. In one example, the amusement park will provide the electronic device with the detachable or attachable therapeutic hand support device 10′ with different applications of amenities for different entertainment attractions, rides, and other events. The user is able to selectively execute the designated application of amenities to reserve a timed ticket for the rides, such that the user is able to spend time elsewhere instead of waiting in line for the rides and events. The user is able to selectively execute the designated application of amenities to make a table reservation of a restaurant in the amusement park and even able to view the menu and to pre-order food from the restaurant in advance. Through the GPS unit, the user is able to view the map in the amusement park and the user location there within to guide the user to go from place to place. The parent user is able to track the location of child in the facility via the GPS unit. In addition, different game applications preloaded in the electronic device 10′ enable the user to play the games with the electronic device with the detachable or attachable therapeutic hand support device 10′, especially during the waiting time or spare time. Parental controls can be set in the electronic device with the detachable or attachable therapeutic hand support device 10′ by the parent users that sets controls for the use of the electronic device with the detachable or attachable therapeutic hand support device 10′ by their children.

When equipment, furniture, instruments, and the detachable or attachable therapeutic hand support device 10′ as described in the above preferred embodiments or other things provided in the facility are functioned with the technology of IoT (Internet-Of-Things), the electronic devices can be preloaded with applications for interacting with and/or controlling such things.

In one embodiment, the casino provides the electronic devices with the detachable or attachable therapeutic hand support device 10′ as described in the above preferred embodiments for the players or users, wherein the applications are different game applications and different applications of amenities preloaded in the electronic devices with the detachable or attachable therapeutic hand support device 10′. Each of the game applications is executed in the electronic devices with the detachable or attachable therapeutic hand support device 10′ for the users playing casino games with each other or against a virtual dealer through the private casino communication network in a real time manner. Each of the applications of amenities is executed in the electronic device with the detachable or attachable therapeutic hand support device 10′ for the users acquiring different services provided by the casino. For example, the user is able to selectively execute the applications of amenities to make a table reservation of a restaurant in the casino. The user is able to set the room configuration in advance, such as switching on the air conditioning system or selectively adjusting the light intensity of the room light before the user enters into the hotel room. It is worth mentioning that since the applications are preloaded in the electronic device with the detachable or attachable therapeutic hand support device 10′ and cannot be deleted in the electronic device with the detachable or attachable therapeutic hand support device 10′ by the user, the user cannot download various applications into the user smart phone, selected by the establishment. When the user checks out the hotel casino, the corresponding electronic device with the detachable or attachable therapeutic hand support device 10′ will be returned back to the hotel casino. The user verification information will be erased in the electronic device with the detachable or attachable therapeutic hand support device 10′, such as deactivated by the user smart phone and/or factory reset by the casino, the user will no longer able to control the room configuration or play the casino game by the user smart phone.

The software operation system and firmware of the electronic device with the detachable or attachable therapeutic hand support device 10′ as described in the above preferred embodiments are automatically updated by the facility. In one embodiment, the casino will update the hardware and software of the electronic devices with the detachable or attachable therapeutic hand support device 10′. For example, the casino/agents/contractors of the casino will update the game applications and the applications of amenities in the electronic devices with the detachable or attachable therapeutic hand support device 10′, such that any new game or new amenities will be updated for the users.

The game application can be configured by the user to set the game expense through an option setting of “playing budget”. For example, the user is able set $1000 as the limitation option for playing the casino games through the electronic device with the detachable or attachable therapeutic hand support device 10′. When the user loses close the limitation, the electronic device with the detachable or attachable therapeutic hand support device 10′ will notify the user, such as through the light indicator and/or audio indicator. It is an option for the user to stop playing the game when exceeding the limitation unless the limitation option is disabled by the user.

It is worth mentioning that the user may charge a predetermined amount of money to the electronic device with the detachable or attachable therapeutic hand support device 10′ logged in by the user to pay the accommodation expense, game expense and other service expenses through the user smart phone, Bitcoin application, or other payment applications in the user smart phone.

As shown in FIG. 33, the electronic device with the detachable or attachable therapeutic hand support device 10′ as described in the above preferred embodiments further comprises a graphical display 14′ for the user to play games. The graphical display 14′ comprises a LED or LCD display panel 141′ foldably coupled on the electronic device 10′ and the holograph/light projector 142′ provided on the electronic device for a holograph or a light projection. Alternatively, the display panel 141′ can also be embodied as a projector screen and the graphical display 14′ further comprises an image projector 143′ for projecting an image on the display panel 141′. Preferably, the image projector 143′ is a 3D hologram/light projector for holograph generation on the display panel 141′.

It is worth mentioning that the display screen 12′ of the electronic device with the detachable or attachable therapeutic hand support device 10′ is a touch screen that the user is able to play any game on the display screen 12′. Therefore, the electronic device with the detachable or attachable therapeutic hand support device 10′ will provide a holographic display when the user plays the game and/or chat with other users through the electronic device with the detachable or attachable therapeutic hand support device 10′. It is an option that the game would be projected on the display panel 141′ in a 3D holographic manner so as to enhance the excitement of the game. For example, when playing a card game, the cards can be digitally displayed on both of the display screen 12′ and the display panel 141′. For privacy purpose, the user is able to select the cards to be displayed on the display screen 12′ only. Other displays, such as text messages, game result, and/or other users' surrogate characters, can be displayed on the display panel 141′. For example, when the user wins the card game, a message of “win” will be displayed on the display panel 141′. It is worth mentioning that the message displayed on the display panel 141′ is the holography.

It is worth mentioning that the graphical display 14′ of the electronic device with the detachable or attachable therapeutic hand support device 10′ can also use another tough screen type display screen to substitute and provide images of the games or applications or function as control console of the electronic device with the detachable or attachable therapeutic hand support device 10′. In addition, holograph can be displayed by the image projector (holograph/light) projector 143′ on its own without the user of a back surface, wherein the light projection style depends on the display panel 141′. Both types can be set to interact with the user for the control of the game, program or device through voice command or motion detection through the microphone 142′ or the motion sensor 146′ respectively and vice versa. The hologram or light projection by the image (holograph/light) projector 143′ can be used for alerts and interacting too. For example, the incoming caller can be displayed on the display screen 141′ and the callers callsign can be a picture, surrogate, avatar, and/or emoji that can be a light projection or a holograph projection and vice versa.

In one embodiment, referring to FIG. 33B, an augmented reality and virtual reality (AR/VR) device 9′ is used as the electronic device with the detachable or attachable therapeutic hand support device 10′ as described in the above preferred embodiments of the present invention, wherein the electronic device with the detachable or attachable therapeutic hand support device 10′, including both display screen 12′ and the control panel 141′ as shown in FIG. 33, is a simulated image projected from the AR/VR device 9′. The AR/VR device 9′ comprises at least a headset 911′ for wearing on the head of the user with speakers 912′ for the user's surround sound hearing, at least a microphone 913′ for receiving voice command, audio reception, voice recognition, and etc. of the user, an augmented reality device 914′ for augmenting image projection, a removal virtual reality device 915′ for providing virtual reality image projection, and a motion sensing device or camera 916′ for detecting motions of the users so that the user may simply operate the display screen 12′ as control panel and the user's motions are detected by the motion sensing device or camera 916′.

In one embodiment, referring to FIG. 33B, the AR/VR device 9′ is interacting with a gaming electronic device with the detachable or attachable therapeutic hand support device 10′ and the player or user. The player can operate the display screen 12′ and the display panel 141′ of the gaming electronic device while interacting with the sideline program to control the game in the AR/VR device 9′. The AR/VR device 9′ provides augmented reality and virtual reality supporting the game electronic device with the detachable or attachable therapeutic hand support device 10′ and motion detection and reception of voice command for operation of the game electronic device with the detachable or attachable therapeutic hand support device 10′. The AR/VR device 9 supported with the sideline program also projects sidelines on the display screen 12′ and/or the display panel 141′ the same as in the physical gaming electronic device with the detachable or attachable therapeutic hand support device 10′, wherein the user is able to command through the use of hands, fingers, pupils, head motion, voice command, and etc. The gaming electronic device with the detachable or attachable therapeutic hand support device 10′, the AR/VR device 9′ supported with the sideline program, and the smart device such as cellular phone, are interacting with each other according to the method and arrangement of operating multi-task interactive electronic devices of the present invention. Furthermore, the electronic device with the detachable or attachable therapeutic hand support device 10′ is also equipped with the sideline program and also functions to aid with posture fatigue while in use and during operations of the (AR/VR) devices as mention before in any typing/control/operational apparatus.

In one embodiment, the AR/VR device 9′ is activated with the same security requirements for the sideline program by the combination of the smart phone and the security system that maintains the same disable function for the security tampering system as mentioned above in accordance with the invention.

In one embodiment, the game program can be seen in the AR/RV eye/head set of the AR/VR device 9′ and the functions of the game are controlled in the gaming electronic device by the sideline program or vice versa. The player can have multiple sidelines operating in the gaming electronic device with the detachable or attachable therapeutic hand support device 10′ as described in the above preferred embodiments as well as in the AR/VR device 9 and vice versa. The sideline program for the gaming electronic device with a detachable or attachable therapeutic hand support device 10′ and the AV/VR device 9′ can both be controlled by voice command. In addition, the motion sensing camera 145′ provided on the front/back side of the display panel 141′ and/or the AR/VR occipital sensor motion camera 916′ can provide the user a 360 degree surrounding motion view of his/her location in the augmented reality and/or virtual reality while the AV/VR device 9′ and the hand support device 10′ are in used.

Also, the gaming electronic device with the detachable or attachable therapeutic hand support device 10′ as described in the above preferred embodiments can provide the users of the AV/VR devices 9′ a surround sound, the ability for the touch screen display to be an additional interactive tool and or the hologram to operate with the input apparatus such as typing apparatus like keyboard or motion sensors 146′. The user is able to select what he or she wants on the display screen 12′ and what he or she wants on the augmented reality and/or virtual reality to include alert, message, game, confreres, amenities, and etc.

In addition, the AR/VR device 9′ and the detachable or attachable hand therapeutic support device 10′ can provide a panic button 901′ just like the gaming electronic device with a detachable or attachable therapeutic hand support device 10′ as described in the above preferred embodiments. In one embodiment, the AV/RV device 9′ and the detachable or attachable therapeutic hand support device 10′ can be activated by the smart phone and/or the gaming electronic device with the detachable or attachable therapeutic hand support device 10′ and vice versa.

In one embodiment, once after the AV/RV device 9′ and or the detachable or attachable therapeutic hand support device 10′ is activated, the AR/VR device 9′ and or the detachable or attachable therapeutic hand support device 10′ can active all other sideline programs in all other gaming electronic devices with the detachable or attachable therapeutic hand support device 10′ without additional step in the sideline program security or may only operate in an one AR/VR device 9′ and one therapeutic hand support device 10′ to one gaming electronic device. The AR/VR device 9′ and or the detachable or attachable therapeutic hand support device 10′ may or may not continue to need the smart phone application authorization. The AR/VR device 9′ and or the detachable or attachable hand therapeutic support device interacting with the hologram and light projection motion controls can provide the user the best entertainment of his/her life. For example, in the AR/VR device 9′, the user can have the holograph/light projection in the gaming electronic device with the detachable or attachable therapeutic hand support device 10′ that provides an additional magical ambience as the user is playing on the gaming electronic device with the detachable or attachable therapeutic hand support device 10′ in the support of the augmented reality and with the portable gaming electronic device with the detachable or attachable therapeutic hand support device 10′ or vice versa.

In another embodiment, as the panic button 901′ is armed on the AR/VR device 9′ and on the detachable or attachable therapeutic hand support device 10′, the hologram and/or the light projection and the gaming electronic device and on the detachable or attachable therapeutic hand support device 10′ are activated to better assist and to identify the location of the user to first responders. It is important to mention that the AR/VR device 9 is one set with multiple functions that the user can detach the virtual reality monitor from the headset allowing the augmented reality controls to activate in addition. The augmented reality visual functions can be turn off allowing the voice command microphone, panic button and the hearing speaker to continue interacting with the electronic device programs with the detachable or attachable therapeutic hand support device 10′.

It is worth mentioning that, through the chatting module 43′, the users are able to chat with each other as a group or individually via the electronic devices with the detachable or attachable therapeutic hand support device 10′ and/or the AR/VR device 9′ through the closed communication network 30′. Also, the user may select his or her status as available or not, such that the user is able accept or reject any chatting request from other users.

As it is mentioned above, the information storage module 41′, which is executed by the computer of the facility, embodied as casino, linked to the electronic devices with the detachable or attachable therapeutic hand support device 10′ for posting a record of competence in the game for each of the users at the respective electronic device with the detachable or attachable therapeutic hand support device 10′ and/or the AR/VR device 9′ and for transferring the record of competence to the user smart phone. For example, the record contains winning/losing records and tax information of each of the users in response to the winning/losing records.

Each of the electronic devices with the detachable or attachable therapeutic hand support device 10′ as described in the above preferred embodiments is preferred to have the panic button 101′ for being activated by the user to notify the facility in case of emergency. It is worth mentioning that the facility can have authorization to open the closed communication network to release the restriction of the access of the electronic devices with the detachable or attachable therapeutic hand support device 10′, such that the users are able to access the electronic devices with the detachable or attachable therapeutic hand support device 10′ through any public communication network. In one embodiment, for example, when the facility is the university, it will allow the students to access the electronic devices with the detachable or attachable therapeutic hand support device 10′ through the public communication to access the library, bookstore, and/or classroom information in the university. In addition, the electronic devices 10 can be sold or rent by the facility to the users.

It is worth mentioning that the electronic device with the detachable or attachable therapeutic hand support device 10′ of the present invention not only serves as a portable game console or processor for the user to play game or to process software or application but also serves as a conference tablet for conference meeting in the facility. Accordingly, the information and/or keynote of the conference meeting will be automatically saved in the electronic device with the detachable or attachable therapeutic hand support device 10′. For example, the conference note will be downloaded in the electronic device with the detachable or attachable therapeutic hand support device 10′. The recorded sound and video will be downloaded in the electronic device with the detachable or attachable therapeutic hand support device 10′. The text messages, voice messages, video messages, and/or other interacting messages for people will be saved in the electronic device with the detachable or attachable therapeutic hand support device 10′. It is worth mentioning that the above information of the conference meeting can also be stored in the “Cloud” storage, such that after the electronic device with the detachable or attachable therapeutic hand support device 10′ is returned back to the facility, the user is able to recall all the information by login in the “Cloud” storage.

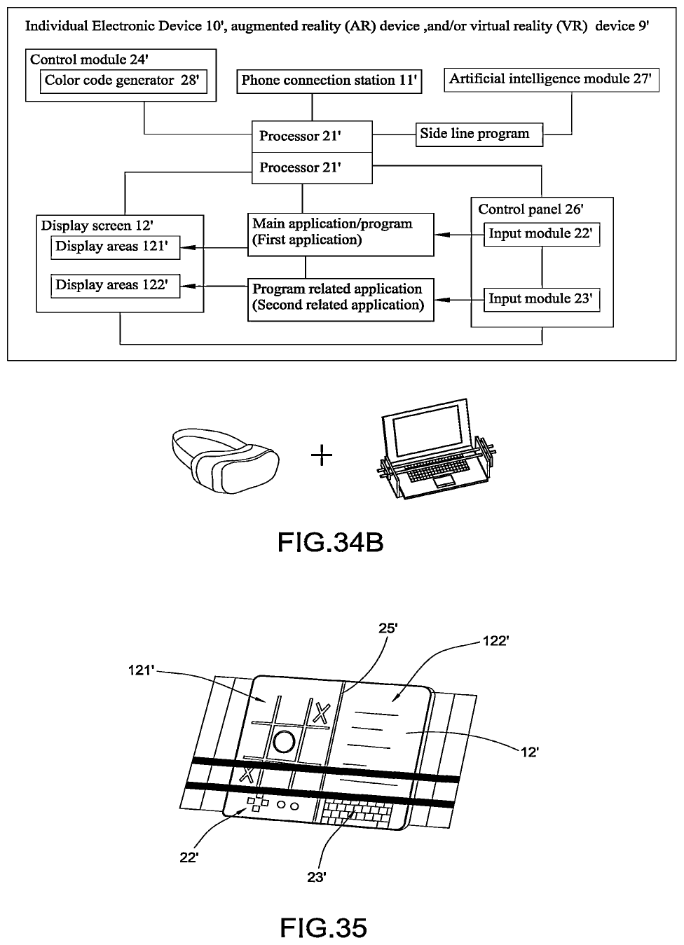

FIG. 34A illustrates a modification of the electronic device with the detachable or attachable therapeutic hand support device 10′ as described in the above preferred embodiments according to the preferred embodiment of the present invention, which is a device for a single touch screen or for multiple touch screen apparatus that is interactive and artificial intelligent that communicates with the operator simultaneously or independent of the games or events being run and without interfering in whatever games or events the operator selects. The electronic device and the detachable or attachable therapeutic hand support device 10′ comprises one or more processors 21′, one or more screen modules operatively linked to the processors 21′, and two or more input modules 22′, 23′ operatively linked to the processors 21′. Accordingly, the one or more processors 21′ are configured to execute two or more applications at the same time.

Before further disclosure of the modifications of the electronic device with the detachable or attachable therapeutic hand support device 10′ as shown in FIG. 34A to FIG. 40, it is appreciated that the electronic device and the detachable or attachable therapeutic hand support device 10′ as illustrated in FIG. 34A to FIG. 40 can substitute the gaming electronic device with the detachable or attachable therapeutic hand support device 10′ as shown in FIGS. 33 to 36D to equip and interact with the AR/VR device 9′. In addition, the controlling and operating of the sidelines 25′ of the sideline program as described below can also be applied and operated in the electronic device with the detachable or attachable therapeutic hand support device 10′ as illustrated in FIGS. 31 to 33D according to the preferred embodiment of the present invention. It is worth mentioning that the sideline(s) 25′ as a tool for the process of this invention in the electronic device with the detachable or attachable therapeutic hand support device 10′ in regards to the sideline program embodied in the AR/VR device 9′. In other words, what the user will observe in the AR/VR device 9′ in accordance with the invention of the sidelines 25′ of the sideline program, with the electronic device with the detachable or attachable therapeutic hand support device 10′. In addition, the sidelines 25′ supported by the sideline program will have the same functions as in the electronic device with the detachable or attachable therapeutic hand support device 10′, in the smart phone application and in the AR/VR device 9′.

Referring to FIGS. 34A to 36C, each of the one or more screen modules has two or more display areas 121′, 122′, dividing by one or more sidelines 25′, for displaying two or more of the applications thereon respectively. Accordingly, the electronic device with the detachable or attachable therapeutic hand support device 10′ as described in the above preferred embodiments comprises one or more display screens 12′. When two or more display screens 12′ are used, two or more of the display areas 121′, 122′ are formed at each of the display screens 12′. When only one display screen 12′ is used, at least two of the display areas 121′, 122′, dividing by one or more sidelines 25′, are shared and formed at the single display screen 12′. The applications are simultaneously controlled by the input modules 22′, 23′ respectively, such that the applications are executed to be displayed on the display areas 121′, 122′ respectively and are independently controlled by the input modules 22′, 23′ at the same time. It is appreciated that the applications are interactively correlated with each other and are configured in one program, such that when the program is run by one of the processors 21′, the applications are simultaneously executed to be displayed on the display areas 121′, 122′ in one of the display screens 12′ respectively. In other words, when the user opens the program, the applications are simultaneously displayed on the display areas 121′, 122′ of the display screen 12′ of the screen module. Accordingly, the input modules 22′, 23′ can be a keyboard, a touch pad, a set of buttons, a voice control input, a joystick, a control console, a 3D camera module, a motion detecting input device, and etc.

According to the preferred embodiment of the present invention, the electronic device with the detachable or attachable therapeutic hand support device 10′ of the present invention further comprises a control module 24′ supported by the sideline program that generates at least one sideline 25′ to be displayed on the display screen 12′ to split or divide the display screen into two of the display areas 121′, 122′ on each sides of the sideline 25′. It is appreciated that the sideline 25′ is a tool that the user can operate to interact and command the functions of the electronic device with the detachable or attachable therapeutic hand support device 10′ (and/or the AR/VR device 9′). In other words, the sideline program with the sideline(s) 25′ generated is the control system to the programs operating in the electronic device with the detachable or attachable therapeutic hand support device 10′ (and/or the AR/VR device 9′)

Referring to FIG. 35 of the drawings, a single quadrilateral display screen 12′ is embodied that, the sideline 25′ is a visual line displayed on the display screen 12′ to split the display screen 12 into two different display areas 121′, 122′. The sideline 25′ can be, generally, a horizontal sideline or a vertical sideline to split the display screen into side-by-side display areas 121′, 122′ or upper-and-lower display areas 121′, 122′. In particular, the sideline 25′ is configured to be movable on the display screen 12 to adjust a size of each of the display areas 121′, 122′ correspondingly, in relation to the electronic device with the detachable or attachable therapeutic hand support device 10′.

It is worth mentioning that when two or more display screens 12′ are used, each of the display screens 12′ is provided with the one or more sideline 25′. In this example one to split that display screen 12′ into two display areas 121′, 122′, such that totally four display areas 121′, 122′ are provided at two display screens 12′. Or, alternatively, if only one of the two display screens is provided with the sideline 25′ to split that display screen 12′ into two display area 121′, 122′ while the other display screen 12′ remains to have one whole display area thereon, so that a total of three display areas will be provided at two display screens 12′, as shown in FIG. 36C.

In addition, two or more sidelines 25′ can also be formed on one display screen 12′ to split the display screen 12′ into three or more display areas 121′, 122′. In other words, two or more margins can be formed on the single display screen 12′ to split the display screen 12′ into three or more display areas 121′ in side by side and/or upper and lower manner. It is important to note that, as shown in FIGS. 35 and 36A-36C, the two display areas 121′, 122′ separated by the sideline 25′ on one display screen 12′ are configured to display the games on one of the display areas 121′, 122′, such as the display area 121′, and the programs on the other display area 122′ correspondingly. The sideline 25′ separates the game programs from other non game programs.

According to the preferred embodiment of the present invention, as shown in FIGS. 34A and 34B, the control module 24′ further comprises a color code generator 28′ that generates different color codes for the sideline 25′ on the display screen 12′ to indicate different program or game selections, i.e. different interactive programs, of the application. For example, the first color code generator 28′ will generate the sideline in red color for the first interactive program and in blue for the second interactive program. Therefore, the user can notify which interactive program he or she has been selected via the color code of the sideline 25′. It is worth mentioning that the color codes can be set by the user via a setting configuration of the control module 24′.

In one embodiment, the electronic device with the detachable or attachable therapeutic hand support device 10′ of the present invention is configured as a tablet computerized device to have a single display screen 12′, such as a touch screen, as shown in FIG. 35. Accordingly, the sideline 25′ is a vertical sideline displayed on the touch screen 12′ to split the touch screen 12′ into two display areas 121′, 122′. Two different applications are executed and displayed on the display areas 121′, 122′. At the same time, two input modules 22′, 23′ are displayed and integrally formed with the touch screen at the display areas 121′, 122′. The two input modules 22′, 23′ can be the same or different depending the control of the application. For example, when the application is a game application, the input module 22′ can be configured as a touch pad displayed and formed on the display area 121′ of the touch screen. When the application is a chatting application, the input module 23′ can be a keyboard displayed and formed on another display area 122′ of the touch screen. The user can simply maintain a finger touching on the sideline 25′ and move leftwards or rightwards, the sideline 25′ is able to be moved to the right or left on the touch screen 12′ to adjust the sizes of the display areas 121′, 122′ correspondingly in respective to the movement of the finger simultaneously. For example, when the sideline 25′ is moved to the left, the left side display area 121′ becomes smaller while the right side display area 122′ becomes larger, vice versa.

According to the preferred embodiment of the present invention, the applications running on the display areas 121′, 122′ respectively are configured to be operated via the input modules 22′, 23′ at the same time. For example, the user is able to play the game via the game application via the touch pad of the input module 22′ by one hand and to chat with other user via the keyboard of the input module 23′ by the other hand, such that the user does not have to switch between the applications to display on the display screen 12′ or between windows opened on the display screen 12′ to show on top of the other windows for operation.

Person skilled in the art would appreciate that tablet type electronic device as shown in FIG. 35 would be a simulated electronic device projected from an AR/VR device 9′ as shown in FIG. 33A. Or, alternatively, the tablet type electronic device with the detachable or attachable therapeutic hand support device 10′ as described in the above preferred embodiments as shown in FIG. 35 would be interacted with an AR/VR device 9′ as described above.

It is worth mentioning that, to the conventional computer, the user may merely operate one application at a time. For example, the user needs to click on the icon shown on the control bar to open that window and then click on that window to activate that window so as to operate the application in that window. Or, the user may open two or more windows to show on the screen with different size at the same time, but the user must first click on the window selected in order to put that window on top of other windows and then click on that front window again to activate that front window so as to operate that activated window. However, according to the preferred embodiment of the present invention, as shown in FIGS. 35 and 36A-36D, the user may use both hands to operate the two or more applications on the two or more display areas 121′, 122′ independently and simultaneously at the same time and more important no other invention can provide both hand support for the purpose of operating both hands at the same time for a single program or multiple programs running at the same time. When more than one input devices are provided with the electronic device with the detachable or attachable therapeutic hand support device 10′ as described in the above preferred embodiments, such as keyboard, touch pad, voice input device such as microphone 142′, and the like, each of the display areas 121′, 122′ will provide an input device selection bar thereon with icons of different input devices, so that the user may simply select the desired input device for operating the application running in that display area 121′, 122′ and make changes of the desired input device anytime. Accordingly, for example, the user may use the touch pad to operate the game running on one of the display area 121′, and use the microphone 142′ and speaker 144′ to operate the chatting application running on the other display area 122′.

In one embodiment, referring to FIG. 34B, the electronic device with the detachable or attachable therapeutic hand support device 10′ as described in the above preferred embodiments further comprises a control panel 26′ foldable coupled at the display screen 12′ to form a laptop-like or notebook type computerized electronic device as shown in FIGS. 36A to 36C, wherein the electronic device illustrated by schematic drawings that merely shows the overall shape of the display the screen 12′ and control panel 26′ without illustrating the detail equipped elements such as storage cavity 111′, microphone 142′, speaker 144′ and so on, as shown in FIG. 33.

Referring to FIGS. 36A to 36C, the input modules 22′, 23′ are provided at the control panel 26′ to independently control the applications displayed on the display areas 121′, 122′ of the display screen 12′. Accordingly, the display screen 12′ can be a touch display screen or a non-touch display screen. There are two or more sidelines 25′ generated on the display screen 12′ and the control panel 26′ respectively. The sideline 25′ formed at the display screen 12′ is a visual sideline to separate the applications displayed on the display areas 121′, 122′ respectively while the sideline 25′ formed at the control panel 26′ is a control sideline to separate the input modules 22′, 23′ thereon.

It is noted that the control panel 26′ can also be a touch screen and the two input modules 22′, 23′ are two display areas shown on the touch screen separated by the control sideline 25′ on the control panel 26′ too. Accordingly, the input devices such as keyboard, input apparatus such as typing apparatus like keyboard, touch pad or virtual images shown on the touch screen type control panel 26′ as the input modules 22′, 23′.

Preferably, one of the input modules 22′ is automatically or manually selected to match with one of the applications correspondingly, such as game application, and one of the input modules 23′ is manually selected by the user to match with other applications.

In one example, the notebook type, tablet type or AR/VR type electronic device with a detachable or attachable therapeutic hand support device 10′ as described in the above preferred embodiments is embodied as a portable game device for the user (player) to play games. The portable game device with a detachable or attachable therapeutic hand support device 10′ as described in the above preferred embodiments for the entertainment industry can provide program functions through the sidelines 25′, wherein the sidelines 25′ is in conjunction and independent to the games or events that the user (operator) is participating to enhance the entertainment experience. In addition, game developers will have the total autonomy to include additional functions in their games or by just utilizing the existing program functions in the sidelines 25′. In particular, the applications include at least a first application, for example a game application, and at least a second related application, for example a game related application. The game related application comprises different sideline programs. It is important that additional functions do not interfere with the sidelines programs because the programs functions in the sidelines 25 are independent of the games or the events being watch or play. It is important to understand the sideline programs are constantly running independent of the games or events running in the display areas 121′, 122′ of the display screen 12′. The sidelines programs are fully operational when wagging or in used in an authorize event. It is also important to understand there is a symbiotic relation between all the sideline commands and the game display area 121′ of the display screen 12′ to provide a smooth flow of the games being played and of the events being watched. For example, the game/event display area 121, 122′ will minimax or enlarge base on the operator sideline positioning. The user can also select multiple games/events viewings being controlled by the program. The types of games played in the game display area 121′ or 122′ may have unlimited futuristic capacities but they need to be approved by the establishment or facility like casino, theme park and etc., and the same is true for events being watched that they need to be approved by the establishment or facility. For examples, games like e-sport, ninja fruit and events like live sport bets, concerts and etc. The programs are especially important when being utilized in a communal settings, no matter if it is an hour event or a 4 years event, it is made for the communication of guest and the participation and enjoyment of the amenities in that establishment or facility.

Referring to FIGS. 36A to 36C, there are two sidelines 25′ provided for every game, application or event, wherein one of the sidelines 25′ is the visual sideline provided on the display screen 12′ to divide it into the two display areas 121′, 122′ to display the game, application or event on the left display area 121′ and the function program on the right display area 122′, and the other sideline 25′ is the control sideline provided on the control panel 26′ to divide it into the two input modules 22′, 23′ for operating the game or event and the function program of the two display area 121′, 122′ respectively at the same time.

It is worth mentioning that the applications of the game or event and the function program displayed on the two display areas 121′, 122′ of the display screen 12′ can be ran by the same processor 21′ of the electronic device with the detachable or attachable therapeutic hand support device 10′ or by more than one processors 21′ of the electronic device on the detachable or attachable therapeutic hand support devices 10′ independently while at least one of the two processors 21′ is configured to operate the positioning of the sidelines 25′ in responsive to the operation and selection of the positions of the sidelines 25 as mentioned above. Of course, the applications can also be ran and controlled by the server computer of the facility with the detachable or attachable hand therapeutic support device, and one or more processors 21′ of the electronic device with the detachable or attachable therapeutic hand support device 10′ is merely provided for communication, setting, computation, operating and controlling the control panel 26′, data transmission between the electronic device with the detachable or attachable therapeutic hand support device 10′ ‘and the server computer of the facility via the networking system 20’, and so on.

Both sidelines 25′, i.e. the visual and control sidelines, are configured to be able to be moved such as by rolling up, down, right, or left for the users' choices according to their own personal selection. Both sidelines 25′ (visual/control) can be configured and preset wide, narrow, short, or enlarge as well as provided with label words and audio. Both sidelines 25′, including the visual and the control sidelines, can be in any setting independent of each other and will continue providing the programmable functions in each corresponding sidelines programmable function, without interfering with the flow of the game or with the less distraction to the game. The program symbiotic relation between the sideline and the game screen on the respective display area 121′, 122′ will automatically give player the biggest game screen viewing field (at all times) base on the user's selection of size of the sideline 25′.

Both sidelines 25′, including the visual and the control sidelines, are configured to have interactive and artificial intelligence. The interactive sideline is color code, with or without audio effect from the speaker 142′, to alert you to the most resent activated program. For example, the players have three program functions in the visual sideline (1) profiles, (2) alerts and (3) promotions. The player may select red for profiles, green for alerts and blue for promotions, so that as the player is playing, the program profile is activate inside the sideline 25′ flashing red and the sideline 25′ itself is also flashing red to indicate that the profile program has a match for the player to see the color code and/or to hear the audio effect. The same is provided for the control sideline too. For example, if the player has already seen a profile and has been communicating in the past and, while he is playing, the control sideline 25′ turns red to indicate the resent text, voice, video or audio message a match profile has been send to the player.

It is also important to note that the inside to the sidelines 25′ can also all be illuminated red in the same example. The interactive sidelines are program functions that do not change even if you change games or watching different events, unless the user edits his or her filter sideline settings to have different results generated by the programs. For example, editing different profiles functions will generate different profile matches. Also, editing a different service functions will generate different food selections, and etc. The same is true for all other program functions, or as many other program functions as the player selected in the sidelines 25′. It is also important to understand the programs can be edit as a onetime change or as a permanent change.

The other interactive function of the sidelines is the audio program for all the programs in the sidelines 25′ and not in the game display area of the display screen 12′ or control screen of the control panel 26′. The way it works is that, as the programs are working, the operator can select any or all programs on or off to be heard as they come on. For example, the user will see a profile color code message on screen in the visual sideline 25′ at the same time the operator will hear it. The operator has the control to hear it louder than the games or events on both cars or on the right or left ear or softer (likewise). The same is true for the control sidelines programs. The operator will also be able to hear the message he or she are sending out or to read the messages shown in the display areas 121′, 122′.

According to the preferred embodiment of the present invention, the electronic device with the detachable or attachable therapeutic hand support device 10′ as described in the above preferred embodiments of the present invention further comprises an artificial intelligence module 27′ running by at least one of the one or more processors 21′ as an intelligent user assistant for assisting the user to operate the sideline program, the first application and the second related application such as player ranking alerts, promotions, browsers, and other services. The artificial intelligence in the sideline 25′ is the program that remembers your choices, likes and dislikes, and activities in the sideline programs, to include pass profile matches. Therefore, if next time you come and a match user was saved from last visit of the sideline programs will notify you and assist you with a record (text, video, or audio) of pass communication to remind the user (operator) and better assist you in communications. It may also remind you of food selection, activities and etc. It is also important to note that an operator may set the interactive and artificial intelligence module 27′ to run automatically or off. The automatic setting saves everything and follows the user's pass choices if there is any history of past activity or learns from the user's current actions for the future. Also, in the automatic settings, the program of the visual sideline 25′ will active the control sideline programs into a symbiotic force reaction to incoming information and will automatically provide the tools to answer in the control sideline 25′ and minimax or enlarge the game/event display area of the display screen 12′. In the manual setting, users must select from novice, skillful, expert or master, and this will enable the operator adequate time.

It is appreciated that the user is always in control of all sidelines 25′. The sidelines 25′ are configured as a function of the program and so are the game being played or events being watched. The user can choice to add another game and play two games at the same time. He or she can determine the presentation of the games by moving the sidelines 25′ and, furthermore, the user can select what game is displayed on top, bottom, right or left, up to twelve games. In other words, users can play up to six games at one time and continue using the program functions provided by the sidelines 25′. The user accomplishes this task by dragging the games and events through the color coded sideline 25′ from the browser inside the sideline 25′. It is important to know that no matter the size of the sidelines 25′, the user selects the program will automatically set the game screen of the display screen 12′ and the control screen of the control panel 26′ to the largest possible size. The user can set fix locations of the sidelines 25′ to fix size to permanently expose all the program functions of the sideline 25′ that he or she wishes to see, while at the same time the program maximizing the game/event screen size at all times.

This is not a picture in picture function to the main screen while this is a complex communicating interactive and artificial intelligence program, that enhances the games, program events, communications, and the entertainment of the events. The control sideline 25′, when manually widen, reveals a (1) keyboard, (2) emojis, (3) panic bottom, and (4) control settings. The control sideline 25′ that can also be fix to size to permanently expose the keyboard and other controls in the control sideline 25′, while at the same time maximizing the control screen size of the control panel 26′.

The program functions are in the sidelines 25′ and these are the tools the device communicates with the players or users, with or without the less interference to the game screen of the display screen 12′. The player or user has the control of which functions he or she wants to use on/off if any or all. For example, some functions in the visual sideline 25′ are (1) ranking of players, (2) alerts, (3) promotions, (4) brows game/pop ups and (5) services. Once again, these programs functions have artificial intelligence. In other words, the electronic device with the detachable or attachable therapeutic hand support device 10′ remembers and selects from past activities but will also remember both sidelines settings, so that the more the user plays or uses, the better the program works for the user. In these programs, the filter controls can also be changed by the players or users to get different selection options from the electronic device with the detachable or attachable therapeutic hand support device 10′ as a onetime selection or as a permanent option.

Person skilled in the art would appreciate that tablet type electronic device with the detachable or attachable therapeutic hand support device 10′ as described in the above preferred embodiments as shown in FIGS. 36A to 36C would be a simulated electronic device projected from an AR/VR device 9′ as shown in FIG. 33. Or, alternatively, the electronic device with the detachable or attachable therapeutic hand support device 10′ as shown in FIGS. 36A to 36C would be interacted with an AR/VR device 9 to control and operate the games, programs and functions as described above. For example, the user can wear the AR/VR device 9′ with the headset 911′, receive sound from the speakers 912′, give voice commands to control and operate the electronic device as well as the sidelines 25′ through the microphone 913′, have augmented reality and/or virtual reality supported for the display screen 12′ and the control panel 26′ of the electronic device with the detachable or attachable therapeutic hand support device 10′ from the AR device 914 and VR device 915′, and/or give motion commands to control and operate the electronic device with the detachable or attachable therapeutic hand support device 10′ as well as the sidelines 25′ through by detecting motions of the user through the motion sensing device or camera 916′.

It is worth mentioning that the sideline 25′ may have two different computer languages operating on opposite sides of the sideline 25′. For example, a computer programmer may select a game engine based on best graphics while another computer programmer may select a different computer language based on the program flexibility. It is appreciated that the sideline program controls are ergonomic human motions optimizing the most speed with the less effort conserving energy for prolong utilization. For example, touch point commands can be demanding in prolong repetition and are seriously near to impossible with muscular, and neurological tremor. It is important to mention that the sideline program is also voice command friendly. The sideline program controls the display of selected pop-ups programs and their location like incoming programs, communications, visual and voice alerts, with the less effort to the user and with the artificial intelligence that the more the user plays, the better the sideline program works for the user.

The ranking of player is a combination of programs that will provide multiple players a communication tool for the games/events that do not have a communication tools platform and will provide additional communication options to the game that have communication tools. In this arrangement, the electronic device with the detachable or attachable therapeutic hand support device 10′ will save match of these profiles for you to rank while in a game/event.

The first tool is a (1) filter survey for player and the other facility guest. In other words, likes, dislikes and personality. (2) The other is a brief surrogate clip video, text, photos, audio, or any combination. (3) The next tool is a general video. (4) The next is a line of sight video this is when the electronic device with the detachable or attachable therapeutic hand support device 10′ has physically been targeted because someone wants to contact you. In all these options, the electronic device with the detachable or attachable therapeutic hand support device 10′ can save, delete and rank the other users after the main user has viewing the profiles. This will provide the electronic device with the detachable or attachable therapeutic hand support device 10′ the ability to assist the user with communications. The program will assist the user to communicate with the user's selected users as they become available in the ranked order the user provided.

Alerts will be set by the user's choice of phone numbers and emails the user wants to take. Emergency notice will be posted to override the game and the nature of the emergency. Promotions will have selected algorithms to generated gifts, coupons, and discounts to better reward the user and will also include advertisement. Services will range from booking restaurant, room services, operator, security, waiter, scheduling theme rides, GPS maps, event tickets, and etc.

In one example, the user is verified for the electronic device with the detachable or attachable therapeutic hand support device 10′ in an authorized manner via the verification code to use the electronic device with the detachable or attachable therapeutic hand support device 10′ as a portable game device within the private communication network, such as private casino communication network. The game application and the game related application are preloaded in the portable game device with the detachable or attachable therapeutic hand support device 10′. Alternatively, the game application and the game related application are saved in a cloud storage that the game application and the game related application can be loaded in the portable game device with the detachable or attachable therapeutic hand support device 10′ through Internet or Intranet. For example, once the portable game device with the detachable or attachable therapeutic hand support device 10′ is wirelessly connected to the communication network, the game application and the game related application will be automatically loaded in the portable game device through the communication network.

It is worth mentioning that the user is able to download the game application and the game related application to his or her personal electronic device, such as a notebook, a tablet, a smart phone, or an AR/VR device, as the portable game device with the detachable or attachable therapeutic hand support device 10′, and connect the personal electronic device to the communication network in order to play. The game application and the game related application are downloaded to the personal electronic device with the detachable or attachable therapeutic hand support device 10′ and its screen is functioned as the display screen 12′ and configured by the downloaded program to form the display areas 121′, 122′ by forming the visual sideline 25′ in the display screen 12′, wherein the game application and the game related application are displayed in the two display areas 121′, 122′ respectively. In addition, the keyboard or mouse pad can be used as the first input module 22′ to control and operate the game application displayed on the display area 121′, and the microphone 142′ or another mouse connected thereto can be used the second input module 23′ to control and operate the game related application displayed on the display area 122′ at the same time.

Likewise, another personal electronic device can be wirelessly linked to the communication network by the electronic device with the detachable or attachable therapeutic hand support device 10′ of the present invention in order to play game, such that the electronic device with the detachable or attachable therapeutic hand support device 10′ of the present invention forms a communication link between the personal electronic device and the communication network.

In view of the electronic device with the detachable or attachable therapeutic hand support device 10′ as described in the above preferred embodiments according to the preferred embodiment of the present invention, as shown in FIGS. 36A-36C, when the game application is loaded and displayed at the display area 121′ of the display screen 12′, the game related application is automatically loaded and displayed at the other display area 122′ of the display screen 12′. The first input module 22′ will be automatically loaded at the control panel 26′. For example, when the user selects to play the “Blackjack” electronic card game, a set of touch buttons are automatically configured at the first input module 22 of the control panel 26′ for the user to select the betting amount, card drawing/holding selections, and etc. At the same time, the user is able to select one of the sideline programs of the game related application, such as chatting platform, intelligent user assistant, ranking, user profile, and etc. When the chatting platform is selected, the virtual keyboard as the second input module 23′ will be automatically selected and displayed on the control panel 26′. Therefore, the user is able to control (play) the electronic card game via the first input module 22′ and to chat with other player users via the second input module 23′ at the same time. It is worth mentioning that the user is able to select different input modules 23′, such as voice input, keyboard, motion detecting input, mouse input, touch pad, touch screen input, holograph control activation, and etc., for the game related application. In addition, other player users who play the same game will be automatically shown in the chatting platform, such that the user is able to chat with other player users at the same time when he or she plays the game.

Accordingly, the phone connection station 11′ is adapted for connecting with the user smart phone. The player verification module 44′ comprises a verification program application to generate a verification code in order to activate the electronic device with the detachable or attachable therapeutic hand support device 10′ and also to execute the user smart phone settings and preferences downloading to the electronic device with the detachable or attachable therapeutic hand support device 10′ automatically. In other words, the smart phone program applications is a set up version for the continue continuity of use on to the portable electronic devices with the detachable or attachable therapeutic hand support device 10′, for the user to use with and without the portable electronic device with the detachable or attachable therapeutic hand support device 10′.

As shown in FIG. 36A, the display screen 12′ is split by the visual sideline 25′ into the two display areas 121′, 122′, such as left display area and right display area. The game application, such as an electronic tic-tac-toe game, is selected and executed on one of the display areas 121′, 122′, for example the left display area 121′. The related game application, including different program selections such as profile, alert, promotions, and browsing, is displayed on the other display areas, i.e. the right display area 122′, for the user to select. At the same time, the first input module 22′ is automatically loaded as a set of directional buttons and selection buttons on the control panel 26′ and the second input module 23′ is automatically loaded as a virtual keyboard on the control panel 26′. It is worth mentioning that the first and second modules 22′, 23′ are separated by the control sideline 25′ on the touch screen type control panel 26′. Therefore, the user is able to play the game via the first input module 22′ by the left hand of the user and to select one of the program selections via the second input module 23′ by the right hand of the user at the same time. The electronic device with the detachable or attachable therapeutic hand support device 10′ is configured to enable user to select the positions of the first input module 22′ and the second input module 23′ that the user is convenient to control and operate the game application and the game related application displayed on the left and right display areas 121′, 122′ of the display screen 12′ respectively at the same time. For example, if the game application is displayed on the left display area 121′, it may be more convenient to set the first input module 22′ configured to control and operate the game application on the left side of the control panel 26′ too while the second input module 23′ is set to position on the right side of the control panel 26′ to control and operate the game related application displayed on the right display area 122′ of the display screen 12′. Of course, it is free for the user to configure according to his or her convenient and desire.

As shown in FIG. 36B, an alternative mode of the above preferred embodiment of the present invention is illustrated, wherein the display screen 12′ is embodied as the bottom touch screen panel and the control panel 26′ is embodied as the upper touch screen panel. Person skilled in the art may already understand that the positions of the display areas 121′, 122′ and the input modules 22′, 23′ are preferably configured to be interchangeable or set according to the desire of the user, especially when both the display screen 12′ and the control panel 26′ are touch screen panels. Some users may prefer to have the game application and its input module 22′ displayed on, for example, the bottom touch screen panel and the game related application and its input module 23′ displayed on, for example, the upper touch screen panel of the electronic device with the detachable or attachable therapeutic hand support device 10′. In other words, the electronic device with the detachable or attachable therapeutic hand support device 10′ is preferably programmed to be capable of interchanging the positions of the applications and their input modules on the screen panels anytime by setting and configuring them.

FIG. 36C illustrates another alternative mode of the above preferred embodiment of the present invention, wherein the display screen 12′ is divided into three display areas 121′, 122′, 123′ by a vertical virtual sideline and a horizontal virtual sideline, while the control panel 26′ is divided into three input modules 22′, 23′, 24′ by a vertical control sideline and a horizontal control sideline 25′ accordingly to control and operate a total of three applications displayed on the three display areas 121′, 122′, 123′ respectively at the same time. As mentioned above, the sidelines 25′ are all configured to be movable by such as rolling upwards, downwards, leftwards, or rightwards to adjust the relative sizes of the display areas 121′, 122′, 123′ and the input modules 22′, 23′, 24′ anytime. In other words, the electronic device with the detachable or attachable therapeutic hand support device 10′ according to the preferred embodiment of the present invention enables the user to select the number of display areas provided on the display screen 12′ and input modules provided on the control panel 26′ by selecting the number and positions of the virtual sidelines 25′ configured on the display screen 12′ and the control sidelines 25′ configured on the control panel 26′.

It is worth to further mention that the surrounding rims of the frame of the display screen 12′ and the control panel 26′ can also be made to form illuminating rims so as to provide lighting effect of different colors as well as flashing with respect to the game(s) playing with the electronic device with the detachable or attachable therapeutic hand support device 10′ to enhance the game effect, indication and entertainment experience. Also, the sound effect from the speaker, the incoming alerts, the sideline programs and the panic button may be corresponding to the lighting effect to strengthen the indication of “ON” status of the electronic device with the detachable or attachable therapeutic hand support device 10′ for enhancing entertainment experience.

As described above, the user is able to move the visual sideline 25′ to adjust the size of each of the display areas 121′, 122′. In particular, the user is able to preset that when the visual sideline 25′ is moved, the control sideline 25′ will be moved correspondingly. Referring to FIG. 37, for example, when the visual sideline 25′ is moved to the right direction, the control sideline 25′ can be preset to be moved correspondingly to the right direction too. Preferably, when the visual sideline 25′ is moved to the one boundary edge of the display screen 12′, the corresponding display area 121′, 122′ will be minimized. For example, when the visual sideline 25′ is moved to the right boundary edge of the display screen 12′, the right display area 122′ will be disappeared, such that only the game application will be displayed on the display screen 12′. Correspondingly, the second input module 23′ will be closed at the same time.

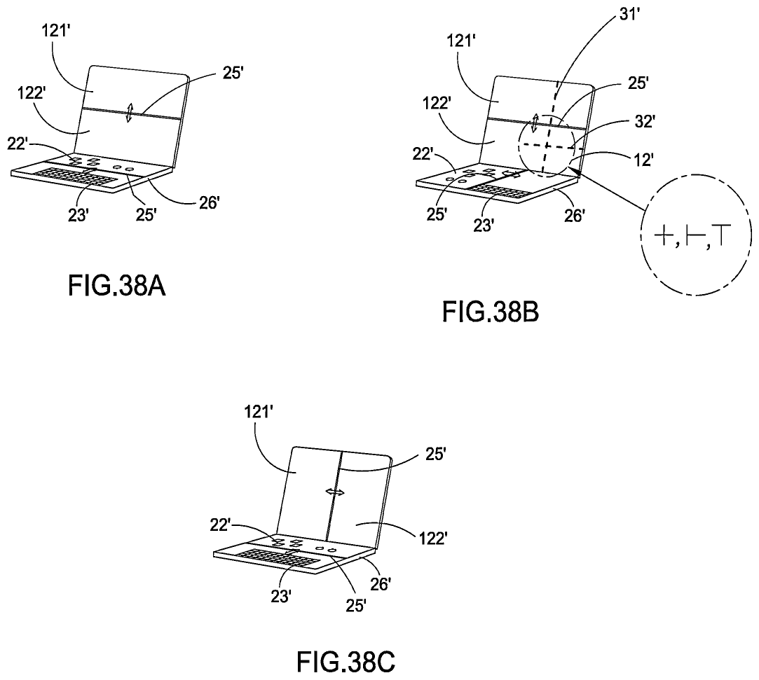

In addition, the visual and control sidelines 25′ can be selectively configured by the user as shown in FIGS. 38A to 38C to either form as a vertical sideline or horizontal sideline. Referring to FIG. 38A, the virtual sideline 25′ is set to be a horizontal sideline on the display screen 12′ to divide into an upper display area 121′ and a lower display area 122′ while the control sideline 25′ is also set to be a horizontal sideline on the control panel 26′ that divides the control panel into an upper input module 22′ and a lower input module 23′, wherein the user may select and set one of the upper and lower input modules 22′, 23′, for example the upper input module 22′, to control and operate the upper display area 121′ and the other input module, for example the lower input module 23′ controls and operates the other display area 122′, vice versus.

FIGS. 38B and 38C illustrate the virtual and control sidelines 25′ can also be configured and set in opposite manner, wherein the electronic device with the detachable or attachable therapeutic hand support device 10′ is illustrated by schematic drawings that merely shows the overall shape of the display screen 12′ and control panel 26′ without illustrating the detail equipped elements such as storage cavity 111′, microphone 142′, speaker 144′ and so on as shown in FIG. 33.

As shown in FIG. 38B, the virtual sideline 25′ on the display screen 12′ is set as a horizontal sideline to divide the display screen 12′ into an upper display area 121′ and a lower display area 122′, and the control sideline 25′ of the control panel 26′ is set a vertical sideline to form a left input module 22′ and a right input module 23′ on the control panel 26′. The user may select one of the left and right input modules 22′, 23′, for example the left input module 22′ to control and operate the upper display area 121′ while the other input module, for example the right input module 23′ to control and operate the lower display area, vice versus. As shown in FIG. 38C, the virtual sideline 25′ on the display screen 12′ is set as a vertical sideline to divide the display screen 12′ into a left display area 121′ and a right display area 122′, and the control sideline 25′ of the control panel 26′ is set a horizontal sideline to form an upper input module 22′ and a lower input module 23′ on the control panel 26′. The user may select one of the upper and lower input modules 22′, 23′, for example the upper input module 22′ to control and operate the left display area 121′ while the other input module, for example the lower input module 23′ to control and operate the right display area, vice versus.

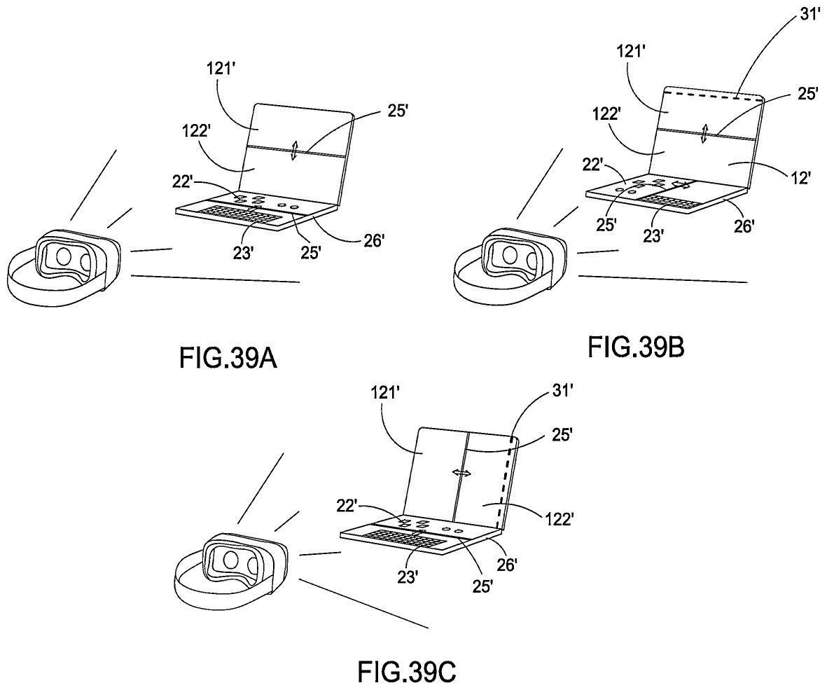

According to the preferred embodiment of the present invention, in order to resetting or repositioning of the sideline 25′ parallelly, as show in FIG. 39A, the user may simply use his or her finger, for example, double clicking or touching and holding for a predetermined period of time on any of the sideline 25′ to activate that sideline 25′ to movable status, and then the user may use a finger to touch and hold that sideline 25′ to slide to the desired position. When the user removes his or her finger from that sideline 25′ at the new position, after a predetermined period of time, such as three seconds, that sideline 25′ will stay at that new position. Similarly, when the control panel 26′ is a touch screen, the parallel resetting or repositioning the sideline 25′ separating the input modules 22′, 23′ on the control panel 26′ can also be changed or reset by the above “T” setting method too.

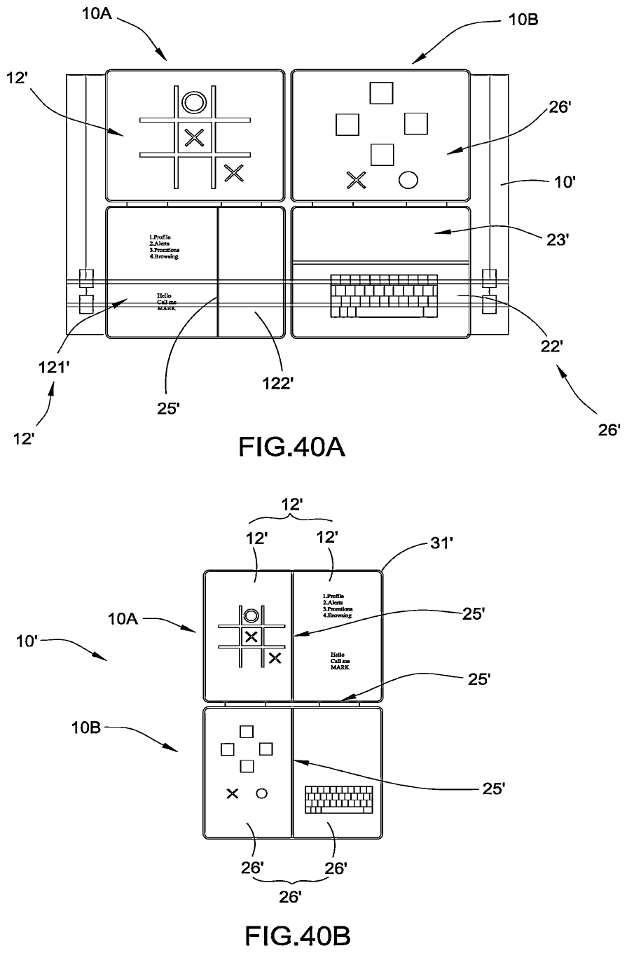

To reset or change the orientation of the sideline 25′, such as from a horizontal sideline to a vertical sideline or from a vertical sideline to a horizontal sideline, there are multiple ways being able to program. For example, the change of the orientation of any desired spot of the sidelines 25′ can be done by a “T” setting method, wherein referring to FIGS. 39B and 39C, the user may simply use his or her finger, for example, to draw a first setting line 31′ from the sideline 25′ and generally perpendicular to the sideline 25′, and then draw second setting line 32′ across the first setting line 31′ and generally parallel to the sideline 25′ to form a “T” setting FIG. 40A. After a certain period of time, the original sideline 25′ disappears and a new sideline 25′ is formed to substitute the original sideline 25′ at the position of the first setting line 31′ and the “T” setting FIG. 30 disappears too. Therefore, the orientation of the sideline 25′ changes perpendicularly.

For example, a horizontal sideline 25′ is reset and changed to a vertical sideline 25′ as shown in FIG. 39B, wherein the first application and second related application displayed on the upper and lower display areas 121′, 122′ originally will be displayed on the left and right display areas 121′, 122′ separated by the new vertical sideline 25′, if the default setting is preset to upper-to-left and lower-to-right format, or alternatively, upper-right and lower-left format. Or, a vertical sideline 25′ is reset and changed to a horizontal sideline 25′, as shown in FIG. 39C, wherein the first application and second related application displayed on the left and right display areas 121′, 122′ originally will be displayed on the upper and lower display areas 121′, 122′ separated by the new horizontal sideline 25′, if the default setting is preset to left-to-upper and right-to-lower format by the user in the electronic device with the detachable or attachable therapeutic hand support device 10′. Of course, alternatively, the default setting preset to right-to-upper and left-to-lower format by the user. Similarly, when the control panel 26′ is a touch screen, the orientation change of the sideline 25′ separating the input modules 22′, 23′ on the control panel 26′ can also be changed or reset by the above “T” setting method too.

FIGS. 39A to 39C illustrate that the electronic device with the detachable or attachable therapeutic hand support device 10′ as shown in FIGS. 38A to 38C can also be interacting with the AR/VR device 9′ as shown in FIG. 33B, wherein the user can wear the AR/VR device 9′ with the headset 911′, receive sound from the speakers 912′, give voice commands to control and operate the electronic device with the detachable or attachable therapeutic hand support device 10′ as well as the sidelines 25′ through the microphone 913′, have augmented reality and/or virtual reality supported for the display screen 12′ and the control panel 26′ of the electronic device with the detachable or attachable therapeutic hand support device 10′ from the AR device 914′ and VR device 915′, and/or give motion commands to control and operate the electronic device with the detachable or attachable therapeutic hand support device 10′ as well as the sidelines 25′ through by detecting motions of the user through the motion sensing device or camera 916′.

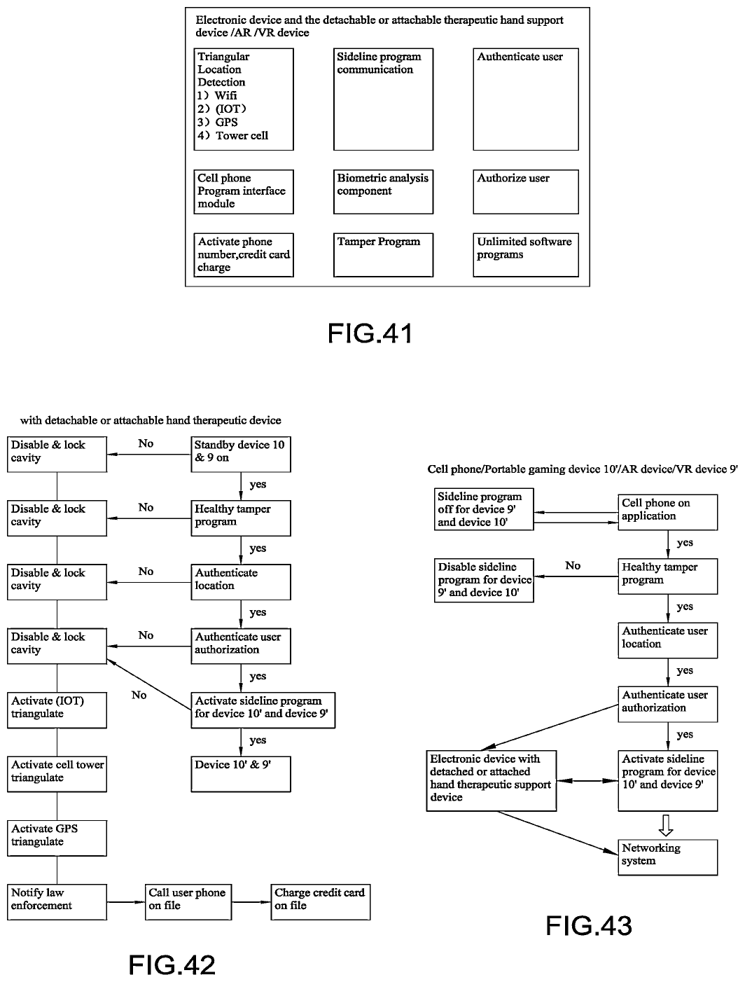

In particular, referring to FIGS. 41-43, the AR/VR device 9′ can be interacts with electronic device with the detachable or attachable therapeutic hand support device 10′ and the players or users. The player may see the display screen 12′ and the control panel 26′ and interact with the sidelines supported by the sideline program in the AR/VR device 9′ for controlling and operating the game in the electronic device with the detachable or attachable therapeutic hand support device 10′. The user of the AR/VR device 9′ is able to slide the sidelines 25′ the same as in the physical portable electronic device with the detachable or attachable therapeutic hand support device 10′. Through the AR/VR device 9′, the user may also be able to command through hand, finger, pupil, head motion, and voice command. The gaming electronic device with the detachable or attachable therapeutic hand support device 10′, sideline program in the AR/VR device 9 and the smart phone. Additionally, the security, artificial intelligence, GPS, cell tow sideline programs and multiple devices communication software and procedures as they interacted with each other forming the arrangement of operating multi-task interactive electronic devices of the present invention.

The AR/VR device 9′ may also replace the physical portable gaming electronic device with the detachable or attachable therapeutic hand support device 10′ by simply projecting an image as a simulated electronic device that also interacts with the sideline program and the smart phone of the user. In one embodiment, the AR/VR device 9′ can be activated with the same security requirements for the sideline program by combination of the smart phone and the security system while maintaining the same disable function for the security tampering system as mentioned above for the physical portable gaming electronic device.

In one embodiment, the game program can be seen in the eye/head set of the AR/RV device 9′ and the functions of the game are controlled in the AR/VR device 9′ by the sideline program or vice versa. The player can have multiple sidelines 25′ operating in the gaming electronic device with the detachable or attachable therapeutic hand support device 10′ and in the AR/VR device 9′ vice versa. The sideline program for the gaming electronic device with the detachable or attachable therapeutic hand support device 10′ and the AR/VR device 9′ can both be activated by voice command. In addition, the motion sensing camera 145′ or 916′ on the back side of the display screen 12′ or on the back posterior occipital region of the AR/VR device 9′ can provide the user a surround motion view of his/her location in the AR/VR device 9′. The gaming electronic device with the detachable or attachable therapeutic hand support device 10′ can provide the AR/VR user surround sound and the ability for the user to also have a 360 view in his/her AR/VR device 9′ through the use of the sideline program while the user is entertaining. The user is able to select what he or she wants to see on the display screen 12′ of the electronic device with the detachable or attachable therapeutic hand support device 10′ and what he or she wants on the AR/VR device 9′ to include alert, message, game, confreres, amenities, and etc. In addition, the AR/VR device 9′ may have the panic button 901′ just like the gaming electronic device with the detachable or attachable therapeutic hand support device 10′.

In one embodiment, the AV/RV device 9′ can be activated by the smart phone and or the gaming electronic device with the detachable or attachable therapeutic hand support device 10′ and vice versa. In another embodiment, the smart phone, portable gaming electronic device with the detachable or attachable therapeutic hand support device 10′ and the AR/VR device 9′ once being activated can up load and down load into other interacting device(s) after the AV/RV device 9′ and other portable gaming electronic devices with the detachable or attachable therapeutic hand support device 10′ as a form of convenience without additional step in the sideline program security or may only be operated in a one AR/VR device 9′ to one gaming electronic device system as a form to added security. The AR/VR device 9′ may or may not continue to need the smart phone application authorization. The AR/VR device 9′ is able to interact with the hologram while the light projection motion controls will provide the user the best entertainment of his/her life. For example, the AR user can have the holograph/light projection in the gaming electronic device with the detachable or attachable therapeutic hand support device 10′ to provide an additional magical ambience as the user is playing on the gaming electronic device in the AR device and, with the portable gaming electronic device, the user can also use the touch screen in the VR device or vice versa. In another embodiment, as the panic button 901 is armed on the AR/VR device 9′, the hologram and light projection on the gaming electronic device with the detachable or attachable therapeutic hand support device 10′ will identify the location of the user to first responders. The AR/VR device 9′ is an all-in-one device that the virtual reality (VR) can be control through the touch screen (display screen 12′ and control panel 26′) of the portable gaming electronic device. The VR device can also be removed and the AR device will activate in addition while the AR device can be turn off. Furthermore, the head set 911′ of the AR/VR device 9′ can continue provide the user a surround sound system and microphone interaction with the electronic portable electronic device with the detachable or attachable therapeutic hand support device 10′.

It is appreciated that the electronic device with the detachable or attachable therapeutic hand support device 10′ as described in the above preferred embodiments according to the present invention is programmed to be able to be used with at least another electronic device with the detachable or attachable therapeutic hand support device 10′ together. Referring to FIGS. 40A-40B, a first electronic device as shown in FIG. 40A and a second electronic device as shown in FIG. 40B are used side by side together and the user is able to configure them into an arrangement and link with the facility through the communication network, wherein four touch screens are present to arrange the display areas and input modules. The user may set the two touch screens of the electronic devices 40A′ as two display screens 12′ and the two touch screens of the electronic devices 10B as two control panels 26′ to control and operate the first application and the second related application displayed on the two display screens 12′ of the electronic device 40A′ respectively. One or more sidelines 25′ are also able to use to separate each of the two display screens 12′ into two or more display areas 121′, 122′ and each of the two control panels 26′ into two or more input modules 22′, 23′ to control and operate the games or applications displayed on the display areas 121′, 122′ respectively.

Referring to FIG. 40B, the first and second electronic devices as shown in FIGS. 40A and 40B can be hinged together to form a single portable electronic device with the detachable or attachable therapeutic hand support device 10′ as described in the above preferred embodiments with two display screens 12′ and two control panels 26′. Accordingly, the two display screens 12′ can be configured to form a combined display screen 12′ and the two control panels 26′ can be configured to form a combined control panel 26′ and the boundary between the two electronic devices with the detachable or attachable therapeutic hand support devices 10A′ and 10B′ is set as sidelines 25′ of the combined display screen 12′ and the combined control panel 26′ respectively.

It is worth mentioning that when the electronic device with the detachable or attachable therapeutic hand support device 10′ is embodied as or interacted with the AV/VR device 9′, the user is preferred to be educated or explained for how the sideline program and the sidelines operated with the AR/VR projections from the AV/VR device 9′ that the user may view in accordance with present invention.

It is worth mentioning that the hand support device or the detachable or attachable therapeutic multi-task support device as illustrated in FIGS. 1 to 44 of the drawings according to the present invention can be permanently or detachably installed on an ergonomic standing and/or sitting up and down table system, such as a standing table converter, sit to stand up desk riser, height adjustable desk and the like. Where the ergonomic table surface can decline or incline independent of the hand support device and where both the table and the hand support device have installed with computer controlling device and corresponding APP or software for settings and functions mentioned in the present invention.

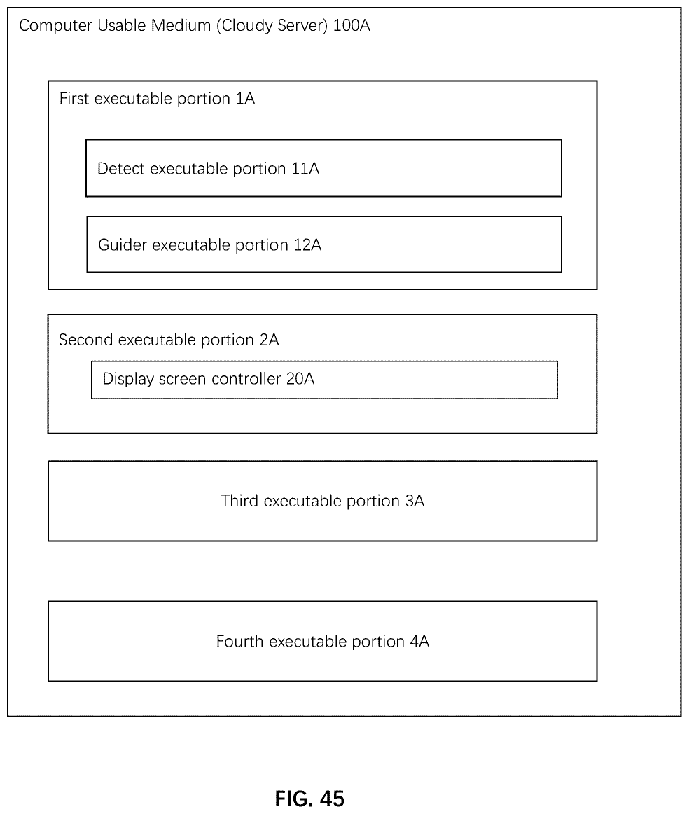

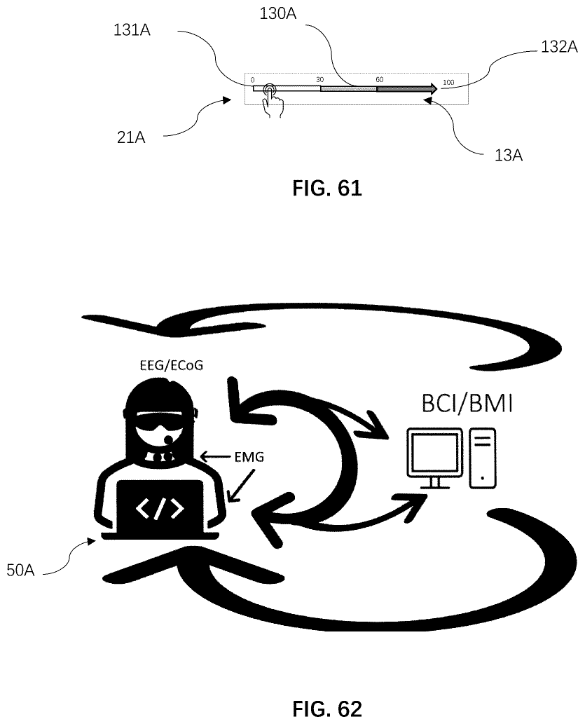

Referring to FIG. 45 to FIG. 74, method and system for displaying sideline operation which input apparatus can be equipped with the multi-task hand support device 10′ as described above are illustrated.

Referring to FIGS. 45 to 47A, a preferred embodiment of the present invention is illustrated. A cloud server comprising at least a computer usable medium 100A is communicatively connected with one or more electronic devices 50A such as, but not limited to, computer, stationary computer, notebook computer, smart device, tablet, and/or VR, MX, XR, or AR apparatus. According to the preferred embodiment of the present invention, the display screen 52A can be a touch screen such as tablet or monitor, a display medium, a projected screen from a projector, a screen of a VR/MR/XR/AR glasses or goggles, or holographic projection, which is supported by programs, camera, light/hologram sensors, projection, hand finger software detection program, and radio motion sensors to detect and response accordingly to hand(s) or finger(s) movement of the user touching on the physical touch screen, or through a virtual display screen, sensors control projections, and/or motion sensors.

The electronic device 50A comprises one or more display screens 52A each configured to be operated by one or more gestures, wherein each gesture produces one or more touch points 521A to operate one or more programs which can be divided by one or more sideline elements 31A. The computer usable medium 100A having computer-readable program code to perform executable operations, wherein the computer-readable program code comprises at least a first executable portion 1A, second executable portion 2A, third executable portion 3A, and a fourth executable portion 4A.

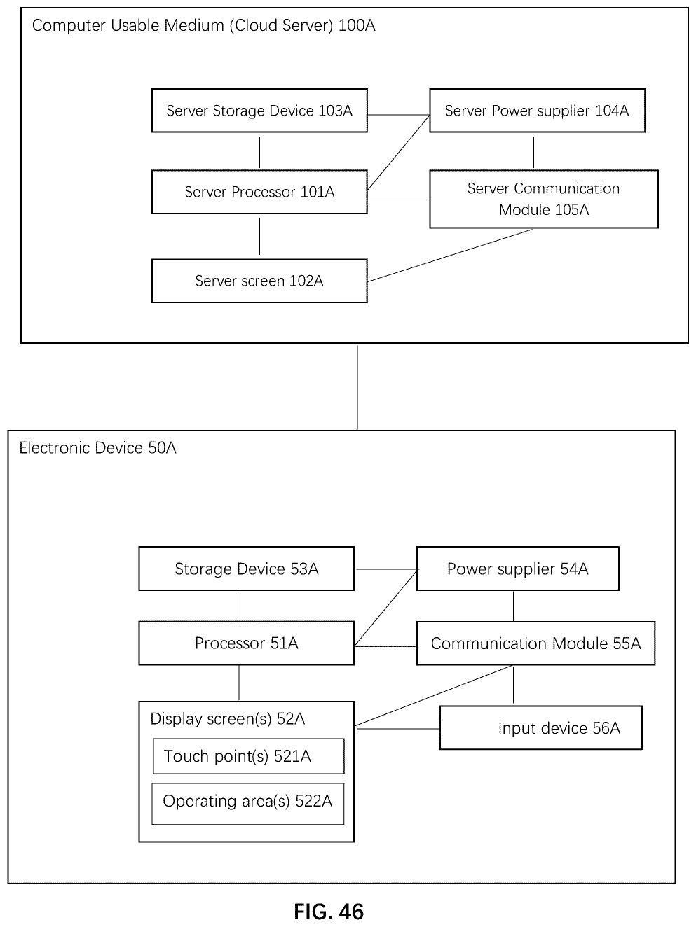

In the preferred embodiment, referring to FIGS. 45 and 46, the electronic device 50A comprises a processor 51A, one or more display screens 52A, a storage device 53A, a communication module 55A, and a power supplier 54A. The power supplier 54A is electronically connected with the processor 51A, the one or more display screens 52A, the storage device 53A, and the communication module 55A commutatively. The power supplier 54A is arranged for supplying electrical power to the process 51A, the one or more display screens 52A, the storage device 53A, and the communication module 55A. The processor 51A is communicatively connected with the one or more display screens 52A, the storage device 53A, the communication module 55A and the power supplier 54A. The processor 51A arranges one or more adjusters 13A configured to selectively display on the one or more display screens 52A, so that the one or more adjusters 13A are able to assist the operation on the one or more display screens 52A. The communication module 55A is communicatively connected with the second executable portion 2A. The one or more display screens 52A detect an operation information such as a touch of a finger of a user thereon, a touch point 521A corresponding to the operation information is detected on the corresponding display screen 52A through the communication module 55A and sent to the second executable portion 2A which comprises a display screen controller 22A configured to analyze the operation information regarding the touch point 521A. Therefore, one or more predetermined touch points 521A configured a gesture of the user on the display screen to control the one or more programs to operate the electronic device 50A.

The communication module 55A is communicatively connected with the one or more display screens 52A and the one or more display screens 52A are interacted with the second executable porting 2A to display and generate corresponding programs as the interfacing and inputting changes from the touch points 521A and are analyzed by the second executable portion 2A based on one or more display adjustment from the first executable portion 1A of the one or more display screens 52A. The one or more display screens 52A are interacted with the second executable portion 2A for analyzing the touch points 521A (gestures) from one or more portions on the one or more display screens 52A. The one or more display screens 52A are interacted with the first executable portion 1A to adjust the displaying contents on one or more displaying portions 523A of the one or more display screens 52A. The one or more display screen 52A are interacted with the third executable portion 3A to provide all the sideline elements 31A available for the user on one or more displaying portions 523A of the one or more display screens 52A.

In other words, for example, the electronic device 50A can be a stationary device such as a desktop computer, slot machine, kiosk, or a portable device such as a notebook computer, a tablet computer, a smart device such as a mobile phone, a PDA, or the like, and etc., that comprises the computer usable medium 100A programmed with the computer-readable program code. Alternatively, the computer usable medium 100A is a cloud server having the computer-readable program code that the electronic device 50A is communicating with the cloud server (computer usable medium) 100A through the communication module 55A of the electronic device 50A wirely or wirelessly through, for example, Internet, cloud servers (private or public), quantum computing, 5G, internet of things (IoT), satellite communication, or other wireless communication network.

It is worth mentioning that the electronic device 50A may or may not have a physical processor 51A, wherein in the event it does not have a physical processor 51A, the server processor in the cloud server 100A will carry out the functions of computing mentioned in this invention.

It is appreciated that the display screen 52A is not limited to a resistance type or capacitance type LCD or LED panel, but also defines any viewable display screen, including but not limited to, projecting screen for projector, virtual screen of virtual reality (VR) headset or goggles, augmented screen of augmented reality (AR) glasses or goggles, mix reality (MR) glasses or goggles, or the likes of extended reality (XR), which is supported by programs and sensors to detect and response accordingly to the hand(s) or finger(s) movement of the user touching with the physical or virtual display screen, or through a virtual display screen, sensor control projections, or motion sensors.

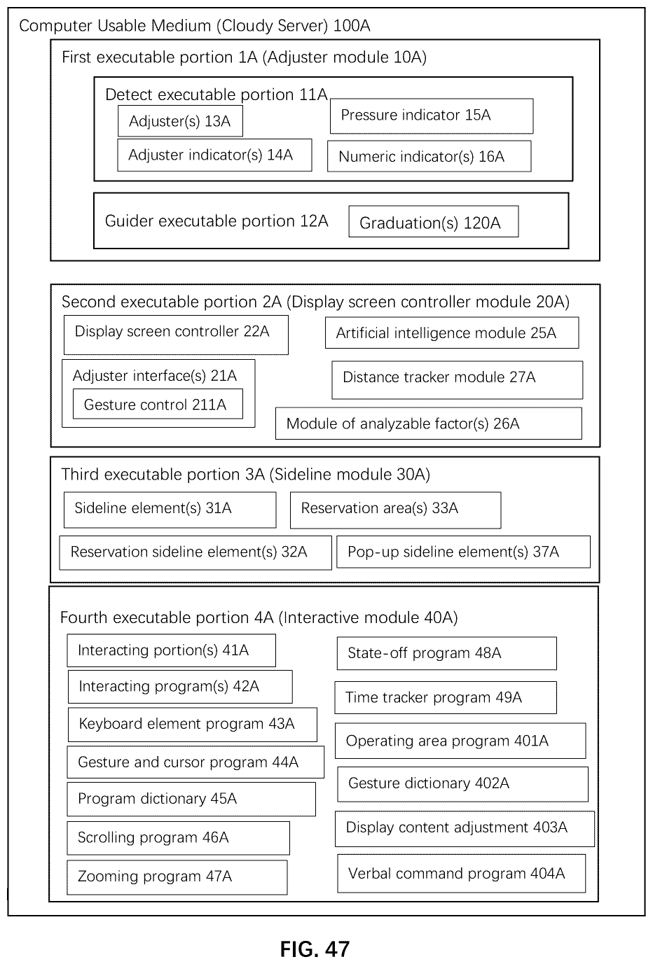

Referring to FIG. 47, the first executable portion 1A is configured for managing the one or more adjusters 13A for providing one or more adjusting operations of one or more displaying contents corresponding to one or more programs on the one or more display screens 52A. The first executable portion 1A is operable to generate one or more adjusters 13A adjusting the one or more displaying contents corresponding to the one or more programs on the one or more display screens 52A, generate one or more adjuster indicators 14A for adjusting the one or more displaying contents corresponding to the one or more programs on the one or more display screens 52A and a pressure indicator 15A at each adjuster indicator 14A on each touch point 521A to illustrate the touching pressure applied the display screen 521A, and allow the one or more touch points 521A to be moved along the one or more adjusters 13A for adjusting the one or more displaying contents corresponding to the one or more programs on the one or more display screens 52A.

The first executable portion 1A comprises a detection executable portion 11A configured to control display adjustments and generate the one or more adjusters 13A according to the operation information received from the second executable portion 2A, the third executable portion 3A, and the fourth executable portion 4A. Furthermore, the first executable portion 1A further comprises a guider executable portion 12A configured to generate one or more graduations 120A with respect to the one or more adjusters 13A respectively. According to each of the graduations 120A, on the corresponding adjuster 13A, the graduation 120A provides a precise guide for controlling the one or more displaying contents, one or more split programs, one or more sideline elements 31A, one or more reservation sideline element 32A, one or more reservation areas 33A, and a time frame of each of the one or more programs on the one or more display screen 52A.

According to the preferred embodiment, the detection executable portion 11A of the first executable portion may also be operable to generate at least a numeric indicator 16A illuminating as a watermark where the value is a percentage of the current viewing page and or a percentage of the hidden area, wherein the user customizes the numeric indicator 16A for size, time, color, location and type of pages or programs. It is worth mentioning that the adjusters 13A and the numeric indicator 16A have time customizable delivery in conjunction to pre-program screen display locations.

The second executable portion 2A is configured for analyzing one or more touch point information, such as module of analyzable factors 26A including location, pressure, thickness, acceleration, time, right or left hand, one hand or both hands, direction, angle, size, interruptions, and etc., and retrieving one or more display screen information from one or more operating areas 522A of the one or more display screens 52A or on one or more displaying portions 523A of the one or more display screens 52A, wherein the one or more adjusters 13A are generated based on the one or more touch point information. The second executable portion 2A is operable to control the one or more adjusters 13A for displaying on the one or more operational areas 522A of the one or more display screens 52A or on the one or more displaying portions 523A of the one or more display screens 52A.

The second executable portion 2A is also configured for analyzing one or more touch point information, including but not limited to module of analyzable factors 26A (location, pressure, thickness, acceleration, time, right or left hand, direction, angle, size, and interruptions) by one or more touch points 521A on the one or more display screens, at the same time or in a timely manner.

In addition, one or more sidelines 31A are generated based on the one or more touch points information, wherein the second executable portion 2A is operable to control the one or more sidelines 31A for dividing on the one or more operational areas 522A of the one or more display screens 52A or the one or more displaying portions 523A of the one or more display screens 52A.

Further, one or more programs are generated based on the one or more touch point information, wherein the second executable portion 2A is operable to control the one or more programs divided by the one or more sidelines 31A, on the one or more operational areas 522A of the one or more display screens 52A or on the one or more displaying portions 523A of the one or more display screens 52A.

The third executable portion 3A is operable to designate one or more sideline elements 31A on the one or more display screens 52A, wherein the sideline element 31A is arranged to divide the corresponding display screen 52A into two or more portions allowing the original displayed program to be displayed and controlled in one portion of the two or more portions and one or more additional programs to be displayed and controlled in another portion of the one or more portions on the respective display screen 52A, wherein if the one or more touch points 521A are positioned at the one or more sideline elements 31A, the one or more adjusters 13A are generated at the one or more sideline elements 31A for controlling movement of the one or more sideline elements 31A. Also, if the one or more touch points 521A are positioned at one or more programs split by the one or more sideline elements 31A, the one or more adjusters 13A are generated for controlling a size of the displaying content on the displaying electronic device 50A and/or within the VR/MR/XR/AR apparatus.

According to the preferred embodiment, the one or more sideline elements 31A generated by the third executable portion 3A is for adjusting one or more reservation areas 33A on the one or more display screens 52A, wherein the one or more reservation areas 33A are configured at one or more edges of the one or more display screens 52A, wherein the one or more sideline elements 31A and the one or more programs of the fourth executable portion 4A are moved from center stage to the edge into the one or more reservation areas 33A while the third executable portion 3A controls the movements of the one or more sideline elements 31A providing the additional displaying area of one or more additional programs.

The third executable portion 3A is also operable to designate one or more programs to be separated with one or more sideline elements 31A on the one or more display screens 52A, wherein each of the one or more sideline elements 31A further divides a surface area of the corresponding display screen 52A into two or more portions for one or more programs displayed respectively, wherein each of the programs displayed in the divided portion of the display screen 52A is referred as a split program. Further, when the one or more touch points are positioned over one or more split programs divided by the sideline element 31A which may be shown on the display screen 52A as a visual boundary, the one or more adjuster interfaces 21A are generated on the one or more split programs to provide the user a percentage of hidden program or a percentage of the current display on the one or more display screens 52A.

According to the preferred embodiment, the one or more interacting portions 41A include a keyboard portion, a pad portion, a mouse portion, a cursor portion, a gesture dictionary portion, a zooming portion, a scrolling portion, an off state element portion, and a gesture operation portion, wherein all the interacting portions 41A and/or interacting element programs 42A of the fourth executable portion 4A are able to switched between each another and with other programmable elements, wherein all the gestures on the gesture dictionary 402A are scalable while the programs for joining the gestures are also scalable. Further, each of the interacting portions 41A is a state-off portion, when a state-off program is triggered, the interacting portion 41A provides gesture operations. When the state-off portion is in a lock option, the state-off has limited gesture options. Further, the interacting programs 42A and the interacting portions 41A can be superimposed by the state-off program 48A, wherein when the state-off program 48A is triggered, the one or more interacting portions 41A and the one or more interacting programs 42A are provided with additional gesture capacity, and that when the state-off program 48A is in the lock option, the state-off program has limited for any gesture option. It is worth mentioning that the superimposed state-off program 48A does not re-adjust the displaying contents.

The fourth executable portion 4A is operable to designate one or more programs on one or more interacting portions 41A on the one or more display screens 52A, wherein the one or more interacting portions 41A interact with the one or more programs displayed on the one or more display screens, wherein when the one or more interacting portions 41A are activated, the one or more interacting portions 41A are superimposed or displayed on the one or more display screens 52A according to one or more positions of the one or more touch points 521A acquired from the second executable portion 2A.

In addition, when the one or more interacting programs 42A are activated, the one or more interaction programs 42A may be superimposed or displayed on the one or more display screens 52A according to one or more positions of the one or more touch points 521A acquired from the second executable portion 2A.

According to the preferred embodiment, the fourth executable portion 4A further provides a gesture dictionary 402A and a program dictionary 45A in combination with the gesture dictionary 402A. The gesture dictionary 402A is arranged for customizing a gesture operation with a program according to the information of the module of analyzable factors 26A for the one or more touch points 521A, wherein each gesture is selected from the gesture dictionary 402A and matched with a selected function of a program or set program function to enable the user to control the respective program by implementing the selected function through the one or more gestures. The program dictionary 45A is arranged in combination with the gesture dictionary 402A for pairing the one or more gestures with a selected program, according to a paired information delivered to the respective display screen 52A for personal and customizing re-production of the gesture, wherein once the gesture is customized, the artificial intelligence module 25A records, learns and delivers the information to the processor 51A or a server processor 101A of the cloud server 100A where the processer delivers the information to the one or more display screens 52A for display. It is worth mentioning that the cloud server may also deliver the information to the one or more display screens 52A for display.

According to the preferred embodiment, the third executable portion 3A generates one or more pop-up sideline elements 37A for providing immediate superimposed alerts and menu options on the one or more reservation areas 33A on the one or more display screens 52A, wherein the one or more pop-up sideline elements 37A and the one or more programs of the fourth executable portion 4A are acquired from in relation to the available menu program options from the fourth executive portion 4, wherein the third executable portion 3A controls the customization of the one or more pop-up sideline elements 37A providing the alerts and computer menu selection displaying while superimposed of one or more additional programs.

In other words, according to a preferred embodiment, the present invention provides a system for gesture operating on one or more interactive portions 41A of one or more display screens 52A of an electronic device, comprising:
the computer usable cloud medium 100A having computer-readable program code to perform executable operations, the computer-readable program code comprising:
the first executable portion 1A for managing the one or more adjusters 13A for providing one or more adjusting operation of the one or more displaying contents corresponding to the one or more programs on the one or more display screens 52A, wherein the first executable portion 1A is operable to:
generate the one or more adjusters 13A for adjusting the one or more displaying contents corresponding to the one or more programs on the one or more display screens 52A; and
generate the one or more adjuster indicators 14A for notifying of the current status of the one or more displaying contents and/or program function corresponding to the one or more programs that require a predetermined function such as zooming on the one or more display screens 52A;
allow the one or more touch points 521A to be moved along one or more adjuster interfaces 21A for adjusting the one or more displaying contents corresponding to the one or more programs on the one or more display screens 52A; and
a second executable portion 2A for analyzing the one or more touch point information, retrieving the one or more display screen information from the one of more display screens 52A, and controlling the one or more adjusters 13A and the adjuster interface 21A generated based on the one or more touch point information for controlling on the one or more display screens 52A.

The first executable portion 1A is configured for analyzing the one or more touch point information, retrieving the one or more touch point information from the one or more display screens 52A and/or from one or more interacting portions 41A, and displaying the one or more adjusters 13A generated based on the one or more touch point information for displaying on the one or more display screens 52A.

According to the preferred embodiment, the first executable portion 1A is further operable to generate the one or more adjusters 13A on display for customizing one or more gestures and the one or more sideline elements 31A that can be dependent on time as a function of the one or more programs on the one or more display screens 52A and allow the one or more touch points 521A to be moved along the one or more adjuster interfaces 21A for adjusting one or more time-dependent contents corresponding to the one or more programs on the one or more display screen 52A. Also, the second executable portion 2A is also operable to allow the one or more touch points 521A to be moved along the one or more adjuster interfaces 21A for controlling the one or more sideline elements 31A corresponding to the one or more programs from the fourth executable portion 4A on the one or more display screens 52A.

According to the preferred embodiment, the second executable portion 2A is operable to analyze the one or more touch points 521A including, but not limited to, a location, a thickness, a pressure, a size, an acceleration, a time, and a direction of each of the one or more touch points 521A. The second executable portion 2A is also operable to analyze whether the one or more touch points 521A on the one or more display screens 52A are operated by a right or left hand of the user, the angle of the touching finger of the user, and interruptions of the touching of the user's finger. The analyzed information is retrieved from the one or more programs on the one or more display screens 52A, wherein the one or more adjuster interfaces 21A are generated base on the information analyzed by the display screen controller 22A and deliver to the first executable portion 1A to display and to adjust the displaying contents 212A with the one or more adjuster interfaces 21A for controlling the size of each of the one or more displaying contents on the one or more display screens 52A from the second executive portion 2A. Also, the information may be recorded and verified by the artificial intelligence 25A and the informational is further analyzed by the display screen controller 22A and delivered to the first executable portion 1A to display and to adjust the displaying contents 212A with the one or more adjuster interfaces 21A for controlling the size and program function of each of the one or more displaying contents on the one or more display screens 52A from the second executive portion 2A.

It is worth mentioning that the artificial intelligence 25A in the controller module 20A is a module configured to learn, store and verify various activities of the one or more touch points 521A from the one or more display screens 52A, the sideline module 30A, the adjuster module 10A, and the interacting module 40A, wherein the gesture actions are anticipated and therefore the artificial intelligence 25A may deliver customary sequential customary information to the display screen control 22A as a form of assistance of the artificial intelligence module 25A.

According to the preferred embodiment, the analyzed information is also retrieved from the one or more sideline elements 31A on the one or more display screens 52A, wherein the one or more adjuster interfaces 21A are generated base on the information analyzed by the display screen controller 22A and delivered to the first executable portion 10A to initiate and adjust the display with the one or more adjuster interfaces 21A for controlling the one or more sideline elements 31A for the controlling movements of the sideline elements 31A corresponding to the one or more programs on the one or more display screens 52A with inputting of one or more touch points 521A.

In addition, the information is retrieved from the one or more display screens 52A to pair the one or more gestures with one of the one or more programs, wherein the one or more adjuster interfaces 21A are generated base on the information from the module of analyzable factors 26A, analyzed by the display screen controller 22A for organization and delivered to the first executable portion 1A to initiate and adjust the display with the one or more adjuster interfaces 21A for controlling, editing, and customizing the time or any of the other module of analyzable factors 26A of the one or more gestures in the process of pairing with different programs.

According to the preferred embodiment, the first executable portion 1A is operable to generate the one or more graduations 120A for indicating a total range in divided levels of the one or more displaying contents respectively, wherein the one or more graduations 120A are attached corresponding to the one or more adjusters 13A, such that the available adjustable state of each of the displaying contents is displayed corresponding to the one or more programs on the one or more display screens 52A.

According to the preferred embodiment, the third executable portion 3A is operable to control splitting of programs of the same language or the different computer languages. The programs can be displayed on the one or more display screens 52A and separated vertically or horizontally, with mirror image, mirror image inversion, axis of symmetry, or non-axis of symmetry. It is important to mention that the first executable portion 1A and the third executable portion 3A work together where the fourth executive 4A organizes multiple programs in the one or more display screens 52A, and provides sideline boundaries for program selection in relation to gesture controls, while the first executable portion 1A provides the customization for the displaying content 523A of the programs and location of each of the one or more sideline elements 31A controlled by the one or more touch points 521A when creating a gesture. The first executable portion 1A sets a location for adjustment and the one or more adjusters 13A for every separated program selected by the user. The information is acquired from the one or more touch points 521A from the second executable portion 2A to provide one or more adjuster interfaces 21A on the one or more display screen 52A for the user to control.

According to the preferred embodiment, the fourth executable portion 4A is operable to receive the one or more touch point information from the second executable portion 2A, wherein the fourth executable portion 4A is operable to pair the touch point information with a computer program. The fourth executable portion 4A is a location where a program is selected from a program dictionary 45A and where one or more gestures are selected from a gesture dictionary 43AA. One or more gestures are selected for a program, then the information is provided to the second executable portion 2A where the user performs personal customization of one or more gestures of the selected gestures one the display screen 52A or any other touch medium or sensory inputting surface or light-wave medium compatible technology. The information is analyzed and recorded by module of analyzable factors 26A and an artificial intelligence module 25A for pressure, thickness, time, acceleration, direction, angle, interruptions, distance, size, one hand or both hand, and location, wherein the information allows the artificial intelligence module 25A to differentiate from past, current and future gesture selections of the one or more touch points 52A in the creation of new gestures for future artificial intelligence assistance.

It is worth mentioning that, not all touch points, once associated with touching of the one or more display screens 52A, the one or more split programs, the one or more sideline elements 31A, the one or more adjuster interface 21A, the one or more sideline reservations 32A, one or more time tracker, and one or more reservation areas 33A, will generate the same response for every identical or similar touch point 521A. For example, one touch point 521A in the reservation area 33A may generate the program in the reservation area 33A to close, while the similar one touch point on the sideline element 31A may move the sideline element 31A accordingly. In other words, the differentiation of gestures also depends on the independency and codependency of the displaying programs in the display screen 52A. For example, the identical gesture, that was first generated in the reservation area 33A with a sideline border and an edge of the electronic device 52A thereby providing the display screen controller 20A an ability to recognize the intended gesture, is only for the reservation area. Also, in a second example, the user specifically touches the sideline element 31A to produce the gesture allowing the sideline module 30A to recognize the operator's intentions.

According to the preferred embodiment, the second executable portion 2A is operable to manage the one or more touch point information produced by the user on the one or more display screens 52A. Furthermore, the second executable portion 2A is operable to receive, record and analyze the one or more touch point information with the module of analyzable factors 26A and the artificial intelligence module 25A, and then to deliver or acquire from the information to the other three executable portions 1A, 3A and 4A. After the one or more touch point information have been identify and recorded, the one or more touch point information oscillates from the other executive portions such as the first, third and fourth executable portions 1A, 3A and 4A accordingly. In the second executable portion 2A, the one or more touch point information is produced on the one or more display screens 52A and received by a display screen controller 22A of the second executable portion 2A, as they are analyzed for pressure, thickness, time, acceleration, direction, angle, interruptions, size, one hand or both hands, and location in the module of analyzable factors 26A. The one or more touch point information is also analyzed by the module of analyzable factors 26A and reviewed by the artificial intelligence module 25A to differentiate from past, current and to learn for future selections of the touch points 521A gesture creations and program assistance anticipation.

According to the preferred embodiment, the first executable portion 1A is operable to generate the one or more adjusters 13A and the one or more graduations 120A according to the operation information of the one or more touch points 521A. The one or more adjusters 13A and the one or more graduations 120A are sent to the second executable portion 2A. The second executable portion 2A is operable to generate the adjuster interface 21A according to the one or more adjusters 13A and the one or more graduations 120A. In the adjuster interface 21A, each of the one or more adjusters 13A is arranged to a position that suitable for operation. The adjuster interface 21A is sent to the processor 51A through the communication module 55A. The processor 51A configures the one or more display screens 52A to display the adjuster interface 21A in the corresponding display screen 52A. After the adjuster interface 21A is displayed in the corresponding display screen 52A, the user is able to interact with the electronic device 50A through a gesture control 211A on the adjuster interface 21A.

According to the preferred embodiment, the first executable portion 1A is embodied as an adjuster module 10A, the second executable portion 2A as a display screen controller module 20A, the third executable portion 3A is embodied as a sideline module 30A, the fourth executable portion 4A is embodied as an interactive module 40A. In other words, the display screen controller module 20A is communicatively connected with the one or more display screens 52A while the one or more display screens 52A are connected with the communication module 55A, the processor 51A, the storage device 53A, the inputting device 56 and the power supply 54A of the electronic device 50A. The display screen controller 20A is configured to receive the operation information from the one or more display screens 52A, that is the information regarding the operation of the user on the one or more display screens 52A. The display screen controller 20A sends the operation information to all the other modules 10A, 30A and 40A accordingly. One or more of the adjuster module 10A, the sideline module 30A and the interactive module 40A generate a response where the information is analyzed by one or more control modules 20A and processed thereby at the same time and then sends the analyzed information to the processor 51A. The processor 51A sends the information for displaying on the corresponding touch screen 52A, where the one or more adjuster interfaces 21A are manipulated to operate the desire controls on the one or more display screens 52A.

The adjuster module 10A is operable to generate a change in display according to the information provided by the one or more adjuster interfaces 21A. The sideline module 30A is operable to generate one or more divisions in the number of program display according to the information provided by the one or more display screens 52A from the one or more touch points 521A. The interacting module 30A generates one or more existing programs and pairs one or more new gestures with a program or program function, to display on one or more display screens 52A and operational by one or more touch points 521A of the user.

The display screen controller 20A is operable to receive the one or more touch point information, wherein the module of analyzable factors 26A differentiates one or more touch point information and the artificial intelligence module 25A is configured to learn, verify, record and anticipate a response. Then, the one or more touch point information are sent through the communication module 55A to the processor 51A to generate on the one or more display screens 52A as one or more of the following: the one or more adjuster interfaces 21A, the one or more adjusters 13A, the one or more adjuster indicators 14A, the pressure indicator 15A, the numeric indicator 16A, the one or more graduations 120A, the one or more sideline elements 31A, the one or more sideline reservations 32A, the one or more reservation areas 33A, the one or more pop-up sideline elements 37A, an interacting program 42A, a keyboard element program 43A, a gesture and cursor program 44A, the program dictionary 45A, a scrolling program 46A, a zoom program 47A, a state-off program 48A, a time tracker program 49A, an operating area program 401A, the gesture dictionary 402, a displaying contents adjustment 403A, and/or etc.

The one or more interface portions 41A are displayed in the one or more displaying portions 523A of the one or more display screens 52A throughout the screen in directed correlation of the information provided by the adjuster module 10A in relation to the number of programs split by the one or more sideline elements 31A, the one or more sideline reservation(s) 32A, the one or more reservation areas 33A, and/or the one or more pop-up sideline elements 37A configured by the sideline module 30A. Additionally, for every sideline element 31A, sideline reservation 32A, reservation area 33A, pop-up sideline element 37A, there are respective programs from the interacting program module 40A with one or more gesture commands associated with each program and where all existing programs are pair with one or more gestures corresponding from the interactive module 40A. The one or more touch points 521A are performed on the one or more display screens 52A in the electronic device 50A, where the gesture dictionary 402A and the program dictionary 45A are scalable by up-grades provided in the internet and or private or public cloud server 100A.

The adjuster interface 21A can be control in various options, for example on a display screen 52A for sizing displayed contents, on a time dependent gesture for customizing, on a sideline element 31A for controlling one or more of the sideline elements 21A. Through the adjuster interface 21A, the user can control the time customization, contents viewing adjustments, one or more programs arranged and displayed on the one or more display screens 52A, the sideline elements 31A, and the display viewing adjustments on the page for one or more programs and on one or more locations of the program through the control on the adjuster interface 21A on the corresponding adjuster 13A of the adjuster module 10A on the corresponding display screen 52A.

Each of the one or more display screens 52A requires the interactive program module 40A to deliver one or more program responses from one or more gestures, wherein each of the one or more gestures which is selected for pairing with the corresponding program and the one or more touch point information, is delivered to the processor 51A by the display screen controller module 20A for the purpose of generating a program response from the program module 40A, but not identical, in the processes of paring gesture and program together the artificial intelligence 26A recorders. Combination of these particular steps are important to acquire various levels of precision for the customization of a gesture with the respective one or more programs because it allows the one or more programs to be displayed with one or more similar gestures with one or more different levels of complexity from each of the criteria from the module of analyzable factors 26A. As mentioned above in respect to the time factor and the adjuster interface 21A customization.

The artificial intelligence module 25A may learn, verify, record, and anticipate when the one or more gestures are differentiated or similar from the module of analyzable factors 26A, and then the information is processed by the processor 51A and delivered to the corresponding modules, that is the adjuster module 10A, the display screen control module 20A, the sideline module 30A, and the interactive module 40A, and finally stored in the storage device 53A of the electronic device 50A and/or in the server storage device 103A of the private or public cloud server 100A.

A brain-computer interface (BCI), sometimes called a brain-machine interface (BMI), is a direct communication pathway between the brain's electrical activity and an external device, most commonly a computer or robotic limb. BCIs are often directed at researching, mapping, assisting, augmenting, or repairing human cognitive or sensory-motor functions. Implementations of BCIs range from non-invasive (EEG, MEG, EOG, MRI) and partially invasive (ECoG and endovascular) to invasive (microelectrode array), based on how close electrodes get to brain tissue. Due to the cortical plasticity of the brain, signals from implanted prostheses can, after adaptation, be handled by the brain like natural sensor or effector channels. The first neuroprosthetic devices implanted in humans appeared in the mid-1990s.

According to one embodiment, the artificial intelligence 25A in the electronic device 50A may provide the computing and algorithm programs for assisting the operations of the Brain Computer Interface (BCI), wherein the Brain Computer Interface (BCI) is implemented as an attachable interface device or a built-in system within the electronic device 50A. Furthermore, the artificial intelligence 25A (algorithms) for computing on the Brain Computer Interface (BCI) may not come from the built-in artificial intelligence 25A of the electronic device 50A. Alternatively, the computing Artificial Intelligence for the Brain computer Interface (BCI) may get deliver to the electronic device 50A or any other capable computing system via internet or cloud servers, for example, artificial intelligence servers from the cloud for the process of operating the Brain Computer Interface.

The user can have one or more gestures similar for various paired programs, while the user is difficult to identically produce the one or more gestures, where the module of analyzable factors 26A and the artificial intelligence module 25A are unable to distinguish one from the other, Therefore, when pairing one or more gestures with a program, the user should perform the selected gesture for multiple times for records and recognition since everyone has different individualizable variants for making one or more gestures.

Two or more different programs may function with one similar gesture. In addition, similar gestures may be distinguished from other similar gestures with time and any of the other module of analyzable factors 26A, mentioned in the display screen controller 22A, in conjunction with the artificial Intelligence module 25A. Further, any of the module of analyzable factors 26A and any of the above mentioned modules can be pre-programed to display the respective adjuster interface 21A for customization of pressure, thickness, time, acceleration, direction, angel, interruptions, size, from both hand or one hand, at the same time or in a timely manner, from the one or more touch points and etc.

According to the preferred embodiment of the present invention, the one or more adjuster interfaces 21A can be generated by the second executable portion 2A for controlling a state of the displaying content and/or gestures, wherein the one or more adjuster interfaces 21A are configured to operate the one or more module of analyzable factors 26A independently simultaneously for customization of pressure, thickness, time, acceleration, direction, distance, angle, interruption, size, from both hand or one hand, concurrently or in a timely manner, from the one or more touch points, etc.

According to the preferred embodiment of the present invention, the one of more graduations 120A generated by the first executable portion 1A is for indicating a state of the displaying content, wherein the one or more graduations are attached along a body of the adjuster corresponding to the one or more adjusters 13A and a current state of a viewing field of each of the displaying contents as displayed corresponding to each of the split programs on the one or more display screens 52A.

In one embodiment, the one or more graduations 120A generated by the first executable portion 1A may also be arranged for:
(i) indicating a state of the time and speed or any other factors provided in the module of analyzable factors 26A, wherein the one or more graduations 120A are attached along the body of the adjuster corresponding to the one or more adjusters 13A so as to illustrate a current state of the time on a time dependent program for each of the time dependent contents that are displayed corresponding to the one or more programs on the one or more display screens 52A;
(ii) indicating a location of the controls for the one or more sideline elements 31A to move the sideline elements 31A accordantly, wherein the one or more graduations 120A are attached along the body of the respective adjuster 13A while the adjuster 13A is located along the sideline element 31A corresponding to provide ergonomic controls on the one or more sidelines elements 31A, wherein the sideline elements 31A are displayed in correspondence to the one or more programs on the one or more display screens 52A; and
(iii) indicating a state of displaying content, a state of display of the sideline elements 31A, and a time for the one or more touch points 521A, wherein the one or more graduations 120A are attached along the body of the respective adjuster 13A and, where the adjuster indicator 14A illuminates/notifies on the graduation 120A, the level the user is currently manual controlling, corresponding to the one or more adjusters 13A, a current state of the viewing field, the sideline location, and the time editing on one or more programs on the one or more display screens 52A.

According to the preferred embodiment, the first executable portion 1A is also operable to generate the one or more numeric indicators 16A with or without the graduations 120A for indicating the state of displaying content, wherein the one or more numeric indicators 16A are adjacent to the one or more adjusters 13A, wherein each of the numeric indicators 16A illuminates/notifies the level the user is currently manual controlling, corresponding to the one or more adjusters 13A, the current state of the viewing field, and for time editing on the one or more programs on the one or more display screens 52A. It is worth mentioning that the numeric indicator 16A can appear on any location throughout the display screen 52A and can also be customized for one or more selected programs with or without the adjuster 13A.

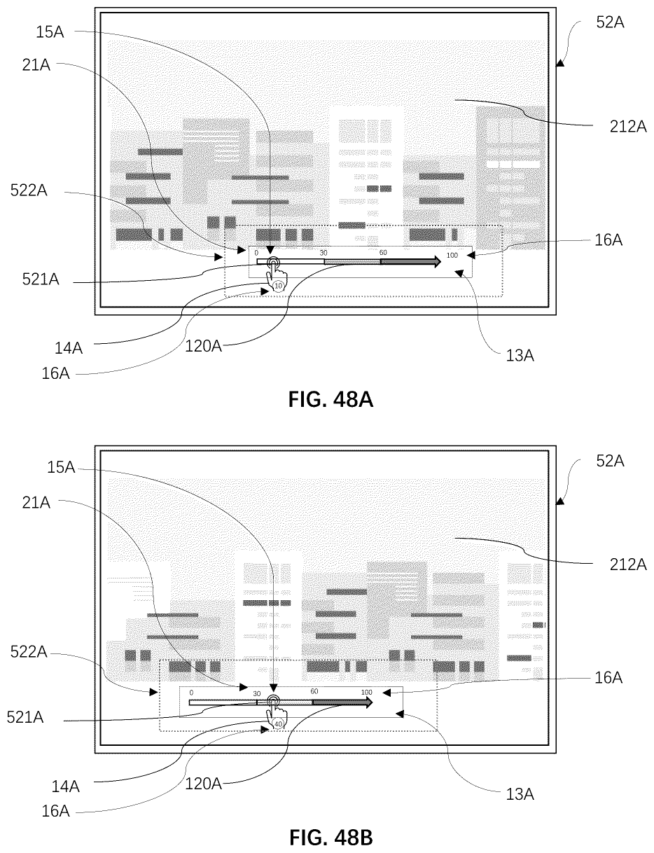

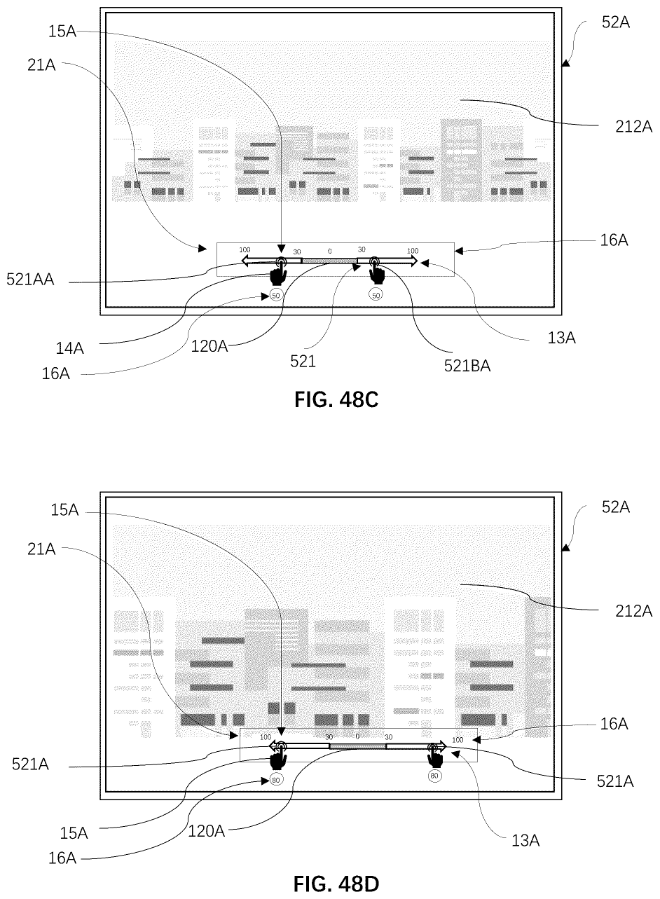

For purpose of further illustration of the preferred embodiment of the present invention, operation examples are provided with reference to FIG. 48A to FIG. 9. Referring to FIG. 48A to FIG. 48B, the adjuster interface 21A is illustrated, wherein a displaying content 212A, such as a picture or a text file, is displayed on the display screen 52A. Considering the displaying contents 212A to automatically displaying and providing watermark of the adjuster 13A with the adjuster interface 21A, the adjuster indicator 14A, the pressure indicator 15A, and/or the numeric indicator 16A on any available displaying contents 212A with available scrolling capacity. In the preferred embodiment, the adjuster 13A with the adjuster interface 21A, the adjuster indicator 14A, the pressure indicator 15A, and/or the numeric indicator 16A are conveniently illustrated when the user produces a gesture to generate a scrolling program on the desire location of the display screen 52A. A left portion of the displaying content is hidden on the page. The hidden content refers to the content that is not currently being displayed on the display screen 52A as shown in FIG. 48A. The user makes a one touch gesture at a desire location, a desired touch point 521A, on the touch screen 52A, and then continuously moves the screen touching finger towards a right side on a linear vector or segment on the display screen 52A, such that the left hidden displaying content is now displayed one the display screen 52A according to the user gesture movement from one or more touch points 521A. In this process, the displaying content 212A is moved towards the right side of the display screen 52A. When the display screen 52A detects one or more fingers of the user touching the display screen 52A, the display screen 52A defines the location and (in this case) the one or more touch points 521A as a “gesture for moving”, a “gesture for zooming” or the like and detects the moving direction of each touch point 521A as an input gesture. When the touch point 521A is continuously moving towards the right side in a linear vector on the display screen 52A, the display screen 52A detects such input gesture as a touch point information and sends the touch point to the display screen controller module 20A. The display screen controller module 20A analyzes the module of analyzable factors 26A, for example one touch point, light pressure, no interruptions, no thickness, unspecified location, with direction, short time, one hand, no angle, and etc. of the touch point information and sends to the artificial intelligence module 25A for recording and learning the gesture format of the particular user to anticipate a desire outcome of gesture information, such as “gesture for moving” or “gesture for zooming” or etc., which is then sent to the interacting program module 40A. For example, if the user continues to move the finger on the display screen 52A towards the right side on a linear vector or segment on the display screen 52A, such that the left hidden displaying content 212A is now displayed on the display screen 52A according to the user gesture movement from one or more touch points 521A. In this process, the displaying content 212A is moved towards the right side of the display screen 52A. When one or more fingers of the user is detected touching the display screens 52A, the display screen 52A defines the location and the one or more touch points 521A as a “gesture for moving”, a “gesture for scrolling”, or etc.

Additionally, referring to FIGS. 48B to 48C, the image can be initially displaying at a customary magnification range and the image with available magnification automatically provides the adjuster 13A with the adjuster interface 21A, the adjuster indicator 14A, pressure indicator 15A, and or the numeric indicator 16A. According to the preferred embodiment, the adjuster 13A as well as the adjuster interface 21A, the adjuster indicator and the pressure indicator 15A, and or the numeric indicator 16A are conveniently illustrated when the user produces a gesture with one single finger with exerted pressure or the like, a moving direction of each touch point 521A is detected as an input gesture. When the one touch point 521A is continuously moving towards the right side or the left side in a linear vector on the display screen 52A, such input gesture on the display screen 52A is detected as a touch point information sending to the display screen controller module 20A. The display screen controller module 20A analyzes the module of analyzable factors 26A, for example one touch point, severe pressure, no interruptions, not thick, location being identified, with direction, short time, one hand, no angle, and etc. It is worth mentioning that the gesture generates the adjuster 13A with the adjuster interface 21A, the adjuster indicator 14A, pressure indicator 15A, and or the numeric indicator 16A accordantly to the touch point 251A based on the current display conditions of the displaying contents 212A. Then the touch point information is sent to the artificial intelligence module 25A for recording and verification and the gesture is formatted to the particular usage to anticipate a desire outcome of gesture information, such as “gesture for scrolling” or “gesture for zooming” or etc., which is then sent to the interacting program module 40A.

The interacting program module 40A (fourth executable portion 4) receives the gesture information and then activates a corresponding gesture program accordingly. In addition, the touch point information and the gesture information are further sent to the adjuster module 10A (first executable portion 1) for an adjustment of the adjacent displaying contents in relation to the touch point 521A, wherein the display screen controller 20A generates the adjuster 13A with the adjuster interface 21A accordingly.

The display screen controller 22A of the display screen controller module 20A (second executable portion 2A) is configured to acquire the analyzed touch point information from the corresponding adjuster interface 21A after the respective display screen 52A displays the corresponding adjuster interface 21A. The adjuster module 10A (first executable portion 1A) generates at least one adjuster 13A according to the analyzed touch point information in the adjuster interface 21A on the corresponding display screen 52A, wherein the user's starting gesture position over the adjuster 13A actives the adjuster interface 21A to display on the display screen 52A and is managed as a starting point of the adjuster 13A. The touch point information acquired by the module of analyzable factors 26A and the artificial intelligence module 25A is shared with the processor 51A and/or server processor 101A with the other modules, including the adjuster module 10A, the sideline module 30A, and the interactive module 40A. The adjuster 13A is displayed at the adjuster interface 21A to reflect the user's gesture moving location and direction as the means to controlling and customizing the programs according to the touch points 521A of the user. It is worth mentioning that the adjuster 13A is possible to pre-program to any other specific locations on the one or more display screens 52A. The touch point information is sent to the adjuster module 10A so that the adjuster interface 21A is also moved accordingly to the location of the adjuster 13A to assist in the operations of the display screen 52A.

According to the preferred embodiment, referring to FIGS. 48A-48D, the adjuster 13A is embodied as a line segment having one or more graduations 120A to inform the operating user of the current level as it pertains to the available levels of function. Preferably, the adjuster 13A is displayed adjacent to the current location of the touch point 521A, as shown in FIG. 48A. The adjuster 13A can be predetermined or customized by the user with any color, size, watermark shape, and length to be visually displayed on the display screen 52A. The adjuster 13A comprises a body having the graduations 120A illustrating multiple levels as scales facilitating the user to control his or her selected programs with respect to his or her available levels of function within the program. The length of the adjuster 13A is the size of the body from a first (left) end to a second (right) end or vice versa from the second end to the first end, proportional to the number of graduations 120A that can be customized by the user in inches, centimeters or millimeters through the adjustable module 10. The graduation 120A orientates the user as to the multiple levels of function on the adjuster interface 21A. According to the preferred embodiment, the graduation 120A further comprises one or more numeric indicators 16A with or without one or more adjuster indicators 14A where the numbers fluctuated in the numeric indicator 16A in relation to the touch point gesture movement by the user on the adjuster interface 21A so as for notifying the user as to the current level for the desire operation function, for example the portion of the image as shown in FIG. 48A or the size of the image as shown in FIG. 48C, in relation to the touch point location on the particular graduation 120A of the adjuster 13A. In other words, the numeric indicators 16A can be provided with or without the adjuster 13A to continue informing the user of the current numeric number value in relation to the available and current function of the location or scale/size or of any other control function for any other program on the display screen 52A allowing the user to accurately perform precision required operations.

The adjuster indicator 14A can be a customizable watermark spot or mark, for example, provided at the location where the finger touching on the display screen 52A (or where the fingertip be detected by the displaying screen when the display screen 52A is formed by a projector or a VR goggle) and is able to move along the body of the adjuster 13A throughout the multiple graduations 120A with various scale sizes and are limited by the length of the body of the adjuster 13A with or without the numeric indicator 16A. Preferably, the adjuster indicator 14A is moved between the first and second ends of the adjuster 13A. In other words, the length of the adjuster 13A refers to the adjusting range between the first and second ends. It is worth mentioning that when the touch point 521A is moving towards the second end of the adjuster 13A on the display screen 52A, the adjuster indicator 14A as well as the numeric indicator 16A are following the movement of the touch point 521A. In other words, with the movement of the touch point 521A, the adjuster indicator 14A changes location in the adjuster 13A and the numeric indicator 16A changes numeric value in relation to the current position of the touch point 521A on the adjuster 13A controlling the adjuster interface 21A.

In an alternative embodiment, the numeric indicator 16A can also display the current function of the program, wherein at least one display screen 52A can have multiple numeric indicator 16A being displayed at the same time for multiple program functions, such as one the numeric indicator 16A for the display screen contents percentage size, the other for the display brightness, the other for the display screen speed, etc. In other words, the numeric indicators 16A are customizable for shape, color, size, locations, selected programs and are not limited.

The adjuster indicator 14A is able to move along the body of the adjuster 13A with the graduations 120A in scale and has the same fluctuation length as the body of the adjuster 13A with or without the numeric indicator 16A. Preferably, the adjuster indicator 14A is moved between the first and second ends of the adjuster 13A. In other words, the length of the adjuster 13A refers to the adjusting range between the first and second ends. It is worth mentioning that when the touch point 521A is moving towards the second end of the adjuster 13A on the display screen 52A, the adjuster indicator 14A as well as the numeric indicator 16A are following the movement of the touch point 521A. In other words, with the movement of the touch point 521A, the adjuster indicator 14A together with the numeric indicator 16A are also moving towards the right side of the display screen 52A. It is worth mentioning that the same applications mentioned above are applicable to the adjuster 13A, the adjuster indicator 14A, the numeric indicator 16A, and the graduation 120A while the adjuster 13A may be positioned vertically from top to bottom or vice versa and/or obliquely.

The pressure indicator 15A is provided at the adjuster indicator 14A, for example, as one or more ring marks around the adjuster indicator 14A, wherein one ring mark represents a low pressure, two ring marks represent middle pressure and three ring marks represent high pressure applied to the display screen 52A by the one or more touch points 521A. In other words, the number of rings produced illustrated how hard the touching finger pressing on the display screen 52A. Of course, the pressure of touching finger can also be illustrated by other ways such as the darkness of the spot mark of the adjuster indicator 14A and/or the that is the lighter the spot mark, the less finger pressure be applied, and that the darker the adjuster 13A that is the lighter the spot mark, the less finger pressure is being applied, and that the darker the spot mark, the heavier the one or more finger pressure is being applied. It is worth mentioning that the pressure indicator 15A can be activated throughout the display screen 52A accordingly of an AR/MR/XR/VR apparatus.

When only one finger of the user touches the display screen 52A, the one finger touch point 521A is a “gesture for moving” that the displaying content 212A such as the image as shown in FIG. 48A is moving along with the movement of the one finger touch point towards the direction of the movement according to the scaling as indicated in the adjuster indicator 14A as well as the numeric indicator 16A of the adjuster 13A. For example, when the one finger touch point 521A is moving towards the second (right) end of the adjuster 13A, at the same time, the displaying content 212A is moved to the right side of the display screen 52A accordingly and the hidden content is moved towards the right side of the display screen 52A and displayed on the display screen 52A, while the adjuster indicator 14A and numeric indicator 16A are fluctuated to represent the new percentage of the total image not being seen and or to represent the total percentage of the image currently in the field view at the same time, as shown in FIG. 48B. When the one finger touch point 521A is stopped, the adjuster indicator 14A and the numeric indicator 16A illustrated on the adjuster 13A also stop and so does the displaying contents 212A stop from moving. As shown in FIG. 48A, the current state of the displaying contents 212A, the adjuster indictor 14A shows a more left position on the scale diagram and the numeric indictor 16A is showing a numeric value and, at the same time, the displaying contents 212A is not moving. In comparison, as shown in FIG. 48B, the current state of the displaying contents 212A has moved more to the right, the adjuster indictor 14A shows a more to the right position on the scale diagram and the numeric indictor 16A shows a numeric value and, at the same time, on the display screen 52A.

Referring to FIG. 48C, when two touch points 521A, one from one hand or from two fingers of one hand being in contact with the display screen 52A, are detected, the adjuster 13A is displayed on the display screen 52A, for example, the two finger touch points 521A is a “gesture for zooming” that the displaying content 212A, such as the image as shown in FIG. 48C is activated to zoom-out when the two touching fingers are moving closer with each other that more panoramic field of view on the display screen 52A is delivered, or is activated to zoom-in when the two touching fingers are moving apart with each other that an enlargement in magnification is delivered on the display screen 52A. The adjuster interface 21A is provided on the display screen 52A, controlled by the two touch points 521A and a linear segment of the adjuster 13A is provided between the two touch points 521A, such that when the two touching fingers of the user are moving closer with each other along the adjuster 13A, the displaying content 212A is enlarged in panoramic field of view (zoom-out) accordingly at the same time and the two adjuster indicators 14A are moving closer in relation to each other on the adjuster 13A. At the same time, the numeric indicator(s) shows the change percentage value to 50s % of enlargement for the panoramic field of view (zoom-out) of the displaying content 212A from the original image size.

Referring to FIG. 48D, when the two touching fingers of the user are moving apart from each other along the adjuster 13A, the displaying content 212A magnification is increased (zoom-in) and, at the same time, the two adjuster indicators 14A are moving apart from each other. The two touch points 521A and the numeric indicator(s) show the percentage of magnification increased (zoom-in) of the displaying content 212A from the original image size thereof. A left or right movement of the two finger touch points 521A towards the direction of the movement according to the scaling as indicated in the adjuster indicator 14A as well as the numeric indicator 16A of the adjuster 13A. When the moving of the two finger touch points 521A has stopped, the adjuster indicator 14A is repositioned and the numeric indicator 16A is illustrated as the new numeric number value on the adjuster 13A and also stopped at the same time as the displaying content 212A stops from zooming in too.

It is appreciated that the present invention provides the adjuster interface 21A, the adjuster 13A, the adjuster indicator 14A and/or the numeric indicator 16A, so that the gesture for zooming can be any other gesture customized by the service provider or the user, for example a two-fingers gesture that when the user uses his or her two fingers, such as index finger and middle finger, side by side touching on the display screen 52A, the corresponding touch point(s) 521A is detected by first executable portion 1A (adjuster module 10A) and controlled by the second executable portion 2A (display screen controller module 20A).

According to the preferred embodiment of the present invention, other gestures can also be similarly customized and operated for the processes of operating various different programs, for example, in the task of editing a music video sound can be controlled with one finger and the brightness on the display video can be controlled with a two-finger touch point gesture, as illustrated in FIGS. 48C-48D, providing the user, additional adjusters operated with a different gesture, wherein the two-finger gesture can be performed by the index finger and middle finger, and etc., side by side touching on the display screen 52A. The corresponding touch point(s) 521A are analyzed by the module of analyzable factor 26A and the artificial intelligence 25A in the second executable portion 2A, and then the information is delivered by the processor 51A and/or the server processor 101A to the first executable portion 1A (adjuster module 10A) to generate the adjuster 13A. At the same time, the fourth executable portion 4A receives the current displaying content information and the displaying content adjustment 403A delivers the information to the adjuster module 10A through the processor 51A and/or the server processor 101A, wherein the information is provided to the second executable portion 2A to generate the adjuster interface 21A on the corresponding display screen 52A to be displayed and be controlled by the touch point 521A through gestures, as the user desires, through the display screen controller module 20A.

According to the preferred embodiment of the present invention, the program control is directly proportional to the manipulation of one or more gestures of one or more adjuster interfaces 21A. The one or more adjuster indicators 14A and the one or more numeric indicators 16A are controlled directly proportional to the scale or size of the manipulation on the adjuster interface 21A by the user. For example, the range of a function of a program availability or lack of availability is directly proportional to the location of the adjuster indicator 14A and to the display of the numeric indicator 16A on the display screen 52A in relation to the gesture of the one or more touch points 521A. In other worlds, as illustrated in FIG. 48C, a shorter distance between the two touch points 521A and a smaller numeric number value by the numeric indicator 16A, and that, as illustrated in FIG. 48D, a larger display distance between the two touch points 521A and a larger numeric number value by the numeric indicator 16A. In other words, the FIG. 48D demonstrates that the user has 80% magnification with only an additional 20% availability, and therefore the two fingers have less available space for separation (zoom-in). The two touch points 521A have more available space to come together from the zoom-in to the zoom-out gestures respectively.

According to the preferred embodiment of the present invention, an image viewing field is directly proportional to the gesture manipulation on the adjuster interface 21A. The display and control of the one or more adjuster indicators 14A and the one or more numeric indicators 16A are directly proportional to the scale or size of the manipulation on the adjuster interface 21A by the user. Also, the image viewing field availability or lack of availability is directly proportional to the location of the adjuster indicator 14A and to the display of the numeric indicator 16A, on the screen or program. In other words, FIG. 48C illustrates a smaller scale/size display of the buildings (zoom-out) and FIG. 48D illustrates a larger scale/size display of the buildings (zoom-in). In other words, the two FIG. 48C and FIG. 48D demonstrate from the zoom-in to zoom-out respectively. As disclosed above, the same process can be accomplished for scrolling on an image as illustrated on FIGS. 48A and 48B.

It is understood that the adjuster 13A may or may not have the adjuster indicator 14A provided nor the graduation 120A in relation to the controlling of the adjuster interface 21A on the adjuster 13A. The numeric indicator 16A can visually notify the user of the level for the selected function on the adjuster interface 21A, where the degree of manipulation is control accordingly by the user with one or more touch gestures side to side, up and down or diagonal without the adjuster indicator 14A nor the graduations 12A on the adjuster 13A. It is appreciated that it is possible to only have the numeric indicator 16A displayed on the display screen 52A of the electronic device 50A.

The image viewing field is directly proportional to the user manipulation on the adjuster interface 21A that the numeric indicator 16A is always displaying the current numeric value, and directly proportional to the touch point 521A positioning on the adjuster interface 21A in relation to the state of manipulation by the user with respect to the display screen 52A and may or may not be displayed on the electronic device 50A when it is an AR/MR/XR/VR apparatus. The numeric indicator 16A is preferred to represent the current state of the program being control, whether it is an image, viewing field, time, sound brightness, or any other functions. The availability or lack of availability control is directly proportionately shown by the numeric indicator 16A as the currently program function provides the available program capacity to the user.

In other words, since the image viewing field is directly proportional to the user manipulation on the adjuster interface 21A, the numeric indicator 16A, displaying a current numeric number, is directly proportional to the adjuster interface 21A at the state of manipulation by the user and is the current representation of the image viewing field availability or lack of availability viewing field directly proportional to the numeric indicator 16A of the current percentage being displayed on the display page in any electronic device 50A including the AR/MR/XR/VR apparatus.

In other embodiments, the user may elect to have a dual foldable touch screen apparatus where the user may operate the VR on the control panel of the electronic device 50A, wherein the display screen of the electronic device 50A is utilized by other individuals for viewing and experiencing the metaverse environment together with the operator who is wearing the VR/MR/XR/AR goggles as a means of sharing the experience together.

In one embodiment, the user may operate the AR/MR/XR/VR through the use of any other electronic device 50A where other users may participate or only observe in the sharing of the experience for the process of the communal experience, wherein the user have options for interacting or not.

It is worth mentioning that an adjusting range of the adjuster 13A is capable of being preconfigured for zooming. For example, the adjuster module 10A configures the adjusting range of each of the one or more adjusters 13A according to a pre-set percentage of a movable distance of the hidden portion and/or of the displaying content. In such a manner, the user doesn't need to move actual or long distance to let the desired hidden portion of the displaying content to display. The user just needs to move a relatively short distance along the adjuster 13A of the adjuster interface 21A according to the desired display on the display screen 52A. Furthermore, the adjuster module 10A generates the one or more graduations 120A on the adjuster 13A according to the percentage of the hidden portion of the displaying content and or a percentage of the displaying contents that further facilitates the user to fast and efficiently determine the display portion or percentage of the displaying content on the display screen 52A. The one or more graduations 120A could be provided from the first (e.g. left) end to the second (e.g. right) end of the adjuster 13A. The one or more graduations 120A notify the user as to the current displaying content accuracy. For example, if a total displaying content such as an image of buildings as shown in FIGS. 48A and 48B, 0-100% is the percentage of portion of the total displaying content from right to left, wherein as shown in FIG. 48A, it is about 10% of right portion of the total displaying content being displayed as the current displaying content 212A on the display screen 52A and 90% hidden portion from the left of the total displaying content not being displayed on the display screen 52A, and, as shown in FIG. 48B, the current displaying content 212A is about a 40% portion of the total displaying content, from right to left, being displayed on the display screen 52A, i.e. the previously hidden portion on the left portion of previously current display portion as shown in FIG. 48A is moved to be the current displaying content 212A to be displayed on the display screen 52A as shown in FIG. 48B. Three graduations 120A may be provided to display 0˜30%, 30-60% and 60-100% along the adjuster 13A. In another example, the adjuster 13A may only have one graduation 120A, not including the maximum and minimum end points on the adjuster 13A, wherein the graduation 120A is located at the 50% graduation marker of the adjuster 13A.

As illustrated in FIG. 48B, the current displaying content 212A has about a 40% used magnification or about 40% scrolling used capacity of the total displaying content, from right to left, being displayed on the display screen 52A. That is the previously hidden portion on the left portion of previously current display portion as shown in FIG. 48A is moved to be the current displaying content 212A to be displayed on the display screen 52A as shown in FIG. 48B. Four graduations 120A may be provided to display 0%, 30%, 60%, and to 100% along the adjuster 13A. In another example, the adjuster 13A may only have one graduation 120A, not including the maximum and minimum end points on the adjuster 13A, wherein the graduation 120A is located at the 50% graduation marker of the adjuster 13A.

Similarly, the adjuster module 10A may configure the adjusting range of each of the one or more adjusters 13A according to a pre-set percentage of a scale or size of the displaying content 212A. In such a manner, the user doesn't need to repeatedly move his or her two fingers (index finger and thumb or any other two touch points 521A) closer or apart from each other so as to achieve the desire scale the image to be displayed on the display screen 52A. The user just selects the distance of the two touch points 521A between any two fingers and moves his or her two fingers closer or apart for a relatively short distance along the adjuster 13A of the adjuster interface 21A according to the desire display scale on the display screen 52A. Furthermore, the adjuster module 10A generates the one or more graduations 120A on the adjuster 13A according to the percentage of the scale or size of the displaying content that further facilitates the user to fast and efficiently determine the scale percentage of the displaying content on the display screen 52A. The one or more graduations 120A could be provided from the first (e.g. left) end to the second (e.g. right) end of the adjuster 13A. For example, the center of the adjuster 13A is 0% referring a minimum scale of the displaying content and both left and right ends of the adjuster 13A are 100% referring a maximum scale of the displaying content. For example, as shown in FIG. 48C, it is about 50% displaying scale or size of the image of the displaying content being displayed as the current displaying content 212A on the display screen 52A, and, as shown in FIG. 48D, the current displaying content 212A is about an 80% displaying scale of the image of the displaying content being displayed on the display screen 52A.

It is worth mentioning that, as shown in FIGS. 48C-48D, one or more adjusters 13A for two touch points 521A are generated when the user is adjusting any scalable program and the user may select a two-touch-point gesture for controlling such functions. Alternatively, the user can produce the same gesture without the adjuster 13A, the adjuster indicator 14A, the numeric indicator 16A, or any graduation 120A, to generate the same response, wherein the distance tracker module 27A on the display screen controller module 20A provides the information to the adjuster module 10A and the first executable portion 1A produces a response accordantly to the user gesture selection and movement.

It is appreciated that the user is allowed to customize the one or more gestures. For example, a simultaneous two finger touch point pre-program gesture from one hand, wherein the two touch points 521A are not limited to the two finger gesture (pinching or depinching) to activate zooming. The two touch points 521A have a programmable two touch point separation distance to generate a percentage display of a program, photo, web page, screen, and/or other displaying contents on the corresponding display screen 52A of the electronic device 50A, wherein the two touch point separation distance between the user's fingers may be in millimeters, centimeters or inches. For example, one inch space separation of the gesture of two finger touch points 521A can be programmed to display 25A percent magnification of the displaying content; two inches space separation can be programed to display 50A percent magnification of the displaying content; three inches finger space separation can be programmed to display 75A percent magnification of the displaying content; four inches space separation can be programmed to display 100A percent magnification of the displaying content; etc. or vice versa that four inches space separation between the two fingers is a zero magnification providing the user with a full panoramic view of the displaying content without any magnification on the display screen 52A. After the initial gesture is produced and the response is generated, the user's two finger space separation can continue to zoom-in or zoom-out in a continuously contact pinching or depinching motion on the display screen 52A, wherein the user may decrease the panoramic view to increase the magnification proportional to the two touch point separation control distance between the two touch points 521A, as mentioned above or vice versa. Such that the user doesn't need to repeatedly produce the two fingers to pinch or depinch gesture apart or together so as to achieve the desire range of the image for displaying on the display screen 52A. The user merely needs to move his or her two fingers closer or more apart for a relatively short distance along the adjuster 13A on the adjuster interface 21A according to the desire display scale or size on the display screen 52A. Furthermore, the adjuster module 10A generates the one or more graduations 120A on the adjuster 13A according to the percentage of the scale or size of the displaying content 212A that will further facilitate the user to a fast and efficiently determinant of the scale percentage of the displaying content 212A on the display screen 52A.

For example, in another embodiment, the center of the adjuster 13A is 0% referring a minimum scale of the displaying content 212A and both left and right ends of the adjuster 13A are 100% referring a maximum scale of the displaying content 212A. For example, as shown in FIG. 48C, it is about 50% displaying scale or size of the image of the displaying content 212A on the display screen 52A. For example, as shown in FIG. 48D, the current displaying content 212A is about 80% displaying scale of the image of the displaying content 212A being displayed on the display screen 52A.

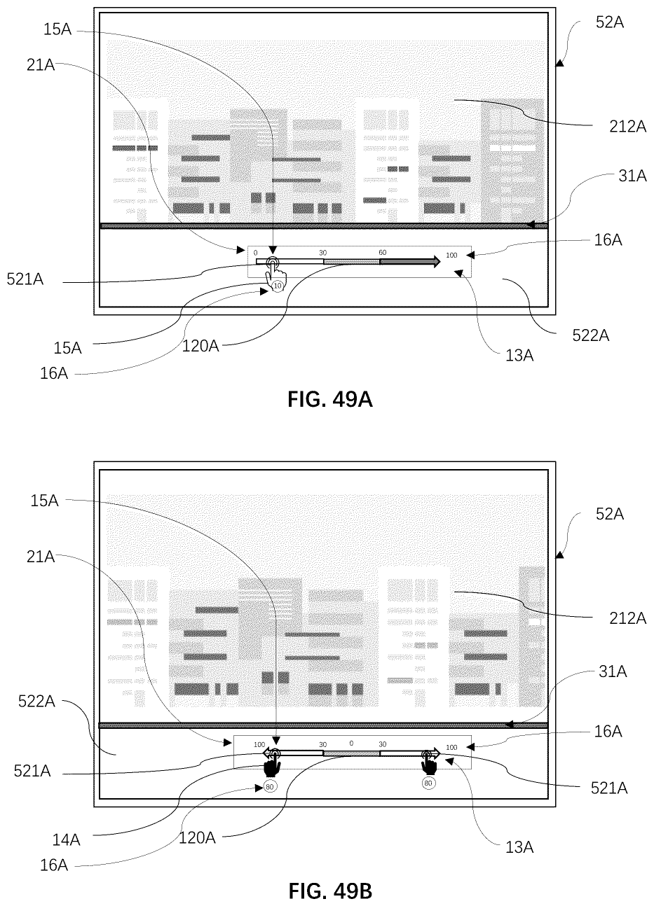

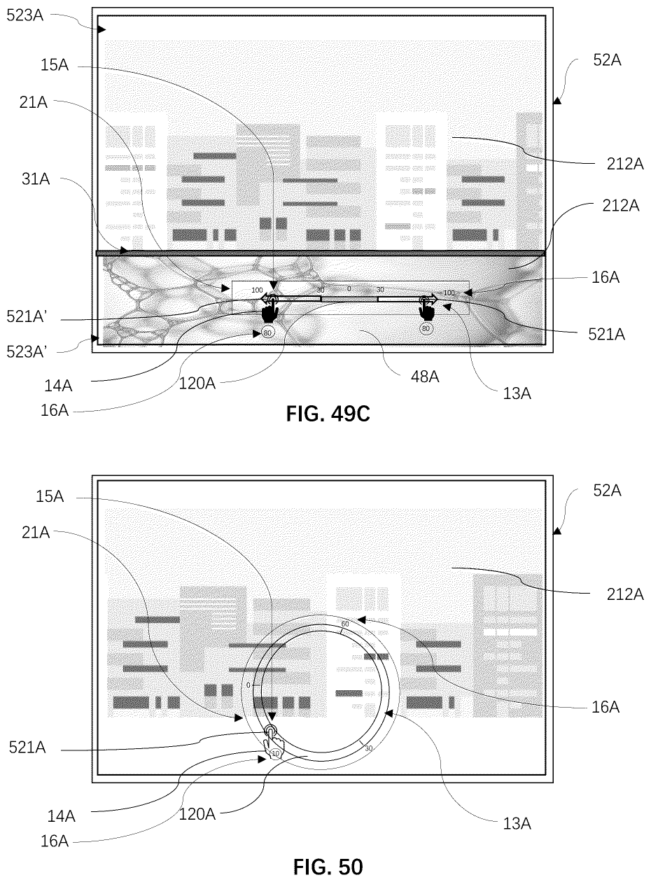

Referring FIGS. 49A-49B, alternative modes of the adjuster interfaces 21A (as shown in FIG. 48A and FIG. 48C) according to the preferred embodiment of the present invention are illustrated respectively, wherein at least one sideline element 31A is generated by the third executable portion 3A (the sideline module 30) on the display screen 52A to divide the display screen 52A into an upper portion and a lower portion, or alternative a left portion and a right portion, allowing the displaying content 212A of the image to be displayed on the upper portion while the adjuster interface 21A and the adjuster 13A to be displayed on the lower portion of the display screen 52A, wherein when the user uses at least one finger touching on the sideline element 31A, the display screen 52A detects and the third executable portion is activated to detect the movement of the touch point 521A touching on the sideline element 31A. The user is allowed to move his or her finger, moving the touch point 521A, up and down so as to move the sideline element 31A up and down accordingly so as to adjust the relative sizes of the upper portion and the lower portion to enlarge or reduce the size the displaying content 212A displayed on the upper portion of the display screen 521A and the size of the adjuster interface 21A displayed on the lower portion of the display screen 521A. Therefore, for user who has a larger hand and fingers, before the user would like to operate “gesture for moving” as shown in FIG. 49A, “gesture for zooming” as shown in FIG. 49B, or other gesture operations, the user may enlarge the size of the adjuster interface 21A and the adjuster 13A for ease of operation, and then after the corresponding adjustment, the user may reduce the size of the adjuster interface 21A and the adjuster 13A by touch moving the sideline element 31A downwards or simply double or triple touch (depending on the gesture customization by the service producer or the user) the sideline element 31A to remove the displayed sideline element 31A as well as the adjuster interface 21A and the adjuster 13A from the display screen 31A. The using of sideline element 31A is also convenient to user who needs to adjust the portion of the displaying content 212A to be displayed or the scale/size of the displaying content 212A very often during operation of the displaying content 212A to avoid the system to automatically remove the adjuster interface 21A and the adjuster 13A from the displaying content 212A as shown in FIGS. 49A and 49B after a predetermined of time, such as five seconds (more or less), when there is no detection of any touching and touch point 521A on the adjuster interface 21A and/or the adjuster 13A that is preconfigured by the system of the invention. It is worth mentioning that the system of removing functions as mentioned above can be applied to all computer programs mentioned in the invention. For example, the keyboard control may also be removed if the display screen 52A stops sensing the touch point 521A on the display screen 52A, the interacting portion 41A, the interacting program 42A, the sideline element 31A, the panel and/or display, and etc.

The adjuster 13A is generated according to the touch point 521A that is the location the user's finger touches on the display screen 52A. More specifically, the adjuster 13A is configured as a starting end at the current location of the touch point 521A and the adjuster 13A extended for a predetermined time and length to form a linear segment, or that the adjuster 13A can also be configured to extended from both sides of the touch point 521A for a predetermined length to form the linear segment.

Referring to FIG. 49C, the state-off program 48A is illustrated to be superimposed on an existing program 523A′ when two or more programs are operating and divided by the sideline element 31A. For example, a two-finger touch point pressure gesture can be produced by the user to generate the state-off program 48A where one touch point 521A comes from the right hand and the other touch point 521A′ comes from the left hand, providing the distance tracker module 27A, the module of analyzable factor 26A and the artificial intelligence module 25A the required information to activate the state-off program 48A. In the state-off program 48A, other operating program currently operating are superimposed by the state-off program 48A, wherein the user can make multiple gestures to generate additional response that are not pertaining to the original superimposed program. In other words, the programs that is superimposed by the state-off program 48A continues to run without any inputting capacity. Additionally, the gestures that are made on the state-off program 48A are for other function options and interactive portion(s) 41A or interactive program(s) 42A. Further, the operating state-off program 48A may be separated by the horizontal sideline element 31A. Also, the superimposed state-off program 48A can have a lock option to have minimal gesture capacity where the display screen 52A displaying the operational area 522A and/or the displaying content 212A is blank or illuminating any selected color to notify the user the state-off lock mode as demonstrated in FIG. 49B, wherein the state-off program 48A is in lock mode and is shown as a blank or translucent display while merely showing the adjuster 13A and the adjuster interface 21A with or without inputting capacity. The advantage to this program option is the user can physically grab that portion of the display screen 52A and the physical electronic device 50A without any worries of inputting erroneous information in the electronic device 50A.

Referring to FIG. 6, an alternative mode of the adjuster interface 21A and the adjuster 13A is illustrated, wherein the adjuster interface 21A and the adjuster 13A are in generated in form of circular segment for guiding of the user to touch and slide his or her touch point 521A circularly for various gesture operations, such as gesture for moving and gesture for zooming. the adjuster interface 21A and the adjuster 13A are preferred to be illustrated to have a predetermined percentage of transparency, such as a watermark form, that can be preset by the system or customized by the user through, for example, the first executable portion 1A. The user is able to move the one or more fingers along the watermarked adjuster 13A. In such a manner, the touch point 521A can be the starting point and moved along the adjuster 13A for adjusting the displaying content on the one or more programs on the one or more display screens 52A. Preferably, the adjuster module 10A configures the adjusting range of the adjuster 13A, wherein the length of the adjuster 13A refers to a customizable range of the displaced viewing field or of the hidden not view field while the adjuster 13A is configured for “gesture of moving’. As shown in FIGS. 48A and 4B, the touch point 521A is capable of moving along the adjuster 13A to the right for moving the left hidden displaying content to move to the right accordingly to be displayed on the display screen 52A. It is worth mentioning that the adjuster 13A can also be pre-programed by the lack of the total viewing field identified by the adjuster module 10. Where the adjuster module 10A recognizes the total viewing field is hidden on the left side of a program or display screen 52A, where the adjuster 13A will be display horizontal automatically from left to right or from right to left and with increment on the graduations 120A toward the right for right hand users or left for left hand users. The same when the total viewing field is hidden by the top side of the program and/or display screen 52A, the adjuster 13A in form of watermark is automatically display from top to bottom. Similarly, as shown in FIGS. 48C and 4D, the two touch points 521A are capable of moving along the adjuster 13A closer or more apart from each other for zooming in (shrinking) or zooming out (enlarging) the displaying content on the display screen 52A.

According to the preferred embodiment of the present invention, in particular, referring to FIGS. 48C and 48D, the user is capable of operating the adjuster interface 21A for operation with gestures to control the one or more programs and displaying content 212A on the one or more display screen 52A. The “gesture for zooming”, including zooming in and zooming out, is described as an example as follows while it is not intent to limit the scope of gesture control of the present invention. Additionally, the one or more circular type gesture can be superimposed on other prior display screen 52A generated responses gesture and vice versa.

The present invention teaches the adjuster 13A, the adjuster indicator 14A, the adjuster interface 21A, and the numeric indicator 16A which are all able to be customized to display on the one or more displaying contents 212A of the one or more displaying portions 523A or the one or more display screens 52A at pre-selected locations, through the corresponding displaying portion 523A or the display screen 52A. The present invention also teaches dedicated gestures for specific programs in this case circular for adjusting displaying contents 212A, wherein triangular customization can be used for controlling two or more programs at the same time, wherein the Y-axis and X-axis in relation to a corresponding program, for example lighting and volume, in relation to battery energy transfer. Additional dedicated square gestures can be dedicated to the transferring or up-loading of information within the programs or from and to other external programs and electronic devices 50A. In other words, the shapes for the adjuster 13A can be also customized by the user as the means to specific functions, with various colors for further differentiation.

With respect to the displaying content 212A displayed in the display screen 52A, when the user uses one or more fingers to touch the display screen 52A at the same time for performing the operation of zoom-in or zoom-out, the display screen 52A provides information of the one or more touch points 521A at the locations where the two fingers of the user touch on the display screen to the touch control module 40A, wherein the module of analyzable factors 26A are activated along with the artificial intelligence module 25A. The information of the one or more touch points 521A can be transmitted by the communication module 55A to the processor 51A to receive the information. The information of the one or more touch points 521A is sent to the interacting program module 40A (fourth executable portion 4) where the touch points 521A are recognized as the “gesture for zooming” and then the information of the two touch points 521A is send to the adjuster module 10A (first executable portion 1) to generate the corresponding adjuster 13A for adjusting the displaying content 212A to zoom in or zoom out. The display screen controller module 20A (second executable portion 2A) generates the adjuster interface 21A according to the adjuster 13A. In one embodiment, the processor 51A arranges the adjuster interface 21A to display on the display screen 52A while the adjuster 13A is displayed on the display screen 52A too. The adjuster 13A is pre-configured by the adjusting range of zoom in or zoom out. The user changes a distance between the two fingers touching on the display screen 52A, i.e. the distance between the two touch points 521A, so as to adjust the scale/size of the displaying content 212A through the adjuster interface 21A. The adjuster indicator 14A provided in the adjuster interface 21A is preferred to be positioned on the adjuster 13A for indicating a current display state of the displaying content 212A. The adjuster indicator 14A is able to be moving along the adjuster 13A. In other words, when the distance between two fingers (touch points 521A) are changed, the positions of the two corresponding adjuster indicators 14A and a number value on the numeric indicator 16A are changed accordingly to reflect the concurrent change on the adjuster interface 21A for any shape adjuster 13A whether it is linear, circular, triangular, or etc.

Additionally, the adjuster 13A may or may not have the adjuster indicator 14A in relation to the graduation(s) 120A on the adjuster interface 21A. The adjuster 13A may merely provide the numeric indicator 16A that can visually notify the user of the level for the selected function in the adjuster 13A, where the adjuster interface 21A can be customized and manipulated accordingly by the user to provide or not provide the adjuster 13A, graduation 120A, the adjuster indicator 15A, and/or the numeric indicator 16A. Alternatively, the adjuster 13A may simply show a decreasing peak and/or an increasing peak while the gesture movement can in any direction, such as vertically, horizontally or obliquely, corresponding to the customization.

More specially, when the display screen 52A is touched by the one or more fingers of one hand or both hands of the user, the display screen controller module 20A detects the two locations of the two fingers of the same hand of the user touching the display screen 52A. In one embodiment, the communication module 55A is configured to transport the information of the touch points 521A (the two locations of the two fingers touching on the display screen 52A) produced on one or more display screens 52A to the display screen controller module 20A from the one or more display screen 52A and sends the information of the touch points 521A (locations of the two fingers) through the communication module 55A to the processor 51A and from the processor 51A to the other auxiliary modules for additional information. Before the information of the touch points 521A is send to the communication module 55A from the display screen controller module 20A, the information of the touch points 521A is defined as the first location of the first finger as the first touch point 521AA, and the other location of the other finger as the second touch point 521AB. Furthermore, the distance between the two touch points 521AA, 521AB is measured and analyzed by a distance tracker module 27A of the display screen controller module 20A and the module of analyzable factors 26A are activated along with the artificial intelligence module 25A. The display screen controller module 20A further obtains a state of the selected displaying content 212A to be displayed on the of the display screen 52A and the adjuster module 10A generates the corresponding adjuster 13A according to the state of the displaying content 212A of the display screen 52A. If the state of the displaying content 212A has been enlarged, for example to a 80% of the maximum state, the adjuster module 10A generates the adjuster 13A and the adjuster indicator 15A accordingly and/or the numeric indicator 16A showing 80%, as illustrated on FIG. 4D.

The adjuster indicator 14A is preferred to but not limited to be provided at the position of the touch point 521A on the adjuster 13A. For example, the adjuster indicator 14A is pre-configured in scalable position to indicate the currently state of the displaying content 212A. Furthermore, the adjuster 13A may be displayed with the one or more graduations 120A generated along the adjuster 13A with respect to the adjuster indicator 14A. The one or more graduations 120A are formed on the adjuster 13A for displaying and to notify the available adjustable range of the adjuster 13A. The adjuster indicator 14A is configured to indicate the current displaying content 212A from the maximum through the minimum in the adjuster 13A. In one embodiment, the screen controller module 20A generates the adjuster interface 21A and sends the adjuster interface 21A to the processor 51A through the communication module 55A. The processor 51A receives the adjuster interface 21A through the communication module 55A. The processor 51A arranges the adjuster interface 21A to display in the display screen 52A. When the distance between the first touch point 521AA and the second touch point 521AB is reduced, the displaying content 212A zooms-out, e.g. by the processor 51A, on the display screen 52A. At the same time, the adjuster indicator 14A is adjusted and the position of the lessor graduation 120A displays the current state of the displaying content 212A on the display screen 52A. When the distance between the two touch points 521AA, 521AB is changed, the adjuster indicator 14A moves directly proportional to reflex the current state of the displaying content 212A on the display screen 52A and is illustrated on the adjuster 13A. Preferably, the adjuster 13A is arranged to pass through the first touch point 521AA and the second touch point 521AB. In other words, the first touch point 521A and the second touch point 521AB are able to move along the adjuster 13A in such a manner that the adjuster 13A is able to guide the user to zoom-in or zoom-out as a timely watermark popup displaying contents.

In an alternate computer cloud medium, the screen controller module 20A can generate the adjuster interface 21A and send the information of the adjuster interface 21A to the server processor 101A through the sever communication module 105A. The server processor 101A receives the adjuster interface 21A through the server communication module 105A. The server processor 101A arranges the adjuster interface 21A to display in the display screen 52A. When the distance between the first touch point 521A and the second touch point 521AB is reduced, the displaying content 212A zooms-out, for example by the server processor 101A, on the display screen 52A. At the same time, the adjuster indicator 14A is adjusted and the position of the lessor graduation 120A displays the current state of the displaying content 212A on the display screen 52A. When the distance between the two touch points 521A, 521AB is changed, the adjuster indicator 14A moves directly proportional to reflex the current state of the displaying content 212A on the display screen 52A and is illustrated on the adjuster 13A. Preferably, the adjuster 13A is arranged to pass through the first touch point 521A and the second touch point 521AB. In other words, the first touch point 521AA and the second touch point 521AB are able to move along the adjuster 13A in such a manner that the adjuster 13A is able to guide the user to zoom-in or zoom-out as a timely watermark popup displaying content.

Furthermore, the user is able to pre-configure a plurality of touch points 521A with respect to the gestures of the user for operating the one or more programs of the system on the display screen 52A. The user is able to pre-configure gestures by programing two or more touch point 521AA and the touch point 521AB. For example, when the two touch points 521A,A 521AB corresponding to the two fingers touch on the physical display screen 52A or the touch points 521A, 521AB are detected with respect to the virtual display screen, the information of the touch points 521AA, 521AB is transported by the communication module 55A to the display screen controller module 20A to be analyzed and recorded by the display screen controller module 20A, where the distance of the two touch points 521AA, 521AB has being preprogramed on the distance tracker module 27A, without additional movement by the two fingers of the users moving apart with each other (separation), the zooming percentage is immediately provided on the image. For example, the user selects a single gesture with two touch points 521A from the gesture dictionary 402A and the zooming program from the program dictionary 45A in the interacting module 40A, the display screen controller module 20A receives the information from the user and pairs the gesture with the respective program. Also, the display screen controller module 20A would analyze the user's gesture and the user's performance(s) as criteria in the pairing of a gesture with a program for the selected touch points 521A to become a gesture.

The user is further provided with additional options to program by the system of the present invention. For example, a two-inch space separation between the two fingers can be program to automatically zoom the image at 50A percent capacity and a four-inch space separation between the fingers automatically zooms the image to maximum capacity 100A percent. The advantage is a gesture of two touch points 521A, pre-program from one to eight inches long ergonomically selected and programmable to generate displaying contents 212A at set percentages based on the users two finger distance selection. Therefore, the distance between the two touch points 521A immediate provides the image resolution at the preselected percentage. In addition, the user may further control the image accordingly by maintaining continuous contact on the display screen 52A from the initial two touch points 521A distance gesture selection, such that with the same two fingers, the user may reduce or increase the space between the two fingers from two inches to one inch providing the user faster control time for zooming, no matter if the user is at the maximum zooming capacity of four inches space separation with the two fingers of the user can zoom-out, or if the user is at the 50% image zoom of two inch space separation between the touch point 521A and the touch point 521AB. Based on the user hand size, finger deformities, and/or finger injuries selection, the percentage of zooming for selected space between the two touch points 521A is parameters a preprogram gesture control. It is important to mention that the user may preconfigure similar zooming capacities for every inch, for example 1A inch for 25%, 2A inches for 50%, 3A inches for 75%, and etc. Once the user has completed all the available programmable options, the artificial intelligence module 25A reviews and records all the module of analyzable factors 26A selected or available and then delivers the information to the interacting module 40A to be recorded in the gesture dictionary 402A and in the program dictionary 45A. This process is essential to provide gesture and program pairing information to the user, such as for editing, reminder, and to prevent duplication of program and gestures pairing, as illustrated in FIGS. 48C and 4D. It is worth mentioning that the two-finger touch point space separation gesture with immediate zooming gesture capacity is provided by the display screen controller module 20A after the module of analyzable factors 26A, the distance tracker module 27A and the artificial intelligence module 25A verification, and then the information is delivered to the processor 51A to displaying content 212A of the selected two finger space separation on the display screen 52A. Where the display screen 52A may have the adjuster 13A, the adjuster indicator 14A and/or the numeric indicator 16A in a watermark form customizable and throughout the screen display 52A, as illustrated in FIGS. 48C to 48D.

In other words, through the process of pre-programing characteristics of the user's gestures, such as touching pressure of each finger of the user, a size of the touch point where the user touches the display screen 52A with his or her generally used touching finger(s), a space between two fingers from one hand or two hands of the user, and the like, the gesture operating on the display screen 52A according to the present invention can be customized. It is also an alternative method for security to the system and the electronic device 50A. Conventionally, the electronic device 50A may merely log in through password or biological security code such as fingerprint or facial recognition with camera which is not actually identification and determination of the owner of the electronic device since the password may be broken and fake fingerprint or facial image may be produced to log in the electronic device 50A. To record and learn the personal operation gestures of the owner of the electronic device 50A through the module of analyzable factors 26A and the artificial intelligence module 25A according to the preferred embodiment of the present invention, the electronic device 50A may also provide a security protection from operation (selection) by a non-owner or unauthorized user by detecting the gesture operating on the one or more display screens 52A, including how is the performance of the gestures of the user and what gesture is performed for the program on the one or more display screens 52A. It is worth mentioning that the monitoring of the gesture production and gesture and program selection for pairing are not limited to the touch-type display screen 52A of the electronic device 50A that the same applications would be applied to non-touch inputting operational controls like extremities neuromuscular wearable sensors inputting and brain electrical impulse sensors inputting interfaces.

According to the preferred embodiment of the present invention, the processor 51A can be configured to adjust the displaying content 212A to the maximum state or to a predetermine two finger separation inch indicator for the zooming percentage. Also, the adjuster indicator 14A is adjusted to indicate in form of watermark of the selected state of the displaying content 212A. The user may not always produce the perfect inches of separation. The adjuster 13A is displayed on the display screen 52A to operate the display screen controller module 40A with the adjuster interface 21A to select precision options of the gestures on the two-finger inch percentage separation and/or approximation. Also, the adjuster indicator 14A is adjusted to indicate in form of watermark of the selected state of the displaying content 212A. Once defining as suitable distance of the two touch points gesture for adjusting the displaying content 212A displaying on the display screen 52A, the preprogram displaying content percentage is acquired by the distance tracker module 27A and is delivered to the processor 51A and then displayed on the display screen 52A. In other words, when the distance between two fingers of the user is identify as suitable distance and the two fingers of the user are touching on the display screen 52A, the displaying content 212A is adjusted to the pre-program display percentage in the display screen 52A, wherein the adjustor indicator 14A indicates the watermark displayed proportional to the state of the current display 212A, as to the current state of the displaying content 212A. In such a manner, the preconfigure distance between the two fingers is able to be defined and continuously operate the displaying content zooming capacities. Furthermore, the artificial intelligence module 25A is configured to record and analyze each gesture operation of the authorized user of the electronic device 50A so as to learn and determine the distance between the two fingers of the user and the corresponding displaying contents 212A to anticipate the users' operational needs.

For example, the display screen controller module 20A determines the separation of the two touch points 521AA, 521AB to be over four inches and that equates to the adjuster module 10A to display maximum display capacity or the lease zoom display capacity on the one or more programs on the one or more display screens 52A. Similarly, the separation of the two touch points 521AA, 521AB to be over two inches is determined to equate to the adjuster module 10A to display 50% and/or half of the full display screen display capacity, vice versa, etc.

In addition, for a two touch point gesture under an inch of separation, the adjuster 13A, the one or more graduations 120A, the adjuster indicator(s) 14A and/or the numeric indictor(s) 16A are generated by the adjuster module 10A between the two touch points 521A at a predesignated location not adjacent to the two touch points 521A with respect to the one or more programs on the corresponding display screen 52A. Where a distance of the two touch point gesture separation has been pre-programmed to display the respective displaying content(s) 212A of the one or more programs on the one or more display screens 52A specifically or in close proximity to a degree of the pre-programed two touch point gesture, without an initial physical movement of the two touch points 521A, based on the degree of the distance between the two touch points 521A. For example, the display screen controller module 20A determines the separation of the two touch points 521A to be under an inch and equates to the adjuster module 10A to display 0% and/or minimal zoom-out display capacity on the corresponding display screen 52A. Also, the two inches of separation of the two touch point gesture can also represent to the adjuster module 10A to display 50% and/or half of the full display screen capacity, vice versa, etc.

The two touch points 521A not only provide the user an immediate image percentage display of the displaying content 212A within a window on the one or more display screens with respect to the one or more programs, but also allow the user to have additional control capacity for, for example, zooming in or zooming out. When a separation between the two touch points 521A from under an inch, to one inch, to two inches and so on. Similarly, two touch points 521A coming together from four inches or more, the user may pinch bring together the two touch points to three inches or two inches space between the two fingers, or vice versa, etc., so as to manipulate the size of the displaying content 212A with respect to the one or more programs on the one or more display screens at the same time or in a timely manner, while the scale or size of the adjuster 13A, the one or more graduations 120A, the adjuster indicator(s) 14A, and the numeric indicator 16A provided between the two touch points 521A would be adjusted correspondingly.

The two touch points 521A, for example between the thumb and the index finger, may provide the user the immediate image percentage for resizing a displaying content 212A. In an alternative pinching or de-pinching inputting gesture of one or more programs divided by one or more sideline elements 31A may provide alternative functions when a similar but different gesture is paired with other programs like the program for volume or the program for brightness to operation within the operation areas 522A divided by one or more sideline elements 31A. In other words, according to the present invention, one or more programs divided by one or more sideline elements 31A may have different volume controls and/or different brightness display controls on the same display screen 52A at the same time or on one or more display screens 52A with respect to the one or more programs, and may allow the user to maintain the same capacity for, zoom-in or zoom-out program with similar pinching and de-pinching gestures where the module of analyzable factor(s) 26A along with the artificial intelligence module(s) 25A, identifying the pinching or depinching gesture as a different gesture paired with zoom program when the pinching and depinching gesture is done with the thumb finger as one touch point 521A and the other touch point 521A is done with the index finger and the middle finger. In other words, the program would recognize the extra middle finger in the performance of the two touch points 521A pinching and depinching gesture for zooming. In addition, the gesture for volume or brightness may differentiate when one touch point 521A is done with the thumb and the other touch point 521A is done with the index finger, the middle finger and the ring finger. It is worth mentioning that it is essential for pairing a gesture with a program and for performing the desire gestures on the display screen 52A with auxiliary tools like the adjuster 13A and the adjuster interface 21A, and etc., for the customization of the gestures when pairing a gesture and a program together.

It is worth mentioning that the adjuster 13A is a tool provided not only for gesture for moving and gesture for zooming, but also for other gesture operations of the one or more programs, including controlling displaying contents 212A, controlling and editing preset or customized gestures, controlling display brightness, controlling audio, controlling the sideline elements 31A, and etc. In other words, the adjuster 13A is a visual aid to the one or more touch points and gestures for controlling any and all programs of the system and the examples provided in this application is not intended to limit scope of the invention. In addition, the adjuster 13A can have various shapes as mentioned above with the one or more graduations 120A or without graduation 120A. Programs and gestures paired with each other may also provide corresponding adjusters 13A and adjuster interfaces 21A to control the module of analyzable factors 26A of the one or more gestures with respect to the one or more programs. For example, when typing a document, the user may want the popup sideline element 37A to be generated faster that the user will have the option to control that feature with the corresponding adjuster 13A for time control of that program through the use of the adjuster 13A and the adjuster interface 21A while the module of analyzable factors 26A help to optimization the user's controls. The user may control the one or more programs on one or more display screen 52A through his or her gestures and the adjuster 13A with the adjuster interface 21A.

Referring to FIGS. 46-47 and FIGS. 49A-49B, the third executable portion 3A (sideline module 30) is communicatively connected with the first executable portion 1A (adjuster module 10A) and the second executable portion 2A (display screen controller module 20A). According to the preferred embodiment of the present invention, the third executable portion 3A is configured to further provide one or more sideline elements 31A defining one or more operating areas 522A on the display screen 52A. When the one or more touch points 521A are detected as the gesture for operating the operating area 522A on the display screen 52A, wherein the display screen control module 20A (second executable portion 2A) identifies the gesture as adequate and compatible. The interactive module 40A (fourth executable portion 4) receives the touch point information and the operating area 522A is activated on the display screen 52A. In the preferred embodiment, the fourth executable portion 41A operates the operating area program 401A thereof to control the operating area 522A to be displayed on the display screen 52A to allow the user to control the operating area program 401A for operating any concurrent operating programs on the display screen 52A. The touch point information, including the location of the one or more touch points 521A, detected by the display screen controller module 20A is sent to the adjuster module 10A to coordinate the one or more touch points 521A with or without the adjuster 13A, with or without the adjuster indicator(s) 14A, with or without the pressure indicator(s) 15A, and/or with or without the numeric indicator(s) 16A provided by the adjuster module 10A in the operating area 522A in relation to the displaying contents 212A, wherein any movement of the one or more touch points 521A in the operating area 522A is determined as gesture inputting taking place by the user to operate the displaying contents 212A on the display screen 52A.

In one embodiment, the interactive module 40A may send the touch point information to the processor 51A through the communication module 55A to display the operation area 522A on the display screen 52A. For example, the user is able to use double tap gesture to activate and display the operation area 522A at the location where the user double taping and the adjuster 13A is formed at the location where the user fingers touching the display screen 52A while the finger touching points on the display screen 52A are detected as the touch points 522A on the adjuster 13A, as shown in FIGS. 49A and 49B.

It is worth mentioning that when the operation area 522A is activated, the operation area 522A is capable of following the continuous touch point as operating gesture performed by the user. More specifically, the operating area 522A is provided with touch point information according to the module of analyzable factor(s) 26A and the artificial intelligence module 14A in relative to the continues motion and selected gestures. For example, the current module of analyzable factors 26A determine gesture acceleration and direction and deliver the touch point information to the adjuster module 10A for contents display then to the interactive module 40A to reposition the operating area 522A based on the projected direction of the one or more touch points 521A on the one or more display screen 52A.

It is important to mention that the interactive module 40A houses most if not all of the operating programs, such as the interacting program 42A, the keyboard element program 43A, the gesture and cursor program 44A, the scrolling program 46A, the zooming program 47A, the state-off program 48A, the time tacker program 49A, the operating area program 401A, as well as the gesture dictionary 402. In the program dictionary 45A, all the available programs for gestures pairing are found like the keyboard element program 43A, the gesture and cursor program 44A, the state-off program 48A, the scrolling program 46A, the zooming program 47A, and the time tracker program 49A, wherein future programs are scalable as new functions to be updated. Additionally, every program may have additional gesture within the initial gesture like the gesture for the mouse to move the cursor is a specific gesture, plus the right and left gesture controls can be imbedded within the same gesture, wherein the additional gestures are also customizable and distinguishable from the mouse gesture movement option, wherein additional gestures are also available for pairing and to be performed while controlling the primary gesture, for example left tap with the index finger and right tap with the middle finger, as the five finger touch point controls the movement of the digital mouse. Furthermore, the left hand or the right hand gestures are interchangeable, wherein the gestures are collectively produced by both hands, where one hand controls the gesture for the movement of the cursor and the other hand controls the gesture of the right and left click/tap, etc.

Referring to FIG. 49C, according to the preferred embodiment, the user can triple tap on a desired location of the screen display 52A and then slide towards a direction, such as horizontally or vertically, to activate the sideline module 30A (third executable portion 3) to form a sideline element 31A on the screen display 52A dividing the screen display into two displaying portions 523A, 523A′ to provide two windows, wherein the sideline element 31A is a horizontal sideline element 31A (as shown in FIG. 49C) extended from the triple taping location if the user performs a triple tapping and then a horizontally sliding, or alternatively the sideline element 31A will be a vertical sideline element 31A if the user performs a triple tapping and then a vertical sliding. The original displaying content 212A is automatically displays in one of the two displaying portions 523A, and the other displaying portion 523A′ provides a reservation area 33A available for the user to as a new window to open another program to display thereon as the second displaying content 212A′.

Further, for displaying two displaying contents 212A, 212A′, the user simply performs a sideline gesture, such as triple tap, on the display screen 52A generating a sideline touch point information which is recognized by the display screen controller module 20A (second executable portion 2A) and sent to the sideline module 30A (third executable portion 3) to generate the sideline element 31A to split the display screen 52A into the two displaying portions 523A, 523A′. Then, when an operating gesture is operated by the user on each of the displaying contents 212A, 212A′ on the two displaying portions 523A, 523A′, the touch point information detected by the display screen controller module 20A is sent the adjuster module 10A to generate an adjuster 13A on that displaying portion 523A or 523A′ for adjustment operating of the displaying content 212A, 212A′ on that displaying portion 523A, 523A′ touched by the user.

The touch point information provided to the adjuster module 10A, the sideline module 30A and the interactive module 40A are analyzed touch points 521A to be produced in more than one way on the display screen 52A, wherein the display screen controller module 20A activates the module of analyzable factors 26A for distinguishing pressure, thickness, time, acceleration, direction, angle, interruptions, size, one hands or both hands, and/or location from one or more touch points and where the information is further reviewed and learned by the artificial intelligence module 25A to differentiate from past, current and for future touch points selections. In other words, the operating gestures according to the present invention can be interpreted as 4-Dimensional gesture in comparison to the convention control gesture which is generally 2-Dimensional gesture produced according to the number of fingers used and direction of the finger moved. According to the preferred embodiment of the present invention, the pressure of the one or more fingers, the movement of one or more fingers and the time duration of gesture contributed four dimensional touch point information for the adjuster module, the display screen controller module 20A, the sideline module 30A, the interactive module 40A for better sensation and control to better operate the programs of the electronic device 50A on the one or more display screens 52A.

Furthermore, referring to FIGS. 48A to 50, the adjuster module 10A generates a graduation indicator 14A and/or a numeric indicator 16A according to the current state of the displaying content, wherein the parameters of the display screen 52A and the preset proportion of the touch point 521A on the adjuster 13A are indicated on the adjuster indicator 14A through the use of the adjuster interface 21A on the adjuster 13A. The graduation 120A is provided on the adjuster 13A to show the moving scale of the displaying content 212A. Through the graduation 120A, the user understands the various available distance for display and the current distance of one or more displaying contents 212A of one or more programs on one or more display screens 52A for one or more program functions. The scales provide a customize percentage through the adjuster 13A. Where the finger of the user requires significant less physical demands to move a displaying content 212A in the display screen 52A in relation to the size of the physical display screen 52A and/or the size of the program/document being larger or smaller to the display area of the physical display screen 52A, wherein the user can easily manipulate the size of the displaying content 212A in any display medium.

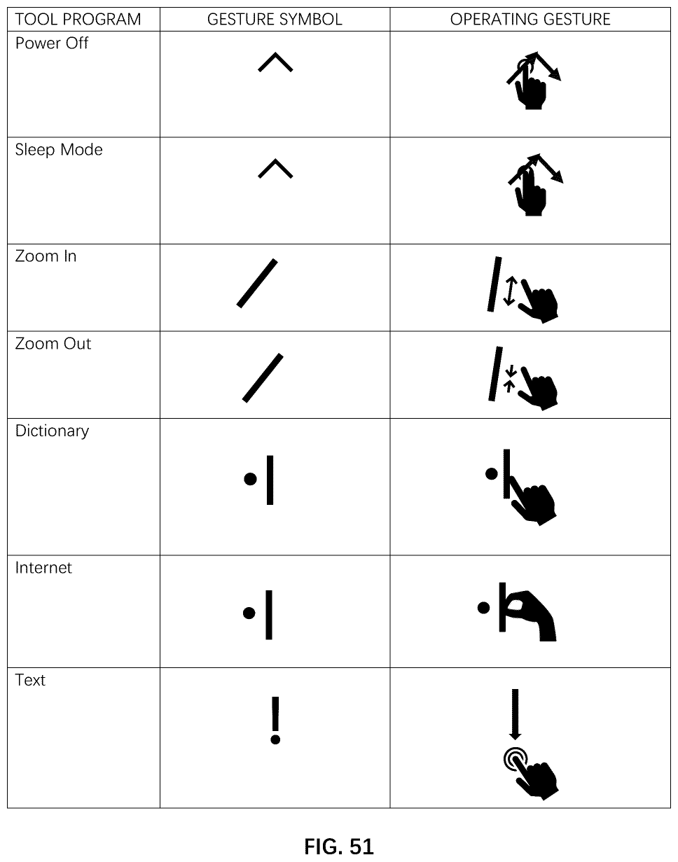

Referring to FIG. 51, an example of portion of the gesture dictionary is illustrated, wherein those operating gestures can be predesignated in the gesture dictionary 402A of the system such as the interactive module 40A. In addition, each operating gesture can be redesignated to any customized gesture by the user of the system or the owner of the electronic device 50A, facilitating the operation and control of the programs for the user as well as providing an innovative way to log in the system and/or the electronic device as authentication gesture password. For example, when a user has to log in the system or a program of the electronic device, the log in window may provide a function box for the user to select a specific function, such as ON/OFF, ZOOM IN, DICTIONARY, TEXT, and etc., and a gesture box for the user to execute the corresponding customized gesture personally designated and preset in the gesture dictionary 402A of the system by the user.

In addition, in the Metaverse technology with the usage of electronic device 50A such as AR/MR/VR devices, the user is provided with a work, home, or any immersive environment when the user must perform tasks as the means for user verification, through the use of gestures, wherein the performance and information of the touch point 521A in the gesture are analyze by the module of analyzable factor(s) 26A and the artificial intelligence module 25A, wherein the task selection and the gesture selection are analyzed and the order of the tasks is quantified by the artificial intelligence module 25A. For example, the user can make the verification immersive environment a library with hundreds of books to pick from where the order of picking up books and the performance of the gestures for the task are analyzable by the module of analyzable factor(s) 26A and the artificial intelligence module 25A.

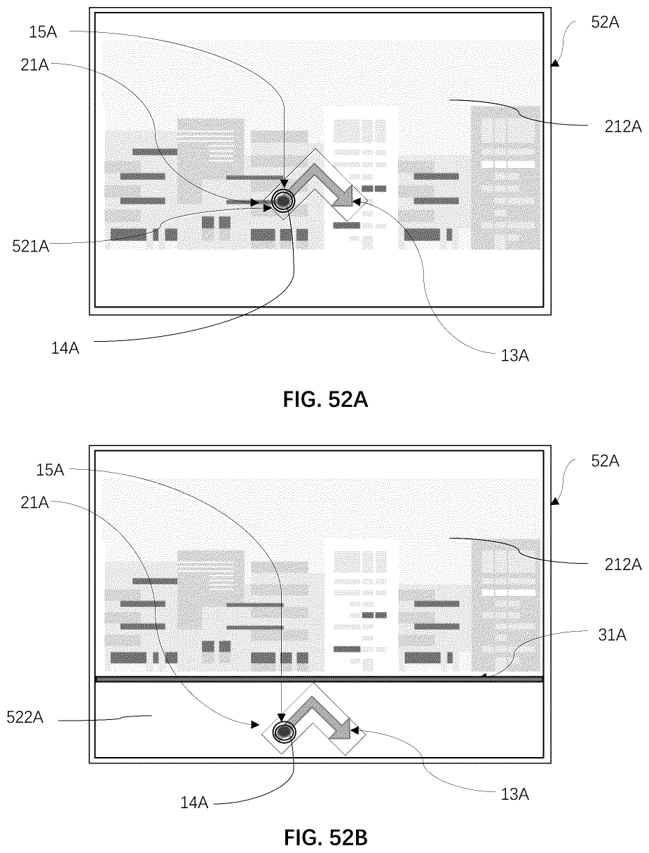

After operation of a program on the display screen 52A, when the user likes to have the electronic device 50A being power off, as shown in FIG. 52A, the user may simply use his or her finger to perform the power off gesture on the display screen 52A, the adjuster 13A will show at the touching location of the touch point 521A on the display screen 52A to guide the movement of the finger. When the touching finger moves inclinedly upwards and then inclinedly downwards to produce a reverse V gesture, the electronic device 50A will be operated to power off correspondingly after confirmation with the user. If the operating area 522A has already been provided on the display screen 521A by the sideline element 31A, the user may simply perform the operating gestures on the operating area 522A, as shown in FIG. 52B.

Similarly, when the user likes to operate the electronic device 50A to a sleep mode, the user may use his or her index and middle fingers perform the sleep mode gesture on the display screen 52A in a close together manner to produce two closed together touch points 521A, the adjuster 13A will show at the touching location of the touch points 521A on the display screen 52A to guiding the movement of the fingers. If the two fingers move inclinedly upwards and then inclinedly downwards to produce a reverse V gesture, the electronic device 50A will be operated to sleep mode correspondingly after confirmation with the user. In an alternative example, the extra finger touch point may be programmed for sleep mode of the electronic device 50A.

Another example is that when the user working on an image displaying content 212A and request to type in some texts on the display image, the user may activate the text program by using one finger to touch and slide on the display screen 521A or the operating area 522A of the display screen 521A downwardly and then promptly press on an adjacent spot with a predetermined pressure to produce an exclamation mark form as the text gesture, as illustrated in FIG. 51, to activate a text program correspondingly for the user to input texts. It is appreciated that the adjuster 13A, as well as the adjuster indicator 14A, the pressure indicator 15A, the numeric indicator 16A, and/or the graduation 120A can be provided or not at the same time with the adjuster 13A to facilitate and guide the user to effectively perform his or her operating gesture to operate and control the one or more operating programs from the module of analyzable factor(s) 26A precisely.

In order to demonstrate the updated features of the invention, the sliding gesture for moving, as shown in FIGS. 48A-48B and FIG. 49A, the pinching and de-pinching gesture for zooming, as shown in FIGS. 48C-4D and FIGS. 49B-49C, and the arrow gesture for power off, as shown in FIGS. 52A-52B, are illustrated as examples, wherein the above examples are only for demonstrating the operations of programs through designated or customized operating gestures but not intending to limit the scope of the claimed invention. Numerous programs available through one gesture with multiple module of analyzable factors and the computation of the artificial intelligence module 25A are workable in the present invention, where one gesture does not have to be bounded to only zooming-in or zooming-out that the pinching and de-pinching gesture with different pressing pressure or with two or more fingers may also be assigned to the operation of other functional programs according to the designation of the user. It is appreciated that in consideration of the module of analyzable factors 26A for each operating gesture, every module of analyzable factor can be program to an individual program from the program dictionary 45A in the interactive module 40A.

More specifically, the display screen controller module 20A detects the pressure, thickness, time, acceleration, direction, angle, interruptions, size, one hand or both hands, and the location of one or more touch points which are module of analyzable factors 26A being reviewed and learned by the artificial intelligence module 25A for differentiating from past, current and for future touch points selections of the touch point 521A on the display screen 52A, so that the first gesture and the second gesture are identified as one or two different gestures and etc. The display screen controller module 20A also identifies the touch points 521A of one or more split programs displayed on one or more displaying portions 523A divided by the one or more sideline elements 31A respectively.

In other words, the display screen controller module 20A activates the module of analyzable factors 26A from the one or more touch points 521A on one or more display screen 52A, so that the first gesture and the second gesture are distinguished as one complete gesture or two different gestures, and etc. The display screen controller module 20A analyzes the touch points 521A for whether each of the one or more split programs requires the adjuster 13A, the adjuster indicator 14A, the pressure indicator 15A, the numeric indicator 16A, the graduation 120A as well as whether the split programs require the one or more sideline elements 31A to divide the display screen 52A into two or more displaying portions 523A and relocation of the displaying contents 212A in the displaying portions 523A respectively. The display screen controller module 20A is also configured to analyze the one or more touch points 521A for one or more superimposed operating areas 522A being operated by the state-off program 48A through the interactive module 40A on one or more programs. For example, as shown in FIGS. 48A-48B, when the touch point 521A is created by the touching finger of the user of the display screen 52A, the adjuster 13A is provided on the display screen 2A under controlled by the adjuster interface 21A that the area of the original displaying content 212A on the display screen 52A and the adjuster 13A form the superimposed operating area 522A for operation through the operating area program 401A of the interactive module 40A. In particular, when the operating area program 401A is activated and superimposed on an existing program (i.e. a state-off program 48A), the portion of the program displayed on the display screen 52A may be locked to prevent that portion of the original running program and the original displaying content 212A from getting erroneous misleading information accidentally inputted while a gesture is being performed and created on the operation area 522A while the adjuster 13A is provided for illustration and guidance of the operating gesture performed by the user and created in the display screen controller module 20A. That portion of the program cannot be operated with touch points 521A or may have very limited touch points 521A operation capacity, while the operation area 522A is operational and furthermore the existing program has options to continue running or to pause on one or more display screen 52A.

The artificial intelligence module 25A learns from the display screen controller module 20A for touch point variants, as well as from the program's selections and task performances, of the selected programs on the sideline module 30A. The artificial intelligence module 25A is also learning from sideline module 30A for splitting of various programs and the programs selection for positioning into the reservation areas 33A and from the adjuster module 10A for recognizing and repositioning of displays contents 212A. In one embodiment, the artificial intelligence module 25A is configured to learn from the display screen controller module 20A by analyzing the one or more touch points 521A from the one or more module of analyzable factors 26A on one or more operating programs such as the reservation area 33A requiring the adjuster 13A being requested, the adjuster indicator 14A being manipulated, and the numeric indicator 16A being requested, and etc. In other words, the artificial intelligence module 25A learns the layout to the adjuster module 10A, to the sideline module 30A and to the interactive module 40A on the one or more display screens 52A to better assist the user with repetitive task.

It is worth mentioning that the present invention is intended to operate independent or with other inputting existing control options such as voice command, external auxiliary touch device like physical track pad, retinal computer-controlled device, eye movement tracking control, AR/MR/XR/VR devices, and etc. It is an objective to demonstrate the numerous options for an all-digital touch point control system and to illustrate a hybrid option for other existing and future inputting systems in the computer industry, or furthermore, in combination with more than one inputting computer systems.

The computer usable medium 100A can be the processor of the electronic device programmed to have computer-readable program code to perform executable operations of the first to fourth executable portions 1A, 2A, 3A, 4A. According to the preferred embodiment, as shown in FIGS. 45A and 46, the computer usable medium 100A is embodied as the cloud server configured to have computer-readable program code to perform executable operations of the first to fourth executable portions 1A, 2A, 3A, 4A such that the electronic device 50A communicatively linked therewith wirely or wirelessly, especially through 5G or above communication network, to run the programs in the cloud server and transmit the output to the electronic device 52A to display the output information through the communication between the server communication module 105 of the cloud server 100A and the communication module 55A of the electronic device 50A. It is appreciated that the cloud server may merely installed with some of the executable portions while the other executable portions, such as the adjuster module 10A, the display screen controller module or the sideline module 30) can be preinstalled in the electronic device 50A to collaboratively operate with the cloud server 100A.

According to the preferred embodiment of the present invention, the third executable portion 3A is configured to generate the one or more sideline elements 31A for organizing two or more applications on the display screen 52A. More specifically, a sideline element 31A divides one or more programs on the display screen 52A into two or more displaying portions 523A, 523A′, as shown in FIGS. 49A-49C, such as a first displaying portion 523A for displaying the displaying content 212A and a second displaying portion 523A′ for displaying the adjuster 13A as the operating area 522A as shown in FIGS. 49A-49B, or the first displaying portion 523A for displaying the original displaying content 212A and the second displaying portion 523A′ for displaying another displaying content 212A′ as shown in FIG. 49C. Once the sideline element 31A is activated to be provided on the display screen 52A, the application is able to display and operate on each displaying portion 523A of the display screen 52A.

It is appreciated that, through the sideline element 31A, the user is able to control displaying contents 212A in the first displaying portion 523A and the second displaying portion 523A′. For example, the user is able to use one finger to produce a touch point on the sideline element 31A, and another finger to touch the first displaying portion 523A, that is a two-touch point gesture with two touch points 521A, wherein the distance tracker module 27A of the display screen controller module 20A predetermines the touch point distance in relation to one hand or two hands, then the artificial intelligence module 25A sends the touch point information to the adjuster module 10. The display screen controller module 20A with the module of analyzable factors 26A and the artificial intelligence module 25A recognize the gesture for zooming program 47A, wherein the touch point information is also sent to the adjuster module 10A. The adjuster module 10A sends the touch point information to the processor 51A through the communication module 55A. The processor 51A sends the touch point information to display the adjuster 13A between the two touch points 521A. Furthermore, the one or more graduations 120A may or may not be displayed with the adjuster 13A. In other words, the user controls the displaying content size of one or more programs and one or more display screens with one or more fingers on the adjuster interface 21A and in the adjuster 13A.

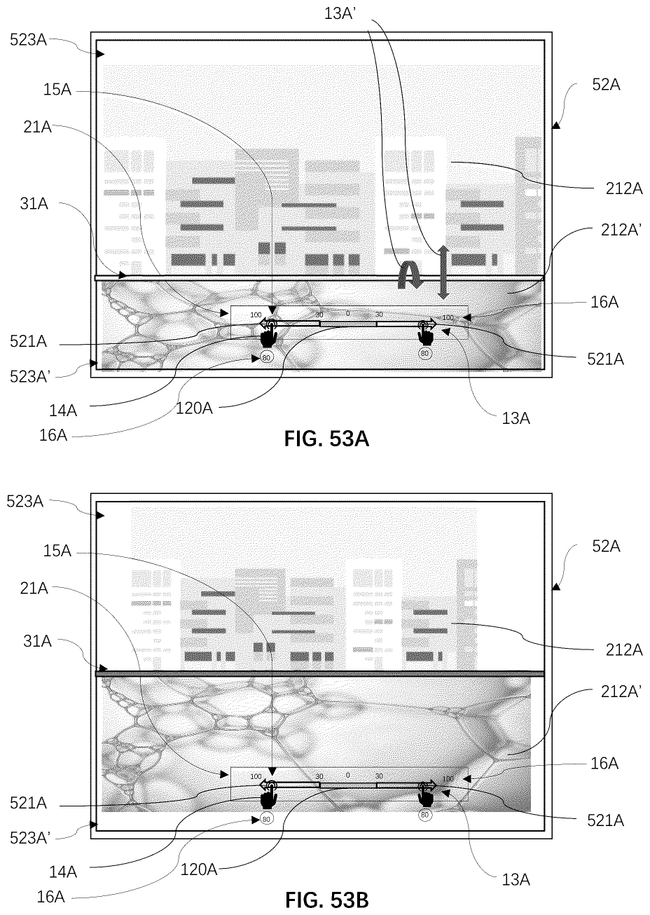

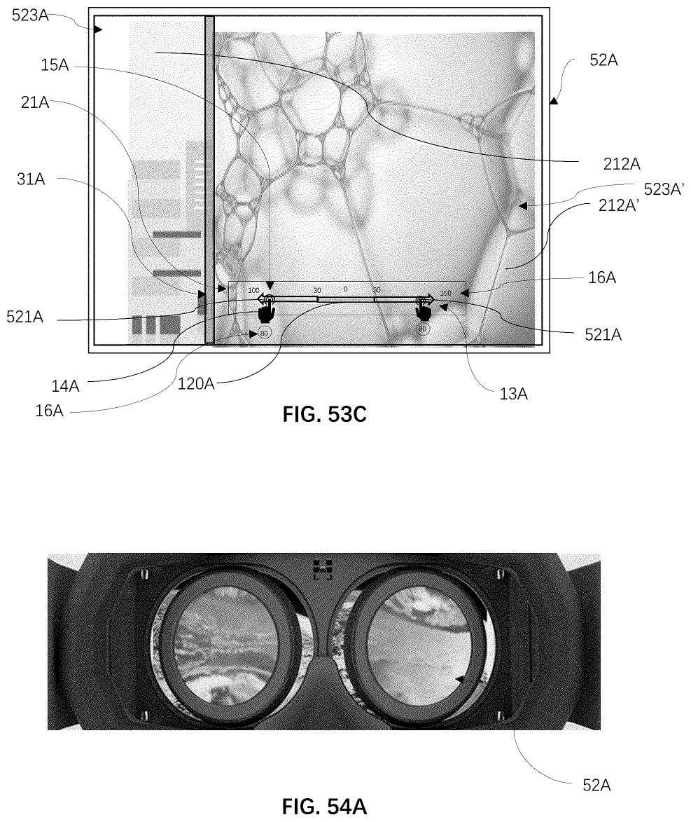

Referring to FIGS. 53A-53C, when two fingers of the user touches the sideline element 31A together at the same time, the display screen controller module 30A detects such sideline operating gesture and change a color of the sideline element, for example, and the user may simply slide the two touching fingers upwardly or downwardly together to move the sideline element 31A to an upper position or a lower position correspondingly to where the two touching fingers stop, as shown in FIG. 53B, when compared with FIG. 53A, or to rotate the two touching fingers with one of them as a center clockwise or anticlockwise to move the sideline element 31A from the horizontal position to a vertical position or vice versa correspondingly, as shown in FIG. 53C, wherein the displaying contents 212A, 212A′ displayed on the displaying portions 523A, 523A′ change positions accordingly. Similarly, to a vertical sideline element 31A as shown in FIG. 53C, the user may also move it leftwards or rightwards by the sideline operating gesture with the two touching fingers touching at the vertical sideline element 31A together to move leftwards or rightwards.

In addition, the sideline operating gesture detected by the display screen controller module 30A may generate the corresponding touch point information that is sent to the adjuster module 10A to generate one or more sideline adjusters 13A′ for sideline element 31A provided adjacent to the sideline element 31A, as shown in FIG. 9A, to guide the user to operate and control the positioning of the sideline element 31A.

It is worth further mentioning that, in one embodiment, through the motion of the touching fingers (and the touch points 521A generated correspondingly) on the sideline element 31A, the user may also able to zoom-in or zoom-out the displaying content 212A in one of the displaying portions 523A′, for example the second displaying portion 523A′, of the display screen 52A without affecting the other first displaying portion 523A on the display screen. As shown in FIG. 53C, if the user wants to adjust the displaying content 212A in the second displaying portion 523A′ of the display screen 52A, the user may simply use, for example, one finger to touch the sideline element 31A and another one finger to touch on the second displaying portion 523A′, wherein two touch points 521A are generated at the sideline element 31A and the second displaying portion 523A′ respectively and the locations of the two touch points 521A and the distance between the two touch points 521A determined by the distance tracker module 27A generate a sideline and zooming touch point information. Then, when the user moves the vertical sideline element 31A leftwards to enlarge the second displaying portion 523A′, the sideline element 31A is moved leftwards and the second displaying content 212A displayed on the second displaying portion 523A′ enlarges at the same time proportional while the magnification of the displaying content 212A′ displayed remains no change. At the same time, the size of first display portion 523A is reduced and the magnification of the display contents 212A remains unchanged. It is appreciated that the adjuster 13A provided on the second displaying portion 523A′ will change accordingly to illustrate the scale and size of the enlarged displaying content 212A′ on the second displaying portion 523A′ of the display screen 52A. Alternatively, reducing or enlarging the displaying portion 523A or 523A′ with the sideline element 31A can increase or reduce the display content magnification 212A or 212A′ accordingly.

More specifically, when the display screen controller module 20A, in combination with the sideline module 30A and the adjuster module 10A, detects a touch point 521A′ located at the sideline element 31A, the display screen controller module 20A will further detect whether any touch point 521A is located on the first displaying portion 523A of the display screen 52A or the second displaying portion 523A′ of the display screen 52A. If another touch point 521A is located on the second displaying portion 523A′ of the display screen 52A, the touch point 523A′ located at the sideline element 21A is defined as a sideline touch point 523A′ and the touch point 521A located in the second displaying portion 523A′ of the display screen 52A is defined a first touch point 521A. The touch point information of the sideline touch point 521A′ and the touch point information of the first touch point 521A are sent to the adjuster module 10. Furthermore, the adjuster module 10A retrieves the displaying content information displayed distance from the second displaying portion 523A′ of the display screen 52A and the information of the second displaying portion 523A′ of the display screen 52A from the display screen controller module 20A. The adjuster module 10A further generates one or more graduations 120A for the adjuster 13A according to the displaying content information displayed on the second displaying portion 523A′ of the display screen 52A, the information of the second displaying portion 523A′ of the display screen 52A and the information of the adjuster 13A. The one or more graduations 120A are displayed with the adjuster 13A. Preferably, the adjuster 13A is a bar segment arranged horizontally between the sideline touch point 521A′ and the first touch point 521A and the one or more graduations 120A are displayed at the adjuster 13A, as shown in FIG. 53C.

As shown in FIG. 53C, alternatively, the adjuster interface 21A displays the adjuster 13A with the one or more graduations 120A which is displayed starting at any position of the sideline element 31A or near the sideline element 31A, or one of the sideline elements 31A when there is more than one sideline elements 31A. The user moves the finger on the sideline element 31A to adjust the size of the displaying content in the second portion 523A of the display screen 52A. The display screen 52A detects the sideline touch point 521A′ is moving, the adjuster module 10A obtains the current graduation location from the corresponding graduation 120A from the sideline element 31A. The sideline module 30A sends the sideline information corresponding to the sideline element 31A and the current graduation 120A to the process 51A to adjust the displaying content 212A′ in the second displaying portion 523A′ of the display screen 52A, the current state of the displaying content 212A′, and the state of the second displaying portion 523A′ of the display screen 52A. If the sideline touch point 521A′ or the first touch point 521A is moved from a smaller graduation 120A to a larger graduation 120A, the displaying content 212A′ displayed in the second displaying portion 523A′ is zooming in. If the sideline touch point 521A′ or the first touch point 521A is moved from a larger graduation 120A to a small graduation 120A, the displaying content 212A′ displayed in the second displaying portion 523A′ is zooming out. In such a manner, the display size of the displaying content 212A′ displayed in the second displaying portion 523A′ of the display screen 52A is adjusted. It's worth mentioning the adjuster 13A can also be arranged vertically for display on the body of the sideline element 31A to control the second displaying portion 523A′ of the display screen 52A, where the displaying content 212A, including the display magnification thereof, of the first displaying portion 522A of the display screen 52A is not affected.

Alternatively, the touch point 521A with pressure over any sideline module 30A will activate the pressure indicator 15A where the information is received by the display screen controller 22A and by the module of analyzable factors 26A, verifying by the module of artificial intelligence 25A and delivering to the communication module 55A then to the processor 51A to activate the sideline module 30A to move according to the user's movements.

More specifically, the current position of the sideline touch point 521A′ refers to the current state of displaying content 212A′ displayed in the second displaying portion 523A′ of the display screen 52A. In other words, the current graduation of the one or more graduations 120A displays the current display state of the displaying content 212A′ of the second displaying portion 523A′ of the display screen. 52A. When the sideline touch point 521A′ is moved to a larger scale, the display screen 52A detects the current position of the sideline touch point 521A′ at the sideline element 31A. According to the current position of the sideline touch point 521A′, the adjuster module 10A sends the current scale of the sideline touch point 521A′ to the display screen controller module 20A. If the adjuster interface 21A is manipulated, the sideline information is sent through the communication module 55A to the processor 51A, and the processor 51A sends the sideline information to be displayed on the display screen 52A through the communication module 55A. The displaying contents 212A, 212A′ of the display screen 52A are adjusted by the adjuster module 10A which further controls the second displaying portion 523A′ of the display screen 52A to be adjusted to the current scale of the graduation 120A. If the current adjuster interface 21A on the graduation 120A is manipulated by, for example, the first touch point 521A from largest number to smallest number on the original scale of the graduation 120A, the displaying content 212A′ of the second displaying portion 523A′ is zoomed out, though the same process as mentioned in this embodiment, earlier. The second displaying portion 523A′ of the display screen 52A is zoomed out according to the current scale of the graduation 120A. The size of the displaying content 212A′ of the second displaying portion 523A′ is adjusted according to the current scale of the graduation 120A.

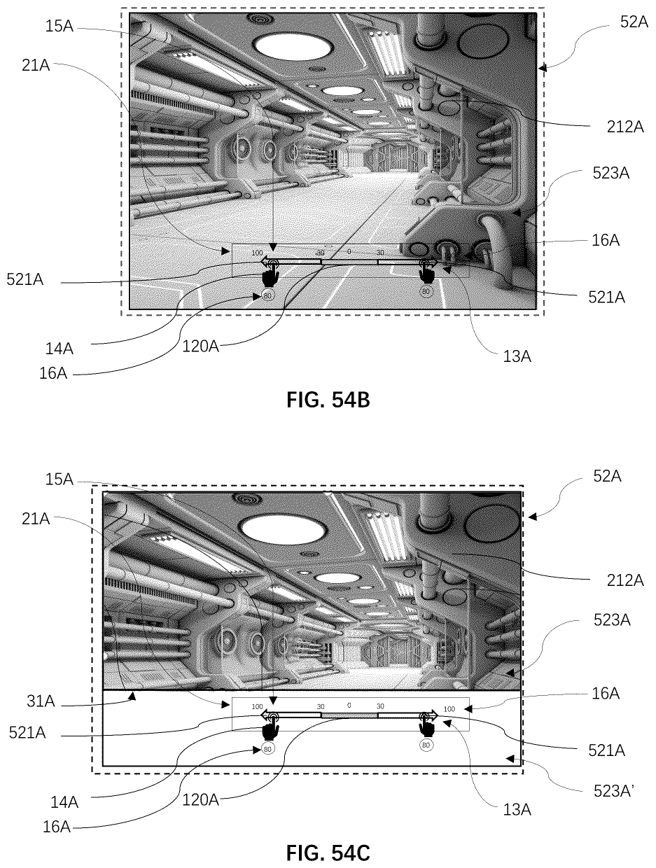

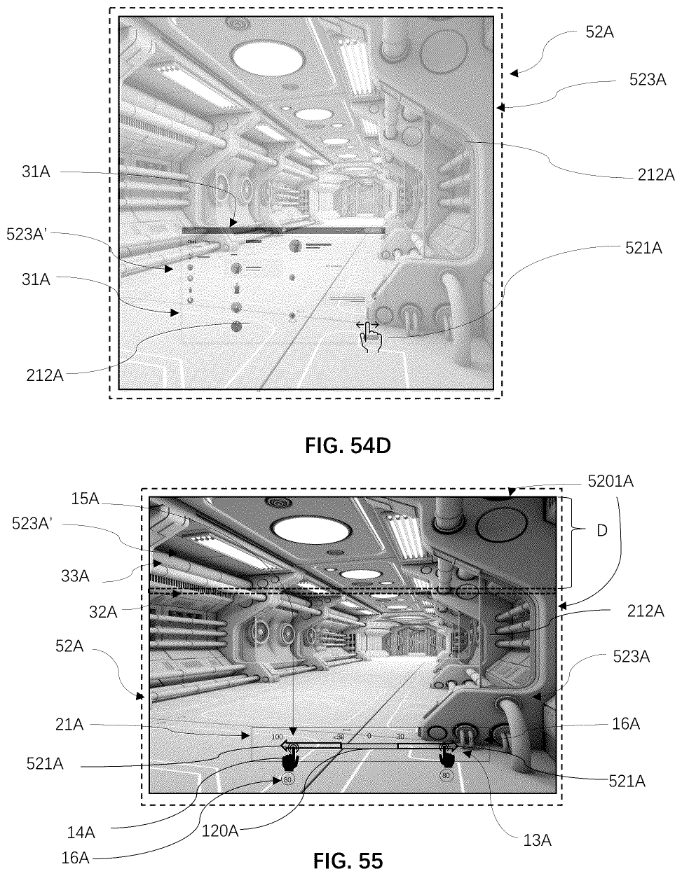

It is appreciated that the electronic device 50A would be embodied as a VR/MR/XR/AR apparatus that the display screen 52A can be a projection screen displaying through projecting device such as a helmet or a goggle as shown in FIG. 54A or other projecting device such as projector, optical display device, augmented reality display device, goggle headset, AR/MR/XR/VR headset or glasses, and etc. Through such headset, the user may view a virtual reality and/or the real reality via one or more cameras of the goggle 52A. Through the goggle type electronic device 50A, the user could view a full display screen 52A, referring to FIG. 54B, wherein the displaying content 212A is fully displayed within the displaying portion 523A. The adjuster 13A, the adjuster interface 21A, the graduation(s) 120A, pressure indicator(s) 15A, the numeric indicator(s) 16A can be activated to display and overlay on top of the displaying content 212A within the displaying portion 523A. The user may also divide the display screen 52A into two or more displaying portions 523A, 523A′, including one displaying portion 523A for the original displaying content 212A and the other displaying portion 523A′ for displaying the adjuster 13A, the adjuster interface 21A and etc., as shown in FIG. 10C. The boundaries of the displaying portion 523A and/or the boundary between the two displaying portions 523A, 523A′ forms the sideline element 31A which can be controlled and operated by the one or more touch points 521A produced through the user's gestures.

In other words, the display screen 52A of the present invention can be embodied as a screen of a tablet, smart device, laptop, a monitor of computer or notebook that is responsive and sensorial to a physical touch thereon by an object such as finger or touch pen, as well as a viewable screen through a headwear, such as glasses or goggle, or a projecting screen through a projector, voice command computer system, hologram or a 3D holography apparatus. The term ‘touch’ recited in the present invention generically refers sensory touch that when a movement of a finger of the user is sensed by the input device 56 of the electronic device 50A, such as a mouse, a touch pad, a touch panel, a resistance-type or capacitance-type touch screen, one or more cameras, laser/light sensor manipulation, hand/finger software detection program, one or more motion sensors, and etc., so as to determine the gesture of the user applied and operated.

Referring to FIG. 54D, the displaying portions 523A, 523A′ can be overlaid in a superimposed manner, wherein the user may activate another displaying portion 523A′ overlaying on top of the original displaying portion 523A, which can be a control panel to display the adjuster 13A and the adjuster interface 21A or, for example, a communication or chatting window that the user may execute communication while viewing and continuing the operation of the program displayed in the original displaying portion 523AA. The overlaying displaying portion 523A′ can be 100% overlaid on top of the original displaying portion 523A or, alternatively, having less than 100% transparency to enable the user to view both displaying portions 523A, 523A′ at the same time as illustrated in FIG. 10D. The user may utilize a touch pad or touch panel to input gestures that generates one or more touch points 521A displayed on the corresponding displaying portion 523A, 523A′ to control and operate the program displayed in that displaying portion 523A, 523A′. For example, the user may double tap on the overlaying displaying portion 523A′ and then use one finger to move the entire overlaying displaying portion 523A′ to another desired position on top of the original displaying portion 523A. The user may also double tap the overlaying displaying portion 523A′ and use two fingers to move towards each other or apart from each other to zoom in or zoom out the displaying content 212A′ of the overlaying displaying portion 523A′.

For purpose of illustration on the figures, FIGS. 54B to 56C, although the display screen 52A may have no boundary to the displaying content 212A being viewed through the projecting device such as goggle headset, the broken lines surrounding frame merely illustrates such AR/MR/XR/VR display screen 52A with headset and does not means there are actual borders for the displaying content 212A.

As shown in FIG. 11A, the sideline module 30A (the third executable portion 3A) is able to provide one or more reservation sideline elements 32A with respect to at least the physical edges of the display screen 52A or each displaying portion 523A of the display screen 52A respectively. The reservation sideline element 32A is a virtual sideline element defaulted by the system or customized by the authorized user of the display screen 52A through the sideline module 30A that may merely be illustrated as hidden lines (as shown in FIG. 11) or elongated transparent bar segment. The reservation sideline element 32A can be set as the respective edge of the display screen 52A, the respective edge of the displaying portion 523A or the sideline element 31A which divides the two displaying portions 523A, 523A′ and forms the boundary edge two adjacent displaying portions 523A, 523A′. Accordingly, upon the activation of the one or more reservation sideline elements 32A through the sideline module 30A on the display screen 52A, when the user moves his or her touching finger on the display screen 52A to perform a gesture to operate the displaying content 212A, such as a picture, to move accordingly toward a direction for viewing different portion of the picture, the picture will not be replaced by the next picture on the display screen 52A within even though the user simply move his or her touching finger all the way to the edge of the display screen 52A or the displaying portion 523A and the movement of the picture will stop at the edge of the display screen 52A or the displaying portion 523A, i.e. the reservation sideline element 32A. It is appreciated that the reservation sideline element 32A is preferred to be defaulted or customized to have a distance D from a corresponding side edge 5201A of the display screen 52A, wherein the portion between the reservation sideline element 32A and the corresponding edge 5201A defines a reservation area 33A.

It is appreciated that when the sideline element 31A is operated with a sideline conversion gesture with one or more touch points 521A, for example swiping the sideline element 31A to a side edge 5201A, such as top, bottom, right or left, from a middle portion of the display screen 52A, a sideline conversion is activated that where the sideline element 21A is converted into a reservation sideline element 32A and produce the reservation area 33A between the side edge 5201A where the sideline element 31A swiping to and the converted sideline element 31A on the display screen 52A. In other words, the reservation sideline element 32A is preferred to divide the displaying content 212A into two displaying portions 523A and 523A′, wherein the user may separately operate and control the two divided displaying portions 523A, 523A′ of the same displaying content 212A independently. For example, the adjuster 13A, the adjuster indictor 14A, the adjuster interface 21A, the pressure indicator 15A, the numeric indicator 16A, and/or the graduation 120A can be activated to be displayed in the displaying portion 523A that merely enables operation and control of the portion of the displaying content 212A displayed in the displaying portion 523A, such as zooming in or out, while the portion of the displaying content 212A displayed in the displaying portion 523A′ being unchanged.

Alternatively, the displaying portion 523A′ displayed in the reservation area 33A can be activated and controlled by the second executable portion 2A (display screen controller module 20A) and the fourth executable portion 4A (interactive module 40A), such that the user may operate and control the displaying portion 523A′ in the reservation area 33A. In addition, the user may expand the portion of the displaying content 212A on the displaying portion 523A′ to the entire displaying screen 52A when the user taps on the reservation sideline element 32A and move the reservation sideline element 32A away from the side edge 5201A of reservation area 33A to enlarge the reservation area 33A. Also, if the user taps on the reservation sideline element 32A and move the reservation sideline element 32A toward the side edge 5201A of the reservation area 33A, the reservation area 33A as well as the portion of the displaying content 212A in the displaying portion 523A′ are reduced correspondingly.

According to the preferred embodiment of the present invention, the reservation area 33A is an area that may also be programmed to run and operate a scale down function at the same time or in a timely manner. Preferably, the reservation area 33A may be reserved at the side edges 5201A of the display screen 52A, such that the reservation sideline element 32A is able to move from left-to-right, right-to-left, top-to-bottom, or bottom-to-top according to the user selection on the display screen 52A. For example, if the reservation sideline element 32A is moved towards the top side edge 5201A of the displaying portion 523A′ on the display screen 52A and the displaying portion 523A′ inside the reservation area 33A reduces its area and becomes smaller. If the reservation sideline element 32A is moved toward a middle of the display screen 52A, i.e. away from the top side edge 5201A, the displaying portion 523A′ in the reservation area 33A enlarges its area and becomes bigger, and at the same time, the size of the displaying portion 523A reduces correspondingly. It is appreciated that when a sideline element 31A is swiped to one of the side edges 5201A of the displaying screen 52A, the sideline element 31A is converted to a reservation sideline element 32A and becomes a boundary to the reservation area 33A between the corresponding side edge 5201A and the reservation sideline element 32A on the display screen 52A. In other words, the sideline element 31A can be converted by a gesture swiping toward one of the side edges 5201A on the display screen 52A. It is worth mentioning that one or more touch points 521A on the reservation sideline element 33A can be moved to the middle of the display screen 52A or more toward the side edge 5201A of the display screen 52A without producing a conversion once the reservation sideline element 33A has been established.

The portion of the displaying content 212A displayed in displaying portion 523A′ of the display screen 52A is fully operational when it is positioned in the reservation area 33A. Either the sideline element 31A or the reservation sideline element 32A is able to adjust a size of the displaying portion 523A′ and the size of the displaying portion 523A at the same time. More specifically, when the display screen controller 40A detects the touch point 521A located in the reservation area 23, the touch point 521A produces a gesture for the state-off program 48A, wherein the information of the one or more touch points 521A is transported by the communication module 55A from the display screen 52A. The display screen controller module 20A may activate the module of analyzable factors 26A and the artificial intelligence module 25A. After the information of the one or more touch points 521A is send to the adjuster module 10A for displaying contents and the interacting module 40A, the state off program 48A is requested and then the information of the one or more touch points 521A is deliver to the processor 51A by the communication module 55A and is displayed on the display screen.

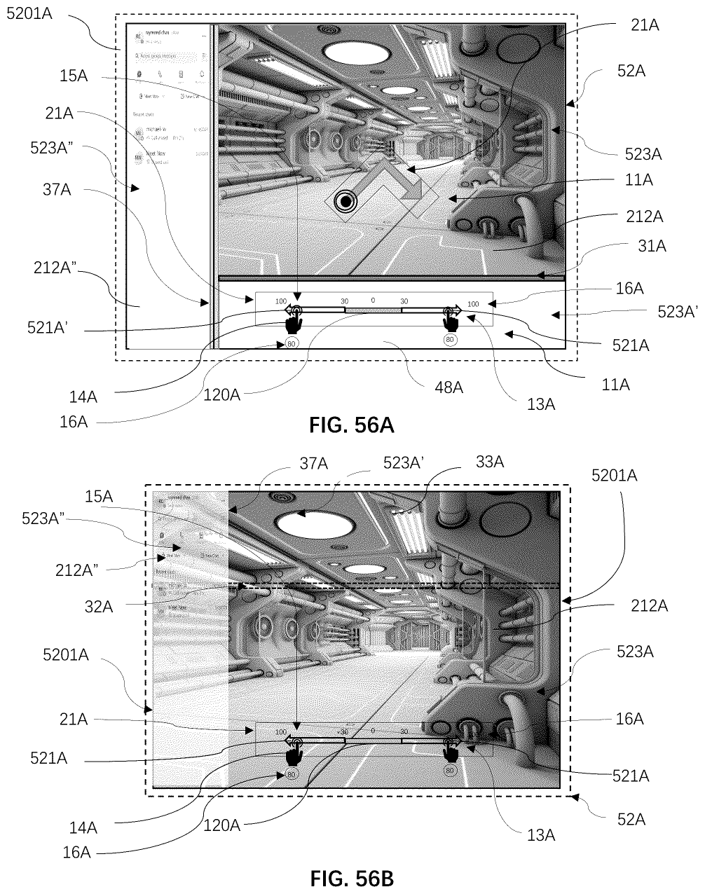

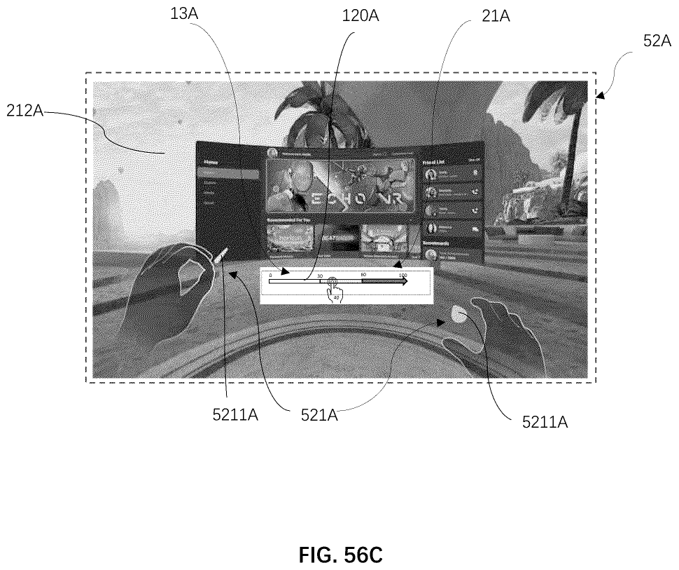

During an operation of one or more programs on one or more displaying portions 523A of the display screen 52A, an email, message, notification, voice call and/or the like may be incoming to the electronic device 50A of the user. Instead of the conventional notification in the toolbar of the display screen 52A to notify the current user of a conventional electronic device, the present invention distinctively allows the user to selectively activate the sideline module 30A (the third executable portion 3) to provide the pop-up sideline element 37A. Referring to FIGS. 47A and 56A, when the function of pop-up sideline element 37A is activated, an incoming call or message detected will trigger the pop-up sideline element 37A to display at a default location, such as a left side portion of the display screen 52A as shown in FIG. 56A, a third displaying portion 523A″ is divided at a left portion of the displaying screen 52A while, for example, the first displaying portion 523A for the displaying content 212A is positioned in a right upper portion of the display screen 52A and the second displaying portion 523A′ for the adjuster 13A, adjuster indicator 14A, the adjuster interface 21A, and etc. are positioned in a right lower portion of the display screen 52A, wherein a pop-up displaying content 212″, such as a messenger dialog, is displayed in the third displaying portion 523A″ allowing the user to timely take an incoming phone call or responding to an incoming message or chat. Of course, the user may also select to deactivate the pop-up sideline element 37A and no pop-up displaying portion 523A″ will pop-up. After the responsive action taken on the pop-up displaying content 212″ on the pop-up displaying portion 523A″, the user may simply execute a swiping gesture to swipe the pop-up sideline element 37A to the closer side edge 5201A of the display screen 52A and the pop-up sideline element 37A to clear away the pop-up displaying portion 523A″ and the displaying portion 523A, as well as the second displaying portion 523A″, if any originally, will return to its original size and location. It is worth mentioning that the shape, size, color, and/or form of the pop-up sideline element 37A is preferred to be different from the sideline element 31A as well as the reservation sideline element 32A, such as the three-lines form for the pop-up sideline element 37A and the two-lines form for the sideline element 31A.

In addition, as described above, the interacting portion 41A and/or interacting element programs 42A of the fourth executable portion 4A (interactive module 40A) can be switched between each other with other programmable elements, wherein one or more of the keyboard portion, the pad portion, the mouse portion, the cursor portion, the gesture dictionary portion, the zooming portion, the scrolling portion, the off state element portion, and the gesture operation portion of the interacting portions 41A displayed on the displaying portions 523A, 523A′ and the pop-up displaying portion 523A″ split by the pop-up sideline element 37A respectively can be operated and controlled independently and all the gestures on the gesture dictionary 402A are scalable while the programs for joining the gestures are also scalable. Also, when the state-off program 48A is triggered to an activation option, each of the interacting portions 41A becomes a state-off portion and provides gesture operations. When the state-off portion is in a lock option, the state-off portion has limited gesture options. Further, the interacting programs 42A and the interacting portions 41A can be superimposed by the state-off program 48A, wherein when the state-off program 48A is triggered, the one or more interacting portions 41A and the one or more interacting programs 42A are provided with additional gesture capacity, and that when the state-off program 48A is in the lock option, the state-off program has limited for any gesture option. It is worth mentioning that the superimposed state-off program 48A does not re-adjust the displaying contents.

It is appreciated that the fourth executable portion 4A (interactive module 40A) is operable to designate one or more programs on one or more interacting portions 41A on the one or more displaying portions 523A of the display screen 52A, wherein the one or more interacting portions 41A interact with the one or more programs displayed on the one or more displaying portions 523A, 523A′, 523A″ of the display screen 52A, wherein when the one or more interacting portions 41A are activated, the one or more interacting portions 41A are superimposed or displayed on the one or more displaying portions 523A, 523A′, 523A″ of the display screen 52A according to one or more positions of the one or more touch points acquired from the second executable portion 2A. In addition, when the one or more interacting programs 42A are activated, the one or more interaction programs 42A may be superimposed or displayed on the one or more displaying portions 523A, 523A′, 523A″ of the display screen 52A according to one or more positions of the one or more touch points 521A acquired from the second executable portion 2A.

It is worth mentioning that, as shown in FIG. 56B, the pop-up displaying portion 523A″ displayed between the pop-up sideline element 37A and the side edge 5201A of the display screen 52A can be superimposed on the displaying portion 523A, wherein the pop-up sideline element 37A can be simply a boundary side of the pop-up displaying portion 523A″. In other words, the pop-up displaying content 212″ of the pop-up displaying portion 523A″ is superimposed on a portion of the displaying portion 523A that can be accomplished by the state-off program 48A to superimposed on the notification existing program and can be accomplished with the same gestures from the reservation area 33A or with different gestures as mentioned in this invention.

Once the display for the state-off program 48A is accomplished and superimposed on the pop-up displaying portion 523A″ and/or on the reservation area 33A. The user has limited gesture controls over the side edges 5201A of the display screen 52A, allowing the portions or areas with the superimposed state-off by the state-off program 48A to be grabbed and manipulated without inputting erroneous data accidentally. Preferably, the gestures for the state-off are gestures with two touch points 521A transported by the communication module 55A where the information is reviewed by the module of analyzable factors 26A, the artificial intelligence module 25A and the distance tracker module 27A inside the display screen controller 22A where the separations between the two touch points 521A are measured to be too great and unable to come from one hand to turn on or off the state-off program 48A in the reservation area 23A and/or in the pop-up displaying portion 523A″. Lastly, the information is sent to the processor 51A through the communication module 55A, where the processor 51A sends the information to the display screen 52A to superimpose on the existing program with the state-off program 48A. As illustrated, the reservation sideline element 32A and the pop-up sideline element 37A may be always provided at the side edges 5201A of the display screen 52A where are the ideal locations for the user to have structural physical area for control and handling of the electronic device 50A that the user may simply tap on the corresponding side edge 5201A and swipe towards a middle portion of the display screen 52A to drag down the reservation sideline element 32A or the pop-up sideline element 37A to function. It is appreciated to consider the reservation areas 33A and the pop-up displaying portion 523A″ already have programs operating, therefore holding or manipulating the display screen 52A by the side edges 5201A is difficult to control the electronic device 50A and can produce erroneous accidental inputting without the aid of the state-off program 48A. Additionally, the state-off program 48A can allow the program to continue running or can pause the existing programs when the state-off is activated, preferably the state-off program 48A when operating is a watermark superimposed illumination of any selected color over the selected programs or at the side edges 5201A in combination with the reservation sideline 32A and or the pop-up sideline element 37A. It is noted that the sideline element 21A, the pop-up sideline element 37A and the reservation sideline element 32A can have the same color or different colors.

According to the present invention, the operation of one or more functions of a program is able to be processed through one or more gestures. For example, one or more touch points 521A on the reservation sideline element 32A with a continuous gesture crossing the sideline element 31A and the last portion of the gesture ending on the displaying contents 212A in the displaying portion 523A or vice versa, wherein the touch points 521A can be recognized as a gesture by the display screen controller 40A and that the module of analyzable factors 26A and the artificial intelligence module 25A understand the information which is delivered through the communication module 55A to the processor 51A to switch the program in the reservation area 33A and the displaying contents 212A in the corresponding displaying portion 523A on the display screen 52A.

It is worth mentioning that the one or more sideline elements 31A ensure the display screen 52A to provide two or more displaying portions 523A, 523A′ to display two or more displaying contents 212A of two or more programs respectively while the user is capable of controlling and operating the two or more programs displaying on the two or more displaying portions 523A, 523A′ divided by the sideline elements 31A with the support of the adjusters 13A and gestures respectively at the same time.

In particular, as illustrated in FIGS. 46-47A and 56A-56B, the processor 51A is programmed to send a notification or incoming message information to the sideline module 20A (the second executable portion 2A) when the notification or an incoming call or message is received. The sideline module 30A (the third executable portion 3A) generates a pop-up information and sends to the display screen 52A. The pop-up sideline element 37A is provided in display screen 52A dividing the display screen 52A into the displaying portion 523A and the pop-up displaying portion 523A″ displaying the notification or message. Preferably, the popup sideline element 37A is arranged to be positioned between one side edge 5201A and the displaying portion 523A of the display screen 52A. As described above, the pop-up sideline element 37A and the pop-up displaying portion 523A″ of the notifications or messages are arranged to display in a superimposed manner, wherein the popup sideline element 37A can be controlled by a superimposed program with time display customization on an existing program as illustrated in FIG. 56B.

It is worth mentioning that the reservation area 33A is formed by splitting the existing program with the reservation sideline element 32A, as shown in FIG. 55, wherein the reservation sideline element 32A can be moved to enlarge and minimize the reservation area 33A, allowing for more than one program to operate completely including the inputting controls. In addition, the smaller display portion 523A′ on the reservation area 33A can interchange with the current enlarge display portion 523A, wherein a continues gesture with one or more touch points 521A starting on one program/display portion 523A′ and finishes at another program/display portion 523A and the crossing of one or more sideline element 31A or reservation sideline element 32A is required.

It should be noted that the alternating of the two programs/display portions 523A is between the first touch program/display portion 523A and the last touch program/display portion 523A and that the continuous gesture may cross over the sideline element 31A, the popup sideline element 37A, or the reservation sideline element 32A. For example, the first touch point 521A may be located inside the reservation area 33A of the display screen 523A′ and the last touch point 521A is moved and located on the displaying contents 212A in the displaying portion 523A as a complete gesture on one or more programs. As shown in FIGS. 56A-56B, on the left side the popup sideline element 37A is a time display customizable notification display. As shown in FIG. 56B, on the left side of the pop-up sideline element 37A is the pop-up displaying portion 523A″ superimposed on the original displaying portion 523A to be displayed for as long as the user desires to have more than one programs on one or more displaying portions 523A, 523A″ at the same time or in a timely manner. Additionally, the user can interchange two programs or displaying contents 212A without crossing one or more displayed sideline elements 31A by customizing one or more gestures in the interacting module 30A by selecting one or more gestures in the gesture program dictionary 402A and selecting the program in the program dictionary 45A for switching two programs.

According to the preferred embodiment of the present invention, the display screen 52A is showing the motions and options of the touch points 521A to produce gestures for switching two programs operating at the same time on one or more displaying portions 523A of the display screen 52A. For example, the displaying content 212A running by the first program displays on the first displaying portion 523A as a more condensed size while larger displaying content 212A′ can be displayed on the second displaying portion 523A′. The user may produce a continuous gesture or a separate but similar gesture for the two programs being selected for switching, wherein either method crosses a sideline element 31A and/or any other physical or digital barrier/separation. The gestures for switching the two programs are preferred to be pre-programmed. For another example, one or more touch points 521A can be initiated inside the reservation area 33A as one of the selected programs to be switched, wherein the initial touch point 521A is a continuous parabola and/or segmented gesture crosses any sideline element 31A and/or any other physical or digital barrier/separation, such that the last portion of the gesture is produced on the other program intended to be switched with, such as three fingers are recognized as a three touch points gesture for switching programs that the initial three fingers are recognized by the display screen controller module 20A, including the module of analyzable factors 26A, the artificial intelligence module 25A, the adjuster module 10A, and the interacting module 40A, wherein the final three fingers are recognized by the same process on another program divided by a sideline element 31A and/or on one or more display portions 523A on the display screen 52A of the electronic device 50A. The information is transferred to the processor 51A by the communication module 55A to process and be displayed on the display screen 52A.

It is appreciated that the one or more touch points 521A can become a gesture for closing programs. Gestures can also work with other programs like the reservation sideline element 32A, such that a total swiping or dragging of the reservation sideline element 32A towards the side edge 5201A of the display screen 52A closer to the reservation sideline element 32A will close the reservation area 33A and the program and displaying content 212A in the reservation area 33A will be closed too while, at the same time, the displaying contents 212A in the main displaying portion 523A is completely enlarged to take over the space of the previous reservation area 33A without affecting the placement and displaying content 212A of the pop-up displaying portion 523A″ divided by the pop-up sideline element 37A (if any).

It is worth to further mention that all closures of programs can be customized for time where the undo gesture can return any past closed programs back to full displacement in a chronological orderly manner on the display screen 52A, no matter if it was closed by a gesture or by the sideline module 30A. The display screen controller module 20A recognizes a program closing gesture through the module of analyzable factors 26A and the artificial intelligence module 25A as the sideline module 30A is activated and then the information is provided and delivered to the adjuster module 10A, display screen controller module 20A and sideline module 30A which recognize and close the corresponding displaying portion 523A, 523A′ or 523A″ and eliminate and remove the sideline element 31A, the reservation sideline element 32A or the pop-up sideline element 37A from the display screen 52A. Also, the information is delivered to the processor 51A by the communication module 55A and processor 51A delivers the information to the display screen 52A to expand the leftover displaying content 212A completely in the current displaying portion 523A of the display screen 52A.

On the other hands, the display screen 52A may provide instruction and/or display of designated options and motions of touch points 521A for closing a program so as to increase the viewing capacity of other display programs and to increase the number of other programs operating at the same time. The user may select the gesture for forming touch points 521A on the display screen 52A for closing programs on the one or more display screens 52A from the program dictionary 45A and one or more gestures from the gesture dictionary 402A in the interacting module 40A for pairing the gesture or gestures. The information of the program and gesture to be paired is preferred to be performed and customized by the user on the display screen 52A. After the user performs the individualized and selected gesture, the display screen controller module 20A runs the module of analyzable factors 26A along with the artificial intelligence module 25A to produce an information of the respective touch points 521A of the selected gesture, that is confirmed by the processor 51A before displaying the selected function on the display screen 52A and stored in the storage device 53A at the same time. Also, the information may also be sent to the server storage device 103A in the cloud server 100A. The closed program is immediately stored on the storage device 53A for immediate return capacity and/or by the server storage device 103A for prolong return capacity. It is worth mentioning that the electronic device 50A may have similar functional features as described above without a built-in processor in the electronic device 50A in which the cloud server processor 101A takes over all the related function required in the electronic device 50A.

The cloud server 100A can be configured to operate all functions of the electronic device 50A, including the moving the sideline element 31A right, left, up or down to the nearest side edge 5201A of the displaying portion 523A on the display screen 52A. The sideline module 30A (the third executable portion 3A) acquires the sideline information from the display screen 52A through the server communication module 105A. The sideline module 30A adjusts the sizes of the first displaying portion 523A and the second displaying portion 523A′. The sideline module 30A generates the adjusted information regarding to the sizes of the first displaying portion 523A and the second displaying portion 523A′. The adjust information is sent to the display screen controller module 20A (the second executable portion 2A). The information is processed through the module of analyzable factors 26A, the artificial intelligence module 25A, and the distance tracker module 27A in the display screen controller module 20A so that the information is generated according to the adjusted information. The control information is sent to the processor 51A through the communication module 55A. The processor 51A processes the control information and sends to the display screen 52A through the communication module 55A. The display screen 52A displays the adjusted size of the first displaying portion 523A and the adjusted size of the second displaying portion 523A′, wherein when the touch point 521A tapped on the sideline element 31A moves right, left, up or down, the position the sideline element 31A moves accordingly with the position of the touch point 521A. Also, when the touch point 521A tapped on the sideline element 31A moves to one of the side edges 5201A of the display screen 52A and then back to a position adjacent to said side edge 5201A, the sideline element 31A can be converted to a reservation sideline element 32A and the area between the converted reservation sideline element 32A and said side edge 5201A of the display screen 52A becomes the reservation area 33A while the reservation sideline element 32A becomes a boundary of the reservation area 33A.

The sideline module 30A (the third executable portion 3A) produces the sideline element 31A to separate one or more touch points 521A and one or more programs. In other words, the sideline element 31A filters gestures along with the module of analyzable factors 26A, the artificial intelligence module 25A, and/or the distance tracker module 27A as they relate to the program controls operating within the boundaries of the sideline element 31A. For example, the pop-up sideline element 37A may have a single touch point 521A to control an opening of a program selection not to be confused with a single touch point 521A on the sideline element 31A to activate the motion of the sideline element 31A as well as a single touch point 521A on the display content 212A of a program to activate the adjuster module 10A (the first executable portion 1A) for zooming. Additionally, the sideline elements 31A provide the locations for more than one touch points 521A to operate on the same program at the same time. For example, a program operating a cursor program on a typing program, where the program is zooming and the program is divided by a sideline element 31A, all at the same time, without inputting erroneous information to the other programs operating at the same time.

According to the preferred embodiment of the present invention, the user interface displayed on the display screen 52A as shown in FIG. 56A comprises a sideline element 31A and a pop-up sideline element 37A and the user interface display on the display screen 52A as shown in FIG. 56B comprises a reservation sideline element 32A and a pop-up sideline element 37A. The sideline element 21A divides the display screen into the first displaying portion 523A and the second displaying portion 523A′ of the display screen 52A, wherein the first displaying portion 523A and the second displaying portion 523A′ run different programs and are divided by the sideline element 31A.

For example, referring to FIG. 56A, a displaying content 212A is displayed in the first displaying portion 523A and a functional program such as a keyboard element program 42A or a detect executable portion 11A (including an adjuster 13A, one or more adjuster indicators 14A, one or more pressure indicator 15A, and one or more numeric indicators 16A) and an adjuster interface 21A are displayed in the second displaying portion 523A′ of the display screen 52A. A detect executable portion 11A may also be activated on the displaying portion 523A for controlling and operating the displaying content 212A on the first displaying portion 523AA. It is worth mentioning that the displaying content 212A in the first displaying portion 523A can be a text file running with a word processing program and the detect executable portion 11A can be replaced by a keyboard element running by the keyboard element program 43AA on the second displaying portion 523A′ for inputting text or editing the text file displayed on the first displaying portion 523AA. Alternatively, when the game program is running and its displaying content 212A is displayed on the first displaying portion 523A, the detect executable portion 11A and the adjuster interface 21A displayed on the second displaying portion 523A′ can be functioned in form of a game control console for controlling and operating the game program running in the first displaying portion 523AA. Further, as shown in FIG. 56B, a pop-up displaying portion 523A″ can be preset to be popped up during an incoming or outgoing call or message or while it is activated by the user. The position and size of such pop-up displaying portion 523A″ can be preset or simply select the default setting by the user. The display screen 52A is split to provide the pop-up displaying portion 523A″ by a pop-up sideline element 37A and the first and second displaying portions 523A, 523A′ reduce their sizes accordingly to allow the pop-up displaying portion 523A″ located between one of the side edges 5201A (such as the left side edge as shown in FIG. 56B) of the display screen 52A and the pop-up sideline element 37A. The user may operate the displaying content 212″ on the pop-up displaying portion 523A″ with the touch points 521A of designated gestures.

For example, referring to FIG. 56B, a displaying content 212A is displayed in the first displaying portion 523A which is the whole area of the display screen 52A and a functional program such as a keyboard element program 42A or a detect executable portion 11A (including an adjuster 13A, one or more adjuster indicators 14A, one or more pressure indicator 15A, and one or more numeric indicators 16A) and an adjuster interface 21A are also superimposed and displayed in the first displaying portion 523A of the display screen 52A. A pop-up displaying content 212″ such as a messenger dialog during an incoming or outgoing call or message is displayed in a pop-up displaying portion 523A″ in a superimposed manner with a predetermined percentage of transparency as shown in FIG. 56B. The overlapping boundary of the pop-up displaying portion 523A″ and the first and second displaying portion 523A, 523A′ becomes and functions as the pop-up sideline element 37A. FIG. 56B also illustrates the reservation area 33A divided by the reservation sideline element 32A which is activated to be horizontally extended at an upper portion of the display screen 52A. The reservation area 33A can be used to display desired displaying content or, preferably, a desired portion of the displaying content 212A displayed on the displaying portion 523A so that the user may independently operate and control the portion of the displaying content 212A within the reservation area 33A and the residual portion of the displaying content 212A displayed on the displaying portion 523A via the touch points 521A through the interacting program 42A or other inputting devices through the keyboard element program 43A, the gesture and cursor program 44A, the scrolling program 46A, and etc.

The sideline element 31A, the reservation sideline element 32A and the pop-up sideline element 37A are programmed to be displayed with the orientation of the display screen 52A that, for example, when the electronic device 50A is clockwise rotated, the sideline element 31A, the reservation sideline element 32A or the pop-up sideline element 37A is clockwise rotated correspondingly, that is the sideline element 31A, the reservation sideline element 32A or the pop-up sideline element 37A is rotated from a vertical position to a horizontal position. In addition, the text edit program, any game and gamer controller module as well as the keyboard element program 43AA are clockwise rotated and adjusted, and vice versa. When the displaying content 212A is a game content of a game program, an adjuster interface 21A for the game program and the text edit program can be displayed in the first displaying portion 523A of the display screen 52A, as illustrated on FIG. 56A. The adjuster interface 21A of the game controller program and the keyboard element program 43AA can also be displayed on the lower displaying portion 523A′ of the display screen 52A, as illustrated on FIG. 56A, so that the adjuster interface 21A for the game controller program is displayed under the displaying portion 523A of the game. Even the electronic device 50A and the displaying screen 52A thereof is rotated to change its orientation, the displaying portions 523A, 523A′, 523A″ will be rotated correspondingly to change their locations arranged by the user, for example, the first displaying portion 523A is remained in an upper position, the second displaying portion 523A′ is remained in a lower position, and the pop-up displaying portion 523A″ is remained in a left side portion of the display screen 52A, i.e. no matter the display screen is in portrait or landscape orientation, so as to provide the user convenience for texting and gaming, to edit program and play the game, the controls/inputting are located always most proximal to the users' body. It is worth mentioning that in the reversal rotation (counterclockwise) of the electronic device 50A, the manifestation of the controls/inputting, the keyboard element 32A and the game control 35 continue to be located most proximal to the users' body. When the electronic device 50A is counterclockwise rotated from a vertical position to a horizontal position, the sideline element 31A is rotated from a vertical position to a horizontal position, accordantly and vice versa.

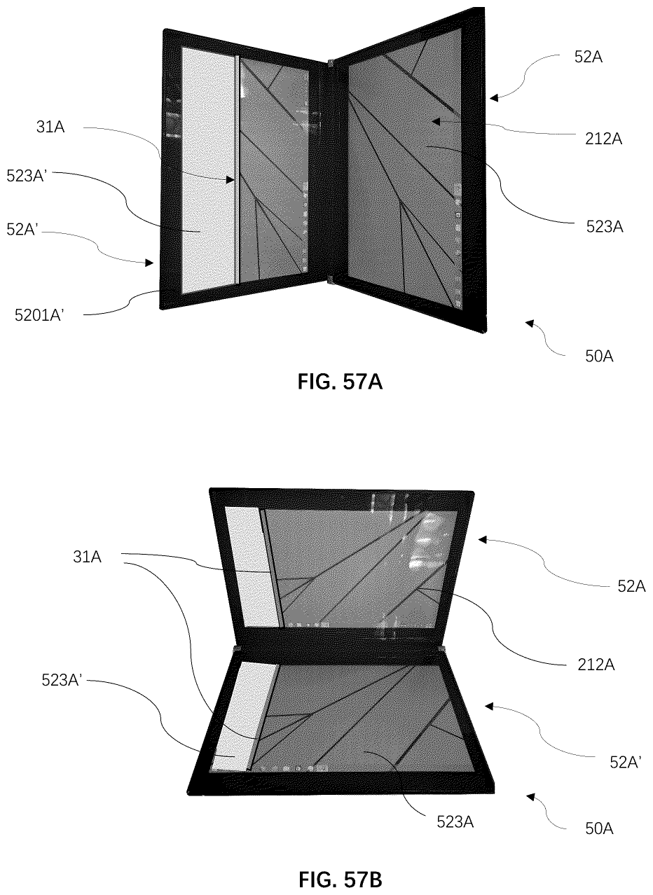

It is important to mention the rotational arrangement is important when one or more display screens 52A, 52A′ are equipped with one electronic device 50A. Referring to FIG. 57A, a vertical sideline element 31A is provided on one display screen 52A′ while a physical vertical separation between the two display screens 52A, 52A′, wherein the two display screen 52A, 52A′ may display a continuous displaying content 212A or different displaying contents 212A and the sideline element 31A divides the display screens 52A, 52A′ to continuously display the displaying content 212A in a first displaying portion 521A and a second displaying portion 521A′ is provided between the sideline element 31A and the closer side edge 5201A′ of the display screen 52A′ having the sideline element 31A. When the two display screens 52A, 52A′ and the electronic device 50A is rotated for 90 degrees, the sideline module 30A also detects the two display screens 52A, 52A′ are operating one single sideline element 31A and the sideline element 31A is defaulted to remain in vertical orientation 21A extended continuously across the two display screens 52A, 52A′ as shown in FIG. 57B, wherein the second displaying portion 521A′ is extended across the two display screens 52A, 52A′ correspondingly while both the two display screens 52A, 52A′ also display the first displaying portion 523A. In other words, when the sideline element 31A is generated vertically or horizontally, its orientation can be defaulted to maintain vertically or horizontally regardless of the change of orientation of the display screen 523A or even more than one display screens 52A, 52A′. It is preferred that the sideline element 31A orientation of vertical or horizontal sideline 21A display are not to be limited with two or more programs on one or more display screen. It is important to understand the sideline not only divides programs, they control the sizes of all the programs operating on one or more display screens.

As shown in FIGS. 57A and 57B, according to a preferred embodiment, the electronic device 50A is embodied as a foldable screen smart phone, notebook or two tablets having two display screens 52A, 52A′ configured in a foldable manner. As shown in FIG. 57A, the sideline element 31A (as well as the reservation sideline element 32A and/or the pop-up sideline element 37A not shown in FIGS. 57A and 57B) can be activated to provide on one of the display screens 52A′ to divide the displaying area of the display screen 52A′ into two displaying portions 523A, 523A′ for displaying two different displaying contents 212A while the other display screen 52A remains to have only one display portion 521A displaying one displaying content 212A or the continuous displaying content 212A with the displaying portion 523A of the display screen 52A′. As shown in FIG. 57B, the sideline element 31A (as well as the reservation sideline element 32A and/or the pop-up sideline element 37A not shown in FIGS. 57A and 57B) can be activated to provide and extend on both display screens 52A, 52A′ to divide the displaying area of the display screens 52A, 52A′ into two displaying portions 523A, 523A′ for displaying two different displaying contents 212A.

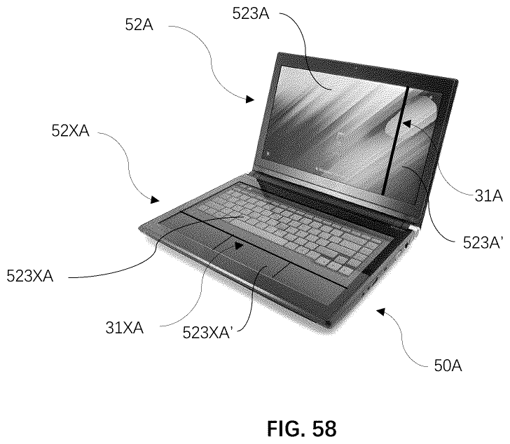

Referring to FIG. 58, the electronic device 50A is embodied to comprise one visual display screen 52A and one control display screen 52XA where the display screens 52A is position over the control display screen 52XA. The display screen controller module 20A is functioned on both display screens 52A, 52XA, where the sideline element 31A can be defined as a visual display sideline on the display screen 52A and is position over the control display screen 52XA and that a control sideline element 31XA can be provided on the control display screen 52XA. The sideline element 31A is provided to divide the displaying area of the display screen 52A into four displaying portions 523XA, 523XA′, 523A, 523A′ for visual displaying as described above and the control sideline element 31XA provided on the control display screen 52XA can divide the control displaying portion 521XA, 523XA′ which is generally for displaying control programs such as keyboard element program 43A and gesture and cursor program 44A to have an additional displaying portion 523XA′ for displaying different control tool. For example, as shown in FIG. 58, the upper control displaying portion 523XA displays a virtual keyboard and the lower control displaying portion 523XA′ displays a touch pad. Alternatively, for example, the sideline element 31A and the control sideline element 31XA can be functioned as a single continuous sideline element extended from the display screen 52A and the control display screen 52XA so that the two control displaying portions 523XA, 523XA′ on the control display 52XA can be used to display corresponding control programs to control and operate the corresponding displaying portions 523A, 523A′ on the display screen 52A, as one vertical sideline element 31A from the control display 52XA to the display screen 52A.

According to a preferred embodiment, the display screen controller module 20A can configure the sideline module 30A to control the sideline element 31A, the reservation sideline element 32A, the pop-up sideline element 37A, and the control sideline element 31XA on the corresponding display screen 52A, 52XA. Also, the sideline module 30A can configure one or more adjusters 13A to control the respective sideline element 31A, the reservation sideline element 32A, the pop-up sideline element 37A, and/or the control sideline element 31XA as sideline adjusters having one or more adjuster interfaces 21A functioning as sideline adjuster interfaces to produce one or more control segments positioned on the respective sideline element 31A, the reservation sideline element 32A, the pop-up sideline element 37A, or the control sideline element 31XA on the display screen 52A, wherein the sideline module 30A produces any type of sideline element on any display screen vertical horizontal or oblique.

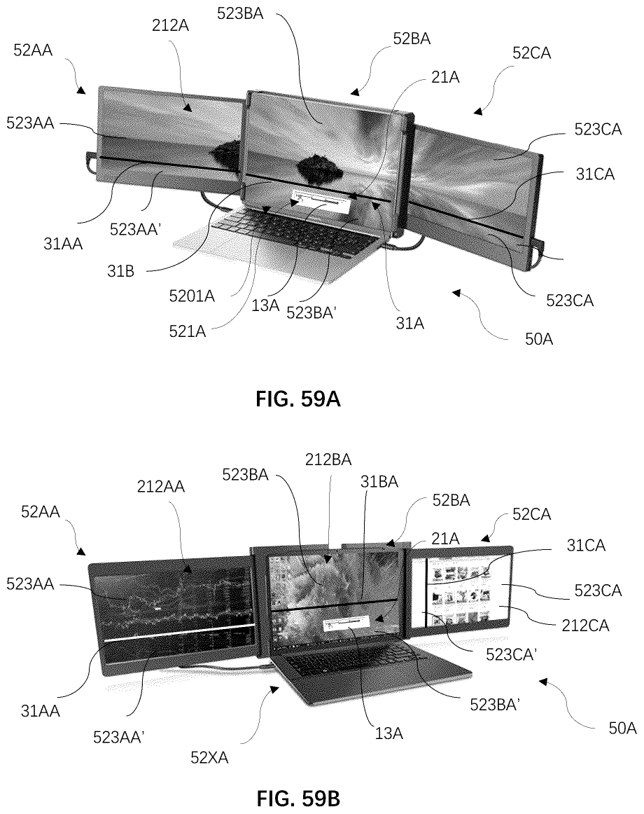

Referring to FIGS. 59A and 59B, the display screen controller module 20A is equipped with the sideline module 30A of the electronic device 50A which comprises more than one individual display screens 52A, for example a first display screen 52AA, a second display screen 52BA, and a third display screen 52CA, such that when the user selects to activate one sideline element 31A through a sideline element activation gesture (for example using two fingers touching a bottom side edge 5201A of one of the display screens 52BA and moving toward a position from the side edge 5201A, a horizontal sideline element as shown in FIG. 59A is generated horizontally at the position where the two fingers stop moving), the sideline element 31A is produced and extended horizontally across a first displaying portion 523AA of the first display screen 52AA, a second displaying portion 523BA of the second display screen 52BA and a third displaying portion 523CA of the third display screen 52CA to form an elongated sideline element 31A extending across the first, second and third displaying portions 523AA, 523BA, 523CA while the first, second and third display screens 52AA, 52BA, 52CA are displaying one single displaying content 212A on the displaying portions 523AA, 523BA, 523CA thereof. The first, second and third display screens 52AA, 52BA, 52CA are divided symmetrically into the first, second and third displaying portions 523AA, 523BA, 523CA and a divided first, second and third displaying portions 523AA′, 523BA′, 523CA′ for displaying different displaying content or an adjuster 13A and an adjuster interface 21A for operating and controlling the entire displaying content 212A on the first, second and third displaying portions 523AA, 523BA, 523CA. The elongated sideline element 31A comprises three sideline element segments 31AA, 31BA, 31CA which are the first sideline element 31AA, the second sideline element 31BA and the third sideline element 31CA provided on the first, second and third display screens 52A, 52BA, 52CA respectively. In addition, a movement of the elongated sideline element 31A by a gesture through one or more touch points 521A on any sideline element segments 31AA, 31BA, 31CA (i.e. the first, second and third sideline elements) will move the entire elongated sideline element 31A at the same time. For example, when the user uses one finger double tapping on one of the sideline element segments 31AA, 31BA, 31CA and then holding and moving upward away from the bottom side edge 5201A, the entire elongated sideline element 31A, including the first, second and third sideline elements 31AA, 31BA, 31CA will move up correspondingly and simultaneously, and the first, second and third displaying portions 523AA, 523BA, 523CA reduce their sizes (displaying areas) simultaneously and the divided first, second and third displaying portions 523AA′, 523BA′, 523CA′ correspondingly increase their sizes simultaneously. Accordingly, the user may operate and control the entire displaying content 212A on the first, second and third displaying portions 523AA, 523BA, 523CA with the adjuster 13A and the adjuster interface 21A. The user is also free to move the adjuster 13A and the adjuster interface 21A to anywhere on the first, second and third displaying portions 523A, 523A′, 523BA, 523BA′, 523CA, 523CA′ by an adjuster and interface moving gesture such as using three fingers forming and holding a triple touch points 521A at the adjuster interface 21A and the adjuster 13A. Also, if the user uses two fingers forming a gesture of two touch points 521A on any sideline element segments 31AA, 31BA, 31CA and moving towards the side edge 5201A that defines the corresponding divided displaying portion 523AA′, 523BA′ 523CA′ with the first, second or third sideline element 31AA, 31BA, 31CA, the entire elongated sideline element 31A, including the first, second and third sideline elements 31AA, 31BA, 31CA is removed from the first, second and third display screens 52AA, 52BA, 52CA.

It is worth mentioning that the color of the sideline element 31A (including the reservation sideline element 32A, the pop-up sideline element 37A and the control sideline element 31XA) can be defaulted to change automatically in higher color contrast with the background color of the displaying content 212A on the corresponding displaying portion 523AA. For example, when a dark color sideline element 31A will change to light color when the background color of the displaying content 212A changes to a dark color, vice versa.

Referring to FIG. 59B, either one of the display screens 52AA, 52BA, 52CA is selected and operated to display a displaying content 212A different from the others, all display screens 52AA, 52BA, 52CA are selected and operated to display different displaying contents 212A, 212B, 212C, or one of the displaying portions 523AA, 523BA, 523CA is selected and operated to have a scale or size different from the other such as enlarging a designated portion of the entire displaying content 212A to be displayed on one displaying portion 523A, 523BA or 523CA, the first, second and third sideline element segments 31AA, 31BA, 31CA become independent sideline elements 31AA, 31BA, 31CA working and functioning independently on the display screens 52AA, 52BA, 52CA respectively. Therefore, each first, second and third sideline elements 31AA, 31BA, 31CA can be displayed, controlled and operated independently with respect to the first, second and third displaying contents 212A, 212B, 212C on the first, second and third display screens 52AA, 52BA, 52CA respectively. Also, each display screen 52AA, 52BA, 53BA may has its own adjuster 13A, adjuster interface 21A and etc. to control its own displaying portion 523A thereon. Each of the first, second and third sideline elements 31AA, 31BA, 31CA divides the corresponding display screens 52AA, 52BA, 52CA to form the divided first, second and third displaying portions 523AA′, 523BA′, 523CA′.

The different types of sideline element 31A, 32A, 37A, 31XA as well as the adjuster 13A, the adjuster interface 21A, graduation 12A, and etc. can be changed in location to different displaying portions 523AA, 523BA, 523CA. For example, the user may simply use two fingers to tap and hold to form a two-fingers touch point 521A on the designated sideline element 31A, reservation sideline element 32A, pop-up sideline element 37A, or control sideline element 31XA, or the designated adjuster 13A, adjuster interface 21A, graduation 120A, or etc. and move the new desired position on desired displaying portions 523A, 523A′, 523BA, 523BA′, 523CA, 523CA′, as shown in FIG. 59B, and then the moved sideline element 31A, 32A, 37A, 31XA or the moved adjuster 13A, adjuster interface 21A, graduation 120A, or other controlling tools can be programmed to function and be operated with respect to the displaying portion 523A, 523A′, 523BA, 523BA′, 523CA, or 523CA′ designated by the user.

Also, referring to FIG. 59A, the user may control the entire elongated sideline element 31A on the display screens 52AA, 52BA, 52CA by controlling one of the sideline element segments (e.g. the first, second and third sideline elements) 31AA, 31BA, 31CA on the respective first, second and third display screens 52AA, 52BA, 52CA. The adjuster 13A and the one or more graduations 120A can be generated according to the touch point 521A located at the corresponding first, second or third sideline element 31AA, 31BA, 31CA in the respective display screen 52AA, 52BA, 52CA. An adjuster interface 21A may also be generated on the first, second or third sideline element 31AA, 31BA, 31CA for controlling the entire elongated sideline element 31A, such as movement of the entire elongated sideline element 31A on the first, second and third display screens 52AA, 52BA, 52CA simultaneously.

Or, alternatively, referring to FIG. 59B, the adjuster interface 21A generated on the first, second or third sideline element 31AA, 31BA, 31CA can control the respective sideline element 31AA, 31BA or 31CA on the respective first, second or third display screen 52AA, 52BA or 52CA, such as movement of that first, second or third sideline element 31AA, 31BA or 31CA on the respective first, second or third display screen 52AA, 52BA, 52CA.

More specifically, when the display screen controller module 20A receives signals upon detection of the touch point 521A on the respective sideline element 31AA, 31BA or 31CA at the display screen 52AA, 52BA or 52CA, the display screen controller module 20A identifies and determines corresponding command and sends to the processor 51A to execute corresponding action(s). For example, when the display screen controller 22A of the display screen controller module 20A identifies and determines the touch point 521A to be a two-finger touch point, but not a single touch point, that is a command gesture of the two-finger touch point specifically performed on the sideline element 31AA, 31BA or 31CA by the user, and the touch point 521A being held and moved upwards from to an upper position on the respective display screen 52AA, 52BA or 52CA, the display screen controller 22A provides the information to the module of analyzable factors 26A, the artificial intelligence module 25A, and the distance tracker module 27A, which are activated in the display screen controller module 20A, and then the sideline module 30A is activated and the entire sideline element 31A as shown in FIG. 59A moves upwards on the display screens 52AA, 52BA, 52CA along with the movement of the two-finger touch point 521A to the new position on the display screens 52A, 52BA, 52CA, or the sideline module 30A is activated and the respective sideline element 31AA, 31BA or 31A as shown in FIG. 59B on the respective display screen 52AA, 52BA or 52CA where the touch point 521A detected moves upwards along with the movement of the two-finger touch point 521A to the new position on that display screen 52AA, 52BA or 52CA.

For another example, when the display screen controller 22A of the display screen controller module 20A identifies and determines a touch point 521A being a command gesture performed at the adjuster 13A on the respective display screen 52AA, 52BA or 52CA by the user and the touch point 521A being held and moved towards a direction along the adjuster 13A, the display screen controller 22A provides the information to the module of analyzable factors 26A, the artificial intelligence module 25A, and the distance tracker module 27A, which are activated in the display screen controller module 20A, and then the sideline module 30A retrieves the location of the touch point 521A and sends the position of the touch point 521A to the adjuster module 10. The adjuster module 10A further acquires the state of the touch point 521A and the state of the respective display screen 52AA, 52BA or 52CA and such information is sent to the processor 51A through the communication module 55A to execute the command corresponding to the touch point 521A detected on the adjuster 13A, such as zooming in or zooming out of the displaying content 212A, 212B, 212C on the respective displaying portion 523AA, 523BA, 523CA accordingly. The user may also set up a gesture through touch point 521A to move the location of the adjuster 13A, such as a four-finger touch point 521A that the user uses four fingers touching on the currently adjuster 13A provided on the respective display screen 52AA, 52BA, 52CA to perform the four-finger touch point as a gesture to command to move the adjuster 13A on the display screen 52AA, 52BA, 52CA. When the user uses four fingers touching on the adjuster 13A and continuously holding and moving to a desired new position linearly and/or rotatingly on the display screen 52AA, 52BA, 52CA (e.g. moving from one displaying portion 523AA, 523BA, 523CA to another display portion 523AA, 523BA, 523CA and rotating from vertical displacement to horizontal displacement, the adjuster 13A will move along with the four-finger touch point 521A to the new horizontal location on the respective display screen 52AA, 52BA, 52CA wherein the four-finger touch point 521A stops the movement.

Alternatively, from right to left, the user can use a one finger touch point 521A to operate all the gestures for the display portion 523A in the display screen 52A on the display screen 52XA. Furthermore, the user may program all the two-finger touch point 521A performed on the display screen 52XA to operate all the functions or programs on the display portion 523BA of the display screen 52BA. Additionally, all the three touch point gestures performed on the display screen 52XA may only operate the display portion 523CA of display screen 52CA, or from left to right (vice versa), etc. Furthermore, all the four finger gestures operate the control display screen 52XA.

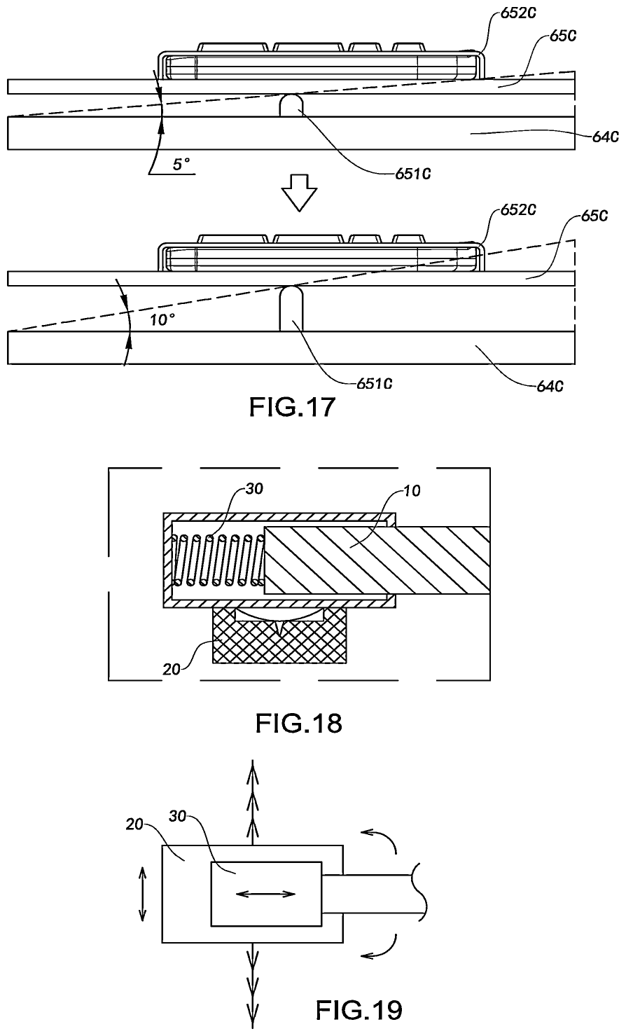

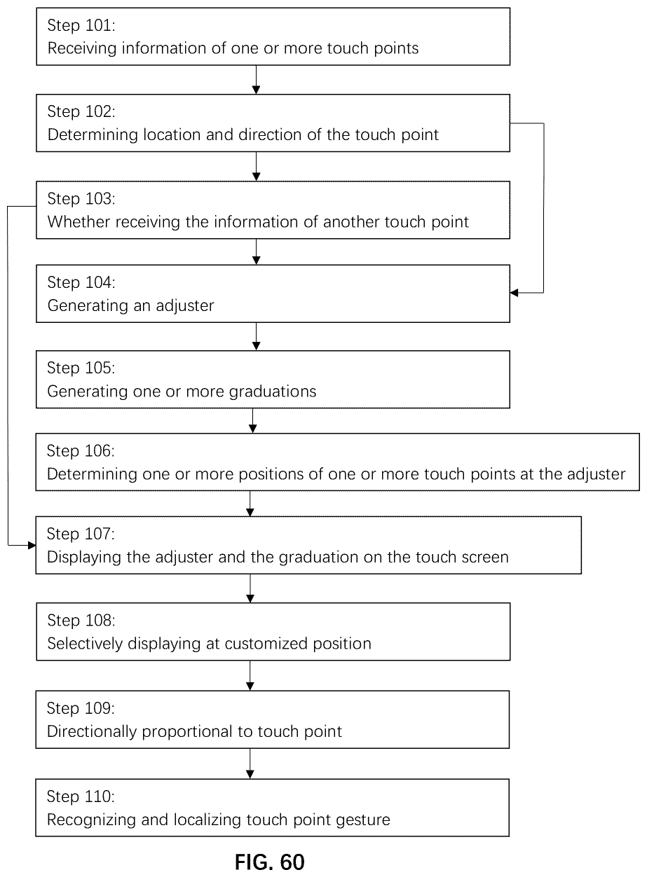

Referring to FIG. 60, a method of generating the adjuster 13A according to the preferred embodiment of the present invention is illustrated, which comprises the following steps:
Step 101: Receiving information of a touch point 521A. The display screen 52A detects one or more touch points 521A, wherein, for example, the touch screen 52A generates a respective touch point 521A at a position of the finger of the user touching the display screen 52A and the adjuster module 10A receives the information of the respective touch point 521A detected and sent from the touch screen 52A. The information is analyzed by the module of analyzable factors 26A, the artificial intelligence module 25A and the distance tracker module 27A of the display screen control module 20A, for movement and direction of each of the one or more respective touch points 521A.
Step 102: Determining location and direction of the touch point 521A. If the touch point 521A doesn't move, go to step 103A. If the touch point 521A is moving, detecting the moving direction of the one or more touch points 521A and sending the moving direction and location of the touch point 521A to the adjuster module 10A, and then go to step 104A.
Step 103: Whether receiving the information of another touch point 521A. If the adjuster module 10A receives the information of another touch point 521A, go to step 104; if not, go to step 107.
Step 104: Generating an adjuster 13A. The adjuster module 10A generates an adjuster 13A according to the position and the moving direction of the touch point 521A detected by the display screen controller module 20A. The adjuster module 10A adjusts the size of the display of one or more programs in one or more display screens 52A with an adjuster 13A and an adjuster interface 21A to control the display proportions. The adjuster 13A comprises an adjuster body 130A, minimum end 131A and a maximum end 132A as shown in FIG. 17. The minimum end 131A and the maximum end 132A are located at the two end of the adjuster body 130A respectively that can be an arrow mark or a watermark with a timely display and customizable from right to left, left to right, up or down, down or up, and etc. A touch point 521A is able to move from the minimum end 131A to the maximum end 132A along the adjuster body 130A. Through the movement of the touch point 521A along the adjuster 13A, the displaying content 212A displayed in the display screen 52A responds according to the moving direction and position of the touch point 521A (and the sign language gesture or the imagining gesture as described below), for examples, enlarging the displaying content 212A or moving the displaying content 212A according to proportion of the touch point 521A moved from the minimum end 131A towards the maximum end 132A. It is important to mention that direction of each of the one or more touch points 521A provides a desire function and position of the respective one or more touch points 521A and a degree of the desire function.
Step 105: Generating one or more graduations 120A for the adjuster 13A. The graduation 120A is a means to inform the user of the available degree in the desire function and to inform the user of the current degree with the current position of the respective touch point 521A. The adjuster module 10A generates the one or more graduations 120A according to the current state of the displaying content 212A or program function, the parameters of the touch screen 52A, the preset proportion and the adjuster 13A. The graduation 120A is attached on the adjuster 13A to show the scale of operation such as zooming in/out for adjusting the displaying content 212A. Through the graduation 120A, the user understands the scale of the current displaying content 212A that is able to be zooming in or zooming out.

Please further referring to FIGS. 47 to 48D, the adjuster module 10A generates a graduation indicator 14A and or a numeric indicator 16A according to the current state of the displaying content, the parameters of the display screen 52A and the preset proportion of the touch point 521A on the adjuster 13A indicating on the adjuster indicator 14A through the use of the adjuster interface 21A on the adjuster 13A. The graduation 120A is provided on the adjuster 13A to show the moving scale of the displaying content 212A. Through the graduation 120A, the user understands the various available distance for display and the current distance of one or more displaying contents 212A of one or more programs on one or more display screens 52A for one or more program functions. The scales provide a customize percentage through the adjuster 13A. Where the finger of the user requires significant less physical demands to move a displaying content 212A in the display screen 52A in relation to the size of the physical display screen 52A and or the size of the program/document being larger or smaller to the display area of the physical display screen 52A, where the user can easy manipulated the size of the displaying content 212A in any medium.
Step 106: Determining one or more positions of one or more touch points 521A at the adjuster 13A. The adjuster module 10A determines the one or more positions of the one or more touch points 521A at the adjuster 13A through comparing a current display state of the displaying content 212A and the graduation 120A of the adjuster 13A.

For example, if the current display state of the displaying content 212A is in minimum state, the first touch point 521A is arranged to the position at the minimum end 131A of the adjuster 13A, the second touch point 521A is arranged to the position between the minimum end 131A and the maximum end 132A of the adjuster 13A. If the current display state of the displaying content 212A is in maximum state, the second touch point 521A is arranged to the position of the maximum end 132A of the adjuster 13A and the first touch point 521A is arranged to the position between the minimum end 131A and the position of the second touch point 521A. If the current display state of the displaying content 212A is between the minimum state and the maximum state, the first touch point 521A and the second touch point 521A are arranged at the adjuster 11A between the minimum end 131A and the maximum end 132A of the adjuster 13A. In addition, the adjuster 13A is arranged to extend through the first and second touch points 521A.
Step 107: Displaying the adjuster 13A and the graduation 120A on the touch screen 52A. The adjuster 13A is displayed to match the moving direction of the touch point 521A. The position of the graduation 120A at the adjuster 13A is arranged to the position of the touch point 521A, such that the touch point 521A is moved along the adjuster 13A to control the displaying content 212A to move or to adjust the size of the displaying content 212A.
Step 108: Selectively displaying at customized position. Alternatively, the adjuster 13A with the graduation 120A, the adjuster indicator 14A, the adjuster interface 21A, and or the numeric indicator 16A can also be display at any other customized position on the display screen 52A, with or without the one or more graduations 120A, the one or more adjuster indicators 14A, and/or the one or more numeric indicators 16A. The adjuster indicator 14A may present on the scale of the graduation 120A of the adjuster 13A with or without displaying the numeric indicators 16A. Through the adjuster interface 21A, the adjuster module 10A is controlled, where the graduation 120A, the adjuster indicator 14A, and/or the numeric indicator 16A, reflects the current state of control on the displaying contents 212A, or the state of the program function on the parameters of the displaying contents 212A divided by one or more sideline elements 31A, on one or more display screens 52A, proportionally. The graduation 120A is provided on the adjuster 13A to show the scale of zoom in/out for adjusting the displaying content 212A. The one or more numeric indicators 16A may also be displayed without the graduation 120A, the adjuster indicator 16A, or the adjuster 13A. The numeric indicator 16A and the adjuster 13A can also be customized anywhere in the display screen 52A as an alternative customizable option. It is worth mentioning that the numeric indicator 16A can provide the user the currents size of the image, therefore the user can decide to enlarge or to diminish the display content 212A. The numerical indicator 16A and the adjuster 13A can be customized with desired size, font, color, time, as a watermark, and as a timed pop-up display. Further, the sideline element 31A can sub-divide one or more adjusters 13A accordingly to the display customizable position (associated with zooming) and/or according to the program function of the adjuster 13A. For example, the program function of the adjuster 13A for volume or brightness are examples on selected running programs (not associated with zooming), etc.
Step 109: Directionally proportional to touch point. The degree, non-displaying or amount of zooming on the display screen 52A is directionally proportional to the one or more touch points 521A on the adjuster interface 21A of the adjuster 13A and reflected on the graduation 120A, by the adjuster indicator 14A, and/or on the numeric indicator 16A, as the touch point 521A is moved along on the adjuster interface 21A of the adjuster 13A on one or more programs displayed in one or more display screens 52A.
Step 110: Recognizing and localizing touch point gesture. Alternatively, a gesture of two or more touch points 521A, can be recognized and localized by the module of analyzable factors 26A, the artificial intelligence 25A, and the distance tracker module 27A of the display screen control module 20A, so as for the adjuster module 10A to generate adjuster 13A, the graduation 120A, the adjuster indicator 14A, and/or the numeric indicator 16A which are all customizable on one or more programs in one or more display screens 52A. Where the gesture has been pre-programmed to display one or more display contents 212A of one or more programs on one or more display screen 52A, specifically, and/or in close proximity to the degree of the pre-program of the gesture of the two or more touch points 521A without additional physical movement from the two or more touch points 521A, based on the degree/distance of separation between every two touch points 521A.

The method of generating the adjuster 13A can be proceeded based on a slideline element 31A according to the preferred embodiment of the present invention, comprising the following steps:
Step 201: Receiving the information of a touch point 521A. The touch screen 52A detects a user using a finger to touch the touch screen 52A. The touch screen 52A generates a touch point 521A at a position of the finger of the user touching the touch screen 52A. The adjuster module 10A receives the information of the touch point 521A sent from the touch screen 52A. The touch screen controller 40A analyzes the information of the touch point 521A to identify which touch screen 52A sends the information of the touch point 521A. In other words, the information of the touch point 521A includes which touch screen 52A generated the touch point 521A and the position of the touch point 521A.
Step 202: Determining whether the touch point 521A is located at the control touch screen 52XA. If the touch point 521A is located at the control touch screen 52XA, go to step 203:
Step 203: Determining whether the touch point 521A is located at a displaying sideline element 31A or a controlling sideline element such as reservation sideline element 32A or pop-up sideline element 37A. If the touch point 521AG is located at the displaying sideline element 31A, go to 204A. If one or more touch points 521A are located at both the displaying sideline element 31A and the controlling sideline element, go to 205. If the touch point 521A is located at the controlling sideline element, go to 206.
Step 204: Moving the displaying sideline element 31A according to the moving direction of the touch point 521A.
Step 205: Moving the displaying sideline element 31A and the controlling sideline element according to the moving direction of the one or more touch points 521A.
Step 206: Moving the controlling sideline element according to the moving direction of the touch point 521A.

The electronic device 50A can be configured to have the program dictionary 45A that may include a gesture dictionary to pair in the interacting module 40A with one or more gestures, from the gesture dictionary, with a program from the program dictionary 45A. Where the pair information is provided to the processor 51A by the communication module 55A. Also, the display screen 52A displays the pair information and is where the user performance personal customization of the selected gesture for pairing with a program. In this Step 108, the performed information is recorded and compared to the selected touch points 521A and/or to the complexity of the two or more touch points 521A selected. After the customization is performed, the one or more touch points 521A of gesture (including the sign language gestures, hand/finger software detection programs, and/or the imagining gestures as described below) are analyzed by the module of analyzable factors 26A, the artificial intelligence 25A, and the distance tracker 27. The program and performed gesture, are then sent to the adjuster module 10A, and the sideline module 20A accordantly. Once the pair information is confirmed, it is sent to the processor 51A via the communication module 55A, where the communication module 55A sends the pair information as a gesture command for the storage device 53A for record and at the same time the gesture information is received on the display screen 52A to generate a response.

It is worth mentioning that the electronic device 50A may be configured with one or more display screens 52A, on or mores sidelines elements 31A, one or more programs, and one or more adjusters 13A on one or more displaying portions 523A, with one or more gestures for generation of one or more functions base on one or more touch points 521A according to the preferred embodiment of the present invention. According to the preferred embodiment of the present invention, a gesture generation method base on a sideline element 31A is illustrated, which comprises a step of:

Receiving the information of one or more touch points 521A at the same time or in a timely manner. An information for splitting one or more programs by one or more sideline elements 31A on one or more display screens 52A with one or more touch points 521A is sent to the module of analyzable factors 26A, the artificial intelligence 25A and the distance tracker module 27A in the display screen controller module 20A, wherein the information is collectively acquired from to the adjuster module 10A, the sideline module 30A, and the interacting module 40A. The display screen controller module 20A confirms the information and then sends to the processer 51A through the communication module 55A, wherein the processor 51A sends the information to be displayed on the display screen 52A. Additionally, the information is shared between the storage device 53A, the power supply 54A, and the input device 56 on the electronic device 50A, wherein the information may be uploaded to the server processer 101A, shared with the server storage device 103, the server communication module 105, the server power supply 104, and lastly sent to the server screen 102A.

For example, the display screen 52A is configured to be operated through one or more touch points 521A from one or more executable portions 1A, 2A, 3A, 4A divided by one or more sideline elements 31A and, as shown in FIG. 58A, can be controlled by one or more control display screens 52XA divided by one or more sideline elements 31XA with one or more touch points 521A controlling one or more programs. The keyboard element program 43A is configured for typing on the control display screen 52A, the operating area program 401A is configured for superimposing the gesture and cursor program 44A in the control display screen 52XA to control the gesture and cursor program 44A on the display screen 52A. The program operated through the control display screen 52XA can control the program in the display screen 52A. Additionally, the program can be superimposed by the state-off program 48A programs where the entire selected program is locked in the area border by the adjacent sideline elements 31A, where the user can physical hold, grab, manipulated or handle the display screen surface area without accidental inputting erroneous information.

Additionally, the programs can be switched on the display screen 52A only and maintain the ergonomic desire control in the program in the control display screen 52XA. Where the display program in the display screen 52A can be color code on the sideline element 31A with the corresponding control program in the control display screen 52XA for orientation arrangement.

It is important to mention that all the programs to be operated and displayed on the one or more display screens 52A and/or control display screen 52XA are interchangeable, scalable, customizable and are in the program list dictionary 45A where all the electable touch points 521A are provided in the gesture list dictionary 402, wherein both programs are in the interactive module 40A and both programs are required to be pair with each other, wherein once it is paired, the gestures are operational at the same time or in a timely manner on one or more split programs in one or more display screens 52A.

It is appreciated that, according to the preferred embodiment of the present invention, the display screen 52A may be a LCD or LED monitor, a touch screen or a projection screen displaying through projecting device such as projector, optical display device, augmented reality display device, goggle headset, AR/MR/XR/VR headset or glasses, and etc. For example, an augmented reality display allows a user to view their surroundings as well as projected images, wherein the projected images can be overlaid on the real world perceived by the user. In a normal set-up, a transparent display screen may be provided in front of a user so that they can continue to see the physical world and the display screen can include a typically glass waveguide and a projector provided to one side, wherein light from the projector is coupled into the waveguide by an input diffraction grating and the projected light is totally internally reflected within the waveguide. Then, the light is coupled out of the waveguide by an output diffraction grating so that it can be viewed by the wearer.

The adjuster 13A, the adjuster interface 21A, the sideline element 31A, the reservation sideline element 32A and the pop-up sideline element 37A of the present invention are especially practical and useful to the display screen 52A embodied as projection screen as mentioned above that does not have a physical boundary like the monitor or touch screen. For example, to operate a touch screen, the physical width and height of the touch screen at least provide a reference border to the user's fingers to touch and operate as a relatively proportional reference. However, to the displaying content 212A projected from the projecting device, the images are projected in front of the eyes of the user and may not have any boundary. Although motion sensors can be provided to detect the movement of the user's hands and fingers so as to detect the touch points 521A operation as described above, the amount of the movement of the user's finger fails to provide any reference to the proportion of the touch point operation. Therefore, the adjuster 13A, the adjuster interface 21A, the sideline element 31A, the reservation sideline element 32A and the pop-up sideline element 37A displayed in the projection screen type display screen 52A in the present invention significantly allow the user to operate and control the electronic device 50A through the projection screen type display screen 52A precisely with scale and proportion references.

It is worth to mention that, referring to FIG. 62, in principle, any type of neurological impulse signal could be used to operate with Brain Computer Interface (BCI) systems, like signals from an Electroencephalogram (EEG), Magnetoencephalography (MEG), Electrocorticography (ECoG), any Brain Implants (Intracortical Microelectrodes), and etc. For example, time-trigger EEG or ECoG response amplitudes and latencies, power within specific EEG or ECOG frequency bands or firing rates of individuals cortical neurons. Additionally, upper and lower extremities neuromuscular signals can be utilized, wherein the signal acquisition from Electromyography (EMG), Acceleromyography (AMG), Mechanomyography (MMG), and etc. are converted into digital control signals. In other words, the sensors on a (AR/MR/XR/VR) headset are the means for acquiring the signal and measuring the signal of the brain in relation to signal acquisition on the head/scalp through the (EEG), or by the sensors (ECOG) in the skull, or by the sensor (neural implant) in the brain, or any type of signal measurement using a particular sensor modality. The EcoG is not limited to 1,024 or 2,048 sensors or any lessor grade grids like 256 sensors or less, in fact the invention is not limited to less than 2,048 sensors.

If the brain signal acquisition is formulated from (EEG/ECOG) the same way, the same way the (EMG) signal acquisition can be formulated from neuromuscular electrical signals on any extremities from the arms, neck or legs with a dermis connector sensor modality for signal acquisition, wherein the acquisition of signs for the (EEG/ECoG) and/or for the (EEG) are amplified or reduced to levels appropriate for electronic processing, including filters for removing electrical noises and for operational through the BCI apparatus or any other similar device, independent or built-in, within the electronic device 50A. Also, all signal acquisitions required mentioned above are for the “Feature Extraction” of the processes of analyzing the digital signals. In other words, to differentiate relevant signals characteristics (type of signal and features in the signals, in relation to the final intent) from a biological content and to express them in a compressed form suitable for translation into output commands. Additionally, the resulting signals are then delivered to the “Feature Translation” algorithm, for example the artificial intelligence module 25A, which converts the signal features into the appropriated commands for the electronic device 50A (Device Output). In other words, the “Feature Translation” algorithm provides the translation for the digital functions of the (Device Output) such as letters selections, cursors control, gestures selection pairing program and/or program function, sideline element movement, and etc., wherein the electronic device 50A provides feedback to the user, thereby providing a close loop arrangement. It is important to mention that the (BCI/BMI) can be operated as an accessory to the electronic device 50A, or in combination with the artificial intelligence module 25A in the electronic device 50A or built-in within the electronic device 50A in combination with the processors 51A or independent without the processors 51A through the cloud sever 100A.

Furthermore, the brain signals (EcoG, EEG), the extremity signals of Electromyography (EMG) and the verbal commands can be used in combination or independently for controlling digital electronic devices 50A. The user may position the electrons on the anterior portion of the neck for the signal acquisition of the neuromuscular system in relation to speaking with or without voice commands. Furthermore, the type of signal acquisitions (EEG, EcoG, EMG) can be from a healthy body part and/or from an injured body part, such as a brain lesion and/or without a brain lesion.

The significant difference in the present invention is the interoperability of utilizing any neurological signal from a human body, wherein one or more signal acquisitions are measurable from the production of one or more gestures performed or from an imagining gesture, including verbal command or an intended vocalization from a healthy human body and/or from a less than optimal human body. The teachings include method and arrangement for interoperability with various types of signal acquisition unique to the individual from the neuromuscular signals and/or from the brain signals, wherein the signal acquisition is processed and converted to signals that are used for inputting digital or robotic commands corresponding to an electronic device 50A. Through the use of physical device like the (BCI), equipped with the electronic device 50A wirely or wireless, or through a built-in device within the electronic device 50A, wherein the computing functions for the conversion of the signal acquisition to the digital base signal are operated online or through the cloud server 100A.

The present invention provides a wireless or wirely electrical signal acquisition for operating electronic devices 50A through the use of any signal acquisition, like Brain Computer Interface devices, or other similar devices at least consisting of four sequential components: (1) signal acquisition, (2A) feature extraction, (3) feature translation, and (4) device output. These 4A components are controlled by an operating protocol that defines the onset and timing of operation, the details of signal processing, the nature of the device commands, and the oversight of performance. Furthermore, the signal is pairing with a performed gesture and/or paired with an intent gesture, wherein either algorithm system performance can be provided from the electronic device 50A and or from the (BCI) algorithm system or any similar like artificial intelligence 25A computing system online or through the cloud server 100A, wirely or wirelessly, through the internet or through the server communication module 105, wherein signal acquisitions for any body part are used as in any traditional Brain Computer Interface (BCI) system. The present invention further provides that the signal acquisition is paired with the gesture and the gesture is paired with a program. Additionally, the signal acquisition can also be paired or not paired with a program or a gesture, and the gesture can be performed or an imaging gesture. An algorithm system incorporated in the electronic device 50A is also capable of anticipating the user next action point without the user producing a performed gesture or and imaging gesture, where the information is acquired from data of past performances including any or all performed or not performed gesture with or without paired signal acquisition. Where the commands are controlled and generated by the electronic devices 50A and are operated to be displayed through one or more display screens 52A, divided by one or more sideline elements, through the usage of one or more processors 51A, operational online, or in a cloud server 100A having a computing processing system with or without the processors 51A in the electronic device 50A.

The Brain Computer Interface system is equipped with a built-in algorithm system, attachable or detachable to the electronic device 50A, with wired or wireless interface with the analyzable factor module 26A and the artificial intelligence module 25A of the electronic device 50A for the signal acquisition of gestures and/or verbal commands. In other words, any type of human electrical impulse recording during a movement, or an imagine movement, for a physical gesture, a verbal command, a verbal intent, any physical movement, or intended command. Accordingly, in the present invention, the interoperability of Brain Computer Interface (BCI) system or the like can be used for inputting on any electronic devices 50A at the same time or in a timely manner on one or more programs divided by the sideline element 31A on the display screen 52A.

The operation of the program of the artificial intelligence module 25A in the electronic devices 50A can be implemented in multiple ways as being interfaced with the Brain Computer Interface (BCI) own algorithm system, or as two independent artificial intelligence modules 25A working together with one for the gesture touch point 521A in the electronic device 50A and the other for the signal acquisition of the imaging gesture or the performed gesture, for the function of the (BCI) to serve in the function of translating and recognizing the desire performed gesture or the imagining gesture, including the performed verbal command or the imagine verbal command, for any form of computerize equipment operations or mechanical robotic machinery interface with the electronic device 50A, operating within the usage of the (BCI/BMI) system through a separate device, through a built-in system or through an interoperability of the selected operations in the internet and or the cloud server 100A.

Furthermore, the present invention provides a system of pairing verbal command with a program or a program function response, through the use of a system like the Brain Computer Interface system with one or more signal acquisitions paired with selected programs or program functions. It is worth mentioning that the verbal function can have more than one words for the desire command to generate the desire response, in the performed vocalization or in the imagine vocalization through the usage of (EEG, EcoG, EMG) signal acquisition for the (BCI), performed within the processors of an electronic device 50A, a Brain Computer Interface (BCI), or without processors in the electronic device 50A, wherein the signal acquisition information is transmitted to the cloud server 100A and converted to the digital data base for the purpose of operating one or more electronic devices 50A, as mentioned in the present invention.

Alternatively, the verbal commands operation system can also come from an electromyography (EMG) signal acquisition from the neck voice track muscular system, wherein the signals acquisition is paired with a selected program or program function in a Brain Computer Interface like devices for operating the electronic device 50A. In this system the signal acquisition is acquired with audible or without audible vocalization of the verbal command. In other words, the system is not dependent on the audio software to recognize the command. The signal acquisition will require similar processing as earlier mention, for the Brain Computer Interface (BCI) system, the major advantage in this system is that the signal acquisition for operating the device is not sound dependent, in other words it will also operating in a noisy environment or without the user producing a audible vocalization. The problem with traditional voice activation programs for verbal commands they require a clean audible sound without noise pollution and in most cases proper enunciation of the selected language, otherwise the verbal command is misunderstood or is non-functional.

Furthermore, Electrooculogram (EOG) signal acquisition for eye movement or the electroretinogram signal acquisition for visual stimuli, these signals can also be process in the same manner as the other signal acquisition where processed for the Brain Computer Interface (BCI) devices. Again, the signal acquisition is paired with a selected program or program function, independent or in combination with other signal acquisition paired with other programs or program function, or not paired to the same program, or not paired to a program function. Wherein any signal from the human body can be used to paired with said programs or programs functions, through the use of a Brain Computer Interface like device, in addition to the gesture pairing with programs or programs functions in the electronic devices 50A, also mention earlier in this invention. The invention teaches the interoperability of signal acquisitions, softwares and hardwares computing systems, etc.

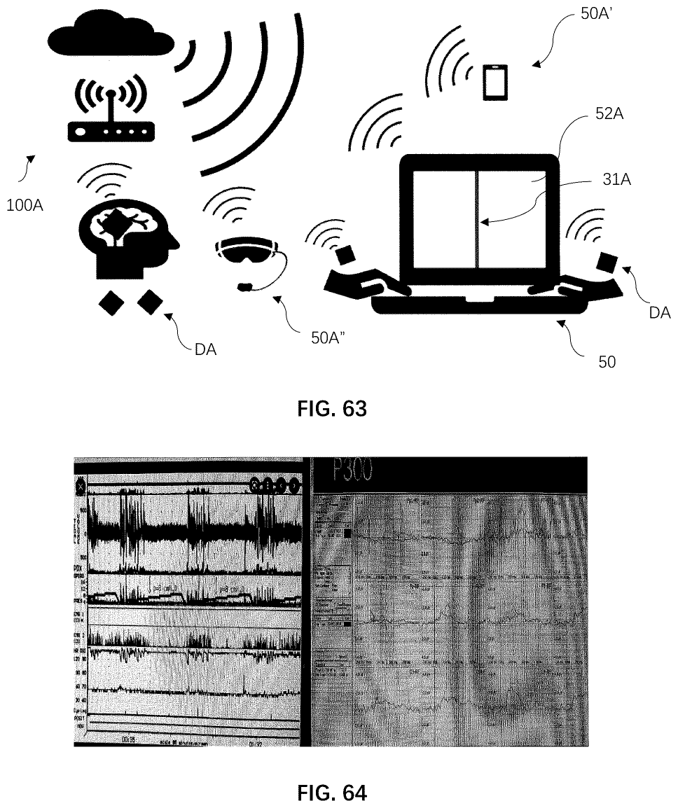

Referring to FIG. 63, the interoperability of performed and imagine signal acquisition on the electronic device 50A, 50A′, 50A″ like a headset, laptop, smartphone where the signal acquisition is delivered and acquired by the electronic device 50A, at the same time or in a timely manner in a close loop system, wirely or wireless, through the internet or through the cloud server 100A, wherein the signals are interdependent on one or more electronic devices 50A, 50A′, 50A″ for operating the one or more electronic devices 50A, 50A′, 50A″. It is worth mentioning that the diamond icons D means sensor transmitting wireless or wired information where the cloud server 100A utilizing the information. In addition, the smartphone type electronic device 50A″ can provide the user the ability to download a verification APP. Thereby, communal devices will not require the user to provide needlessly repetitive information, including the user gesture selections and program selections for the present invention or any part in the present invention. Alternatively, the user selects the degree of information willing to share with non-personal electronic devices 50A. For example, a personal electronic device 50A, 50A′, 50A″ will have a robust security system to prevent unwanted usage from uninvited individuals, wherein the gestures, the program selections, where any (signal acquisition) verifications are analyzed for the type of gesture performed or imagine, with or without the touch point 521A, with or without infrared base gestures with or without electromagnetic sensors on one or more electronic devices 50A, 50A′, 50A″ with one or more physical display screens 52A of the notebook computer type electronic device 50A or smartphone type electronic device 50A′ or virtual (non-physical) display screen 52A through one or more projecting devices 50A″ such as AR/VR/MR/XR goggle and being operated by an operation device such as control console, laptop, tablet, smartphone, or etc.

To individual consumers, who have physical limitation or incapable due to physical fatigue, medical conditions, or lack of interests, may not willing to perform body motions or verbal vocalizations currently required to operate haptic animation surrogate technology in the Virtual Reality environment. According to the preferred embodiment of the present invention, such users have the option to operate in a simulated environment, with or without full body wearables inputting sensors. In other words, the present invention significantly delivers exponentially more inputting flexible options for operating programs, in a more convenient approach to the user selection of electrodes utilization for the desire signal acquisition for operating 2-Dimensions display, 3-Dimensions display, and 4-Dimensional display in the electronic device 50A. The touch point gestures paired program is able to be operated in combination with the signal acquisition head electrodes or skull implants, electromagnetic sensors, infrared based technology, where the user selects the desire wearables inputting system without interference from the none selected inputting system, and that all inputting systems may also operated together, through the internet, the internet of things (IoT), quantum computer or the cloud server 100A.

It is worth mentioning that the smartphone type electronic device 50A′ may also provide the computing power for the AR/VR/MR/XR, just like Brain Computer Interface systems and, furthermore, may acquiring selected signals, receiving selected signals, delivering selected signals and/or processing selected signals from all the signal acquisitions delivered to the smartphone type electronic device 50A′, to generate a digital response in a close loop system within the AR/VR/MR/XR apparatus or in the display screen 52A. In other words, the smartphone type electronic device 50A′ like any other stationary or portable computer may have a compatible attachable, detachable, or built-in amplified that can receive (EMG, EEG, ECOG) signals from the user where the signals are processed by the smartphone type electronic device 50A′ in the same process as in a Brain Computer Interface. Alternatively, the smartphone type electronic device 50A′, with or without a signal amplifier, may receive a signal acquisition from the user or the process digital signal from the internet, cloud server 100A, or from quantum computing device after that signal acquisition has been processed. For example, the smartphone type electronic device 50A′ may receive the signal acquisition, and then the smartphone type electronic device 50A′ is the transmission source for delivering the signal acquisition information to the cloud server base Brain Computer Interface program, for the processing of that signal in the cloud server 100A, to a digital base signal and back to the smartphone type electronic device 50A′ in a transceiver like simulation.

It is also worth mentioning that the laptop type electronic device 50A may also provide the computing power for the AR/VR/MR/XR apparatus (electronic device) 50A″, just like Brain Computer Interface systems and, furthermore, may acquiring selected signals, receiving selected signals, delivering selected signals, and/or processing selected signals from all the signals acquisitions delivered to the laptop type electronic device 50A, to generate a digital response in a close loop system within the virtual display screen 52A of the AR/VR/MR/XR apparatus 50A″ and/or in the physical display screen 52A of the laptop type electronic device 50A or smartphone type electronic device 50A′. In other words, the laptop type electronic device 50A like any other stationary or portable computer may have a compatible attachable, detachable, or built-in amplified means that can receive (EMG, EEG, ECOG) signals from the user, wirely, wirelessly or remotely, where the signals are processed by the laptop type electronic device 50A in the same process as in a Brain Computer Interface. Alternatively, the smartphone type electronic device 50A′, with or without a signal amplifier, may receive the signal acquisition from the user and from the cloud server 100A as process digital signal, or from quantum computing device, after that original signal acquisition has been processed. For example, the laptop type electronic device 50A may first receive the signal acquisition, where the laptop type electronic device 50A is the transmission source for delivering the signal acquisition information to the cloud server base Brain Computer Interface program of the cloud server 100A, for the processing of the original signal acquisition into the digital base signal and that, once the digital base signal is produced, that signal is returned back to the laptop type electronic device 50A in a transceiver like simulation to generate a response on the display screen 52A.

FIG. 64 illustrates various neurological impulses in a signal acquisition created by the human body. It is appreciated that these human neurological impulses are unique to each individual and are created while an action is imagined or an action is physically performed. It is an Electromyography (EMG) and the Electrocorticography (ECoG), wherein the sideline element 31A may separate the selected signals acquisition in used. Where a dual signal acquisition system can be used for security purpose to ensure and to verify the intended individual is operating the personal electronic device 50A thereof, or the individual is the correct user guest for the electronic device 50A. The illustration in FIG. 64 is an example of any electrical impulses from any biological source that can be used for the security system and in the operations of a Brain Computer Interface system or similar and future devices. Through the gestures performed by the user, the action of vocalization commands, and the continuous repetition of the neurological impulses, a verbal command program 404A and the artificial intelligence module 25A in the Brain Computer Interface or in the electronic device 50A are provided multiple analyzable inputting information able to operate independently, or in combination scalable and customizable on the electronic devices 50A with a Brain Computer Interface (BCI) attachable or detachable or without a physical Brain Computer Device (BCI). Once the signals are programed and operated, the user will have quicker interaction time in the electronic device 50A reducing workload, though the usage of a Brain Computer Interface (BCI) or with any other digital operational computing process.

Furthermore, the algorithm in the BCI or in the electronic device 50A independent or simultaneously is capable to utilize the electrical impulse signals of the brain in conjunction with the electrical impulse signals from the extremities wherein both signal acquisitions can be operational during the operations and compliments with each other as a security system. For example, when the user is turning on the programs in the electronic device 50A, or wants to open a selected program requiring verification, for the appropriate user, the combination of the brain signals and extremities signs verifies the user, in either the non-physical intent (imagine) gestures and or in the physical performed gestures, considering an authorize user wherein the (EMG) and the (EEG/ECOG) signals are recognized and analyzed by the algorithm system as appropriate for the correct gesture perform or gesture intent/imagine. This is an example of a robust security system for any electrical security system and/or operations of the one or more electronic devices 50A, wherein both signals authenticate the user. In the event the brain signal and the extremity signal acquisition are not compatible with past programmed signals acquisition from either (EMG) and/or the (EEG/ECOG) the electronic device 50A or any other electrical security system or any computing devices using this type of security system, the electronic device 50A will not operate. The cause of the non-operation can be due to wrong user, wrong gesture (intent or performed) or wrong gesture performed or imagine. For example, when the user produces or imagines the wrong gesture for the desire program, the wrong (EMG) and (EEG/ECOG) signal acquisition are not recognized. The system recognizes signal acquisition from stored and programmed signals in the past, the non-compatible signal imagined or performed are not stored and they are not recognized and thus would not be paired with the gesture as well as the program. Such non-compatible signal acquisition will not generate a response nor operate any program of any electronic device 50A. In other words, the system will only recognize the gesture that the user pairing with a selected program performed or imagined from the brain signal or the extremity signals paired with the physical moments or the imaging gesture. In another example, a combination system for user gestures and for verification can include several gestures sequentially performed physically or non-physically (imaging gestures).

For example, in a combination pattern for a security process with unlimited gestures, one out of three signal acquisitions is not a performed gesture, that one signal acquisition is an imaginary signal acquisition, wherein it also requires the corrected gesture imagine. In other words, it is not physically performed or physically vocalized. All signal acquisitions have a selected order regardless of the process whether or not the signal acquisition came from a physically performed gesture or from an imaging gesture. In this system, the imagining gestures are only known by the user and the (BCI) signal processing system, cameras and viewers do not recognize the imagining gesture nor the order of the imagining gesture in relation to the combination of performed gestures and imagining gestures, when both the performed and imagined are used as the combination for a security process of any computer-readable program code. Additionally, the sideline element 31A can provide the user the separation for operations of multiple programs with the (EMG) and the (EEG/ECOG), in relation to multiple sensors delivering the signal acquisitions, manually or automatically, within the program and for designated programs. In other words, through the sideline element 31A, the user can operate multiple programs, multiple computer languages, multiple displays, multiple window programs, at the same time or in a timely manner, on any two more displaying contents 212A separated by one or more sideline elements 31A. The user is able to perform a physical gesture or an imagining gesture separated by a sideline element 31A, where the gesture is paired with the program or program function, wherein the signal acquisition for the gesture is also paired with the selected gesture and the selected program or selected program function to generate a close loop response on the electronic device 50A or robotic apparatus.

It is worth mentioning that all physical or imagine signal acquisitions from an EEG, EcoG and/or EMG are paired with their corresponding gestures, and vice versa that they may not be paired with their corresponding gestures. For example, the signal acquisition can be paired with two different programs, one from the performed vertical gesture and the other from the imagine vertical gesture. Having two different programs for one gesture is possible when utilizing one or more interoperability systems because, in the electronic device 50A, the display screen 52A can be configured to recognize the performed vertical gesture through the display screen controller module 20A. Also, to the imagining vertical gesture, neither the electronic device 50A nor the display screen 52A may recognize the imagining vertical gesture to generate a response, and thus the signal acquisition of the EMG, or on the EEG/ECOG can be paired with a different program or program function in relation to the same performed vertical gesture such that even there is no touch point 521A detected on the display screen 52A, the module of analyzable factor(s) 26A and the artificial Intelligence module 25A of the electronic device 50A may still be activated with the paired imaging gesture. It is appreciated that the imagine signal acquisition from the EMG or from the EEG/ECOG is fully operational and, therefore, the signal acquisition for the Brain Computer Interface (BCI) can generate the imagining vertical gesture from the EMG or from the EEG/ECOG type of signal acquisition from the Brain Computer Interface and respond in the electronic device 50A and the displaying contents 212A on display screen 52A. Furthermore, in this example, when utilizing the EMG or the EEG/ECOG, the users may also have multiple signal acquisition (EMG/EEG/ECOG) operating at the same time in a timely manner, for example one from the EEG, the other from the EMG, each paired with a different program or program function that where not selected for pairing with a performed gesture, etc.

The artificial Intelligence module 25A provided in the electronic device 50A, the Brain Computer Interface or the cloud server 100A can be activated by all the signal acquisitions. Furthermore, the signal acquisition can be provided by an attachable (BCI), detachable (BCI) system or a built-in (BCI) system in the electronic device 50A, wherein the processor 51A can be physically built in the electronic device 50A or the computing can be performed through the server processor 10A of the cloud server 100A.

It is worth mentioning that the electronic device 50A can process all the information coming and going to the AR/VR/MR/XR apparatus, with or without any sign acquisition from a (BCI) device, wherein the close loop process can be any combination of the Brain Computer Interface (BCI) device and the electronic device 50A, with or without the AR/VR/MR/XR apparatus, or a Brain Computer Interface (BCI) in the electronic device 50A and the AR/VR/MR/XR apparatus.

The signal acquisition (EMG) from a physical gesture can be performed and paired with a selected program or program function, and to also have the same (EMG) imagining non-physical (gesture) signal acquisition paired with a different pre-selected program or program function. Furthermore, the same signal acquisition being imagined or performed may be paired with the same program or program function. This method provides the user to have a more robust security system with a performed gesture and an imagining gesture rather than just having one portion for pairing the signal acquisitions with a program or program function through the usage of (EMG) signal acquisition Brain Computer Interface.

The signal acquisition (EEG/ECOG) from a non-physical gesture can be performed and paired with a selected program or program function. The same (EEG/ECoG) imagining non-physical (gesture) can produce different selected signal acquisition one form the EEG while the other from the EcoG for operating different programs or program functions. Furthermore, the same signal acquisition imagined or performed may be paired with the same program or program function. This method provides the user to have more than one options for pairing signal acquisitions with programs or program functions through the usage of (EEG/ECOG) signal acquisition.

The signals acquisition from the (EMG) and from the (EEG/EcoG) may be paired with the same program and program function. When both signal acquisitions are coming from more than one inputting system like the BCI with EMG or lithe BCI with EEG/EcoG at the same time, they can be complimented with each other without interference as a form of a robust security system. Additionally, the signal acquisition from the (EMG) can be paired with a selected program function and the signal acquisitions from the (EEG/EcoG) can be paired with a different program function where both signal acquisitions generated two responses at the same time. It is appreciated that the (EMG) signal acquisition may produce a selected program while the (EEG/EcoG) signal acquisition produces a different program function on the same program one the same gesture at the same time, as a form of multitasking.

Accordingly, the present invention discloses several inputting means, at least including a first means is the touch point gesture, a second means is the touch point gesture incorporated with the module of analyzable factors 26A and the artificial Intelligence module 25A on the display screen 52A of the electronic device 50A, a third means is the infrared camera sign-language gesture reader, a fourth hand/finger software detection program, and the fifth means is the signal acquisition of the (EMG, EcoG, EMG) of the (BCI) device, which can be performed or imagining gesture for operating, a combination or independently of the touch point gesture and the imagine signal acquisition. The hand/finger software detection program, without the infrared camera sign-language gesture can be operated or a combination of the touch point gesture and the infrared camera sign language gesture reader and the (EMG, EcoG, EMG) signal acquisition. The electronic device 50A may also be capable of incorporating the touch point gesture with the infrared camera for infrared sign-language gesture reading without any (EMG) signal acquisition for the (BCI) for operation.

In addition, for the gesture pairing for the selected program or program function, there are multiple options for the user's individualized gesture, including the gesture of one or more touch points 521A on the display screen 52A or control display screen 52XA detected in the electronic device 50A and the gesture from the infrared camera and/or from the EMG or from the EEG/ECOG. In other words, the same gesture can have been paired with different programs or programs functions. Further, multiple inputting means may identify the same gesture at the same time for a robust security system. In addition, with the combinations of inputting means, the user can select the desire gesture for the selected combination of inputting means. With redundant inputting means recognition of the same gesture, the user can select different programming for the same gesture from one of the other redundant inputting means, as the means for multitasking to generate two or more responses at the same time or in a timely manner.

For example, the present invention provides multiple inputting apparatus with the most compatible ergonomic delivery convenient and portable, for example the electronic device 50A, the infrared camera sign language gesture reader together with the (BCI) for the EMG and or for the EEG/ECOG, wherein each inputting option can have at less one different option of pairing a gesture with a program or program function. Furthermore, each gesture can be performed or imagined for the Brain Computer Interface (BCI) system. Additionally, multiple other inputting devices can have gesture interoperability for pairing with a program or a program function in the electronic device 50A. The invention teach one or more Brain Computer Interface (BCI) system for each signal acquisition, one for the (EMG), and the other for (EEG) and the other for the (ECOG) or for other desire signal acquisition for other biological signals requiring the function of a Brain Computer Interface system or similar devices. It is possible for one Brain Computer Interface device to alternate between signal acquisition just like a remote server may provide alternating digital signal corresponding to the gesture and the user selection. Each signal acquisition is processed by the Brain Computer Interface (BCI), wherein the Brain Computer Interface computing can come from the cloud or the internet. The Brain Computer Interface device can come from an attachable adjacent device or from a Built-in Brain Computer Interface within the electronic device 50A. Through the electronic device 50A, the physical size of traditional Brain Computer Interface device can be reduced. The electronic device 50A can be the control source for signal acquisition in real time, wherein the harvesting for any signal acquisition can be done through the electronic device 50A with an attachable or build-in Brain Computer Interface (BCI) with available options for automatically or manually calibrating the energy and frequency on the wirely or wireless electrodes sensors for acquiring adequate signal acquisition for gathering the signal acquisition to operate with any “Brain Computer Interface” (BCI) device, thereby any remote Brain Computer Interface computing system can receive and process remote signals of (EMG, EEG/ECOG) through the internet, or the cloud server 100A in private or public manner.

It should be noted that the gestures according to the present invention are not limited to the gestures performed by physical finger(s) touching on the display screen 52A to generate the one or more touch points 521A, but also referring to gestures preformed with the EMG, or with the EEG/EcoG. Any neurological impulses performed by body movement or imagine can function as a gesture including verbal commands with respect to the gestures. It is appreciated that a structural and non-structural tactile system with motion sensors, motion cameras, hand/finger software detection program and/or infrared cameras detector can be operated independently or together collectively for natural optimization of multitasking, wherein multiple programs or program functions are generated and operated in a desire systematic processes. In other words, the user can select the pairing of a gesture with a program or program function for each individual inputting devices, and that not all inputting devices have the same pairing of the gesture with the same program or program function. Furthermore, some inputting devices will not recognize the gesture thereby not generating a response. Some inputting devices will recognize the same gesture and have the same pairing for the selected program or program function, so that it provides the selected programs and program functions a robust security and the desire operational precision for selected gestures.

It is worth further mentioning that the pairing can be operated between the one or more touch points 521A of gesture and a program or program function, between the sign language gesture (infrared system) and a program or program function, and/or between the imagining gesture (Brain Computer Interface) and a program or program function.

Hand gloves are an optional tool in relation to multitasking. The goal is to deliver a wearable optimization for gesture production and utilization on the display screen 52A or without a physical structure, an imagine or as a form of sign language gesture. The glove provides additional verification of gestures with light, tactile and thermal delivery where the infrared camera and the display screen 52A receive the information together or independently. Additionally, the power source of the glove can be incorporated with the (EMG) device. In other words, both the (EMG) and the glove may have separate power supply and or may share the same power supply. The glove can be made of a flexible fiber optic mesh of various size or of one size with stretching capacity to fit all sizes.

It is appreciated that the fiber optic mesh is a branching intertwine elongated structure running along the user's hand as a flat, circular, square or any other shape continuous tubing structure from above the wrist, with or without the (EMG) sensor and power supply. It is worth mentioning that, alternatively, the fiber optic mesh is a transparent web like structure beneath or above a glove with pores like medium surface. Optical fiber lines and the micro light electrical tape or line do not reduce light emitting with any joint flexion while continuously conducting light. Furthermore, it is possible for heat transfer through a micro light Emission source with or without optical fiber lines when it is in close proximity with human skin that allows thermal warming to the user while increasing the gesture verification thorough the infrared camera and without reducing sign language gesture performance, that is the usage of a grid multiplexed gesture.

According to the present invention, gesture for generating touch points 521A would be tactile gesture, verbal/audio software program, camera for motion control, infrared detection, hand/finger software detection program processes of interoperability operating on 2-Dimensional, 3-Dimensional, 4-Dimensions and non-structural sensory control systems to interface and operated multitasking in 2-Dimensional, 3-Dimensional, and 4-Dimensional display programs independently or in conjunction with each other, and with other imputing systems, and etc.

Considering verbal commands in a noisy environment without the Brain Computer Interface, verbal commands may loss its function. Therefore, voice commands with (BCI) allow the user to continue operating the electronic device 50A without affecting the inputting capabilities. The touch point 521A, according to the present invention, does not limit to a finger touching on a touch screen type display screen 52A, but also includes a physical finger of the user or a virtual operation finger tool 5211A of a performed or imagining gesture of the user that is detected in a touching action with respect to the virtual type display screen 521A as shown in FIG. 56C, wherein the display screen 521A may refer to projecting display screen 521A projected from the projecting device in front of the user's eyes.

Therefore, in the Step 101A of the method of generating the adjuster 13A, the display screen 52A may also detects one or more touch points 521A and the projecting display screen 521A generates a responsive performed or imagining touch point 521A, with or without tactile, at a position of the touching action of the finger or virtual operation finger tool 5211A with respect to the virtual type display screen 52A, proportional to a location of the displaying portion 523A of the display screen 52A, the display program and the display medium, and the adjuster module 10A receives the information of the respective touch point 521A, the sign language gesture, the imagining gesture performed or imagined detected and sent from the touch screen 52A, the infrared camera for sign language gesture and/or the Brain Computer Interface for the imagining gestures.

In addition, receiving information from the infrared camera from the user's finger thermo-heat (sign language gesture), with or without a glove or sleeve sensors, with or without the hand/finger software detection program. The touch screen 52A or display medium generates a respective touch point 521A displaying at a position in relation to the display screen 52A in relation to the finger's thermos-heat with or without a glove or sleeve sensors, and then the adjuster module 10A receives the information of the infrared gesture location detected and sent from the infrared camera and/or from the gloves or sleeves sensors. The information is analyzed by the module of the analyzable factor 26A, the artificial intelligence module 25A and the distance tracker module 27A of the display screen control module 20A, for function, movement and direction of each of the one or more sign language performed gestures.

Furthermore, the receiving information from the Brain Computer Interface (BCI) system is initiated by a signal acquisition from an imagining gesture. The display screen 52A receives one or more imagining gestures, wherein, for example, the touch screen 52A generates a respective touch point 521A at a position of the display screen 52A, and the adjuster module 10A receives the information of the imagine gesture detected sent from the Brain Computer Interface (BCI), wherein the information is analyzed by the module of analyzable factors 26A, the artificial intelligence module 25A and the distance tracker module 27A of the display screen control module 20A or in the Brain Computer Interface for functioning, movement and direction of each of the one or more imagining gestures.

In the Step 102A, in addition to the touch point 521A, if the sign language and/or imagining gesture doesn't move, go to step 103A. If the sign language gesture and/or the imagining gesture is moving, detecting the moving direction of the sign language and/or imagining gesture, thereby sending the moving direction and location of the sign language and/or the imagining gesture to the adjuster module 10A, and then go to step 104A.

In the Step 103, the another touch point 521A can be from the infrared camera, the hand/finger software detection program, and/or the Brain Computer Interface.

In the Step 105, the sign language gestures and the imagining gestures also require significant less physical demands.

In the Step 109, the sign language gesture can be proportionally tracked by the infrared camera and/or hand/finger software detection program. Further, the imagining gesture can produce signal acquisition variants for various adjusters 13A, sideline element 31A, including selected numbers of the numeric indicators 16A.

In the Step 110A, the touch point gesture also includes the sign language gesture and/or one or more imagining gestures which can also recognized and localized by the module of the analyzable factors 26A, the artificial intelligence 25A, and the distance tracker module 27A of the display screen control module 20A after the information is provided by the infrared camera. Hand/finger software detection program, and/or the Brain Computer Interface so as for the adjuster module 10A to generate the adjuster 13A, the graduation(s) 120A, the adjuster indicator 14A, and/or the numeric indicator 16A which are all customizable on the one or more program in the one or more display screens 52A or display medium, wherein the gesture has been pre-programmed to display one or more display contents 212A of one or more programs on one or more display screen 52A, specifically for the pre-program gesture of the desire program or function, without additional physical movements.

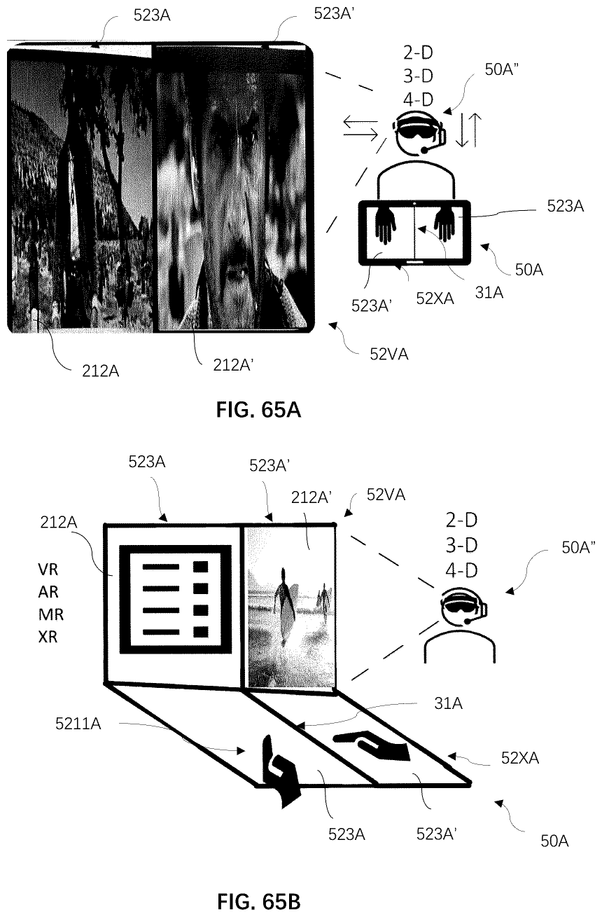

Referring to FIG. 65A, the user may simply perform a sideline gesture, such as triple tap, or imagining a triple tap, on the control display screen 52XA of an electronic device 50A as an operating and controlling apparatus to generate a sideline touch point information which is recognized by the display screen controller module 20A (second executable portion 2A) and sent to the sideline module 30A (third executable portion 3) to generate the sideline element 31A to split both the control display screen 52XA and the virtual display screen 52VA projected by the headset 50A″ in front of the user's eyes (as shown in the left side screen of the FIG. 65A) into two displaying portions 523A, 523A′. Then, when an operating gesture is operated by the user on each of the two displaying portions 523A, 523A′ of the control display screen 52XA, the touch point information detected by the display screen controller module 20A is sent the adjuster module 10A to generate an adjuster 13A on the respective displaying portion 523A or 523A′ of the virtual display screen 52VA for adjustment operating of the display content 212A, 212A′ on that displaying portion 523A, 523A′ according to the touch points 521A produced by the touching gesture of the user. It is worth mentioning that gesture produced by any single touch point 521A or a combination of touch points 521A can be pre-programed as mentioned earlier to be paired with the gesture and cursor program 44A to generate a division between two or more programs in the one or more display screens 52A, 52VA.

The touch point information provided to the adjuster module 10A, the sideline module 30A and the interactive module 40A are analyzable touch points 521A, with the option of being produced in more than one way on the display screen 52A, 52VA, wherein the display screen controller module 20A activates the module of analyzable factors 26A for distinguishing pressure, thickness, time, acceleration, direction, distance, angle, interruptions, size, one hands or both hands, and/or location from one or more touch points 521A, wherein the information is further reviewed and learned by the artificial intelligence module 25A to differentiate from past, current and for future touch points selections. In other words, the operating gestures according to the present invention can generate Augmented Reality, Virtual Reality, Extended Reality, and Mix Reality and control said displays. For example, any of the module of analyzable factors 26A, like the pressure of the one or more fingers, the movement of one or more fingers and the time duration of gesture are differentiations from the Augmented Reality, Virtual Reality, Extended Reality, and Mix Reality. Furthermore, the 2-Dimensional touch point 521A can also be divided by the sideline module 30A for the 3-Dimension and for the 4-Dimension Augmented Reality (AR), Mix Reality (MR), Extended Reality (XR) and Virtual Reality (VR) to interchange information for the adjuster module 10A, the controller module 20A, the sideline module 30A and the interactive module 40A for verification of the selected gesture and control of the selected programs or program function in the electronic device 50A on the display screen 52A and the virtual display screens 52VA, as illustrated in FIG. 65B. Furthermore, the artificial intelligence module 25A may understand similar or identical gestures performed or imagined and can generate different responses according to the type of program operating in the Augmented Reality, Virtual Reality, Extended Reality, and/or Mix Reality.

The present invention discloses a process for the user to operate programs, avatars, surrogates, or emoji, and etc., in the display screen 52A, such as a 2-Dimensions display screen 52A or a 3-Dimensional gesture holographic apparatus, as a process to multitask on various operation systems (OS) such as Windows programs, Microsoft operations, Apple IOS, iPadOS, watchOS, macOS, tvOS, Google Android, and/or etc. with or without the sideline module 30A.

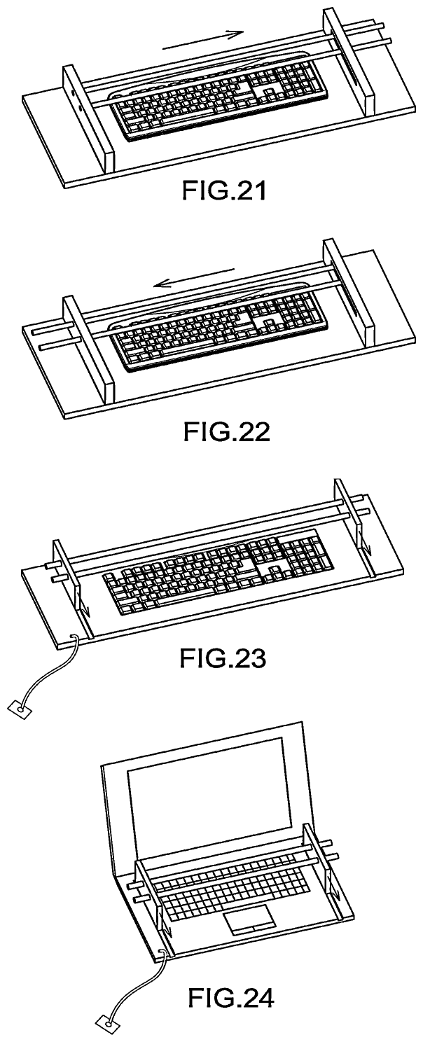

As shown in FIGS. 65A and 65B, the gestures are also customizable and controllable with verbal functions. Therefore, the user is able to work, party and engage more efficiently in the 2-Dimensional control display screen 52XA, while observing the program in a 3-Dimensions or 4-Dimensions environments through the virtual display screen 52VA. FIG. 21A illustrates the user operating on an independent control display screen 52XA of the electronic device 50A and viewing the displaying contents 212A, 212A′ through the virtual display screen 52VA through the VR/AR/MR/XR apparatus such as the goggle headset 50A″ worn on the user's head. FIG. 65B illustrates that the control display screen 52XA is also a virtual displaying content viewing through the goggle headset 50A″ for the user is able to operate via gestures detected by the infrared camera from the user's finger thermo-heat (sign language gesture), with or without a glove or sleeve sensors, the virtual operation finger tool 5211A, or the imagining gesture performed or imagined by the Brain Computer Interface. In other words, the present invention provides means for the user's performing or imagining gestures without having to physically perform the display posture and/or vocalization during a communication posture and verbal generated display in the simulated world.

Referring to FIGS. 65A and 65B, the electronic device 50A is configured to allow the user to operate the sideline module 30A on the control display screen 52XA to produce the sideline element 31A splitting the control display screen 52XA into two or more displaying portions 523A, 523A′ to separate two or more programs operating in the two or more displaying portions 523A, 523A′ respectively. Of course, two independent display screens 52A can be configured to be used for operating two programs respectively, wherein the two display screens 52A can be physically separated and are interconnected, interchangeable and interfaced with the Virtual Reality, Augmented Reality, Mixed Reality and/or Extended Reality apparatus, such as the headset 50A″. In other words, the control display screen 52XA can be functioned as a full touch screen inputting control for digital displays and to control the Virtual Reality (VR), Augmented Reality (AR), Mixed Reality (MR), Extended Reality (XR) apparatus as well as the VR/AR/MR/ER displaying contents 212A, 212A′ on the virtual display screen 52VA. Furthermore, the user controls the 2-Dimensional, the 3-Dimensional and or the 4-Dimensional virtual display screen 52VA independently or in combination through the use of gestures and verbal commands divided by the sideline element 31A. For example, the sideline element 31A can be divided into one portion of the display screen 52XA to operate a 2-Dimension program and another portion of the display screen 52XA to operate a 3-Dimension program at the same time or in a timely manner. Through the gestures and the verbal commands of the user, the user can control two or more display screens 52XA or two or more displaying portions 523A, 523A′ of the display screen 52XA with both hands to perform gestures and verbal commands or independently operated each display screen 52XA or each displaying portion 523A, 523A′ with the gestures and the verbal commands.

It is worth further mentioning that the headset 50A″ for the Augmented Reality, Virtual Reality, Mix Reality, or Extended Reality can have electromagnetic sensor inputting system to provide real time head motion tracking, specially for time sensitive gestures in addition to sign language gesture or gesture base controls performed or imagined with the (BCI) systems. The performed head motions (gestures) electromagnetic sensor is operated within the X, Y, and Z coordinates for the orientation of the yaw, pitch, and roll. According to the present invention, AC electromagnetic solid-state sensors for measuring both the position and the orientation natively can be used as the means for additional gestures, with or without verbal commands, with or without eyeball control cameras system, and with or without retinal control camera system within the headset 50A″ for the Augmented Reality, Virtual Reality, Extended Reality or Mix Reality.

In other words, the system for gesture and displaying sideline operation of the present invention understands the touch point 521A for the gesture and cursor program 44A and the display screen 52A generates and controls the selected program, wherein the verbal command can operate the right and left click, when the user pairs verbal commands with gestures respectively. This is a novel approach to manual control and verbal control operating at the same time on the display screen 52A or control display screen 52XA. Furthermore, any neuromuscular impulse like voice control can also operate the Brain Computer Interface (BCI) through the signal acquisition of (EMG, EcoG, EMG) signals.

According to the preferred embodiment of the present invention, multiple options of operating various programs in the one or more electronic devices 50A through the gestures on at least one display screen 52A is provided, wherein each of the display screens 52A interfaces with the one or more electronic devices 50A and/or interacts with other electronic devices 50A having at least a processor 51A or through the processor 101A of the cloud server 100A. Digital systems with computer usable medium having computer-readable program code can be embodied in the medium to perform executable operations, wherein the computer-readable program code comprises at least one processor or without a processor, such that the user can operate visual responses to 4-Dimensions, 3-Dimensions and/or in 2-Dimensions through the inputting of voice commands and/or touch point gestures imagined or performed. In other words, the user can perform a gesture and control said gesture for the movement and operation of the program. For example, the user can produce a gesture through the touch point 521A for the virtual mouse, wherein the user can verbalize right click or left click while the user is manipulating the gesture and cursor program 44A throughout movements with the touch point gesture originally provided on the touch screen type display screen 52A and operate the right and left click controls thought verbal commands program 77A. The same process or similar processes can be accomplished for other programs such as the sideline module 30A, the numeric indicator 16A, the keyboard element program 43A, the state-of program 48A, and etc.

The interactive module 40A of the electronic device 50A may also provide the verbal command program 404A which can be a voice command program like Siri, wherein the user can make the command for “adjuster” and the programs response operability to the user's verbal command in relation to the touch point 521A for the location on the display screen 52A. Also, through the combination of the touch point 521A and the verbal command, the adjuster 13A is generated to interface the desire command at the desire location or for the desire programs. In another example, the system understands the gesture for the gesture and cursor program 44A that the display screen 52A generates and controls the selected program correspondingly, wherein the verbal command can operate the right and left click while the user also pairs verbal commands with gestures. In other words, the manual control and the verbal control can be operated at the same time or in a timely manner on the display screen 52A.

For example, as shown in FIG. 48A, when the user's one or more fingers perform a gesture, such as touching on the touch screen type display screen 52XA, one or more touch points 521A are detected on the display screen 52A and the display screen control module 20A acquires the touch point information, wherein the module of analyzable factors 26A and the artificial intelligence module 25A provide the gesture information to the processor 51A, and then the adjuster module 10A, the interactive module 40A and the sideline module 30A receive the gesture information.

Similarly, as shown in FIGS. 65A and 65B, when the artificial intelligence module 25A recognizes the gesture information is a voice dependent gesture, the verbal command program 404A of the interactive module 40A is activated. In other words, the touch point gesture is localized on the display screen 52A and the verbal command module 404A generates the user selection based on one or more locations of the one or more touch points 521A that are identified by the display screen controller module 20A. For example, the user may speak out for an “adjuster” 13A, or a number, such that the verbal command program 404A is recognized by the artificial intelligence module 25A and provides the information to the server processor 101A or the processor 51A of the electronic device 50A. Thereby, the verbal command program 404A generates an adjuster 13A accordingly at the location of the touch point gesture. Similarly, if the user speaks out for an “sideline” 31A, or a number, the verbal command program 404A is recognized by the artificial intelligence module 25A and provides the information to the server processor server 101A or the processor 51A of the electronic device 50A. Thereby, the verbal command program 404A generates a sideline element 31A accordingly at the location of the touch point gesture.

In view of above the interoperability of the touch point gesture on the display screen 52A incorporated with the verbal commands program 404A is illustrated that is an example of precision and scalable control options for multitasking with two or more imputing programs in one or more electronic devices 50A. In other words, a combination for inputting can be used for any desire program in any location of the display screen 52A including in the VR/AR/MR/XR apparatus.

The present invention may further equip with an arrangement of operating gestures for controlling the moments and verbal functions of simulated characters, without the user conducting any body movement or any verbal function. In other words, the gestures are controlling the display audio and animation. Gestures provide the user the ability to increase productivity and tolerate demanding long work hours, as well as to reduce physical demand and vocal demands. For example, a corporation may train its customers and employees to operate gestures when communicating via the VR/AR/MR/XR apparatus. Therefore, the consumer will get the same visual experience and the same auditory experience with a live customer service employee through the VR/AR/MR/XR apparatus, wherein the employee does not need to vocalize most of the words in the communication nor does he or she need to perform all the displayed body postures or body language that are displayed during the communication.

Referring to FIG. 65B, considering all the optional programs and functions mentioned above are generated by a gesture, a verbal common, and a headset with motion control, wherein the user can produce a gesture or a verbal command for the visual display shot, clip, or animation, of an avatar or surrogate. Also, the user can control any type of frame like, long shot, a medium shot, a close-up shot, or extreme close up, and etc., or the level of the angle of the frame eye level angle, high angle or low angle, including the degree of lighting from low key lighting to the high key lighting and any degree in between. When the user gets closer to the display screen 52A with the VR headset 50A″, the virtual reality headset 50A″ senses the change in motion and display the frame program for the location of the headset 50A″ accordingly. For example, if the user moves the headset 50A″ away from the display screen 52A, the long shot frame is generated. Similarly, when the headset 50A″ is moved closer to the electronic device 52A, the extreme close up shot is generate. A headset motion control may be interfaced with the electronic device 50A. In addition, for example, the bending of the neck with the headset 50A″ may operate the lighting, wherein the bending the neck to the left may generate a low key lighting, bending the neck to the right may generate a high key lighting, and the positioning the headset 50A″ in neutral may generate natural white balance. Certainly, the user may further select various programs to be operated with the headset 50A″.

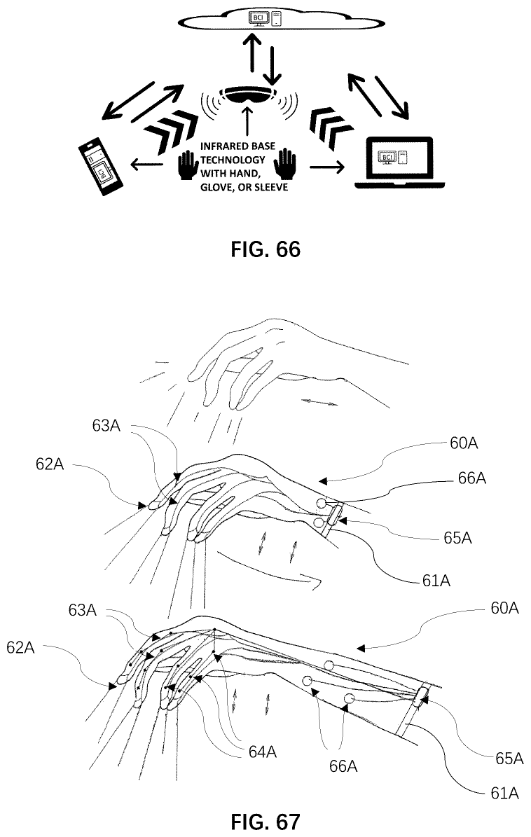

Referring to FIG. 66, human hands are able to be equipped as a powerless and multiplexed infrared light source for gesture pairing with programs and program functions for controlling various electronic devices 50A (such as smartphone, laptop, AR/VR/MR/XR apparatus, and etc.). In other words, the multiplexed infrared light source can be equipped as the gestures in relation to operating functions in an electronic device 50A, wherein the multiplexed infrared light source system will not interfere with the analyzation of the one or more touch points 521A on the display screen 52A, nor with the signal acquisition from the Brain Computer Interface, and etc. In addition, the interoperability for operating the VR/AR/MR/XR headset 50A″ through the one or more electronic devices 50A with or without infrared base cameras, with or without glove or sleeve, with or without hand/finger software detection program, and with or without Brain Computer Interface system. For example, the interoperability between the infrared base control system and the inputting system of the display screen 52A with or without the performed or imagined touch point 521A. That is the present invention is capable of operating a plurality of electronic devices 50A independently and collectively with multiple inputting systems.

Alternatively, the hands can be equipped with a powerless infrared light source. The human fingers are natural Infrared emitters due to the thermal emission from the hands, especially in an emitting or receiving infrared base encryption/decryption system for reflection based information encryption/decryption. The human hand not only can serve as a single integrated infrared light source but also can serve as a multiplexed infrared light source due to its unique composition. Each hand has five fingers, each of which can serve as an independent infrared light source and can be controlled and grouped at will, allowing for gestures production and interpretation.

The human hand can also be equipped with one or more powerless light sources for infrared-based encryption/decryption. In other words, the encryption/decryption mechanism that relies on infrared reflection can be used for complex signal recognition. The intensity distribution of the thermal radiation from a typical hand at normal body temperature, and the infrared peak of the human hand is well within the atmospheric transmission window, as well as the detection range of the infrared detectors, that are able to illuminate structurally designed gratings. Such design may generate infrared diffraction patterns by different gesture. For example, the infrared alphabet based on hand-grating interaction (IA-HG) and the three-finger alphabet (TFA) inputting systems are referenced. Also, the direct capture and recognition of gestures can be performed by thermal or infrared camera imaging process. It is worth mentioning that, according to the present invention, the gestures are not limited to three finger gestures. In other words, the infrared light source gesture from the hands may be recognized and analyzed by the electronic device 50A with or without touch points 521A produced on the display screen 52A. Furthermore, the infrared gestures inputting system is based on the five fingers of each hand operating with the Brain Computer Interface for performed or imagined infrared gestures to control the electronic device 50A with or without a physical touch screen 52A. Additionally, the hand/finger software detection program is able to increase the accuracy of gestures, through the camera, internet of things, the internet or cloud servers.

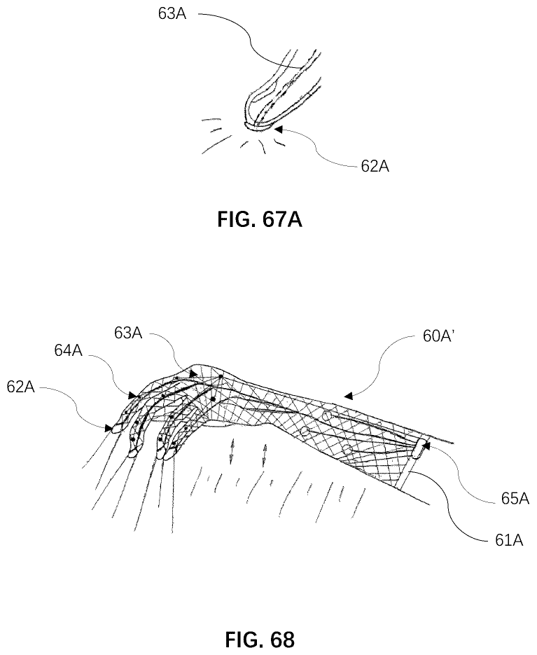

Referring to FIGS. 67 and 67A, a hand cover 60A such as a glove or a sleeve configured to be worn on a hand of a user is an optional gesture tool in relation to multitasking. The goal is to deliver a wearable for optimization for gesture decryption and utilization on a physical display screen 52A or on a virtual display screen 52VA with performed gestures or imagining gestures as a form of sign-language gesture. The hand cover 60A comprises at least one or more multiplexed infrared light source 61A at a distance for verification of gestures with infrared camera, one or more transparent optic lenses support 62A such as tactile soft gel lenses provided at tips of the fingers respectively for comfort pressure support from continuous inputting, and a plurality of elongated fiber optic element 63A extended between the at least one light source 61A such as infrared light source and the transparent optic soft gel lens support 62A. The transparent optic soft gel lens support 62A magnifies the infrared intensity distribution with the micro light element 67 from the light source 61A and also transmitted through the fiber optic elements 63A. It is important to mention that the light source 61A has different adaptations for the fiber optic element 63A and the micro light element 67A, with the fiber optic element 63, the light is emitted by the light sources 61A, and with the micro light element 67A, the light source 61A is the electrical control to deliver energy to the micro light element 67A. Alternatively, the hand cover 60A is equipped with a micro light element 67A, with or without the fiber optic elements 63A, wherein the micro light element 67A can deliver thermal energy directly and indirectly to the user and to the infrared camera on any electronic device 50A. It is worth mentioning that both the fiber optic element 63A and the micro light element 67A can work together or separately and both provide transillumination of the fingers (infrared emissions) for the infrared camera. The hand cover 60A may further comprise one or more electrodes (EMG) sensors 66A, distributed proximal to the wrist and the forearm, wirelessly or when wired acquiring the powered from the power source 65A, which is configured in such a manner that the electronic device 50A receives the information together or independently in relation to the electrode (EMG) sensors 66A (signal acquisition) and the infrared lighting camera decryption. Additionally, the electrode (EMG) sensors 66A are able to be relocated throughout the body part with or without adhesive made of hypoallergenic material.

It is worth mentioning that the power source 65A can power directly or indirectly the light source 61A, the fiber optic elements 63A, the micro light element 67A, and the electromagnetic sensor 64A. Alternatively, the light source 61A or the power source 65A can provide the user the customized options for the type of inputting for the glove or sleeve. Additionally, all the wire connections may directly connect to the power source 65A or the light source 61A, or may be a combination between the light source 61A and the power source 65A. Furthermore, the light source 61A or the power source 65A may be equipped with the manual or automatic amplifier to deliver the signal acquisition from the electrode (EMG) sensor 66A. It is possible if the electro (EMG) sensor 66A is wireless, the power source 65A and the amplifier are build-in the electro (EMG) sensor 66A.

The inputting system of the electromagnetic sensor 64A provides real time finger and hand tracking, especially for time sensitive gestures in sign language base decryption or in touch point controls, including (BCI) systems for the performed gesture that require X, Y, and Z coordinates for the orientation of the yaw, pitch, and roll. According to the preferred embodiment of the present invention, the AC electromagnetic sensors 64A as a solid-state for measuring both the position and the orientation natively. The electromagnetic sensors 64A have various shapes and various electromagnetic waves distinguishably programed for each phalanges including the hand. The electromagnetic sensors 64A can work adjacent with fiber optics elements 63A because the fiber optic elements 63A are immune and impervious to electromagnetic sensors 64A waves. With the fiber optic elements 63A and the electromagnetic sensor 64A, the combination of the fiber optic elements 63A provides the user infrared multiplexed light source, allowing the infrared base control system to identify the gestures at a significant distance without the performed gesture getting lost within the environment distribution of the thermal radiation from the background of the ambient temperature. The electromagnetic sensors 64A provide the gestures the X, Y, and Z orientation to further enhance the gesture recognition with or without the transparent optic soft gel lens 62A transillumination magnification connection at the tips of the fingers. It is worth mentioning that the one or more transparent optic soft gel lenses 62A of said glove or sleeve 60A are capable of activating the touch screen 52A in the electronic device 50A for gesture operations and recognition by the module of analyzable factors 26A and by the artificial Intelligence module 25A for operating said AR/VR/XR/MR visual display screen 52VA including the display screen 52A of any electronic device 50A.

Alternatively, referring to FIG. 68, the fiber optic elements 63A can be positioned above or below a flexible mesh type hand cover 60A′ that is a branching intertwine elongated structure running along the user hand or arm as a glove or a sleeve, wherein the fiber optic elements 63A are extended within, above or below as flat, circular, square or any other shape with a continuous interconnected tubular structure, originating above the wrist or the sleeve, with or without the electrode (EMG) sensor 64A and power source 65A wirelessly or wirely. It is worth mentioning that, alternatively, the distribution of the fiber optic elements 63A is transparent within the web like structure beneath or above a porous medium as a glove or as a sleeve. In addition, the fiber optic elements 63A is excellent in light (wavelength) transmission which is customizable and able to be distributed with any joint flexion. The hand cover 60A′ can be made of any flexible medium material, such as hypoallergenic flexible rubber, nylon, polymer, silk, and etc., while mesh of various sizes and thickness or of one size with stretching capacity for fitting all sizes. It is worth mentioning that the hand cover 60A′ provides the support over the electrode (EMG) sensors 66A for the proper (skin contact) to acquire the signal acquisition needed by the Brian Computer Interface (BCI).

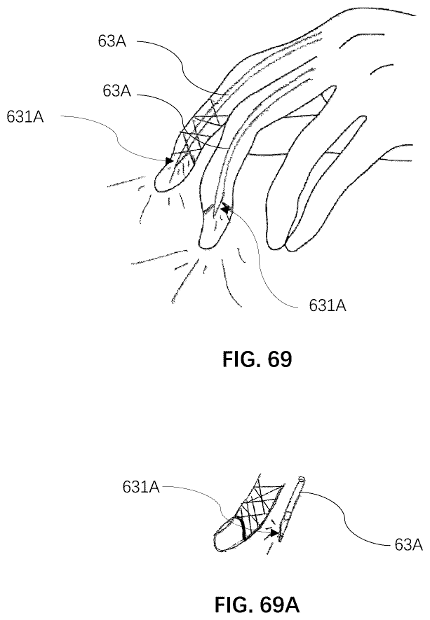

Referring to FIGS. 69 and 69A, Whether the fiber optic glove or the fiber optic sleeve are used as the hand cover 60A, all the branching of the fiber optic elements 63A and positioning for the micro light element 67A are preferred to be congregated at the ends near the finger tips of the user's hand with or without the optic lens 62A, wherein an end of each of the fiber optic elements 63A is cut with an angle to form an inclined light emitting end 631A so as to further increase a surface area of the light emitting end 631A of the optic fiber element 63A while in contact with the user skin for the magnification of a humans hand as an infrared light source. Additionally, the micro light element 67A can be positioned to have contact with the skin directly or indirectly. Also, the micro light element 67A may be connected with an extension of the fiber optic element 63A, wherein the fiber optic element 63A makes contact with the skin, as illustrated in FIG. 69. This is in contrast to FIG. 69 that both the fiber optic element 63A and the micro light element 67A are connected to the transparent optic soft gel lens 62A.

It is worth mentioning that the fiber optic elements 63A of the present invention are utilized to transmit infrared light, wherein the fiber optic elements 63A are not limited to one light wavelength or type or material, etc. The fiber optic elements 63A are capable of working for transmitting light with various wavelengths such as UV light, visible wavelength blue, green, red light and infrared light onto the skin. The brightness of the fiber optic element 63A can be customized and the type of wavelength can be controlled to transmit light with one or more wavelengths at the same time on the same display medium. In other words, infrared light is not the only type of light source for decryption in a sign-language base gesture. Furthermore, the optic lens 62A positioned at the tip of the finger can be made of a soft gel optic material as a physical barrier for tactile finger tip comfort during typing. Alternatively, the fiber optic elements 63A and micro light element 67A extended through the glove or the sleeve medium originating from to the wrist/forearm and terminated at the finger tip or at the transparent optic soft gel lens 62A adjacent to the distal phalanx fat region hyponychium (finger pulp).

Also, the glove/sleeve type hand cover 60A equipped with fiber optic elements 63A and the electrode (EMG) sensor 64A to form a glove/sleeve fiber optic system that allows the user to operate gestures at a further distance in relation to the infrared camera and/or the IA-HG (TFA) system, wherein the ambient temperature may distort the human natural infrared delivery of gesture commands on any infrared base system. As mentioned above, such glove/sleeve fiber optic system can deliver more direct infrared light to any infrared operational system while it is not limited to one type of fiber optic, for example Clearcurve and Durafiber nylon fibers, and etc.

Regarding the computing capacity processing from the electronic device 50A, the BIC processing system, the infrared camera processing system, and the AR/MR/XR/VR processing system, the hand finger software detection program and any other software operations are preferably processed through the electronic device 50A, received by the electronic device 50A, delivered by the electronic device 50A to the cloud server 100A through the internet, attached to the electronic device 50A, interfaced with the electronic device 50A, and displayed on the display screen 52A while being controlled by the performed or imagining gestures. Furthermore, the user may select the control system with one or more signal acquisitions, such the EEG, EMG or ECoG for the Brain Computer Interface system. In other words, the user can perform or imagine a gesture from any of the signal acquisition, even though the user may be connected to more than one different signal acquisition electrodes while utilizing the Brain Computer Interface and operating the AR/MR/VR/XR headset or the display screen 52A of the electronic device 50A. The user can also select a specific program with any type of signal acquisition to operate with the BCI system. For example, the user can select to only operate the Mix Reality (MR) through the EEG sensor signal acquisition on the headset with the Brain Computer Interface. The same is true for the ECoG wherein the user produces a gesture to turn off the EEG for the Brain Computer Interface control system, and turn on the ECOG sensory signal acquisition for the Brain Computer Interface control system in the Virtual Reality (VR) headset to operate selected programs. The same is true for the EMG wherein the user produces a gesture to turn off the EEG and/or the ECoG control system in the headset, wherein the Brain Computer Interface with the EMG signal acquisition control system is turned on and the Brain Computer Interface with the EEG and or ECOG is not operated for the Augmented Reality (AR) programs. It is worth further mentioning that, in this example, neither signal acquisition from the EMG, EEG or ECOG interferes with each other to operate in a Brain Computer Interface system. Furthermore, either the EMG, EEG or ECoG signal acquisition will require one Brain Computer Interface system for each different sign acquisition. It is understood that none of the Brain Computer Interface computing system is required to be physical present as attachable or detachable hardware for the processing of the close loop function between the AR/VR/MR/XR apparatus and the any other electronic device 50A. Once the signal acquisition is acquired by the electronic device 50A, the electronic device 50A can send the signal acquisition to the cloud where the Brain Computer Interface servers in the cloud returning a digital signal to the electronic device 50A that sends the signal acquisition or generates a response in the AR/VR/MR/XR apparatus and interfaced with the electronic device 50A. The electronic device 50A can receive the signal acquisition from the EMG, EEG and/or ECOG electrodes wirely or wirelessly. The electronic device 50A can also process one or more selected signal acquisitions with a built-in (BCI) software or through an attachable Brain Computer Interface system, or neither thereby the electronic device sends the signal acquisition to the cloud server 100A where one or more signal acquisitions are processed back to the electronic device 50A or to the AR/VR/MR/XR apparatus to generate a response in a close loop operational control system.

The electronic device 50A, such as tablet, laptop or computer, can be the computing source for the AR/VR/MR/XR apparatus that receives the initial digital sample after the signal has been amplified and past through a low-pass fielder. The electronic device 50A may then activate the built-in (BCI) software application or a download permissible version of the (BCI) software program. Alternatively, the electronic device 50A may also deliver the information to the cloud (BCI) server 100A through internet for the computing and to generate a response in the electronic device 50A, the AR/VR/MR/XR apparatus, or in the display screen 52A. The electronic device 50A, such as tablet, laptop or computer, may have all the software capacities for each type of the Brain Computer Interface so as to reduce the EEG/ECOG and EMG weight requirements for operating the Brain Computer Interface. The wearable AR, VR, MR, XR apparatus may only consist of the power source, a differential amplifier and the transmission source therein, providing the least weight capacity. An independent device may be attached or built in the tablet, laptop or computer type electronic device 50A that can provide the digital information such as a USB. Furthermore, the electronic device 50A may or may not further amplify the signal and/or pass the signal through a low-pass filter for the process of converting the signal into digital samples. The same is true for the wearable EMG signal acquisition within the gloves or sleeves with the power source, the differential amplifier and the transmission source to deliver the signal to a separate device attached or built in to the electronic device 50A such as tablet, laptop, computer, or to deliver via a portable USB to further amplify the signal where the signals are passed through a low-pass filter (aliasing) for the processes of converting the signal into digital samples. In the last step of converting the signal to a digital sample, the signal acquisition can be delivered to a separate attachable or remote device for converting the signal to a digital sample as the means for reducing the weight capacity on the total wearable sensor equipment in the EEG, ECOG, or EMG system in relation to BCI interoperability wearables.

Further, the display screen 52A of any electronic device 50A can interface for delivering the most interchangeable, interoperable and wearable biometrics systems to control any digital program. Through the interface between the electronic device 50A, the display screen 52A and the AR/VR/MR/XR headset system. The system of the present invention may operate more than one signal acquisition at the same time, wherein the user may have the EEG, ECOG and EMG electrodes at the same time with the least weight capacity because the user will only have the electrodes, the differential amplifier, the transceiver, and the power supply as wearable electrodes for signal acquisition. All the other computing processes for the BCI will come from the electronic device 50A, an attachable BCI, online or cloud base servers. The infrared technology, with the infrared camera on the electronic device 50A for processing and decryption of gestures, with or without tactile touch points 521A on the display screen 52A, is an innovative form of operating two controlling programs with the least amount of wearable equipment.

In addition, the infrared programing with or without the hand cover 60A docs not interfere with any of the signal acquisition required by the Brain Computer Interface inputting system, the electromagnetic sensor inputting system and/or the fiber optic light source inputting system. In other words, the user can have multiple control systems at the same time with the least weight capacity for operating one or more programs at the same time in the electronic device 50A, with or without the touch points 521A on the display screen 52A and the computing processors. Furthermore, the sideline element 31A can be displayed on the projecting virtual display screen 52VA of the AR/VR/MR/XR apparatus, providing additional more organization and arrangement for multiple displays contents 212A for multitasking in 2-Dimension, in 3-Dimension or in 4-Dimension at the same time or in a timely manner. In other words, the sideline element 31A separates 2-Dimension programs from other 2-dimension programs and/or from 3-Dimension programs, and etc.

In view of above, the present invention provides a systematic interface organization for multiple control options with or without touch points 521A on the physical display screen 52A or in a non-physical virtual display medium with or without the sideline element 31A to alternate back-and-forth between display contents 212A without having to interchange control inputting systems equipment, during the process of operations of the electronic devices 50A. Every inputting device can be operated together at the same time or in a timely manner and the user can stop any inputting device during the operations of the BCI system, the fiber optic system, the infrared system, and the electromagnetic sensor system.

The electromagnetic sensor inputting system provides real time finger and hand tracking, especially for time sensitive gestures in sign language base decryption or in base controls of the touch point 521A, including (BCI) systems for the performed gesture that required X, Y, and Z coordinates for the orientation of the yaw, pitch, and roll. According to the present invention, AC solid-state type electromagnetic sensors 64A are utilized for measuring both the position and the orientation natively. This is an additional inputting system for gestures that doesn't requires to correct data due to drift.

The electromagnetic sensors 64A having various shapes and with various electromagnetic waves distinguishably programed for each phalanges including the hand(s). The wave type electromagnetic sensors 64A can work adjacent with fiber optics because fiber optics are immune and impervious to electromagnetic waves. In a combination of the fiber optic 63A and electromagnetic sensor 64, the fiber optic 63A provides the user infrared multiplexed light source intensity for allowing the infrared base control system to identify the gestures at a significant distance without the performed gesture getting lost within the environment distribution of the thermal radiation from the background of the ambient temperature, and at the same time the electromagnetic sensors 64A provide the gestures the X, Y, and Z orientation to further enhance the gesture recognition with or without the soft gel lens connection at the tip of the fingers, and at the same time provide direct and indirect thermal heat to the user finger tips. It is worth mentioning that the plastic soft gel lens 62A of the glove or sleeve are capable of activating the one or more touch screens 52A in the electronic device 50A for gesture operations and recognition by the module of analyzable factors 26A and by the artificial Intelligence module 25A for operating said AR/VR/XR/MR visual display including the display screen 52A of any electronic device 50A.

The order, position and arrangement of one or more inputting systems may work together with or without a glove or sleeve. Alternatively, all inputting systems can operate with one glove or with one sleeve, on either hand. The fiber optic element 63A and the micro light element 67 of the glove or sleeve can have various locations over the skin of the fingers and hands, including a first location distal from the distal interphalangeal (DIP) joint, a second location proximal from the distal interphalangeal (DIP) joint and a third location distal from the metacarpophalangeal (MCP) joint of each finger in relation to the human hand/finger anatomy. The fiber optics 63A equipped in the present invention are preferred to be flexible and produced as straight elongated structures providing the glove or sleeve a natural anatomical position for the finger joints. Therefore, the fiber optics 63A significantly assist with the extension of the finger joints as it delivers infrared red light from the light source 61A. Another type inputting system, with or without a glove or sleeve, in or on the glove/sleeves, may be utilized including electromagnetic sensors located above the skin bellow each finger joint, including the metacarpophalangcal (MCP) joint. The electromagnetic sensor 64A can also be positioned over the skin in the posterior region of the metacarpal bones. The one or more electromagnetic sensors 64A are in use at the same time with the X, Y, and Z orientation dimensional space in relation to the performed gestures. The electromagnetic sensor 64A may be positioned adjacent and/or proximal to the fiber optic 63A along the finger and hand. Lastly, as shown in FIG. 23, the electrode (EMG) sensors 66A are positioned most proximal to the opening of the glove also the electrode (EMG) sensors 66A may be positioned proximal to the wrist along anywhere on the volar, dorsal, radial or ulnar surface area of the wrist or forearm as the means for placement and reposition of the electrode (EMG) sensors 66A for optimum location for EMG signal acquisition on a glove or sleeve.

It is worth mentioning that the fiber optic infrared system can work alone, with or with a glove or sleeve, the signal acquisition of the EMG electrodes 66A can also work alone with or without a glove or sleeve and the electromagnetic sensors 64A can also work alone with or without glove or sleeve independently with other inputting systems and without interference. The infrared light is adapted for operating the gestures without a glove or sleeve and utilizing the natural human hand as a powerless infrared light source for infrared base inputting systems. The fiber optic elements 63A with infrared light with a glove or with a sleeve enable the infrared base inputting systems to be operated when the user is at a distance or within extreme weather/climate. In other words, too hot where the hand gestures blend with the warm environment, or too cold where the gestures are not recognized because the cold environment has produce vassal constriction in the users hand/fingers. Thereby, both the hot and the cold environment severely reduce performance of the infrared base inputting systems. The EMG signal acquisition for utilization of performed gestures or imagining gestures is optimization. The electromagnetic sensors 64A are not interfering with the fiber optic elements 63A of the infrared base system and providing the X, Y, and Z orientation for gestures operation in the non-physical (stimulated or virtual) medium such as projecting display screen 52A or with the physical display screen 52A. The material of the glove or sleeve is preferably a washable translucent thin pores mesh stocking like, elastic fibers or rubber material hypoallergenic with a second glove or sleeve cover for the cold weather. The inputting systems of the present invention can be operated together or independently with a glove or a sleeve on the display screen 52A and/or for operating the AR, VR, MR, XR apparatuses.

Furthermore, a haptic system can be incorporated into the above mentioned inputting systems. The haptic system alone is inheriting and limiting in relation to work-flow and, as the sensory components, further improving in the haptic system, it will become more limiting. For example, imagining a haptic sensory for a stabbing pain, the user will be overcome with that sensation of pain and that will render the user powerless unable to concentrate on anything else. The haptic system without interoperability for multitasking does not improve the user's physical environment for work, academically, professional, or in the labor force. The haptic sensory is more for specific moments, for the entertainment, and not for day to day work. The electronic device 50A with interoperability and multiple interface control systems will never lose their value, like the display screen 52A in the present invention with multiple control options for continuous work may surpass any future system with a single inputting system.

Furthermore, continuous repetitive actions prolong without tactile counter surface become a source of fatigue and may develop an injury. Tactile counter surface provides sensory, proprioception and sensory stimulation. Lack of sensory stimulation may lead to a reduction of endorphins that is not health for prolonged wellness. Haptic inventions without tactile counter pressure surface area are not suitable for prolong utilization, and contribution to the reduction of quality of life by decreasing ergonomics optimization. Diminish sensory proprioception will decrease environmental sensory awareness and increase the possibilities for accidental injuries (slip and falls). According to the preferred embodiment of the present invention not one single type of inputting system will provide the complete operation product for prolong day to day work force, and that the system mimics the human body in relation to alternating different inputting systems to produce the desire physical activity. For example, the human body is masterful in activating different muscles for producing the similar physical task, naturally (muscle memory), subconsciously or consciously, the human body will activate multiple different muscle groups to perform the similar task in the event the user is injured, fatigue, or physical limitations. In the same way, the organization and arrangement for more than one inputting system to operate the electronic device 50A for prolong utilization in a robust, convenient, customizable and flexible approach to complete the desire task.

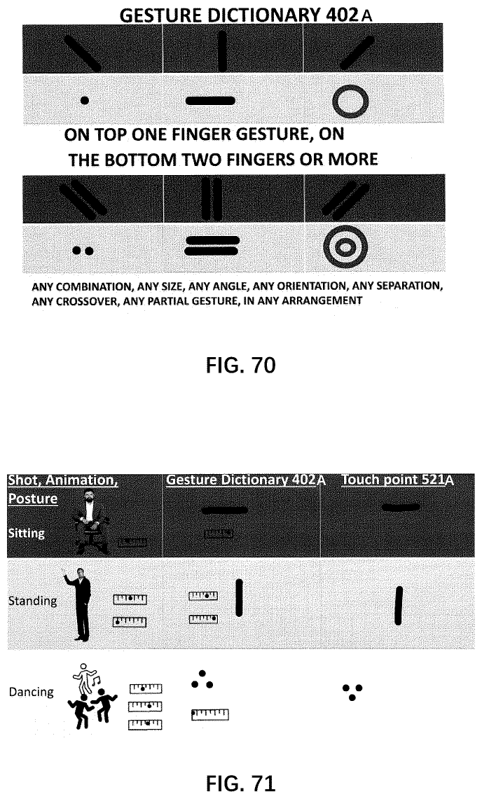

As shown in FIG. 70, various gestures type and variants available to the user in the gesture dictionary 402A is illustrated as example, wherein the user has various options for creating gestures in the gesture dictionary 402, with one or more touch points (fingers), where the fingers generate a traceable outline analyzed by the module of analyzable factors 26A for any combination, any size, any angle, any orientation, any separation, any crossover, any arrangement, any length, and any partial performance of the above mention touch point characteristics for any gesture. It is not limited to one hand, one control system or one display screen 52A. The user may perform a gesture selected from the gesture dictionary 402A to interface with the interactive module 40A and pair-up with the multiple control programs available in the electronic devices 50A for controlling and operating on 2-Dimension, 3-Dimension or 4-dimension, such as the infrared base control programs like the signal acquisition base Brain Computer Interface programs or through the display screen 52A. A touch point gesture is the gesture to be performed would produce one or more touch points 521A. Certainly, the gesture can be any gesture combination, with any gesture separation, of any gesture size, any gesture angle, any gesture orientation, any gesture crossover, any gesture arrangement. The program of the gesture dictionary 402A provides a running list of all the programs paired with each specific gesture while the artificial intelligence module 25A informs the user of any repeated gestures for the electronic device, the Brain Computer Interface, and/or the infrared base control. For example, the user may have a gesture for the Home Screen or for displaying multiple programs from which he or she can select. According to the preferred embodiment of the present invention, a paired gesture can be provided for scaling the number of gesture variants for efficiency. Additionally, the BCI system along with the artificial intelligence module 25A are configured to learn the signals from the fiber optic system, the hand/finger gesture software detection program, from the electromagnetic sensor system for improvement recognition and from the gestures independently or in combination for operating on the one or more electronic devices 50A.

Referring to FIGS. 71-74, most electronic devices 50A are incorporated with verbal control systems, wherein the audio is an existing option for engaging with the user. The user may also designate numbers, word and/or phrase when speaking to input verbal commands for selected programs. Pairing a touch point gesture with a verbal command program 404A is another form of two digital existing programs interoperability for controlling and operating the one or more electronic devices 50A. When the selected gesture is paired with other programs in the verbal command program 404, the user can verbalize, perform, and/or imagine a gesture to generate the desire response, at the desire location. When the gesture is paired with the verbal command program 404, the gesture continues to be analyzed by the module of analyzable factors 26A and by the artificial intelligence module 25A for operating the gesture in the same process according to the present invention. The user's words may or may not be transmitted or be recorded, wherein the spoken words may operate independently or collectively with the performed or imagining gesture to produce a response. Furthermore, the ear pieces and/or headsets may also interface with the display screen 52A for the delivery of additional inputting information in the one or more electronic devices 50A. The customization, arrangement and organization of gestures pairing with the verbal command program 404A are interchangeable. The verbal command program 404A may have different customizable touch point gestures to be paired with different voice command in the verbal command program 404A, and may be performed like any of the verbal command program like “SIRI”.

For example, as shown in FIG. 48A, when the user makes contact with the touch screen type display screen 52A to produce the one or more touch points 521A on the display screen 52A, the display screen control module 20A acquires the information, the module of analyzable factors 26A and the artificial intelligence module 25A provide the information which is received in the adjuster module 10A, the interactive module 40A and the sideline module 30A, wherein the gesture is recognized by the artificial intelligence module 25A as a voice dependent gesture and the one or more touch points 521A produced by the gesture are localized and require the verbal command program 404A to generate a final response. For example, the user speaks out the word “adjuster” or a number, the verbal command is recognized by the artificial intelligence module 25A and the information is provided to the server processor 101A and/or the processor 51A in the electronic device 50A. Thereby, the verbal response is generated accordingly to the verbal command of the adjuster 13A at the location of the touch point 521A. It is worth mentioning that the same process or similar process can be accomplished for other programs for operation of the sideline element 31A and the numeric indicator 16A, the keyboard element program 43A, the state-of program 48A, and etc. The electronic device 52A may also have voice recording or voice command programs like “Siri”, wherein the user can make the command for “adjuster” and the programs response operability to the user's voice command in relation to the touch point 52A at the location on the display screen 52A for any program.

FIG. 71 illustrates an animation posture list program, wherein the first vertical column shows the selected shots, frame, clip, or skit, (animation posture) and the middle column shows the selected gestures from the gesture dictionary 402, while both columns contain adjuster interface 21A for customization. In the last column, the user performs the paired gesture and is recorded and learned by the artificial intelligence module 25A. It is important to mention that the columns are separated by the sideline element and/or a window program for the selected program function, as illustrated on FIGS. 27-30A.

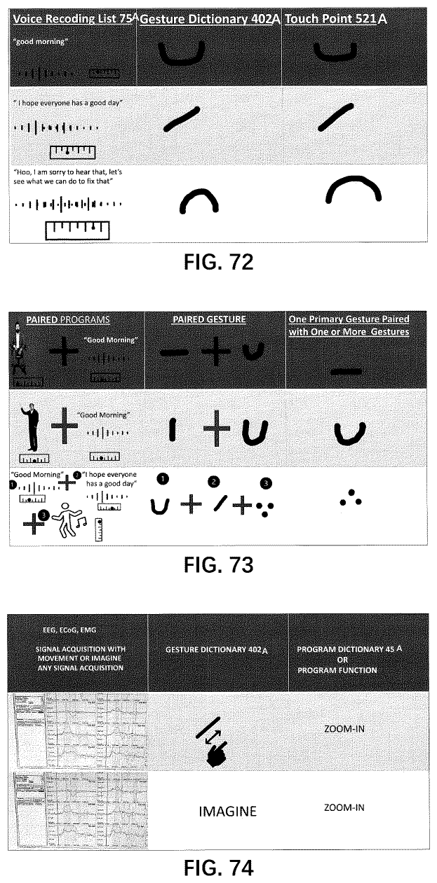

FIG. 72 illustrates a voice recording list program, wherein the first vertical column shows the selected word, sentence or phrase (voice recording), the middle column shows the selected gestures from the gesture dictionary 402A both columns contained adjuster interface for customization, and the last column shows the user performs the paired gesture and is recorded and learned by the artificial intelligence module 25A.

FIG. 73 illustrates a paired program list where the first vertical column shows the selected shots, frame, clip, or skit, (animation posture) and the word, sentence or phrase (voice recording) and the middle column shows the two or more selected gestures from the gesture dictionary 402A for their corresponding function, while both columns contained adjuster interface for customization. In the last column, the user performs the primary gesture and is recorded and learned by the artificial intelligence module 25A.

FIG. 74 illustrates inputting options for gesture pairing with their corresponding sign acquisition when performed or imagined to generate a selected response. The first column represents the signal acquisition the type (EMG, EEG, EcoG). The second column is the signal acquisition paired with the performed gesture or an imagine gesture. The third column is the type of program from the program dictionary, paired with the selected performed or imagining gesture. Notice a program function can have performed and/or an imagining gestures at the same time with the same signal acquisition to produce the same program or program function, this is an example of a robust security system for that program or program function.

Referring to FIG. 71, an animation posture list is illustrated, wherein the user can also pair a performed or imagining gesture with other verbal command programs for controlling an animation posture or animation functions from the animation posture list. In other words, when the selected verbal command, the performed gesture or the imagining gesture are paired with a program in the animation posture list 76, the user through the vocalization, imagining or performed gesture generates the selected customized animation, shot, frame, clip or skit on one or more display screens 52A, wherein the artificial intelligence module 25A recognizes the verbal commands, the performed gesture or the imagining gesture in on one or more display screens 52A, on one or more display projection mediums, in any electronic device 50A.

Referring to FIG. 72 a voice recording list 75A is illustrated, wherein the operators' words are pre-recorded to become audible when a specific gesture is performed. In other words when a gesture is paired with a pre-recorded word, phrase, sentence, statement or story, the pre-recording becomes audible to any party selected for communicating. Also, a performed or imagining gesture may deliver during voice recording during, before or after, any operating program, wherein the recorded words may also be overwritten by the operator through the use of the performed or imagining gesture, like when using a (walkie talkie). In other words, the operator performs the gesture to speak, as a speaker control, like the handheld walkie-talkie. When the selected gesture is performed by the user and the user's voice is transmitted to other individuals, avatars, surrogates, in 4-dimension, in 3-dimension or in 2-dimension of the electronic device 50A in real time or in a timely manner.

Referring to FIG. 73, the paring gesture program is illustrated, wherein individual gestures are arranged in a left to right order with or without verbalization from the verbal commands program 404A. The arrangement of two or more performed or imagining gestures are organize, like Morse code in a telegram, wherein different customizable performed or imagining gestures are joined with multiple other different gestures, wherein the gestures are performed or imagined and the responses of each gesture are generated in the chronological order left to right according to the order of the gestures, wherein the gestures may come from the animation posture list and/or from the voice recording list 75. In the paring gesture program, the user is provided with the tools to join one or more touch point gestures from the program of the animation posture list from the voice recording list 75. It is worth mentioning that the gestures from the voice recording list 75A and the gestures from the animation posture list have been pre-programed and paired with a program corresponding the program function for each individual program, before any of the selected gestures can be joined in the pairing gesture program. Furthermore, in the pairing gesture program, gestures from the animation posture 76 and from the voice recording list 75A can be rearranged, interchangeable, added or removed. In other words, the gestures are customized to deliver the desire animation from the animation posture list 76 with or without verbalization from the voice recording list 75, in a pre-selected order, as illustrated in FIG. 73. It is important to mention that the goal of the paring gesture program 78 is to reduce demanding physical activities. Therefore, the user does not need to performing or imaging every gesture from the voice recording list 75A or from the animation posture list, in the order most desire. Once the two or more gestures are organized in the sequence pre-selected, the user can designate a gesture as the primary gesture. When the user performs or imagines the primary gesture, the entire chain of gestures from the voice recording list 75A or from the animation posture list are generated according to the pre-selected order in the paring gesture program, thereby creating a customized animation, shot, frame, clip or skit with verbalization or without verbalization or vice versa.

FIGS. 71-73 illustrate various gestures pairing with designated functions/programs, wherein the voice recording list 75A is for the pre-recording of various audio, text and or emojis with various lengths, interchangeability, customization from the gesture dictionary 402. The animation posture list is for the pairing of one shot, frame or animation program with one or more gestures from the gesture dictionary 402. The pairing of gestures with selected functions takes place in a pairing gesture program, wherein two or more gestures can have more than one function/program organizations. The adjuster 13A with the adjuster interface 21A provide the timing, volume, brightness and all the scalable controls for the editing function/programs in the final display, and etc., wherein the gesture responses are generated at the same time or in a timely manner, in the order the user produces two or more gestures for the selected function/program paired with the order of the selected gesture in the pairing gesture program.

Further, one or more gestures can be designated as a primary gesture even if the gestures are incorporated within the program of the voice recording list 75A or the program of the animation posture list in any particular order within the pairing gesture program. The one or more paired gestures can be selected as the primary gesture to eliminate the task of producing multiple gestures and to eliminate having to remember the sequence of gestures for a created customized animation, shot, frame, clip or skit with verbalization or without verbalization or vice versa. Also, the primary gesture can be a verbalize, number, symbol or another gesture not replicated. The module of analyzable factors 26A and the artificial intelligence module 25A can safeguard the user from duplicated gestures and notify the user of replications. For example, a half circular/parabola 2-Dimensional (U shape) gesture is produced on the display screen 52A and the program of the voice recording list 75A recognizes the gesture. For, example, a U-shape gesture is paired with the voice recording list 75A to transmit “good morning” and/or text “good morning”, as a preprogram voice recording or text greeting. In an alternative example, a long body shot or frame is displayed on the display screen 52A when the user produces a vertical gesture where an animation posture list recognizes the gesture. FIG. 73 illustrates the pairing of two or more gestures taking place as an example while FIG. 72 illustrates the voice recording list and FIG. 71 illustrates the animation posture list. The user may select one or more gestures from the voice recording list 75A and/or from the animation posture list. Then, the user arranges the gestures in a desired order to display the functions/programs accordingly to the order selected with the gestures. The gestures in the gestures dictionary 402A can be edited in both the animation posture list and the voice recording list 75A as shown in FIG. 72 and in the voice recording list 75A as shown in FIG. 71 with the adjuster 13A for selected additional functions to create the final display with audio and animation.

An alternative example for pairing the gestures is that the user must first open the page to the pairing gesture program where past gesture combinations are provided with their corresponding programs and are provided for reference and for editing the frame, audio, shot or animation. The user may also open-up multiple programs, like the program of the voice recording list 75A or the program of the animation posture list through the sideline element 31A, at the same time or in an orderly manner divided by the sideline element 31A. Where each gesture is paired with a specific function according to each program in the program of the animation posture list 76 or in the program of the voice recording list 75. Then after the user completes the selection for the desire program function the user selects the gestures to use from the voice recording list 75A and/or the animation posture list, the desired functions may be reviewed and edited with the adjuster 13A for every animation and audio, timing speed, volume, and etc. The final display program contains the expression and verbal tones to display in the 2-Dimensions, 3-Dimension and 4-Dimensional digital environment, wherein the full displays are reflections of the user's own expression and style of greeting, or any other customized animation or action frame as desired, as illustrated in FIG. 73. The electronic device 50A can be independently operated or utilize in-combination with other programs, for example the verbal commands programs 404, motion controls and/or neurological electrical impulses with the Brain Computer Interface (BDI), hand/finger gesture software detection program or any other non-interfering inputting system, in a scalable manner, etc.

The customized shot, clip, frame and or animation may also be paired with selected gesture. The vocal auditory function for the user to select specific pre-recorded verbal phrase, line, statements, linguistics, such as “good morning”, “I hope everyone has a good day”, or “welcome everyone and good morning” preset in the gesture shot, clip, scrip and digital video system program are paired with each individual gesture designation. Furthermore, one primary gesture can be designated as the primary gesture and the programming will provide vocal auditory function for the user to select specific pre-recorded verbal phrase, line, statements, linguistics, such as “good morning”, “I hope everyone has a good day”, or “welcome everyone and good morning”, and etc., wherein both the voice recording list 75A and the posture animation list are scalable.

Furthermore, the same type of gesture pairing can be applied to text communication, wherein preselected text phrases or words are paired with one or more gestures to be transmitted to other individuals, avatars, surrogates, in 4-dimension, 3-dimension or 2-dimension electronic device 50A when the user performs the desire gesture for the desired wording. Additionally, vocalized words can be converted into a text format and transmitted to other individuals, avatars, surrogates, in 4-dimension, 3-dimension or 2-dimension electronic device 50A.

Referring to FIG. 72, an example of portion of the gesture dictionary 402A is illustrated, wherein the operating gestures can be predesignated from the gesture dictionary 402A of the system such that the gestures are recognized by the artificial intelligence module 25A and the information is sent to the interactive module 40A, wherein the interactive module 40A displays the pairing gesture program and all its functions. In addition, each operating gesture can be predesignated to any customized gesture within the system and/or customizable by the owner of the electronic device 50A, so as to facilitate the operation and control of the one or more digital programs. The prerecording, editing, interruption and cutting of a 2-Dimensional, 3-Dimensions and or 4-Dimensions digital display with or without verbal response provide the user with the control over the visual and audio elements for a customizing a shot, frame, and or audio. Gestures provided the users an innovative way to interact within the system and/or any electronic device 50A, as the means to operate within the digital simulation environment.

An arrangement of operating gestures is provided for controlling the moments and verbal functions of simulated characters, without the user doing the body movements, or saying the verbal functions. In other words, the gestures are controlling the display and the audio. Gestures provide the user the ability to increase productivity, endurance, to reduce physical demand, and vocal demands on the operator. For example, corporations may train their customers and employees to operate gestures when communicating in virtual reality (VR). Therefore, the consumers will get the same visual experience and the same auditory experience with a live customer employee in virtual reality (VR), wherein the employee dose not need to vocalize most of the words in the communication nor does he or she need to perform all the displayed body postures or body language during the communication.

In the digital environment both engaging display and the response display are important for the complete communication. For example, when a user is greeting someone, the user can select and perform a pre-recorded digital posture shot for greeting someone in the digital environment or someone else in another digital program in one or more electronic devices 50A, wherein the greeting shot is composed of a digital program action animation, from start to finish with or without audio elements. This will constitute an engaging display. Alternatively, a pre-recorded digital response program may also be intertwined with digital posture response with or without audio element in the shot used for secondary responses. For example, “Thank you for asking” or “I am great can you please help me” represents a response display. Now, considering in both the engaging display or in the response display, the user can select the framing for the action with a long shot, a medium shot, a close-up shot, or an extreme close-up shot through the gesture, the adjuster interface 21A, the adjuster 13A, or a verbal command program.

It should be noted that 2-Dimensions, 3-Dimensions, and 4-Dimensional motions and movements can be controlled with 2-Dimensional gesture interface controls, wherein the user can have various selections of body movement, facial expression, and audio including accent, tone and articulation, with visual display. Considering a user will have the option and capacity to interact in a simulated digital environment with the least effort, through the 2-Dimensional display screen 52A with the operation with one or more touch points 521A, the user can select a gestures and perform a combination of gestures with a complete pattern of sequential gesture with the adjuster interface 21A to interact with other characters, avatars, emojis, and more important to control personalized avatar, surrogate, characters, emoji, and any like digital animation. FIGS. 70 to 72 illustrate the same process of pairing gesture as with programs, wherein the user can have scalable cut and paste animation shots, clips, and or audio list for controlling any programs like, avatar and or surrogate programs and or functions. Furthermore, the gestures, for any programs, are interchangeable.

The gesture with the adjuster 13A will provide the users the frame and the angels of the frame the digital visual display and responding display as they are intended to be seen and heard by others. The gesture generates the frame selected while the adjuster 13A and the adjuster interface 21A control the frame angle. For example, eye level angle (more personal equal) to other avatar or surrogate eye level with others digital displays, high angle to show smaller from the other avatar or surrogate as a sign of (weakness), and low angle to show over other avatar or surrogate as a sing of (strength from above). In conjunction, the gestures will provide high key lighting (open effect) or low key lighting (suspicion effect). Preferably, all the optional programs and function mentioned may be generated by a gesture and/or a verbal common that can link with an adjuster interface 21A through the adjuster 13A, wherein the user can control any type of frame like, a long shot, a medium shot, a close-up shot, and etc., or the level of the angle of the frame eye level angle, high angle or low angle, including the degree of lighting from low key lighting to high key lighting and any degree in between, with verbal commands.

For example, when a user is attending a virtual reality (VR) shop, simulated interactive programs are recorded through the usage of wearables sensory devices and the user is able to produce various facial and body language interaction in the simulated environment, wherein the recordings are sale to the user and the user can cut and paste them onto his or her personal electronic device 50A. For example, the hand posture, the body language, the dancing style, the clothings and the facial expressions are interchangeable while the programs provide vocal verbal function for the user to select a specific pre-recorded audio, such as “good morning”, “I hope everyone has a good day”, “welcome everyone and good morning”, or etc. The selected gesture is paired with the shots, frame and/or animation in the posture animation list, and that gesture is paired with another gesture from the voice recording list 75, to give the animation frame audio. Therefore, the user pairs two or more gestures based on the function while the gestures are paired with in relation to the function the gestures are paired with.

FIGS. 71-72 illustrate the gesture pairing, the (U/parabola) shape gesture corresponding with the “good morning” recording in the voice recording list 75. Additionally, the horizontal shape gesture is paired with the sitting posture/animation in the animation posture list. The user may pair two or more gestures together in a customizable order with a selected arrangement in the pairing gestures program, as shown in FIG. 72. Furthermore, the adjuster 13A and the adjuster interface 21A provide the user the tools to edit the animation speed, timing, audio, and etc. The user may also control other digital function at the same time by vocalizing the command through the verbal command program 404A.

FIGS. 65A to 73 illustrate the complexity of touch point gestures, verbal command, EMG, BCI, head motion controls, and retinal control system working together or independently. Furthermore, the sideline module 30A is configured to be operable with the operation systems of Windows and Apple for separating programs and program functions, such as providing the sideline element 31A for dividing two or more programs and providing organization and arrangement with all kinds of inputting controls. For example, at any location where the user touches the display screen 52A, the display screen controller module 20A (the second executive portion 2A) receives the information the touch point 521A is analyzed by the module analyzable factors 26A and the artificial intelligence module 25A. Then, the information is sent to the processor 51A and the communication module 55A is configured to send the information to the first, to the third, and to the fourth executive portions 1A, 3, 4, wherein the gesture is recognized by the interactive module 40A as a paired verbal command program and the touch point 521A is recognized by the third executive portion 5 which is activated for generating the sideline element 31A. Alternatively, once the user says “sideline” the adjuster module 10A is activated to display a sideline element 31A while the gesture has been paired with the verbal command program 404A. Furthermore, other multiple inputting control programs can be paired the same way. In one example, a motion head control program can be paired with a gesture. In another example, the user may produce a gesture for the sideline element 31A and the user may rotatably move a head of the sideline element 31A to a desired location so as for relocating of the sideline element 31A with or without the adjuster 13A. In addition, the verbal command programs 404A can be configured to control the directions or locations of the sideline element 31A or any other program.

For example, the user may produce the touch point gesture for the posture display of the avatar or surrogate and then the user speaks a verbal command, such as “eye level”, selected from the gesture dictionary 402A to activate the verbal command program 404A. Accordingly, the electronic device 50A generates an eye level digital display among other avatar and or surrogates in the electronic device 50A. Alternatively, the control of the frame can also be operated in relation to the location of the headset 50A″, wherein the headset angle is leaning forward or backward. The furthermost forward lean with the headset 50A″ generates the extreme close-up and the furthermost backward lean with the headset 50A″ generates the long shot, wherein multiple interchangeable gestures are paired and operated with other independent or codependent programs like the verbal commands programs 404A for independently turning on the volume of the contents on the display screen 52A or in combination with where the user wants to display a close shot on the display screen 52A. In other words, the verbal commands programs 404A and other programs can oscillate with the user's gesture requests and verbal requests in real time.

Alternatively, referencing FIGS. 65A and 65B, all the optional programs and functions can be generated by a gesture, a verbal comment and a headset 50A″ with a motion controller, where the user may produce a gesture or a verbal command for the visual display shot, frame, clip, or animation of an avatar or surrogate. The user can control any type of frame, such as a long shot, a medium shot, a close-up shot, an extreme close up, and etc., or the level of the angle of the frame eye level angle, high angle or low angle, including the degree of lighting from low key lighting to the high key lighting and any degree in between. When the user gets closer to the display screen 52A with the virtual reality devices headset 50A″, the virtual reality headset 50A″ senses the change in motion and displays the frame program for the location of the headset 50A″ correspondingly. For example, if the user moves the headset 50A″ away from the display screen 52A, the long shot frame is generated, and when the headset 50A″ moves closer to the electronic device 52A the extreme close up shot is generated. The headset motion control is interfaced with the electronic device 50A. Also, the bending of the neck with the headset 50A″ operates the lighting, for example bending the neck to the left can generate a reduced low key lighting, bending the neck to the right can generate a high key lighting, and positioning the headset 50A″ in neutral can generate a neutral white balance. It is worth further mentioning that, according to the present invention, the user may select the programs to be operated with the headset 50A″.

The customer service industry would appreciate the gesture organization and operations for verbal customization and posture display according to the present invention because corporations will have the full control of their company images and promoting their brand while providing a live operator. The employees preselect the verbal and postural options during communications with the customers in the Virtual Reality environment. The user is able to speak independently from the pre-program gestures and voice gesture controls, wherein a selected gesture can provide the user the physical control at any time for any type of preprogram verbal response or verbal recording and therefore the user is able to personally vocalize at will. In other words, the user may project a natural flow during the oral communication process without the customer noticing any changes in the flow of the communication, even in the most complex dialogues. Employee's posture display is as important to companies as verbal communication is in customer service that body language and posture displace can be controlled in the Virtual Reality through the use of gestures by the user. For example, a straight vertical gesture can be paired with a shot, a clip or a frame for standing up or for stay up. Likewise, a horizontal gesture can be used for sitting posture shot, clip or frame. Additionally, the posture programs with the verbal recordings can be copyrighted for employees in a specific industry where the brand name and logo are not allowed to copy in the simulated world and are specific to a company brand. The employees operating the avatar and or surrogate will have preselected posture and verbal displays from the employer to ensure the company's name brand is not tarnished, with poor vocalization and or appearances by the customer service employees in the 2-Dimension, 3-Dimension and or 4-Dimension environment.

The user is not discouraged from utilizing wearable sensor electrodes throughout his or her body for interfacing with the electronic devices 50A, with a partial or complete bodysuit. In contrast, the present invention provides a more convenient approach, ergonomically for communicating, socialization and operating in the virtual world. According to the preferred embodiment of the present invention, the user can operate the performed or imagine gestures for controlling, with or without utilizing sensor wearables throughout an event. Current technology requires a bodysuit with multiple sensors and multiple cameras, this can be physical demanding from the extra weight from the bodysuit sensors, and from the require motion to controls the program with the cameras. For example, a user in a virtual world simulation of a zoo with a complete bodysuit sensor wearable is physically required to perform walking in order for the program to continuously display all the digital animals available for display. If the operator stops moving, the program will not understand he wants to keep seeing the animal display in the virtual reality without all the body motions. In other words, if the operator stops, the digital display becomes focused on providing the animation for the location where he stopped at. The operator is forced to maintain the physical walking activity in order to complete the observation of all the animals in the virtual reality zoo. Considering this virtual reality programs, they are not practical nor convenient for day-to-day socialization, communication or work.

In view of above, the present invention substantially allows operating the movements and postures displays with gestures and verbal controls, wherein the gestures, the verbal controls and the animation controls can be paired with each other in the pairing gesture program. Where the animation posture programs, the gestures from the gesture dictionary 402A and the voice recording list 75A are scalable. Additionally, the user can perform a touch point 521A gesture to speak live whenever he or she wants, thereby providing the user the full control of communicating in a natural flow.

While the ability to multitask in Brain Computer Interfaces devices is currently not an option with the Brain Computer Interface alone, the present invention provides multitasking with sidelines (including the sideline element 31A, the reservation sideline element 32A, and the pop-up sideline elements 37A), verbal commands, neurological electrical impulses signals, electromagnetic sensors, infrared control, and gestures performed or imagined on one or more display screens 52A of one or more electronic devices 50A with or without the use of processor 51A in the electronic device, through the internet or through cloud servers 100A. Furthermore, the present invention provides the arrangement and organizational process when linked with other technologies like eyeball motion control in the Virtual Reality, in the Augmented Reality, in the Mix Reality and in the Extended Reality through the usage of all kinds of headset 50A″ such as googles or waveguide glasses devices. The present invention further provides interoperability for operating electronic devices 50A with current and future inputting systems, without deviating from the current technology.

For example, preferably, all the operational options in the sideline module 30A are able to be operated through the BCI with the EEG/EcoG and the electronic device 50A combination, or the BCI with the EMG and the electronic device 50A combination, or through both mention combinations at the same time or in a timely manner as a robust security system and to increase precision control.

In addition, the artificial intelligence module 25A of the present invention is arranged to recognize the operational gestures as they relate to the 2-Dimensional programs, 3-Dimensional and or 4-Dimensional program in the electronic devices 50A. Furthermore, the user does not have a need to alternate back and forth with bulky wearables sensors to operate a 2-Dimensional program while at the same time or in a timely manner operating 3-Dimensional programs divided or not divided by the sideline element 31A. In other words, the same 2-Dimensional gestures in the 2-Dimensional program or in the 4-Dimensional program can have similar functions, or can have totally different functions, the artificial intelligence module 25A is configured to anticipate and the module of analyzable factors 26A in the electronic device 50A is configured to analyze the performance and recognize the computer language and/or the digital operations.

Infrared control system and hand finger software detection programs on the physical type display screen 52A of the electronic device 50A and/or on virtual type display screen 52A or display medium of the AR/MR/VR/XR apparatus may work together or independent as the means to further analyze the gestures being performed by the user and to analyze gestures with or without a physical touch surface. For example, the infrared control system on the VR/MR/AR/XR apparatus can be activated with a gesture and turn-off with a different gesture, providing ongoing gesture pairing programming together or independent of the electronic device 50A, with or without the Brain Computer Interface system and the electromagnetic sensor orientation, as mentioned above.

According to the present invention, the one or more electronic devices 50A can be controlled and operated with different gestures that are paired with selected programs or program functions. The Brain Computer Interface/Brain Machine Interface signal acquisition can be accomplished by performed gestures or imagining gestures paired with corresponding program or program function. Also, the retinal motion control, the infrared control system and the verbal command system are paired with corresponding program or program function. Each inputting system can have independent gestures paired with different programs or program functions. For example, a vertical gesture can be paired with a program or program function within the 2-Dimension program of the display screen 52A and the same vertical gesture may generate a different response in the 3-Dimension program divided by the sideline element 31A. Furthermore, the same gesture can be recognized with a paired program in the Brain Computer Interface system of the EMG or EEG/ECOG and that the same gesture may not be recognized in the inputting system of the electromagnetic sensors 64A or in the infrared base inputting system for that particular program. The artificial intelligence module recognizes when an inputting system is intentionally program for a specific response in a specific program and/or not in every available program.

The artificial intelligence module 25A may not recognize gestures from none functioning inputting system performed or imagined. In other words, the pairing of programs or programs functions with a gesture or any other inputting system can have redundant inputting options or not. For example, the Brain Computer Interface may not be programed with the gesture of vertical touch point 521A. A physically performed gesture or an imagining gesture may be paired with a different program in a different inputting system. In other words, the display screen 52A of the electronic device 50A is working together with the Brain Computer Interface, where the touch points gestures can have their own pairing with selected programs or program functions and, at the same time, any signal acquisition may also be paired with selected programs during physically performed or imagined. Also, not all programs need to be paired with the touch point gestures. In other words, the gesture is performed on the display screen 52A but not due to the touch point 521A on the display screen 52A, the response is produced on the display screen 52A because of the imagined or performed gesture sending a signal acquisition from the EEG, EcoG, and or EMG, independent or together with the infrared camera system, or from the hand finger software detection program within the display screen 52A of the electronic device 50A.

Alternatively, multiple inputting systems can recognize and verify the imagined or performed gesture to generate a response. In addition, each inputting system can have dedicated gestures paired with a selected program or program function, in each independent inputting system. The more inputting systems are merged the more programs or program functions that can be paired with the specific gestures. The more inputting systems are used, the more programs pairing capacity increases the number of programs that each gesture can be paired with, so as for allowing multitasking capacity and for multiple operations at the same time or in a timely manner. In an alternative example, the same vertical gesture is recognized by the Brain Computer Interface to produce a different response when the signal acquisition is produced by the performed gesture or by the imagining gesture. In another example, the same vertical gesture is recognized by the Brain Computer Interface to produce a different response when the signal acquisition comes from the EMG vs the EEG or the ECoG electrodes.

When the display of a selected program or program function is paired with an imagining or performed gesture, the module of analyzable factors 26A and the artificial intelligence module 25A may recognize the performed touch point 521A on the display screen 52A, wherein the gesture (signal acquisition) information may or may not be provided to the artificial intelligence module 25A in the Brain Computer Interface (BCI) of the electronic device 50A. Alternatively, the recognition of the artificial intelligence module 25A of the gestures may come from an attachable Brain Computer Interface device or from a built-in Brain Computer Interface system within the interface of the cloud servers with the electronic device 50A. Alternatively, any inputting operation may require a touch point 521A and a signal acquisition from the (EEG, ECOG, EMG) or any other inputting system to generate a response. Alternatively, when the signal acquisition is the only desire inputting system for pairing with a selected program or program function, there may be other inputting systems with the same gesture paired with the same program or program function being turned off. Furthermore, the infrared system and hand finger software detection program can be turned on or off selectively to recognize gestures and to provide an additional pairing option for programs and program functions. For example, the same vertical gesture can perform two or more different functions at the same time when inputting with two or more imputing devices. Alternatively, the same vertical gesture can be paired with two or more different functions, for example, the first one from touch point gesture, a second one from the Brain Computer Interface when the gesture being performed or imagined, a third one from the infrared control system sign language, a fourth one from the electromagnetic sensor 64, and a fifth from the hand finger software detection program in the physical computer, in the (AR/MR/XR/VR), or in a projection medium.

FIG. 74 illustrates the availability for one program to have more than one way of inputting gesture to generate the same response. Likewise, a gesture can be programed with two different programs, one imagining gesture and the other performed gesture. Vice versa, any signal acquisition can be paired with one or more programs according to the gesture and the type of gesture inputting system. Additionally, other imputing system without a touch point 521A (gesture) can be paired together with any of the touch point inputting systems described in the invention, for a more robust security system. For example, a verbal command program 404A (non-gesture inputting system) can be paired with a gesture base inputting system to be performed at the same time or in a timely manner. It is worth mentioning that the infrared base camera can be gesture dependent, the display screed 52A can be gesture dependent, the hand/finger software program can be gesture dependent, the (AR/MR/XR/VR) screens 52VA can be gesture dependent, the glove and sleeve 60 apparatus can be gesture dependent, the electromagnetic sensor 64A can be gesture dependent, the transparent optic soft gel lens 62A can be gesture dependent, the fiber optic element 63A can be gesture dependent, the micro light element 67 can be gesture dependent, the (EEG, ECOG, EMG) signal acquisition performed or imagined can be gesture dependent, the Brain Computer Interface can preferably be gesture dependent, the electronic device 50A can be gesture dependent, and the display medium light projection non-physical holograms can be gesture dependent. In other words, any gesture imputing system mentioned in the invention can be paired with each and other (non-gesture inputting systems) for a more robust security system and scalable interoperability.

The artificial intelligence module 25A is arranged to recognize the current operation in 2-Dimension, 3-Dimension or 4-Dimension. The gestures may have the same pairing for the selected programs or program functions in each dimension. Alternatively, the gestures may have different gestures pairing for selected programs and programs functions in each selected dimension. Furthermore, each inputting system may or may not have the same gesture pairing for selected programs and program functions.

It is worth mentioning that 4-Dimensional (time and space) like tesseract will become more uncomplicated concepts in the digital environment, just like time playing a role in our physical world. Time in the 4-Dimensional programs will have a bigger roll in the simulated world. Through the simulated world, students, researchers and the general public may have access to better conceptualize the 4-Dimension and other higher dimensions. The simulated world will provide humans the digital display to further conceptualize other higher dimension and therefore give us a better understanding of our own physical world as it relates to higher dimensions. In the 4-dimension, the user will be able to operate non-touch physical controls, wherein the gestures, sign language and verbal commands are imagined or performed and are time dependent or not, just like in the 2-Dimension imputing controls.

For interfacing operability with multiple inputting device software and hardware, the user can operate within the simulated environment and, for example, produce a shot, frame, and/or animation. In other words, the user can cut and paste characteristic, linguistics previously performed animation and customize said frames and where the users' activities are copied, transferred or deleted by the user. For example, the user can edit a smile of the avatar and make personal customization changes to his or her own personal avatar, within set regulations.

Reactive programming is a design paradigm that relies on asynchronous programming logic to handle real-time immediate updates to otherwise static content. It is possible and will increase user imputing device options and gestures for efficiency as it relates to the interoperability of multitasking for any of the corresponding embodiments. Furthermore, responsive programming can be operated for any of the selected programs with more than one gesture, with one or more imputing devices. It is noted that both programming options are ideal for improving the work flow and not one system can replace the other.

According to the present invention, the Big Data is not limited to the applications of the reactive programing, the responsive programming, the first executable portion 1A, the second executable portion 2A, the third executable portion 3A, and the fourth executable portion 4A with processors (internet) or without processor (cloud base computing) to enable organization in developing, deploying, operating and managing the Big Data infrastructure.

In view of the method and system for displaying sideline operation as described above and shown in FIGS. 45-74, the gesture operating is preformed by hand and the multi-task hand support device as illustrated in FIGS. 1 to 44 is preferred to be equipped for providing assistance with repetitive finger or wrist movement and other activating of hands and/or wrists during typing and gestures for inputting and operating of the programs, while providing various therapeutic and medicinal treatments.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A hand support device for an input apparatus for system for gesture operation of one or more electronic devices, comprising
a hand supporting bridge unit comprising at less one elongated bar; and
a support base assembly comprising a first base unit and a second base unit adapted for arranging at a first side and a second side of the input apparatus such as typing apparatus like keyboard, wherein said hand supporting bridge unit bridges across said first unit and said second base unit adapted for supporting wrists/forearms of an user of the electronic device while using the input apparatus such that hands of the user are able to hang down from the wrists supported by said hand supporting bridge unit to reach the input apparatus while providing a transversal support for upper extremities of the user over the input apparatus in wrist support typing posture.

2. The hand support device, as recited in claim 1, wherein said hand supporting bridge unit comprising at least one elongated bar which is supported by said first base unit and said second base unit over the input apparatus in a horizontal manner for supporting wrists/forearms of the user of the electronic device while using the input apparatus such that hands of the user are able to hang down from the wrists supported by said at least one bar to reach the input apparatus for the wrist/forearms support typing posture.

3. The hand support device, as recited in claim 2, further comprising a pillar and bar joint assembly which a first pillar and bar joint movably inserting a first end of said at least one bar into said first base unit, and a second pillar and bar joint movably inserting a second end of said at least one bar into said second base unit in a movable manner, wherein said first and second ends of said at least one bar are arranged at said first base unit and second base unit respectively so as to extend along a length of the input apparatus and perpendicular to the two sides, left and right, of the input apparatus.

4. The hand support device, as recited in claim 3, wherein said first pillar and bar joint is arranged to lock releasably and independently the first end of said at least one bar at a desire position, and said second pillar and bar joint is arranged to lock releasably and independently said second end of said at least one bar at a desire position.

5. The hand support device, as recited in claim 4, wherein two or more elongated bars are arranged between said first base unit and said second base unit by means of said pillar and bar joint assembly, where at least one of said bars with said pillar produces a 90 degrees angle respectively.

6. The hand support device, as recited in claim 4, wherein said first pillar and bar joint and said second pillar and bar joint are ball-and-socket joints connecting movably said at least one bar to said support base assembly, wherein said first base unit has a first guiding slot coupled with said first pillar and bar joint in a movable manner, and said second pillar and bar joint has a second guiding slot coupled with said second pillar and bar joint in a movable manner for ensuring said first and second ends of said at least one bar to move freely along said first and second base units respectively.

7. The hand support device, as recited in claim 5, wherein said first guiding slot provides a first gear rail arranged at a bottom thereof and said first pillar and bar joint has a first periphery matched to said first gear rail in said first guiding slot, and said second guiding slot has a first gear rail arranged at a bottom thereof and said second pillar and bar joint has a second periphery matched to said second gear rail in said second guiding slot.

8. The hand support device, as recited in claim 6, wherein said first guiding slot provides a first gear rail arranged at a bottom thereof and said first pillar and bar joint has a first periphery matched to said first gear rail in said first guiding slot, and said second guiding slot has a first gear rail arranged at a bottom thereof and said second pillar and bar joint has a second periphery matched to said second gear rail in said second guiding slot.

9. The hand support device, as recited in claim 2, wherein said first base unit and said second base unit are arranged to be adjustable in height.

10. The hand support device, as recited in claim 2, wherein said first base unit and said second base unit are arranged to be collapsible.

11. The hand support device, as recited in claim 2, further comprising a plurality of sleeves and braces provided on said at least one bar in a movable manner, where said sleeve and said braces slide over a surface area of said transverse bridge and diameters of said sleeves and braces are larger than said at least one bar, thereby said sleeves and said braces provide additional comfort, therapeutic, and health benefits to the user.

12. The hand support device, as recited in claim 11, wherein said sleeves are has a length ranging from 1 inch to 8 inches and said braces vary in sizes and shapes, wherein at least a section each of said sleeves and said brace is made material selected from the group consisting of fiber organic composites, hypoallergenic material, glass, gels, wood, plastic, ceramic composites, rubber, carbon, metallic alloys, medicinal chemicals, and combinations thereof.

13. The hand support device, as recited in claim 2, further comprising a first swing pillar and a second swing pillar mounted rotatably at said first base unit and said second base unit respectively, wherein said at least one bar is supported between said first swing pillar and said second swing pillar.

14. The hand support device, as recited in claim 13, further comprising a pillar and bar joint assembly comprising a first pillar and bar joint movably inserting one end of said bar into said first swing pillar, and a second pillar and bar joint movably inserting said other end of said bar into said second swing pillar in a movable manner.

15. The hand support device, as recited in claim 2, further comprising a mid-bar divider comprises a first mid-bar divider and a second mid-bar divider, wherein said first mid-bar divider and said second mid-bar divider are arranged to slide with each other, wherein said at least one bar has a first half bar and a second half bar, wherein said first half bar extends between said first base unit and said first mid-bar divider, while said second half bar extends between said second base unit and said second mid-bar divider.

16. The hand support device, as recited in claim 15, wherein said mid-bar divider and said second mid-bar divider have opposite frictionless surfaces parallel to each other, while both said right and left mid bar divisions provide adjustable opposing force on each other for support and connection.

17. The hand support device, as recited in claim 11, further comprising a first base and a second base, wherein said first base unit sits movably on said first base and said second base unit sits movably on said second base.

18. The hand support device, as recited in claim 17, further comprising a first incline slope control arranged at said first base, and a second incline slope control arranged at said second base, wherein said first base unit sits at said first incline slope control and said second first base unit sits at said second incline slope control for additional direction guide.

19. The hand support device, as recited in claim 17, wherein said first base comprises a first lift to incline and decline said first base and said second base comprises a second lift to incline and decline said first base and said second base.

20. The hand support device, as recited in claim 18, wherein said first lift and said second lift provide at less a 0 to 40 degrees downgrading or upgrade slope.
